(19) United States
(12) Patent Application Publication
(10) Pub. No.: US 2010/0075965 A1
(43) Pub. Date: Mar. 25, 2010

(54) PI3 KINASE INHIBITORS AND METHODS OF THEIR USE

(75) Inventors: Zhi-Jie Ni, Emeryville, CA (US); Sabina Pecchi, Emeryville, CA (US); Matthew Burger, Emeryville, CA (US); Wooseok Han, Emeryvile, CA (US); Aaron Smith, Emeryville, CA (US); Gordana Atallah, Emeryville, CA (US); Sarah Bartulis, Emeryville, CA (US); Kelly Frazier, Emeryville, CA (US); Joelle Verhagen, Emeryville, CA (US); Yanchen Zhang, Emeryville, CA (US); Ed Iwanowicz, Emeryville, CA (US); Tom Hendrickson, Emeryville, CA (US); Mark Knapp, Emeryville, CA (US); Hanne Merritt, Emeryville, CA (US); Charles Voliva, Emeryville, CA (US); Marion Wiesmann, Emeryville, CA (US); Darren Mark Legrand, West Sussex (GB); Ian Bruce, West Sussex (GB); James Dale, West Sussex (GB); Jiong Lan, Emeryville, CA (US); Barry Levine, Emeryville, CA (US); Abran Costales, Emeryville, CA (US); Jui Liu, Emeryville, CA (US); Teresa Pick, Emeryville, CA (US); Daniel Menezes, Emeryville, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: Novartis AG

(21) Appl. No.: 12/279,148

(22) PCT Filed: Feb. 14, 2007
(86) PCT No.: PCT/US07/62157

§ 371 Date: Apr. 1, 2009

Related U.S. Application Data
(60) Provisional application No. 60/773,476, filed on Feb. 14, 2006.
(60) Provisional application No. 60/876,729, filed on Dec. 22, 2006.

(51) Int. Cl. A61K 31/5377; C07D 471/04; C07D 487/04; C07D 413/14; A61K 31/437; A61K 31/5025; A61P 35/00

(52) U.S. Cl. 514/235.8; 546/121; 544/236; 544/121; 514/300; 514/248

(57) ABSTRACT

Phosphatidylinositol (PI) 3 kinase inhibitor compounds, their pharmaceutically acceptable salts, and prodrugs thereof; compositions of the new compounds, either alone or in combination with at least one additional therapeutic agent, with a pharmaceutically acceptable carrier; and uses of the new compounds, either alone or in combination with at least one additional therapeutic agent, in the prophylaxis or treatment of proliferative diseases characterized by the abnormal activity of growth factors, protein serine/threonine kinases, phospholipid kinases, G-protein coupled receptors, and phosphatases.

### BACKGROUND OF THE INVENTION

1. Field of the Invention

This invention relates to new phosphatidylinositol (PI) 3-kinase inhibitor compounds, their pharmaceutically acceptable salts, and prodrugs thereof. This invention also relates to compositions of these compounds, either alone or in combination with at least one additional therapeutic agent, and optionally in combination with a pharmaceutically acceptable carrier. This invention still further relates to methods of use of these compounds, either alone or in combination with at least one additional therapeutic agent, in the prophylaxis or treatment of a number of diseases, in particular, those mediated by one or more of abnormal activity of growth factors, receptor tyrosine kinases, protein serine/threonine kinases, G protein coupled receptors and phospholipid kinases and phosphatases.

2. Background

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃) that, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

In many cases, PIP2 and PIP3 recruit Akt, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified and increased protein expression of their gene products has been demonstrated in several human cancers. Furthermore, mutations and translocation of p85α that serve to up-regulate the p85-p110 complex have been described in a few human cancers. Finally, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase and the upstream and downstream components of this signaling pathway is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Ks would be of particular value in the treatment of proliferative disease and other disorders.

### SUMMARY OF THE INVENTION

The preferred embodiments provide new phosphatidylinositol 3-kinase (PI3K) inhibitor compounds, pharmaceutical formulations that include the compounds, methods of inhibiting phosphatidylinositol 3-kinase (PI3K), and methods of treating proliferative diseases.

Thus, there is provided a compound of Formula (A)

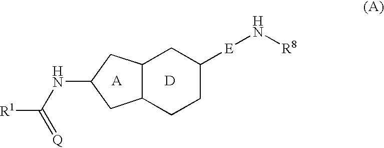

wherein:

ring AD is 5,6-bicyclic heteroaryl ring, where A is a 5-membered aromatic heterocyclic ring containing one or more O, S and N ring atoms and is fused to ring D, which is a 6-membered heteroaryl ring containing one, two or three nitrogen ring atoms, where ring D is substituted by R², R³, R⁴ and R⁵;

E is a pyridyl, pyrimidyl or pyrazinyl group substituted by R⁶, R⁷ and R⁹

Q is O or S;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino, or a pharmaceutically acceptable salt or solvate thereof, including stereoisomers and tautomers thereof.

Specifically, in one preferred embodiment, this invention is directed to compounds or stereoisomers, tautomers, or solvates thereof or pharmaceutically acceptable salts thereof of Formula I and the related compositions and methods wherein Formula I is:

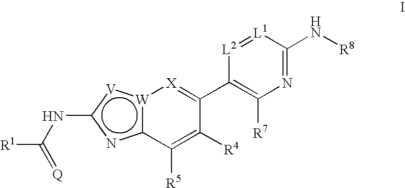

wherein:

Q is O or S;

X is CR³ or N;

W is C or N;

V is CR², O or S;

L¹ is CR⁹ or N;

L² is CR⁶ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

In another preferred embodiment, this invention is directed to compounds or stereoisomers, tautomers, or pharmaceutically acceptable salts thereof of Formula Ia and the related compositions and methods wherein Formula Ia is:

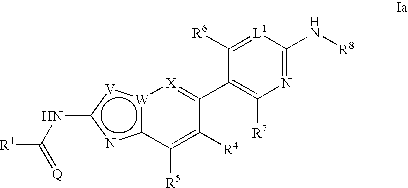

wherein:

Q is O or S;

X is CR³ or N;

W is C or N;

V is CR², O or S;

L¹ is CR⁹ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

In other embodiments provided are compounds or stereoisomers, tautomers, or solvates thereof or pharmaceutically acceptable salts thereof of Formula II and the related compositions and methods wherein Formula II is:

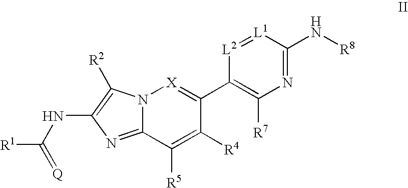

wherein:

Q is O or S;

X is CR³ or N;

L¹ is CR⁹ or N;

L² is CR⁶ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

In other embodiments provided are compounds or stereoisomers, tautomers, or pharmaceutically acceptable salts thereof of Formula IIa and the related compositions and methods wherein Formula IIa is:

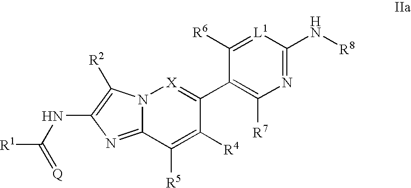

wherein:

Q is O or S;

X is CR³ or N;

L¹ is CR⁹ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, hydroxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

The preferred embodiments are directed to compounds or stereoisomers, tautomers, or solvates thereof or pharmaceutically acceptable salts thereof of Formula III and the related compositions and methods wherein Formula III is:

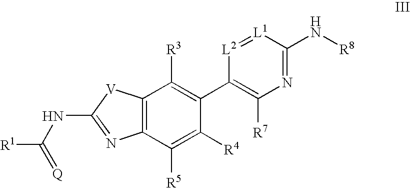

wherein:

Q is O or S;

V is O or S;

L¹ is CR⁹ or N;

L² is CR⁶ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino; R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

Other preferred embodiments are directed to compounds or stereoisomers, tautomers, or pharmaceutically acceptable salts thereof of Formula IIIa and the related compositions and methods wherein Formula IIIa is:

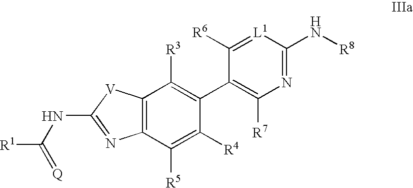

wherein:

Q is O or S;

V is O or S;

L¹ is CR⁹ or N;

R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino; R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

In a more preferred embodiment of a compound of Formula (A), the present invention provides compounds or stereoisomers, tautomers, or solvates thereof or pharmaceutically acceptable salts thereof of Formula (IV),

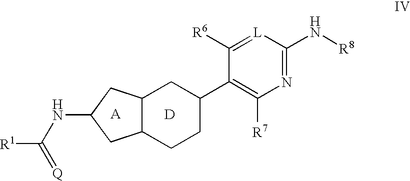

wherein,

ring AD is selected from

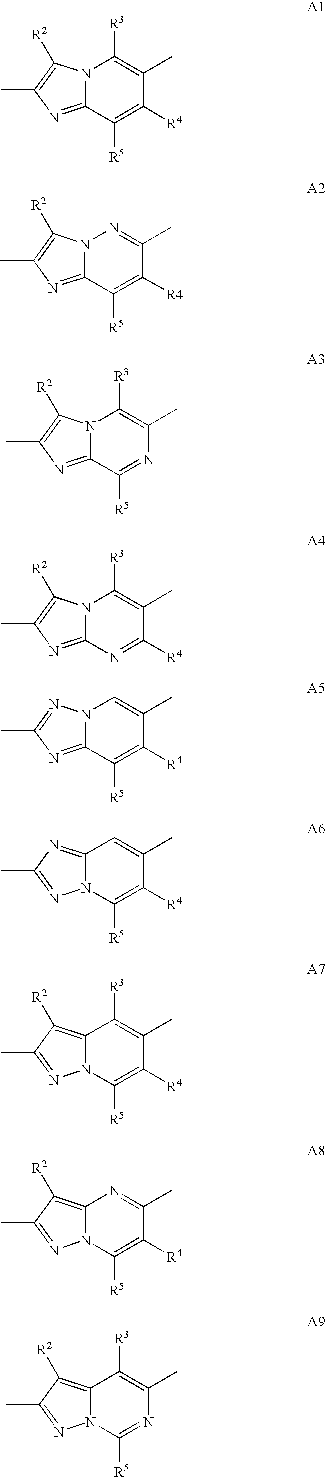

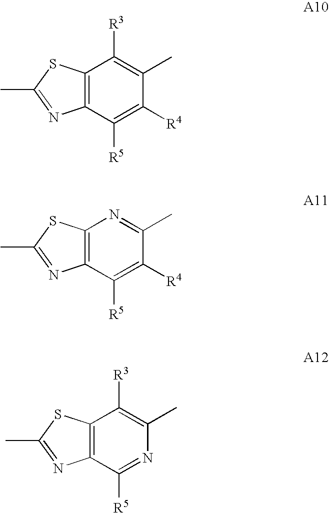

Q is O or S;

L is CR⁹ or N;

R¹ represents —Z—Y—R¹⁰;

Z is —NHCH₂C(R¹¹)R¹²—;

Y is a bond or —CON(R¹³)—;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

R¹⁰ is C₁-C₆-alkylaminocarbonyl, C₁-C₆-alkoxycarbonyl, where each alkyl is independently optionally substituted by one or more halo, hydroxyl or C₁-C₆-alkoxy groups groups, or R¹⁰ is a mono-cyclic heteroaromatic ring having one or more ring heteroatoms selected from the group consisting of oxygen, nitrogen and sulphur, said ring being optionally substituted by one or more halo, hydroxyl, C₁-C₆-alkyl or C₁-C₆-alkoxy groups, where said alkyl and alkoxy are optionally further substituted by one or more halo, hydroxyl or C₁-C₆-alkoxy groups;

R¹¹ and R¹² are independently selected from hydrogen, halo, hydroxy and C₁-C₆-alkyl where said alkyl group is optionally substituted by one or more halo, hydroxyl or C₁-C₆-alkoxy groups; and

R¹³ is hydrogen or C₁-C₆-alkyl.

A further preferred embodiment of the present invention provides compounds or stereoisomers, tautomers, or solvates thereof or pharmaceutically acceptable salts thereof of Formula V:

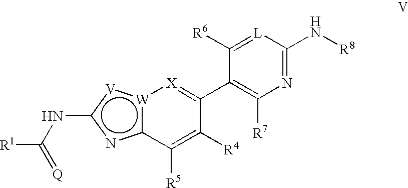

wherein:

Q is O or S;

X is CR³ or N;

W is C or N;

V is CR², O, N, or S;

L is CR⁹ or N;

R¹ represents —Z—Y—R¹⁰;

Z is —NHCH₂C(R¹¹)R¹²—;

Y is a bond or —CON(R¹³)—;

R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;

R⁴, R⁵, and R⁶ are independently selected from the group consisting of hydrogen, halogen, cyano, nitro, amino, substituted amino, alkoxy, substituted alkoxy, alkyl, and substituted alkyl;

R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and

R^{8a} is selected from the group consisting of alkyl, substituted alkyl, alkoxy, substituted alkoxy, amino, substituted amino, and alkylamino.

R¹⁰ is C₁-C₆-alkylaminocarbonyl, C₁-C₆-alkoxycarbonyl, where each alkyl is independently optionally substituted by one or more halo, hydroxyl or C₁-C₆-alkoxy groups groups, or R¹⁰ is a mono-cyclic heteroaromatic ring having one or more ring heteroatoms selected from the group consisting of oxygen, nitrogen and sulphur, said ring being optionally substituted by one or more halo, hydroxyl, C₁-C₆-alkyl or C₁-C₆-alkoxy groups, where said alkyl and alkoxy are optionally further substituted by one or more halo, hydroxyl or C₁-C₆-alkoxy groups;

R¹¹ and R¹² are independently selected from hydrogen, halo, hydroxy and C₁-C₆-alkyl where said alkyl group is optionally substituted by one or more halo, hydroxyl or C₁-C₆-alkoxy groups; and

R¹³ is hydrogen or C₁-C₆-alkyl.

### BRIEF DISCRIPTION OF THE FIGURE

FIG. 1 shows antitumor activity of Compound 57 against subcutaneous A2780 ovarian xenograft tumors. Female nude mice (6-8 weeks of age; Charles River) were implanted subcutaneously with A2780 (5×106 cells/mouse in 0.1 ml HBSS) cells into the right flank. Mice were randomized when tumors reached ˜200 mm³ (n=10/group) and were treated with either vehicle (100% PEG400) or Compound 57 formulated in the vehicle, daily p.o. at the indicated doses (mg/kg) on days 1-6. Tumor volumes were measured (SE is the standard error of the mean).

### DETAILED DESCRIPTION

Phosphatidylinositol-3-kinase (PI3K) mediates the signal from various growth factors to regulate cell proliferation and survival. A Serine/Threonine (Ser/Thr, or S/T) protein kinase, termed Akt, is identified as a downstream target of PI 3-kinase. This protein kinase is recruited to the cell membrane by interaction of its pleckstrin homology domain with PI3K products, phosphatidylinositol-3,4,5-triphosphate (PIP₃), and phosphatidylinositol-3,4-diphosphate (PIP₂), where it is activated by phosphorylation of its catalytic domain by 3-Phosphoinositide-dependent Kinase-1 (PDK-1). Akt is further activated by phosphorylation of a serine in its C-terminal hydrophobic motif by another kinase (PDK-2). The activation of Akt acts downstream to regulate additional kinases many of which are implicated in cellular processes that control survival, proliferation, metabolism and growth translation. PI3K can also drive cellular processes that impact transformation, cellular proliferation, cytoskeletal rearrangement and survival through a parallel pathway that does not involve Akt (Hennessy et al., Nat. Rev. Drug Disc. 4:988-1004 (2005)). Two of these pathways are activation of the small GTP-binding proteins Cdc42 and Rac1 and activation of the serum and glucocorticoid-inducible kinase (SGK). Cdc42 and Rac1, which regulate cytoskeletal movement and cell motility and can function as oncogenes when over-expressed, are also linked to the RAS pathway. Thus, PI3K activity generates 3′-phosphatidylinositol lipids that act as a nodal point to stimulate a diversity of downstream signaling pathways.

That these pathways impact cellular properties proliferation, survival, motility and morphology that are often disrupted in cancer, proliferative diseases, thrombotic diseases and inflammation, among others, dictates that compounds inhibiting PI3K (and isoforms thereof) have utility, either as a single agent or in combination, in the treatment of these diseases. In cancer, deregulation of the PI3K/Akt pathway is extensively documented, including overexpression of the PIK3CA gene, activating mutations of the PIK3CA gene, overexpression of Akt, mutations of PDK-1, and deletions/inactivation of PTEN (Parsons et al., Nature 436:792 (2005); Hennessy et al., Nat. Rev. Drug Disc. 4:988 (2005); Stephens et al., Curr. Opin. Pharmacol. 5:1 (2005); Bonneau and Longy, Human Mutation 16:109 (2000) and Ali et al., J. Natl. Can. Inst. 91:1922 (1999)). Recent findings indicate that PIK3CA is frequently mutated (>30%) in various solid tumors in humans (Samuels and Ericson, Curr. Opin. Oncology 18:77 (2005)) and the most frequent of these mutations promote cell growth and invasion (Samuels et al., Cancer Cell 7:561 (2005), and are transforming (Kang et al., Proc. Natl. Acad. Sci. USA 102:802 (2005), Zhao et al., Proc. Natl. Acad. Sci. USA 102:18443 (2005)). Thus, inhibitors of PI3K, particularly of the p110α isoform encoded by PIK3CA and its mutations, will be useful in the treatment of cancers driven by these mutations and deregulations.

In its compounds aspects, the embodiments provide novel compounds that act as inhibitors of serine/threonine kinases, lipid kinases, and, more particularly, as inhibitors of phosphatidylinositol 3-kinase (PI3K) function. The compounds provided herein can be formulated into pharmaceutical formulations that are useful in treating patients with a need for an inhibitor of PI3K, especially, in particular embodiments, to provide compositions and methods for reducing cellular proliferation and increasing cell death in the treatment of cancer.

Throughout this application, the text refers to various embodiments of the present compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### Definitions

The terms used in the claims are defined below.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)ₛ—bstituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined, together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkyenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkyenyl, aryl, substituted aryl, heteroaryl, substituted heteroaryl, heterocyclic and substituted heterocyclic are as defined herein.

“Amidino” refers to the group —C(═NR²⁵)R²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkenyl” refers to alkenyl groups having from 2 to 6 carbon atoms and preferably 2 to 4 carbon atoms and having at least 1 and preferably from 1 to 2 sites of alkenyl unsaturation. Such groups are exemplified, for example, by vinyl, allyl, and but-3-en-1-yl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Alkynyl” refers to alkynyl groups having from 2 to 6 carbon atoms and preferably 2 to 3 carbon atoms and having at least 1 and preferably from 1 to 2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Cyanate” refers to the group —OCN.

“Thiocyanate” refers to the group —SCN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)amino” refers to the group —NR²⁰—C(O)O-alkyl, substituted —NR²⁰—C(O)O-alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-cycloalkenyl, —NR²⁰—C(O)O-substituted cycloalkenyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Cyano” and “carbonitrile” refers to the group —CN.

“Cycloalkyl” refers to cyclic alkyl groups of from 3 to 10 carbon atoms having single or multiple cyclic rings including fused, bridged, and spiroring systems. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” and “heteroaromatic” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or unsaturated group having a single ring or multiple condensed rings, including fused bridged and spiro ring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Imino” refers to the group —CH═NR^{a} wherein R^{a} is hydrogen, alkyl, substituted alkyl, hydroxy, alkoxy, substituted alkoxy, amino, or substituted amino.

“Nitro” refers to the group —NO₂.

“Oxo” refers to the atom (═O).

“Spirocycloalkyl” refers to divalent cyclic groups from 3 to 10 carbon atoms having a cycloalkyl ring with a spiro union (the union formed by a single atom which is the only common member of the rings) as exemplified by the following structure:

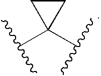

“Spirocyclyl” refers to divalent cyclic groups having a cycloalkyl or heterocyclyl ring with a spiro union, as described for spirocycloalkyl.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Solvate” or “solvates” refer compounds or a salt thereof that are bound to a stoichiometric or non-stoichiometric amount of a solvent. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Prodrug” refers to any derivative of a compound of this invention that is capable of directly or indirectly providing a compound of this invention or an active metabolite or residue thereof when administered to a subject. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. An general overview of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

An embodiment of the present invention provides a pharmaceutical composition comprising a pharmaceutically acceptable carrier and a therapeutically effective amount of a compound of Formula A, a stereoisomer, tautomer, or pharmaceutically acceptable salt thereof, where ring AD is suitably selected from

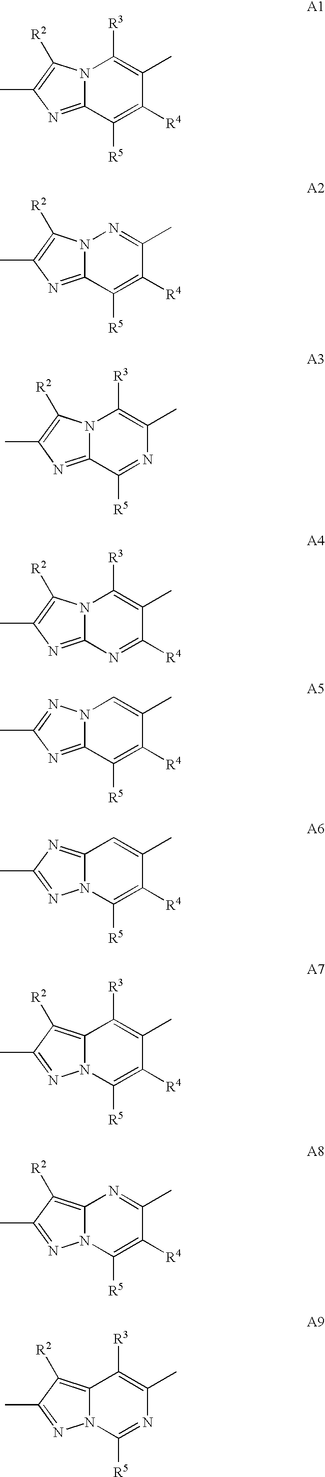

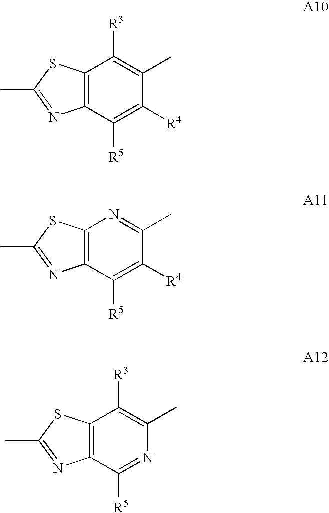

In another suitable embodiment of a compound of Formula A, E is suitably selected from the group

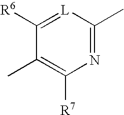

where L is N or CR⁹.

In another embodiment and in combination with any of the embodiments disclosed, provided is a compound having one or more of (a)-(g):
(a) R⁸ is hydrogen;
(b) L² is N or CR⁶ where R⁶ is H;
(c) R⁷ is hydrogen, alkyl, or amino;
(d) X is N or CR³ where R³ is hydrogen, alkyl, hydroxy, or alkoxy;
(e) R⁴ is hydrogen, halo, or alkyl;
(f) R⁵ is hydrogen, halo, or alkyl; and
(g) Q is O.

In one embodiment, compounds of Formula I, Ia, II, and IIa have one or more of (a)-(g).

In another embodiment, compounds of Formula I, Ia, II, and IIa are provided having (a)-(g).

An embodiment provides for compounds of Formula II wherein R¹ is methyl or trifluoromethyl.

An embodiment provides for compounds of Formula II, wherein R¹ is methyl.

An embodiment provides for compounds of Formula II, wherein R² is selected from the group consisting of hydrogen, chloro, bromo, methylamido-N-phenyl, fluorophenyl, phenyl, phenylalkynyl, aminomethylalkynyl, and amidophenyl.

An embodiment provides for compounds of Formula II, wherein R² is bromo or amidophenyl.

An embodiment provides for compounds of Formula II, wherein X is CR³, more particularly, R³ is hydrogen.

An embodiment provides for compounds of Formula II, wherein R⁴ is hydrogen.

An embodiment provides for compounds of Formula II, wherein R⁵ is hydrogen.

An embodiment provides for compounds of Formula II, wherein R⁴ and R⁵ are both hydrogen

An embodiment provides for compounds of Formula II, wherein R⁶ is hydrogen.

An embodiment provides for compounds of Formula II, wherein R⁷ is hydrogen.

An embodiment provides for compounds of Formula II, wherein R⁸ is hydrogen or acetyl.

An embodiment provides for compounds of Formula II, wherein R⁸ is hydrogen.

An embodiment provides for compounds of Formula II, wherein R⁹ is selected from the group consisting of hydrogen, trifluoromethyl, methoxy, fluoro, methyl, and bromo.

An embodiment provides for compounds of Formula II, wherein R⁹ is selected from the group consisting of hydrogen, trifluoromethyl, and methoxy.

An embodiment provides for compound, stereoisomer, tautomer, or a pharmaceutically acceptable salt thereof selected from Table 1 or 3.

Turning to Formula III and IIIa, provided are preferred R¹, R³, R⁴, R⁵, R⁶, R⁷, R⁸ and R⁹ groups.

An embodiment provides for compounds of Formula IIIa, wherein R¹ is selected from the group consisting of methyl, methoxy, morpholinyl-N-propyl, piperidyl-N-methyl, morpholinyl-N-methyl, piperidyl-N-ethoxy, piperidyl-N-propyl, methylamino, and morpholinyl-N-ethoxy.

An embodiment provides for compounds of Formula IIIa, wherein R¹ is selected from the group consisting of methyl, morpholinyl-N-propyl, piperidyl-N-propyl, and methylamino.

An embodiment provides for compounds of Formula IIIa, wherein R³ is hydrogen.

An embodiment provides for compounds of Formula IIIa, wherein R⁴ is hydrogen.

An embodiment provides for compounds of Formula IIIa, wherein R⁵ is hydrogen.

An embodiment provides for compounds of Formula IIIa, wherein R⁶ is selected from the group consisting of hydrogen, trifluoromethyl, and methyl.

An embodiment provides for compounds of Formula IIIa, wherein R⁶ is hydrogen.

An embodiment provides for compounds of Formula IIIa, wherein R⁷ is hydrogen.

An embodiment provides for compounds of Formula IIIa, wherein R⁸ is hydrogen, propyl, tetrahydropyranyl, piperidyl, and acetyl.

An embodiment provides for compounds of Formula IIIa, wherein R⁸ is hydrogen.

An embodiment provides for compounds of Formula IIIa, wherein R⁹ is selected from the group consisting of hydrogen, methyl, fluoro, trifluoromethyl, methoxy, cyano, and dimethylaminomethyl.

An embodiment provides for compound, stereoisomer, tautomer, or a pharmaceutically acceptable salt thereof selected from Table 2.

In another embodiment of a compound of Formula (IV) or Formula (V), ring AD is suitably ring A1

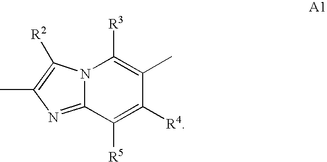

In another embodiment of a compound of Formula A or Formula (IV) or Formula (V), Q is suitably O.

In another embodiment of a compound of Formula (IV) or Formula (V), X is suitably CH or N.

In another embodiment of a compound of Formula (IV) or Formula (V), W is suitably N.

In another embodiment of a compound of Formula (IV) or Formula (V), V is suitably CH.

In another embodiment of a compound of Formula (IV) or Formula (V), L is suitably CR⁹, where R⁹ is suitably hydrogen, halo, hydroxyl, C₁-C₆-alkyl, C₁-C₆-alkoxy, cyano, nitro, amino, C₁-C₆-alkylamino, di-C₁-C₆-alkylamino, aminocarbonyl, C₁-C₆-alkylaminocarbonyl, di-C₁-C₆-alkylaminocarbonyl, oxocarbonyl, C₁-C₆-alkylcarbonylamino, C₁-C₆-alkylcarbonyl(C₁-C₆-alkyl)amino, hydroxycarbonyl, C₁-C₆-alkoxycarbonyl, C₁-C₆-alkylsulfonyl, aminosulfonyl, C₁-C₆-alkylaminosulfonyl, di-C₁-C₆-alkylaminosulfonyl, sulfonylamino, C₁-C₆-alkylsulfonylamino, C₁-C₆-alkylsulfonyl(C₁-C₆-alkyl)amino, where said alkyl and alkoxy are optionally further substituted by one or more halo, hydroxyl or C₁-C₆-alkoxy. (or heterocycle, e.g. imidazole)

In another embodiment of a compound of Formula (IV) or Formula (V), R⁹ is more suitably C₁-C₆-alkyl, optionally substituted by halo, e.g. fluoro, e.g. trifluoromethyl, or R⁹ is cyano.

In another embodiment of a compound of Formula (IV) or Formula (V), Z is suitably —NH—CH₂—CH₂—, i.e. ethyleneamino.

In another embodiment of a compound of Formula (IV) or Formula (V), where Y is —CON(R¹³)—, R¹³ is suitably hydrogen.

In another embodiment of a compound of Formula (IV) or Formula (V), where R¹ represents —Z—Y—R¹⁰, Y represents a bond and R¹⁰ is a mono-cyclic heteroaromatic ring, the ring is suitably an optionally substituted tetrazolyl, imidazolyl, oxazolyl, oxadiazolyl or isoxazolyl group, where the optional substituent is suitably C₁-C₆-alkyl, e.g. methyl, ethyl or isopropyl, optionally substituted by halo, e.g. fluoro, e.g. 2-fluoroethyl.

In another embodiment of a compound of Formula (IV) or Formula (V), where R¹ represents —Z—Y—R¹⁰, Y represents CON(R¹³) and R¹⁰ is a mono-cyclic heteroaromatic ring, the ring is suitably an optionally substituted isoxazolyl group, where the optional substituent is suitably C₁-C₆-alkyl, e.g. methyl, ethyl or isopropyl.

In another embodiment of a compound of Formula (IV) or Formula (V), where R¹ represents —Z—Y—R¹⁰, Y is a bond, R¹⁰ also suitably represents C₁-C₆-alkylaminocarbonyl, e.g. t-butylaminocarbonyl, C₁-C₆-alkoxycarbonyl, e.g. t-butoxycarbonyl, where each alkyl is independently optionally substituted by one or more halo, hydroxyl or C₁-C₆-alkoxy groups groups,

In another embodiment of a compound of Formula (IV) or Formula (V), R¹ is preferably 2-(2-ethyl-2H-tetrazol-5-yl)-ethylamino, 2-(2-isopropyl-2H-tetrazol-5-yl)-ethylamino, 2-(5-ethyl-tetrazol-2-yl)-ethylamio, 2-[2-(2-fluoro-ethyl)-2H-tetrazol-5-yl]-ethylamino, 2-(1-ethyl-1H-imidazol-4-yl)-ethylamino,

In another embodiment of a compound of Formula (IV) or Formula (V), R⁴, R⁵, R⁶, R⁷ and R⁸ are suitably hydrogen.

In other embodiments, there is provided a compound of Fomula V selected from the group consisting of Formula Va:

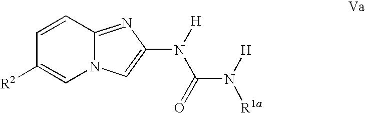

where R¹ is NHR^{1a} and R² are shown in the table below, the method of preparation being described hereinafter. The Examples are in their free base form.

| Ex. | R² | R^{1a} |
|---|---|---|
| 1 | 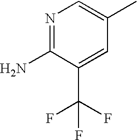 | 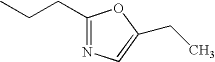 |
| 2 | 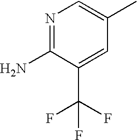 | 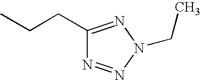 |
| 3 | 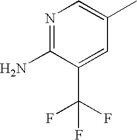 | 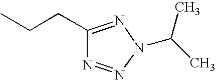 |
| 4 | 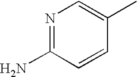 | 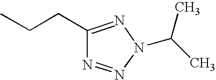 |
| 5 | 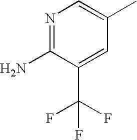 | 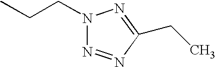 |
| 6 | 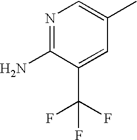 | 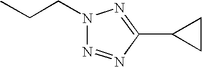 |

Another embodiment provides a pharmaceutical composition comprising a pharmaceutically acceptable carrier and a therapeutically effective amount of a compound of Formula A, I, Ia, II, IIa, III, IIIa, IV, V, or Va, a stereoisomer, tautomer, or pharmaceutically acceptable salt thereof.

Another embodiment provides a pharmaceutical composition comprising a pharmaceutically acceptable carrier and a therapeutically effective amount of a compound, stereoisomer, tautomer, or solvate or pharmaceutically acceptable salt thereof selected from Table 1 or 3.

Another embodiment provides a pharmaceutical composition comprising a pharmaceutically acceptable carrier and a therapeutically effective amount of a compound, stereoisomer, tautomer, or pharmaceutically acceptable salt thereof selected from Table 2.

### Indications

In other aspects, the preferred embodiments provide for methods for manufacture of PI3K inhibitor compounds. It is further contemplated that, in addition to the compounds of Formulas A, I, Ia, II, IIa, III, IIIa, IV, V, and Va, intermediates, and their corresponding methods of syntheses are included within the scope of the embodiments.

Another embodiment provides a method of inhibiting phosphorylation of Akt comprising administering a compound of Formula A, I, Ia, II, IIa, III, IIIa, IV, V, or Va to a human in need thereof. Another embodiment provides a method of treating cancer responsive to inhibition of phosphorylation of Akt, comprising administering such a compound. Another embodiment provides a method of inhibiting phosphorylation of Akt comprising contacting a cell with such a compound.

Another embodiment provides for a method for inhibiting phosphorylation of a substrate selected from phosphatidylinositol (PI), phosphatidylinositol phosphate (PIP), or phosphatidylinositol diphosphate (PIP₂), comprising exposing said substrate and a kinase thereof to a compound of Formula A, I, Ia, II, IIa, III, IIIa, IV, V, or Va.

Another embodiment provides a method of inhibiting phosphorylation of Akt comprising orally administering a compound of Formula A, I, Ia, II, IIa, III, IIIa, IV, V, or Va to a human in need thereof. In a more particular embodiment the human is suffering from cancer. In a more particular embodiment the cancer is responsive to treatment with a compound that inhibits phosphorylation of Akt. In another embodiment the compound is orally bioavailable.

Another embodiment provides a method of treating cancer comprising orally administering a compound of Formula A, I, Ia, II, IIa, III, IIIa, IV, V, or Va wherein said compound is capable of inhibiting activity of pAkt.

In some embodiments of the method of inhibiting PI3K using a PI3K inhibitor compound of the embodiments, the IC₅₀ value of the compound is less than or equal to about 1 mM with respect to PI3K. In other such embodiments, the IC₅₀ value is less than or equal to about 100 μM, is less than or equal to about 25 μM, is less than or equal to about 10 μM, is less than or equal to about 1 μM, is less than or equal to about 0.1 μM, is less than or equal to about 0.050 μM, or is less than or equal to about 0.010 μM.

Some embodiments provide methods of inhibiting phosphorylation of Akt using a compound of the embodiments having an EC₅₀ value of less than about 10 μM with respect to inhibition of pAKT. In another more particular embodiment, the compound has an EC₅₀ value of less than about 1 μM with respect to inhibition of pAKT. In a more particular embodiment still, the compound has an EC₅₀ value of less than about 0.5 μM with respect to inhibition of pAKT. In an even more particular embodiment, the compound has an EC₅₀ value of less than about 0.1 μM with respect to inhibition of pAKT.

In certain embodiments, a compound is capable of inhibition of phosphorylation of Akt. In certain embodiments, a compound is capable of inhibition of phosphorylation of Akt in a human or animal subject (i.e., in vivo).

In one embodiment, a method of reducing pAkt activity in a human or animal subject is provided. In the method, a compound of the preferred embodiments is administered in an amount effective to reduce pAkt activity.

In some embodiments of the method of inhibiting PI3K using a PI3K inhibitor compound of the embodiments, the IC₅₀ value of the compound is between about 1 nM to about 10 nM. In other such embodiments, the IC₅₀ value is between about 10 nM to about 50 nM, between about 50 nM to about 100 nM, between about 100 nM to about 1 μM, between about 1 μM to about 10 μM, or is between about 10 μM to 25 μM, or is between about 25 μM to about 100 μM.

Another embodiment provides methods of treating a PI3K-mediated disorder. In one method, an effective amount of a PI3K inhibitor compound is administered to a patient (e.g., a human or animal subject) in need thereof to mediate (or modulate) PI3K activity.

The compounds of the preferred embodiment are useful in pharmaceutical compositions for human or veterinary use where inhibition of PI3K is indicated, for example, in the treatment of cellular proliferative diseases such as tumor and/or cancerous cell growth mediated by PI3K. In particular, the compounds are useful in the treatment of human or animal (e.g., murine) cancers, including, for example, lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

Agents of the invention, particularly those which have selectivity for PI3 kinase gamma inhibition, are particularly useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the embodiments are applicable include asthma of whatever type of genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics (“wheezy infant syndrome”).

Compounds of the invention that are selective for one PI3 Kinase isoform (α, β, γ, δ) over a different isoform are compounds that preferentially inhibit one isoform. For example, a compound may preferentially inhibit the alpha isoform over the gamma isoform. Alternatively, a compound may preferentially inhibit the gamma isoform over the alpha isoform. To determine a compound's selectivity, the compound's activity is determined according to the Biological Methods described herein. For example, the IC₅₀, EC₅₀, or Ki value of a compound is determined for two or more PI3 Kinase isoforms, e.g, alpha and gamma, according to the procedures described for Biological Methods 1-4. The obtained values are then compared to determine the selectivity of the tested compound. Preferably, the compounds of the invention are selective for one isoform over a second isoform by at least two-fold, five-fold, or ten-fold. Even more preferably, the compounds of the invention are selective for one isoform over a second isoform by at least fifty-fold or 100-fold. Even more preferably, the compounds of the invention are selective for one isoform over a second isoform by at least 1000-fold.

Other inflammatory or obstructive airways diseases and conditions to which the embodiments are applicable include acute lung injury (ALI), adult respiratory distress syndrome (ARDS), chronic obstructive pulmonary, airways or lung disease (COPD, COAD or COLD), including pulmonary fibrosis, chronic bronchitis or dyspnea associated therewith, emphysema, as well as exacerbation of airways hyperreactivity consequent to other drug therapy, in particular other inhaled drug therapy. The embodiments are also applicable to the treatment of bronchitis of whatever type or genesis including, e.g., acute, arachidic, catarrhal, croupus, chronic or phthinoid bronchitis. Further inflammatory or obstructive airways diseases to which the embodiments are applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, abestosis, chalicosis, ptilosis, siderosis, silicosis, tabacosis and byssinosis.

Having regard to their anti-inflammatory activity, in particular in relation to inhibition of eosinophil activation, agents of the preferred embodiments are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-realted disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Agents of the embodiments are also useful in the treatment of inflammatory or allergic conditions of the skin, for example psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Agents of the embodiments may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematogical disorders (e.g. haemolytic anaemia, aplastic anaemia, pure red cell anaemia and idiopathic thrombocytopenia), systemic lupus erythematosus, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), interstitial lung fibrosis, psoriatic arthritis and glomerulonephritis (with and without nephritic syndrome, e.g. including idiopathic nephritic syndrome or minal change nephropathy).

Another embodiment provides a method for inhibiting leucocytes, in particular neutrophils and B and T lymphocytes. Exemplary medical conditions that can be treated include those conditions characterized by an undesirable neutrophil function selected from the group consisting of stimulated superoxide release, stimulated exocytosis, and chemotactic migration, preferably without inhibiting phagocytic activity or bacterial killing by the neutrophils.

Another embodiment provides a method for disrupting the function of osteoclasts and ameliorating a bone resorption disorder, such as osteoporosis.

Another embodiment provides treatment of diseases or conditions with agents of the embodiments, such as, but not limited to septic shock, allograft rejection following transplantation, bone disorders such as but not limited to rheumatoid arthritis, ankylosing spondylitis osteoarthritis, obesity, restenosis, diabetes, e.g. diabetes mellitus type I (juvenile diabetes) and diabetes mellitus type II, diarrheal diseases.

In other embodiments, the PI3K-mediated condition or disorder is selected from the group consisting of: cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

As described above, since PI3K serves as a second messenger node that integrates parallel signaling pathways, evidence is emerging that the combination of a PI3K inhibitor with inhibitors of other pathways will be useful in treating cancer and proliferative diseases in humans.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI3K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)).

A variety of human malignancies express activitating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway. For example, gefitinib inhibits the growth of an adenocarcinoma cell line in in vitro assays. Nonetheless, sub-clones of these cell lines can be selected that are resistant to gefitinib that demonstrate increased activation of the PT3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Akt also abrogates its role to arrest the cell cycle (Viglietto et al., Nat Med. 8:1145 (2002)). Accordingly, in one aspect, the compounds of Formula A, I, Ia, II, IIa, III, IIIa, IV, V, or Va may be used in the treatment of hormone dependent cancers, such as breast and prostate cancers, to reverse hormone resistance commonly seen in these cancers with conventional anticancer agents.

In hematological cancers, such as chronic myelogenous leukemia (CML), chromosomal translocation is responsible for the constitutively activated BCR-Abl tyrosine kinase. The afflicted patients are responsive to imatinib, a small molecule tyrosine kinase inhibitor, as a result of inhibition of Abl kinase activity. However, many patients with advanced stage disease respond to imatinib initially, but then relapse later due to resistance-conferring mutations in the Abl kinase domain. In vitro studies have demonstrated that BCR-Ab1 employs the Ras-Raf kinase pathway to elicit its effects. In addition, inhibiting more than one kinase in the same pathway provides additional protection against resistance-conferring mutations. Accordingly, in another aspect of the embodiments, the compounds of Formula A, I, Ia, II, IIa, III, IIIa, IV, V, or Va are used in combination with at least one additional agent, such as Gleevec®, in the treatment of hematological cancers, such as chronic myelogenous leukemia (CML), to reverse or prevent resistance to at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In addition to cancer and proliferative diseases, there is accumulating evidence that inhibitors of Class 1A and 1B PI3 kinases would be therapeutically useful in others disease areas. The inhibition of p110β, the PI3K isoform product of the PIK3CB gene, has been shown to be involved in shear-induced platelet activation (Jackson et al., Nature Medicine 11:507-514 (2005)). Thus, a PI3K inhibitor that inhibits p110β would be useful as a single agent or in combination in anti-thrombotic therapy. The isoform p110β, the product of the PIK3CD gene, is important in B cell function and differentiation (Clayton et al., J. Exp. Med. 196:753-763 (2002)), T-cell dependent and independent antigen responses (Jou et al., Mol. Cell. Biol. 22:8580-8590 (2002)) and mast cell differentiation (Ali et al., Nature 431:1007-1011 (2004)). Thus, it is expected that p110β-inhibitors would be useful in the treatment of B-cell driven autoimmune diseases and asthma. Finally, the inhibition of p110β, the isoform product of the PI3KCG gene, results in reduced T, but not B cell, response (Reif et al., J. Immunol. 173:2236-2240 (2004)) and its inhibition demonstrates efficacy in animal models of autoimmune diseases (Camps et al., Nature Medicine 11:936-943 (2005), Barber et al., Nature Medicine 11:933-935 (2005)).

The preferred embodiments provide pharmaceutical compositions comprising at least one compound of Formula A, I, Ia, II, IIa, III, IIIa, IV, V, or Va together with a pharmaceutically acceptable carrier suitable for administration to a human or animal subject, either alone or together with other anticancer agents.

Another embodiment provides methods of treating human or animal subjects suffering from a cellular proliferative disease, such as cancer. The preferred embodiments provide methods of treating a human or animal subject in need of such treatment, comprising administering to the subject a therapeutically effective amount of a compound of Formula A, I, Ia, II, IIa, III, IIIa, IV, V, or Va, either alone or in combination with other anticancer agents.

In particular, compositions will either be formulated together as a combination therapeutic or administered separately. Anticancer agents for use with the preferred embodiments include, but are not limited to, one or more of the following set forth below:

### A. Kinase Inhibitors

Kinase inhibitors for use as anticancer agents in conjunction with the compositions of the preferred embodiments include inhibitors of Epidermal Growth Factor Receptor (EGFR) kinases such as small molecule quinazolines, for example gefitinib (U.S. Pat. No. 5,457,105, U.S. Pat. No. 5,616,582, and U.S. Pat. No. 5,770,599), ZD-6474 (WO 01/32651), erlotinib (Tarceva®, U.S. Pat. No. 5,747,498 and WO 96/30347), and lapatinib (U.S. Pat. No. 6,727,256 and WO 02/02552); Vascular Endothelial Growth Factor Receptor (VEGFR) kinase inhibitors, including SU-11248 (WO 01/60814), SU 5416 (U.S. Pat. No. 5,883,113 and WO 99/61422), SU 6668 (U.S. Pat. No. 5,883,113 and WO 99/61422), CHIR-258 (U.S. Pat. No. 6,605,617 and U.S. Pat. No. 6,774,237), vatalanib or PTK-787 (U.S. Pat. No. 6,258,812), VEGF-Trap (WO 02/57423), B43-Genistein (WO-09606116), fenretinide (retinoic acid p-hydroxyphenylamine) (U.S. Pat. No. 4,323,581), IM-862 (WO 02/62826), bevacizumab or Avastin® (WO 94/10202), KRN-951, 3-[5-(methylsulfonylpiperadine methyl)-indolyl]-quinolone, AG-13736 and AG-13925, pyrrolo[2,1-f][1,2,4]triazines, ZK-304709, Veglin®, VMDA-3601, EG-004, CEP-701 (U.S. Pat. No. 5,621,100), Cand5 (WO 04/09769); Erb2 tyrosine kinase inhibitors such as pertuzumab (WO 01/00245), trastuzumab, and rituximab; Akt protein kinase inhibitors, such as RX-0201; Protein Kinase C(PKC) inhibitors, such as LY-317615 (WO 95/17182), and perifosine (US 2003171303); Raf/Map/MEK/Ras kinase inhibitors including sorafenib (BAY 43-9006), ARQ-350RP, LErafAON, BMS-354825, AMG-548, and others disclosed in WO 03/82272; Fibroblast Growth Factor Receptor (FGFR) kinase inhibitors; Cell Dependent Kinase (CDK) inhibitors, including CYC-202 or roscovitine (WO 97/20842 and WO 99/02162); Platelet-Derived Growth Factor Receptor (PDGFR) kinase inhibitors such as CHIR-258, 3G3 mAb, AG-13736, SU-11248 and SU6668; and Bcr-Abl kinase inhibitors and fusion proteins such as STI-571 or Gleevec® (imatinib).

### B. Anti-Estrogens

Estrogen-targeting agents for use in anticancer therapy in conjunction with the compositions of the preferred embodiments include Selective Estrogen Receptor Modulators (SERMs) including tamoxifen, toremifene, raloxifene; aromatase inhibitors including Arimidex® or anastrozole; Estrogen Receptor Downregulators (ERDs) including Faslodex® or fulvestrant.

### C. Anti-Androgens

Androgen-targeting agents for use in anticancer therapy in conjunction with the compositions of the preferred embodiments include flutamide, bicalutamide, finasteride, aminoglutethamide, ketoconazole, and corticosteroids.

### D. Other Inhibitors

Other inhibitors for use as anticancer agents in conjunction with the compositions of the preferred embodiments include protein farnesyl transferase inhibitors including tipifamib or R-115777 (US 2003134846 and WO 97/21701), BMS-214662, AZD-3409, and FTI-277; topoisomerase inhibitors including merbarone and diflomotecan (BN-80915); mitotic kinesin spindle protein (KSP) inhibitors including SB-743921 and MKI-833; proteasome modulators such as bortezomib or Velcade® (U.S. Pat. No. 5,780,454), XL-784; and cyclooxygenase 2 (COX-2) inhibitors including non-steroidal antiinflammatory drugs I (NSAIDs).

### E. Cancer Chemotherapeutic Drugs

Particular cancer chemotherapeutic agents for use as anticancer agents in conjunction with the compositions of the preferred embodiments include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®, US 2004073044), doxorubicin hydrochloride (Adriamycin®), Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

### F. Alkylating Agents

Alkylating agents for use in conjunction with the compositions of the preferred embodiments for anticancer therapeutics include VNP-40101M or cloretizine, oxaliplatin (U.S. Pat. No. 4,169,846, WO 03/24978 and WO 03/04505), glufosfamide, mafosfamide, etopophos (U.S. Pat. No. 5,041,424), prednimustine; treosulfan; busulfan; irofluven (acylfulvene); penclomedine; pyrazoloacridine (PD-115934); 06-benzylguanine; decitabine (5-aza-2-deoxycytidine); brostallicin; mitomycin C (MitoExtra); TLK-286 (Telcyta®); temozolomide; trabectedin (U.S. Pat. No. 5,478,932); AP-5280 (Platinate formulation of Cisplatin); porfiromycin; and clearazide (meclorethamine).

### G. Chelating Agents

Chelating agents for use in conjunction with the compositions of the preferred embodiments for anticancer therapeutics include tetrathiomolybdate (WO 01/60814); RP-697; Chimeric T84.66 (cT84.66); gadofosveset (Vasovist®); deferoxamine; and bleomycin optionally in combination with electorporation (EPT).

### H. Biological Response Modifiers

Biological response modifiers, such as immune modulators, for use in conjunction with the compositions of the preferred embodiments for anticancer therapeutics include staurosprine and macrocyclic analogs thereof, including UCN-01, CEP-701 and midostaurin (see WO 02/30941, WO 97/07081, WO 89/07105, U.S. Pat. No. 5,621,100, WO 93/07153, WO 01/04125, WO 02/30941, WO 93/08809, WO 94/06799, WO 00/27422, WO 96/13506 and WO 88/07045); squalamine (WO 01/79255); DA-9601 (WO 98/04541 and U.S. Pat. No. 6,025,387); alemtuzumab; interferons (e.g. IFN-a, IFN-b etc.); interleukins, specifically IL-2 or aldesleukin as well as IL-1, IL-3, IL-4, IL-5, IL-6, IL-7, IL-8, IL-9, IL-10, IL-11, IL-12, and active biological variants thereof having amino acid sequences greater than 70% of the native human sequence; altretamine (Hexylen®); SU 101 or leflunomide (WO 04/06834 and U.S. Pat. No. 6,331,555); imidazoquinolines such as resiquimod and imiquimod (U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612); and SMIPs, including benzazoles, anthraquinones, thiosemicarbazones, and tryptanthrins (WO 04/87153, WO 04/64759, and WO 04/60308).

### I. Cancer Vaccines:

Anticancer vaccines for use in conjunction with the compositions of the preferred embodiments include Avicine® (Tetrahedron Lett. 26:2269-70 (1974)); oregovomab (OvaRex®); Theratope® (STn-KLH); Melanoma Vaccines; GI-4000 series (GI-4014, GI-4015, and GI-4016), which are directed to five mutations in the Ras protein; GlioVax-1; MelaVax; Advexin® or INGN-201 (WO 95/12660); Sig/E7/LAMP-1, encoding HPV-16 E7; MAGE-3 Vaccine or M3TK (WO 94/05304); HER-2VAX; ACTIVE, which stimulates T-cells specific for tumors; GM-CSF cancer vaccine; and Listeria monocytogenes-based vaccines.

### J. Antisense Therapy:

Anticancer agents for use in conjunction with the compositions of the preferred embodiments also include antisense compositions, such as AEG-35156 (GEM-640); AP-12009 and AP-11014 (TGF-beta2-specific antisense oligonucleotides); AVI-4126; AVI-4557; AVI-4472; oblimersen (Genasense®); JFS2; aprinocarsen (WO 97/29780); GTI-2040 (R2 ribonucleotide reductase mRNA antisense oligo) (WO 98/05769); GTI-2501 (WO 98/05769); liposome-encapsulated c-Raf antisense oligodeoxynucleotides (LErafAON) (WO 98/43095); and Sirna-027 (RNAi-based therapeutic targeting VEGFR-1 mRNA).

The compounds of the preferred embodiments can also be combined in a pharmaceutical composition with bronchiodilatory or antihistamine drugs substances. Such bronchiodilatory drugs include anticholinergic or antimuscarinic agents, in particular ipratropium bromide, oxitropium bromide, and tiotropium bromide, and β-2-adrenoreceptor agonists such as salbutamol, terbutaline, salmeterol and, especially, formoterol. Co-therapeutic antihistamine drug substances include cetirizine hydrochloride, clemastine fumarate, promethazine, loratadine, desloratadine diphenhydramine and fexofenadine hydrochloride.

The effectiveness of an agent of the invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The agents of the invention are also useful as co-therapeutic agents for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance. Accordingly the invention includes a combination of an agent of the invention as hereinbefore described with an anti-inflammatory, bronchodilatory or antihistamine drug substance, said agent of the invention and said drug substance being in the same or different pharmaceutical composition. Such anti-inflammatory drugs include steroids, in particular glucocorticosteroids such as budesonide, beclamethasone, fluticasone, ciclesonide or mometasone, LTB4 antagonists such as those described in U.S. Pat. No. 5,451,700, LTD4 antagonists such as montelukast and zafirlukast, dopamine receptor agonists such as cabergoline, bromocriptine, ropinirole and 4-hydroxy-7-[2-[[2-[[3-(2-phenylethoxy)propyl]-sulfonyl]ethyl]-amino]ethyl]-2(3H)-benzothiazolone and pharmaceutically acceptable salts thereof (the hydrochloride being Viozan®-AstraZeneca), and PDE4 inhibitors such as Ariflo® (GlaxoSmith Kline), Roflumilast (Byk Gulden), V-11294A (Napp), BAY19-8004 (Bayer), SCH-351591 (Schering-Plough), Arofylline (Almirall Prodesfarma) and PD189659 (Parke-Davis). Such bronchodilatory drugs include anticholinergic or antimuscarinic agents, in particular ipratropium bromide, oxitropium bromide and tiotropium bromide, and beta-2 adrenoceptor agonists such as salbutamol, terbutaline, salmeterol and, especially, formoterol and pharmaceutically acceptable salts thereof, and compounds (in free or salt or solvate form) of Formula I of PCT International patent publication No. WO 00/75114, which document is incorporated herein by reference, preferably compounds of the Examples thereof, especially a compound of Formula

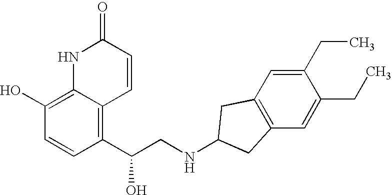

and pharmaceutically acceptable salts thereof. Co-therapeutic antihistamine drug substances include cetirizine hydrochloride, acetaminophen, clemastine fumarate, promethazine, loratidine, desloratidine, diphenhydramine and fexofenadine hydrochloride. Combinations of agents of the invention and steroids, beta-2 agonists, PDE4 inhibitors or LTD4 antagonists may be used, for example, in the treatment of COPD or, particularly, asthma. Combinations of agents of the invention and anticholinergic or antimuscarinic agents, PDE4 inhibitors, dopamine receptor agonists or LTB4 antagonists may be used, for example, in the treatment of asthma or, particularly, COPD.

Other useful combinations of agents of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N—[[4-[[[6,7-dihydro-2-(4-methylphenyl)-5H-benzocyclohepten-8-yl]carbonyl]amino]phenyl]-methyl]tetrahydro-N,N-dimethyl-2H-pyran-4-aminium chloride (TAK-770), and CCR-5 antagonists described in U.S. Pat. No. 6,166,037 (particularly claims 18 and 19), WO 00/66558 (particularly claim 8), and WO 00/66559 (particularly claim 9).

The compounds of the preferred embodiments can also be combined in a pharmaceutical composition with compounds that are useful for the treatment of a thrombolytic disease, heart disease, stroke, etc., (e.g., aspirin, streptokinase, tissue plasminogen activator, urokinase, anticoagulants, antiplatelet drugs (e.g, PLAVIX; clopidogrel bisulfate), a statin (e.g., LIPITOR or Atorvastatin calcium), ZOCOR (Simvastatin), CRESTOR (Rosuvastatin), etc.), a Beta blocker (e.g., Atenolol), NORVASC (amlodipine besylate), and an ACE inhibitor (e.g., lisinopril).

The compounds of the preferred embodiments can also be combined in a pharmaceutical composition with compounds that are useful for the treatment of antihypertension agents such as, ACE inhibitors, lipid lowering agents such as statins, LIPITOR (Atorvastatin calcium), calcium channel blockers dush as NORVASC (amlodipine besylate). The compounds of the preferred embodiments may also be used in combination with fibrates, beta-blockers, NEPI inhibitors, Angiotensin-2 receptor antagonists and platelet aggregation inhibitors.

For the treatment of inflammatory diseases, including rheumatoid arthritis, the compounds of the preferred embodiments may be combined with agents such as TNF-α inhibitors such as anti-TNF-α monoclonal antibodies (such as REMICADE, CDP-870) and D2E7 (HUMIRA) and TNF receptor immunoglobulin fusion molecules (such as ENBREL), IL-1 inhibitors, receptor antagonists or soluble IL-1R [symbol] (e.g. KINERET or ICE inhibitors), nonsterodial anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX (celecoxib), PREXIGE (lumiracoxib)), metalloprotease inhibitors (preferably MMP-13 selective inhibitors), p2×7 inhibitors, α2 [symbol] inhibitors, NEUROTIN, pregabalin, low dose methotrexate, leflunomide, hydroxyxchloroquine, d-penicillamine, auranofin or parenteral or oral gold.

The compounds of the preferred embodiments can also be used in combination with the existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, lumiracoxib and etoricoxib, analgesics and intraarticular therapies such as corticosteroids and hyaluronic acids such as hyalgan and synvisc.

The compounds of the preferred embodiments may also be used in combination with antiviral agents such as Viracept, AZT, acyclovir and famciclovir, and antisepsis compounds such as Valant.

The compounds of the preferred embodiments may also be used in combination with CNS agents such as antidepressants (sertraline), anti-Parkinsonian drugs (such as deprenyl, L-dopa, Requip, Mirapex, MAOB inhibitors such as selegine and rasagiline, comP inhibitors, such as Tasmar, A-2 inhibitors, dopamine reuptake inhibitors, NMDA antagonists, Nicotine agonists, Dopamine agonists, and inhibitors of neuronal nitric oxide synthase), and anti-Alzheimer's drugs such as donepezil, tacrine, α2δinhibitors, NEUROTIN, pregabalin, COX-2 inhibitors, propentofylline or metrifonate.

The compounds of the preferred embodiments may also be used in combination with osteoporosis agents such as EVISTA (raloxifene hydrochloride), droloxifene, lasofoxifene or fosomax and immunosuppressant agents such as FK-506 and rapamycin.

In another aspect of the preferred embodiments, kits that include one or more compounds of the preferred embodiments are provided. Representative kits include a PI3K inhibitor compound of the preferred embodiments (e.g., a compound of Formula Formula A, I, Ia, II, IIa, III, IIIa, IV, V, or Va) and a package insert or other labeling including directions for treating a cellular proliferative disease by administering a PI3K inhibitory amount of the compound.

### Administration and Pharmaceutical Composition

In general, the compounds of preferred embodiments will be administered in a therapeutically effective amount by any of the accepted modes of administration for agents that serve similar utilities. The actual amount of the compound of preferred embodiments, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of compounds of Formula A, I, Ia, II, IIa, III, IIIa, IV, V, or Va may range from about 0.05 to about 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of the preferred embodiments will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the preferred embodiments is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula A, I, Ia, II, IIa, III, IIIa, IV, V, or Va in combination with at least one pharmaceutically acceptable excipient. Acceptable excipients are non-toxic, aid administration, and do not adversely affect the therapeutic benefit of the compound of Formula I, II, or III. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the preferred embodiments in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula A, I, Ia, II, IIa, III, IIIa, IV, V, or Va based on the total formulation, with the balance being one or more suitable pharmaceutical excipients. Preferably, the compound is present at a level of about 1-80 wt %.

### General Synthetic Methods

The compounds of preferred embodiments can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of preferred embodiments contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of the preferred embodiments, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chem or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the preferred embodiments may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Accordingly, in one embodiment the preferred embodiments provides a method for synthesizing a compound, stereoisomer, tautomer, or a pharmaceutically acceptable salt of Formula I,

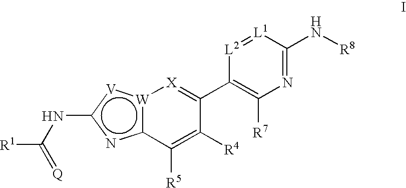

wherein the method comprises coupling a compound having the Formula:

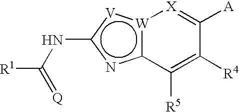

with a compound having the Formula:

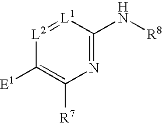

in the presence of a catalyst;

wherein:

A is a halogen or other suitable leaving group;

E¹ is a boronic ester or boronic acid; and

Q, V, W, X, L¹, L², R¹, R⁴, R⁵, R⁷, and R⁸ are previously defined for Formula I.

In one embodiment provided is a method for synthesizing a compound, stereoisomer, tautomer, or a pharmaceutically acceptable salt of Formula IIIa,

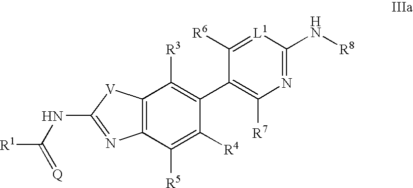

wherein the method comprises coupling a compound having the Formula:

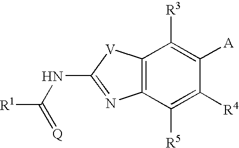

with a compound having the Formula:

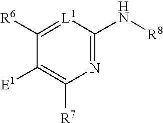

in the presence of a catalyst;

wherein:

A is a halogen or other suitable leaving group

E¹ is a boronic ester or boronic acid;

Q, V, L¹, R¹, R³, R⁴, R⁵, R⁶, R⁷, and R⁸ are previously defined for Formula IIIa.

Compounds of preferred embodiments can be made by employing palladium mediated coupling reactions, such as Suzuki coupling. Said couplings can be employed to functionalize a heterocycle or aryl ring system at each position of the ring system providing said ring is suitably activated or functionalized.

Suzuki coupling (Suzuki et al., Chem. Commun. (1979) 866) can be used to form the final product and can be effected under known conditions such as by treatment with functionalized boronic esters as in the following schemes where, for illustrative purposes, compounds of Formula II and III are shown and where E¹ is a boronic ester:

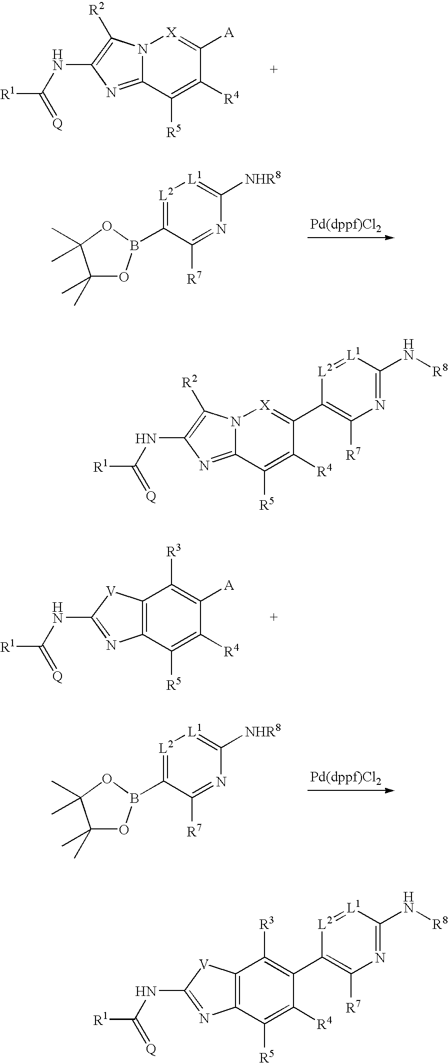

The pyridinyl, pyrazinyl, or pyrimidinyl starting materials can be obtained commercially and functionalized as shown in the scheme below. The pyridinyl, pyrazinyl, or pyrimidinyl cores can comprise substituents that can be converted to desired functional groups and can comprise substituents with protecting groups, which can be removed in an appropriate setting.

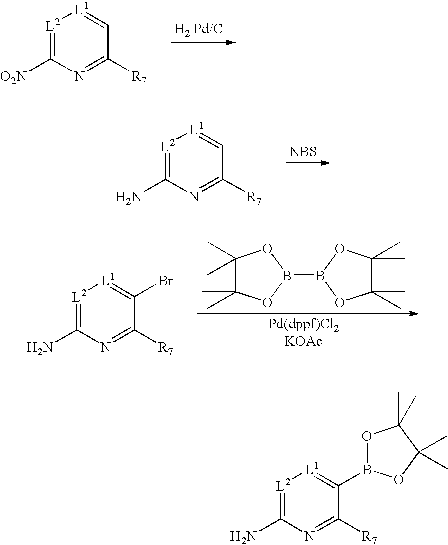

These methods can be adapted for preparing compounds of Formula A, I, Ia, II, IIa, III, IIIa, IV, V, or Va. For compounds of Formula Ia, the methods include reacting a halo-imidazopyridine with a pyridinyl or pyrimidinyl group containing a reactive boronic ester substituent, in the presence of a palladium catalyst. For compounds of Formula III, the methods include reacting a halo-benzothiazole with a pyridinyl or pyrimidinyl group containing a reactive boronic ester substituent, in the presence of a palladium catalyst.

In an embodiment, the palladium catalyst is palladium dichloride. In an embodiment, the palladium catalyst is dichloro(1,1-bis(diphenylphosphino)ferrocene) palladium(II)-dichloromethane adduct (Pd(dppf)Cl₂-DCM).

More particular syntheses of compounds of the preferred embodiments, particularly those of Formula A, I, Ia, II, IIa, III, IIIa, IV, V, or Va are provided in the following Methods and Examples:

The compounds of the invention, particularly compounds of Formula (A) and Formula (IV) may be prepared from compounds of Formula (VI)

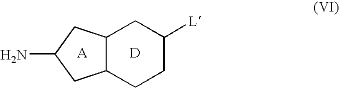

where L′ is a halogen or other suitable leaving group followed by derivatisation of the amino group and Suzuki coupling as previously described.

Compounds of Formula (VI), which are represented by compounds of Formula (VII)

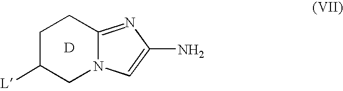

may be prepared by methods known or obvious to those skilled in the art, for example according to the following Scheme where L′ is represented by Br.

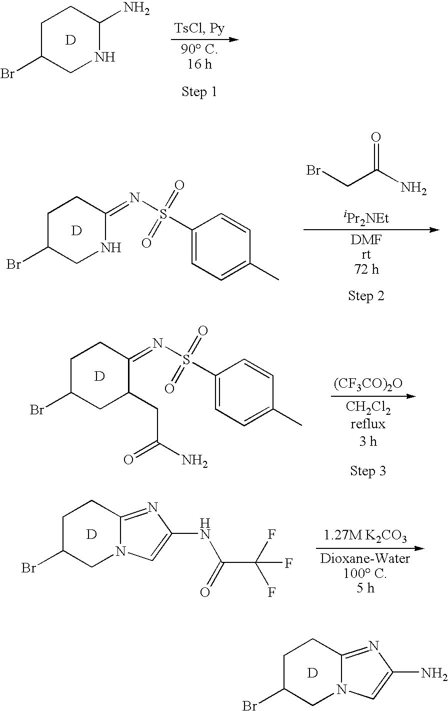

Compounds of Formula (VI), which are represented by compounds of Formula (VIII)

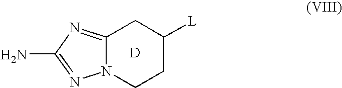

may be prepared by methods known or obvious to those skilled in the art, for example according to the following Scheme where L represents Br, e.g. as described in WO2006/038116.

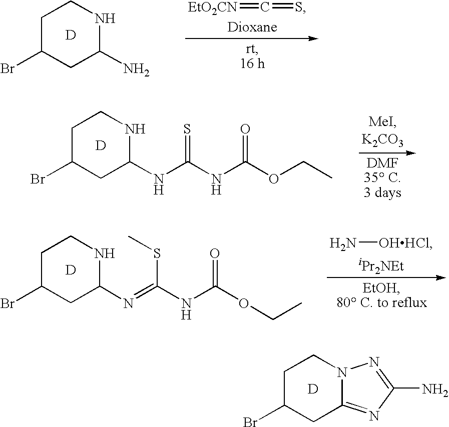

Compounds of Formula VI-VIII may be further substituted and derviatised at the nitrogen group to prepare compounds of the invention by methods well-known to those skilled in the art. For example, compounds of Formula IV where R¹ is Z—Y—R¹⁰ and the preferred groups thereof, may be prepared according to the analogous methods described in WO05/021519.

### EXAMPLES

Referring to the examples that follow, compounds of the preferred embodiments were synthesized using the methods described herein, or other methods, which are known in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

GCMS analysis is performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.). The spectral reference was either TMS or the known chemical shift of the solvent. Some compound samples were run at elevated temperatures (e.g., 75° C.) to promote increased sample solubility.

The purity of some of the compounds is assessed by elemental analysis (Desert Analytics, Tucson, Ariz.).

Melting points are determined on a Laboratory Devices Mel-Temp apparatus (Holliston, Mass.).

Preparative separations are carried out using a Flash 40 chromatography system and KP-Sil, 60A (Biotage, Charlottesville, Va.), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings.

### Abbreviations

ACN acetonitrile
CDI 1,1′-carbonyldiimidazole
DCM dichloromethane
DIC N,N′-diisopropylcarbodiimide
DIEA diisopropylethylamine
DME 1,2-dimethoxyethane
DMF dimethylformamide
DMSO dimethyl sufoxide
DPPF 1,1′-bis(diphenylphosphino)ferrocene
EDCI (EDC) 1-(3-dimethylaminopropyl)-3-ethylcarbodiimide hydrochloride
EtOAc ethyl acetate
EtOH ethanol
HATU 2-(7-aza-1H)-benzotrizole-1-yl-1,1,3,3-tetramethylisouronium hexafluorophosphate
HOBt hydroxybenzotriazole
MeOH methanol
NBS N-bromosuccinimide
NCS N-chlorosuccinimide
NMP N-methyl-2-pyrrolidone
RT (rt) room temperature
TEA triethylamine
THF tetrahydrofuran
TFA trifluoroacetic acid

The following methods were used for compounds of Formula A, I, Ia, II, IIa, III, IIIa, IV, V, or Va:

### Method 1

### Preparation of 6-iodoimidazo[1,2-a]pyridin-2-amine

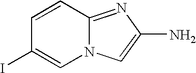

To a solution of 2,2,2-trifluoro-N-(6-iodoimidazo[1,2-a]pyridin-2-yl)acetamide (Hamodouchi, C.; Sanchez, C.; Ezquerra, J. Synthesis 1998, 867; 4.8 g, 13.5 mmol) in THF, MeOH, and H₂O (1:1:1, 45 mL, 0.3 M) was added anhydrous K₂CO₃ (18.6 g, 0.135 mol) at room temperature. The reaction mixture was refluxed for 12 h. After cooling down, the reaction mixture was diluted with EtOAc (150 mL) and H₂O (100 mL). The organic layer was separated, washed with brine (100 mL), dried over anhydrous Na₂SO₄, filtered, concentrated and dried in vacuo to give 6-iodoimidazo[1,2-a]pyridin-2-amine as a brown solid (1.8 g, 51%). The crude product was used for the next step without further purification. LC/MS (m/z): 259.9 (MH⁺), Rₜ: 1.23 min; HPLC Rₜ: 1.05 min.

### Method 2

### Preparation of 6-chloroimidazo[1,2-b]pyridazin-2-amine

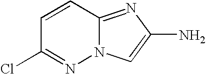

According to Method 1,6-chloroimidazo[1,2-b]pyridazin-2-amine was obtained from N-(6-chloroimidazo[1,2-b]pyridazin-2-yl)-2,2,2-trifluoroacetamide (Hamodouchi, C.; Sanchez, C.; Ezquerra, J. Synthesis 1998, 867) in 66% yield. LC/MS (m/z):168.9 (MH⁺), Rₜ: 1.29 min; HPLC Rₜ: 1.14 min.

### Method 3

### Preparation of N-(6-iodoimidazo[1,2-a]pyridin-2-yl)acetamide

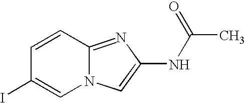

To a solution of 6-iodoimidazo[1,2-a]pyridin-2-amine (1.0 g, 3.8 mmol) in CH₂Cl₂ (13 mL) was added Et₃N (0.587 mL, 4.2 mmol), DMAP (46 mg, 0.38 mmol), and Ac₂O (0.396 mL, 4.2 mmol) sequentially at room temperature. The reaction mixture was stirred for 5 h and the precipitate was filtered off, washed and dried to yield N-(6-iodoimidazo[1,2-a]pyridin-2-yl)acetamide as a brown solid (0.95 g, 83%). LC/MS (m/z): 302.0 (MH⁺), Rₜ: 1.40 min; HPLC Rₜ: 1.60 min; ¹H NMR (CD₃OD, 300 MHz) δ 8.72 (m, 1H), 8.05 (s, 1H), 7.61 (d, 1H, J=9.6 Hz), 7.45 (d, 1H, J=9.9 Hz), 2.18 (s, 3H).

### Method 4

### Preparation of 6-chloroimidazo[1,2-b]pyridazin-2-amine

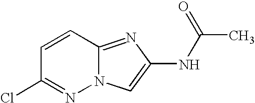

According to Method 3, N-(6-chloroimidazo[1,2-b]pyridazin-2-yl)acetamide was obtained from 6-chloroimidazo[1,2-b]pyridazin-2-amine in 99% yield. LC/MS (m/z): 211.0 (MH⁺), Rₜ: 1.77 min; HPLC Rₜ: 2.06 min; ¹H NMR (DMSO-d⁶, 300 MHz) δ 8.25 (s, 1H), 8.57 (d, 1H, J=9.3 Hz), 7.45 (dd, 1H, J=0.9 and 9.3 Hz), 2.10 (s, 3H).

### Method 5

### Synthesis of N-(6-Bromo-imidazo[1,2-a]pyridin-2-yl)-2,2,2-trifluoro-acetamide

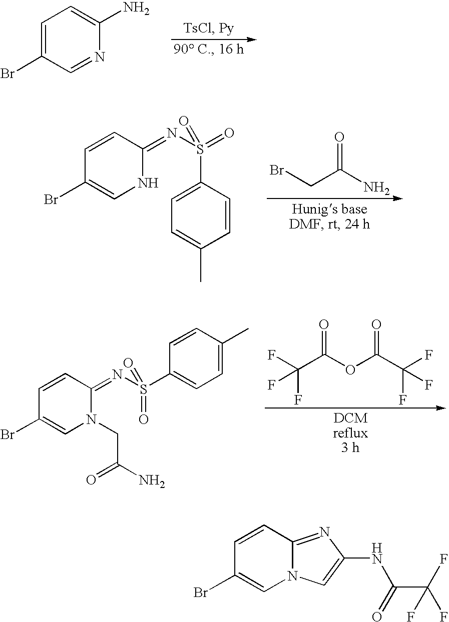

### Step 1: N-[5-Bromo-1H-pyridin-(2Z)-ylidene]-4-methyl-benzenesulfonamide

Tosyl chloride (52.9 g, 277.4 mmol) was added slowly to a stirred solution of 2-amino-5-bromopyridine (40.0 g, 231 mmol) in dry pyridine (240 mL) at 0° C. The reaction was heated at 90° C. for 16 hours. The mixture was then concentrated in vacuo and water (500 ml) was added. The resulting mixture was stirred for 30 minutes at room temperature. The title compound was removed by filtration and dried in a vacuum oven at 50° C.

### Step 2: 2-{5-Bromo-2-[(Z)-toluene-4-sulfonylimino]-2H-pyridin-1-yl}-acetamide

N-[5-Bromo-1H-pyridin-(2Z)-ylidene]-4-methyl-benzenesulfonamide (80 g, 244.5 mmol) was suspended in anhydrous DMF (350 ml). Hünig's base (46.8 ml, 268.9 mmol) was added, followed by 2-bromoacetamide (37.12 g, 268.9 mmol) and the mixture was stirred at room temperature for 72 hours. The reaction was poured into water (1000 ml) and stirred for 1 hour. The product was collected by filtration, washed with more water (300 ml) and dried in a vacuum oven at 50° C. to afford the title compound.

### Step 3: N-(6-Bromo-imidazo[1,2-a]pyridin-2-yl)-2,2,2-trifluoro-acetamide

Trifluoroacetic anhydride (100 ml) was added slowly to a stirred suspension of 2-{5-bromo-2-[(Z)-toluene-4-sulfonylimino]-2H-pyridin-1-yl}-acetamide (20 g, 52 mmol) in anhydrous dichloromethane (250 ml). The reaction was heated at reflux for 3 hours and then concentrated in vacuo to afford a yellow solid consisting of the tosic acid salt of the title compound. The solid was suspended in aqueous sodium bicarbonate solution and stirred for 15 minutes to yield the title compound. ¹H NMR (CDCl₃): 7.37 (1H, d), 7.43 (1H, d), 8.15 (1H, s), 8.43 (1H, s), and 10.2 (1H, s).

### Method 6

### Synthesis of 6-bromo-imidazo[1,2-a]pyridin-2-ylamine

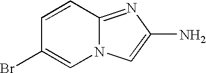

A stirred solution of N-(6-bromo-imidazo[1,2-a]pyridin-2-yl)-2,2,2-trifluoro-acetamide (Method 5) (9.0 g, 29.2 mmol) in DME (90 ml) and aqueous potassium phosphate (1.27 M, 80.5 ml, 102.3 mmol) was heated at 90° C. overnight. The mixture was allowed to cool and the two layers were separated. The aqueous layer was extracted with EtOAc and the organic layers were concentrated under vacuum to a brown oil. Iso-hexane was added to the residue to afford a solid. Excess iso-hexane was decanted off and the remaining DME was azeotroped with THF (2×50 ml) to afford the title compound as a solid, LC/MS (m/z): 211.9 (MH⁺)

### Method 7

### Preparation of methyl 6-iodoimidazo[1,2-a]pyridin-2-ylcarbamate

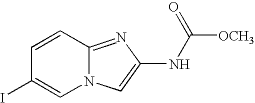

To a solution of 6-iodoimidazo[1,2-a]pyridin-2-amine (0.5 g, 1.9 mmol) in THF (6 mL) was added DIEA (0.664 mL, 3.8 mmol) and methyl chloroformate (0.162 mL, 2.1 mmol) sequentially at room temperature. The reaction mixture was stirred for 15 h, the precipitate was filtered off, washed and dried to yield a mixture of 6-iodoimidazo[1,2-a]pyridin-2-ylcarbamate (LC/MS (m/z): 317.9 (MH⁺), Rₜ: 1.65 min; HPLC Rₜ: 1.81 min) and 1,3-bis(6-iodoimidazo[1,2-a]pyridin-2-yl)urea(LC/MS (m/z): 544.9 (MH⁺), Rₜ: 2.09 min; HPLC Rₜ: 2.56 min). The crude product was used for the next step without further purification.

### Method 8

### Synthesis of (6-Bromo-imidazo[1,2-a]pyridin-2-yl)-carbamic acid phenyl ester

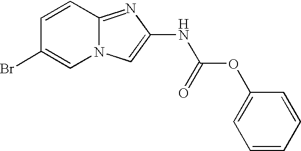

To a solution of 6-bromo-imidazo[1,2-a]pyridin-2-ylamine (6.2 g, 29.2 mmol) in THF (400 ml) was added 2,4,6-trimethylpyridine (5.8 ml, 43.9 mmol). The reaction mixture was cooled to 0° C. (ice-bath) and a solution of phenyl chloroformate (3.85 ml, 30.7 mmol) in THF (50 ml) was added dropwise over 15 minutes. The reaction mixture was stirred overnight and then quenched with water and stirred for a further 5 minutes to afford a suspension. The solid was collected by filtration and dried under vacuum (40° C.) overnight to afford the title compound. LC/MS (m/z): 331.99 and 333.99 (MH⁺).

### Method 9

### Preparation of 4-(piperidin-1-yl)butanoic acid

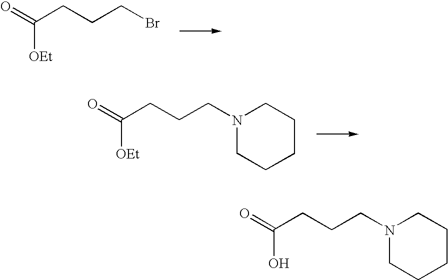

A solution of ethyl 4-bromobutanoate (3.9 g, 20 mmol) and piperidine (4.2 mL, 42 mmol) in ACN (30 mL) was heated at 100° C. for 3 h. The ACN was removed under reduced pressure, and the residue was diluted with ethyl acetate, washed with water and brine, dried over sodium sulfate, filtered and concentrated to give ethyl 4-(piperidin-1-yl)butanoate (3.82 g, 96%). LC/MS (m/z) 200.1 (MH⁺), Rₜ: 0.31 min.

A mixture of ethyl 4-(piperidin-1-yl)butanoate (2 g, 10 mmol) and con. HCl (40 mL) was heated at 100° C. for 16 hours. Water and excess HCl were removed to give a white solid, which was triturated with ethanol and filtered. The solid was washed with ethanol and dried to give 4-(piperidin-1-yl)butanoic acid as it HCl salt (1.52 g, 73%). LC/MS (m/z) 172.1 (MH⁺), Rₜ: 0.32 min.

### Method 10

### Preparation of 4-morpholinobutanoic acid

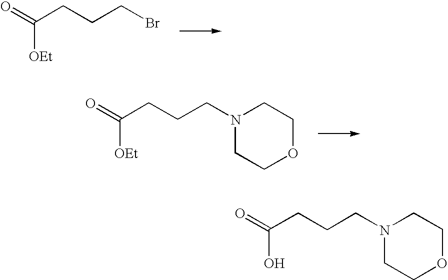

A solution of ethyl 4-bromobutanoate (3.9 g, 20 mmol) and morpholine (3.67 mL, 42 mmol) in ACN (30 mL) was heated at 100° C. for 3 h. ACN was removed, and the residue was diluted with ethyl acetate, washed with water and brine, dried over sodium sulfate, filtered and concentrated to give ethyl 4-morpholinobutanoate (3.78 g, 93%). LC/MS (m/z) 202.1 (MH⁺), Rₜ: 0.78 min.

A mixture of ethyl 4-morpholinobutanoate (2 g, 9.9 mmol) and conc. HCl (20 mL) was heated at 100° C. for 6 hours. Water and excess HCl were removed, and the residue was triturated with ethanol to give a white solid. The solid was filtered, washed with ethanol, and dried to afford 4-morpholinobutanoic acid as its HCl salt (1.2 g, 58%). LC/MS (m/z) 174.1 (MH⁺), Rₜ: 0.34 min.

### Method 11

### Preparation of N-(6-iodoimidazo[1,2-a]pyridin-2-yl)-4-morpholinobutanamide

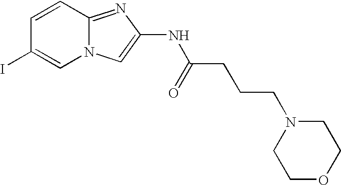

A mixture of 4-morpholinobutanoic acid HCl salt (50 mg, 0.24 mmol), 6-iodoimidazo[1,2-a]pyridin-2-amine (50 mg, 0.19 mmol), EDCI (60 mg, 0.31 mmol), and DIEA (0.114 mL) in DCM (4 mL) was stirred overnight. The mixture was diluted with ethyl acetate, washed with water, saturated aqueous sodium bicarbonate, brine and dried over sodium sulfate, filtered and concentrated to give N-(6-iodoimidazo[1,2-a]pyridin-2-yl)-4-morpholinobutanamide (76 mg, 95%). LC/MS (m/z): 414.9 (MH⁺), Rₜ: 1.59 min.

### Method 12

### Preparation of N-(6-iodoimidazo[1,2-a]pyridin-2-yl)-4-(piperidin-1-yl)butanamide

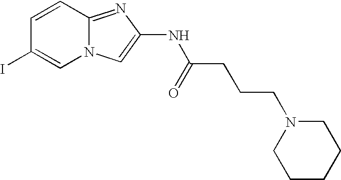

N-(6-iodoimidazo[1,2-a]pyridin-2-yl)-4-(piperidin-1-yl)butanamide was prepared according to Method 11 from 4-(piperidin-1-yl)butanoic acid-HCl salt in 85% yield. LC/MS (m/z): 412.9 (MH⁺), Rₜ: 1.70 min.

### Method 13

### Preparation of 4-hydroxy-N-(6-iodoimidazo[1,2-a]pyridin-2-yl)butanamide

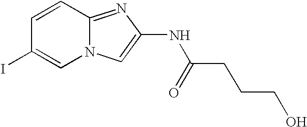

To a suspension of 6-iodoimidazo[1,2-a]pyridin-2-amine (100 mg, 0.39 mmol) in DCM at room temperature was added dimethylaluminum chloride (1M in hexane, 0.72 mL, 0.72 mmol). After 10 minutes, gamma-butyrolactone (0.06 mL, 0.63 mmol.) was added. The mixture was stirred at room temperature overnight, then poured into methanol (30 mL). The solution was concentrated to obtain the desired product, which was used without further purification. LC/MS (m/z): 346.0 (MH⁺), Rₜ: 1.63 min.

### Method 14

### Preparation of tert-butyl 4-(2-methoxy-2-oxoethoxy)piperidine-1-carboxylate

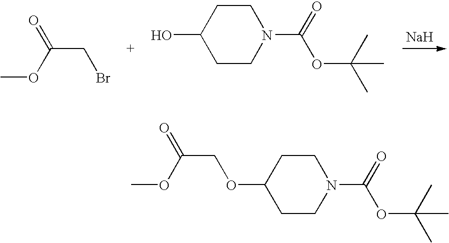

To a solution of the tert-butyl 4-hydroxypiperidine-1-carboxylate (2.75 g, 18 mmol) in tetrahydrofuran (50 mL) was added sodium hydride (384 mg, 15 mmol) at 0° C. under nitrogen atmosphere. After the mixture was stirred at that temperature for 1 hour, methyl 2-bromoacetate (2.02 g, 10 mmol) was added dropwise. And the mixture was stirred at room temperature for two days. The reaction mixture was then diluted with ethyl acetate (300 mL) and washed with sat. aq. ammonium chloride solution, brine, dried over MgSO₄, filtered, and evaporated under reduced pressure to give crude product, which was purified by silica gel column chromatography (ethyl acetate and hexane) to give the titled compound. LC/MS (m/z): 296.1 (M+Na), Rₜ: 2.55 min.

### Method 15

### Preparation of 2-(1-(tert-butoxycarbonyl)piperidin-4-yloxy)acetic acid

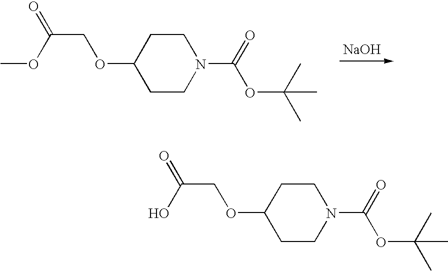

To a solution of the tert-butyl 4-(2-methoxy-2-oxoethoxy)piperidine-1-carboxylate (280 mg, 1 mmol) in 2 mL of methanol, 1 mL of tetrahydrofuran and 1 mL of water was added aq. sodium hydroxide (10 N, 2 mL, 20 mmol). After the reaction mixture was stirred at room temperature for 4 hours, pH was adjusted to 7 by the dropwise addition of 3N HCl. The resulting mixture was extracted with ethyl acetate (3×100 mL). The combined organic layers were washed with brine, then dried over MgSO₄, filtered, and evaporated under reduced pressure to give the title compound, which was used in the next step without further purification.

### Method 16

### Preparation of N-(6-bromobenzo[d]thiazol-2-yl)acetamide

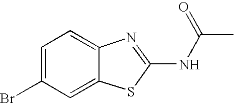

To a solution of 2-amino-6-bromobenzothiazole (3.0 g, 13.04 mmol) in THF (30 mL) was added acetic anhydride (4 mL, 42.3 mmol). The solution was stirred at room temperature for 2 days. THF was removed to give a white solid, which was recrystallized from hot ethyl acetate to give N-(6-bromobenzo[d]thiazol-2-yl)acetamide (2.49 g, 71%). LC/MS (m/z) 270.9/272.9 (MH⁺), Rₜ: 2.57 min.

### Method 17

### Preparation of N-(6-bromobenzo[d]thiazol-2-yl)-4-morpholinobutanamide

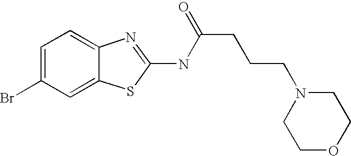

A mixture of 4-morpholinobutanoic acid (250 mg, 1.2 mmol), 2-amino-6-bromobenzothiazole (230 mg, 1.0 mmol), HATU (456 mg, 1.4 mmol) and DIEA (0.53 mL, 3.0 mmol) in THF (20 mL) was stirred at room temperature overnight. The THF was removed, and the residue was diluted with ethyl acetate, washed with saturated ammonium chloride (aq.), brine, dried and concentrated to give N-(6-bromobenzo[d]thiazol-2-yl)-4-morpholinobutanamide. LC/MS (m/z) 384.0/386.0 (MH⁺), Rₜ: 2.13 min.

### Method 18

### Preparation of N-(6-bromobenzo[d]thiazol-2-yl)-4-(piperidin-1-yl)butanamide

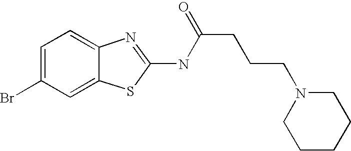

N-(6-bromobenzo[d]thiazol-2-yl)-4-(piperidin-1-yl)butanamide was prepared in a similar fashion as Method 17. LC/MS (m/z) 381.9/384.0 (MH⁺), Rₜ: 2.30 min.

### Method 19

### Synthesis of 6-bromo-7-methylbenzo[d]thiazol-2-amine and 6-bromo-5-methylbenzo[d]thiazol-2-amine

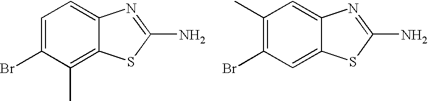

To a solution of 4-bromo-3-methylaniline (1 g, 5.38 mmol) and tetrabutylammonium thiocyanate (1.6 g, 5.38 mmol) in DCM at room temperature was added benzyltrimethylammonium tribromide (2.1 g, 5.38 mmol). The reaction mixture was stirred at room temperature overnight and the white solid thus formed was filtered off, triturated with DCM (15 mL) water (15 mL), filtered, washed with EtOH (2×), and dried to give 6-bromo-7-methylbenzo[d]thiazol-2-amine. LC/MS (m/z): (244.9, MH⁺), Rₜ: 2.04 min; ¹H NMR (DMSO-d⁶, 300 MHz) δ 7.59 (1H, d, J=8.8 Hz), 7.23 (1H, d, J=8.8 Hz), 2.42 (s, 3H).

The first filtrate from the reaction was washed with saturated sodium bicarbonate, water, and brine, dried and concentrated to give a residue, which was triturated with DCM to give 6-bromo-5-methylbenzo[d]thiazol-2-amine (453 mg, 35%). LC/MS (m/z): (244.9, MH⁺), Rₜ: 2.04 min; ¹H NMR (DMSO-d⁶, 300 MHz) δ 8.22 (1H, bs), 7.94 (1H, bs), 2.34 (s, 3H).

### Method 20

### Synthesis of N-(6-bromo-7-methylbenzo[d]thiazol-2-yl)acetamide

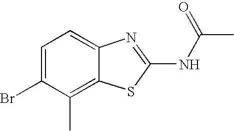

N-(6-bromo-7-methylbenzo[d]thiazol-2-yl)acetamide was prepared according to Method 16 from 6-bromo-7-methylbenzo[d]thiazol-2-amine. LC/MS (m/z): (286.9, MH⁺), Rₜ: 2.79 min; HPLC Rₜ: 3.65 min.

### Method 21

### Synthesis of N-(6-bromo-5-methylbenzo[d]thiazol-2-yl)acetamide

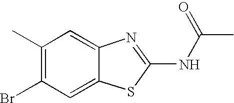

N-(6-bromo-5-methylbenzo[d]thiazol-2-yl)acetamide was prepared according to Method 16 from 6-bromo-5-methylbenzo[d]thiazol-2-amine. LC/MS (m/z): (286.9, MH⁺), Rₜ: 2.80 min; HPLC Rₜ: 3.66 min.

### Method 22

### Synthesis of 6-bromo-7-fluorobenzo[d]thiazol-2-amine

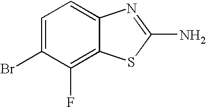

6-Bromo-7-fluorobenzo[d]thiazol-2-amine was prepared according to Method 19 from 4-bromo-3-fluoroaniline. LC/MS (m/z): (248.0, MH⁺), Rₜ: 2.24 min; HPLC Rₜ: 2.72 min; ¹H NMR (DMSO-d⁶, 300 MHz) δ 7.85 (2H, bs), 7.45 (1H, dd, J=7.4 and 8.7 Hz), 7.15 (1H, d, J=8.7 Hz).

### Method 23

### Synthesis of N-(6-bromo-7-fluorobenzo[d]thiazol-2-yl)acetamide

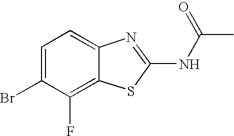

N-(6-bromo-7-fluorobenzo[d]thiazol-2-yl)acetamide was prepared according to Method 16 from 6-bromo-7-fluorobenzo[d]thiazol-2-amine. LC/MS (m/z): (288.9, MH⁺), Rₜ: 2.73 min; HPLC Rₜ: 3.68 min.

### Method 24

### Synthesis of 6-bromo-4-fluorobenzo[d]thiazol-2-amine

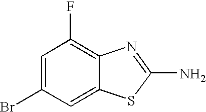

6-Bromo-4-fluorobenzo[d]thiazol-2-amine was prepared according to Method 19 from 4-bromo-2-fluoroaniline. LC/MS (m/z): (248.9, MH⁺), Rₜ: 2.29 min; HPLC Rₜ: 2.86 min.

### Method 25

### Synthesis of N-(6-bromo-4-fluorobenzo[d]thiazol-2-yl)acetamide

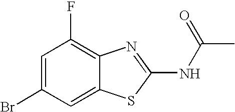

N-(6-bromo-4-fluorobenzo[d]thiazol-2-yl)acetamide was prepared according to Method 16 from 6-bromo-4-fluorobenzo[d]thiazol-2-amine. LC/MS (m/z): (288.9, MH⁺), Rₜ: 2.30 min; HPLC Rₜ: 3.63 min.

### Method 26

### Synthesis of 6-bromo-[1,2,4]triazolo[1,5-a]pyridin-2-ylamine

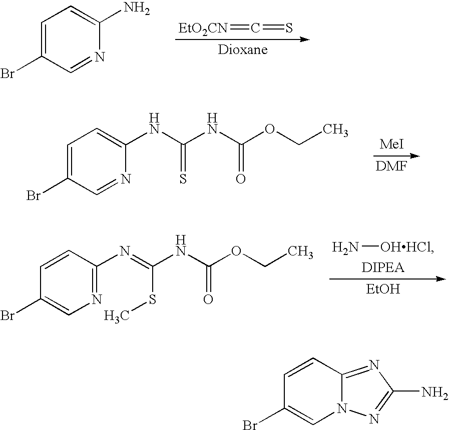

### Step 1:

5-Bromo-pyridin-2-ylamine (2.5 g, 14.5 mmol) was dissolved in dry dioxane (30 ml) and carbethoxy isothiocyanate was added via syringe. The reaction mixture was stirred at room temperature overnight and then concentrated in vacuo. The residue was purified by chromatography on silica eluting with 1:1 EtOAc/iso-hexanes to afford the title compound as a white solid.

### Step 2

The product from step 1(2.0 g, 6.58 mmol) was dissolved in dry DMF (15 ml) and K₂CO₃ (1.18 g, 8.55 mmol) was added followed by methyl iodide (0.49 ml, 7.90 mmol). The resulting mixture was stirred at 35° C. for 3 days. The reaction was allowed to cool to room temperature, concentrated in vacuo and water (40 ml) followed by 1:1 EtOAc/iso-hexanes (150 ml) was added. The aqueous phase was separated and the organics were washed with water (2×40 ml) and brine (30 ml). The combined organic portions were dried (MgSO₄), filtered and concentrated in vacuo. Purification by chromatography on silica gel, eluting with 20% EtOAc/Iso-hexanes afforded the title compound.

### Step 3: 6-Bromo-[1,2,4]triazolo[1,5-a]pyridin-2-ylamine

Hydroxylamine hydrochloride (0.328 g, 5.13 mmol) was suspended in EtOH (60 ml). DIPEA (0.81 ml, 5.13 mmol) was added and the reaction mixture was stirred for 10 minutes at room temperature. This solution was then transferred by syringe and added to a suspension of the product from step 2 (0.726 g, 2.28 mmol) in EtOH (10 ml). The reaction mixture was stirred at room temperature for a further 10 minutes and then heated to 80° C. (using a reflux condenser connected to a trap containing bleach) for 2 hours then allowed to cool to room temperature overnight. The mixture was concentrated to approximately 20% volume then DCM (75 ml) was added and the mixture was washed with water (50 ml) and brine (50 ml). The organic extracts were dried (MgSO₄), filtered and concentrated in vacuo to afford the title compound as a white solid.

### Method 27

### Preparation of N-(6-bromo-5-methylimidazo[1,2-a]pyridin-2-yl)-2,2,2-trifluoroacetamide

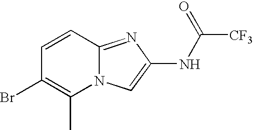

### Step 1: N-(5-bromo-6-methylpyridin-2-yl)-4-methylbenzenesulfonamide

A solution of 5-bromo-6-methylpyridin-2-amine (10.0 g, 53.5 mmol), p-toluenesulfonyl chloride (30.5 g, 160.4 mmol) in pyridine (120 mL) was heated at 85° C. for 18 hours. Upon cooling, the dark brown solution was added to water (1.5 L). The solution was decanted away from a sticky solid, and the sticky solid was dissolved in ethyl acetate, transferred and the volatiles were removed in vacuo. A 1:1 ethyl acetate/hexanes solution (200 mL) was added and upon sonicating a brown solid formed and was filtered off. This brown solid was mainly bistosylated. The ethyl acetate/hexane filtrate was concentrated yielding crude N-(5-bromo-6-methylpyridin-2-yl)-4-methylbenzenesulfonamide (8.86 g, 49%). LC/MS (m/z): 340.9 (MH⁺), Rₜ: 2.94 min; HPLC Rₜ: 4.07 min.

### Step 2: Z)-2-(5-bromo-6-methyl-2-(tosylimino)pyridin-1(2H)-yl)acetamide

To a solution of N-(5-bromo-6-methylpyridin-2-yl)-4-methylbenzenesulfonamide (8.86 g, 26.1 mmol) in DMF (100 mL) was added DIEA (5.44 mL, 31.3 mmol) and then 2-iodoacetamide (5.79 g, 31.3 mmol). The solution was stirred under argon for 15 hours at which time more 2-iodoacetamide (0.58 g, 3.13 mmol) was added. After stirring for an additional 18 hours the dark brown solution was added to water (1.5 L). The solution was decanted away and the residue was dissolved in ethyl acetate (200 mL). The ethyl acetate solution was washed with 1:1 10% NaHSO₃/NaHCO_{3(sat.)} and then NaCl_{(sat.)} (50 mL). After drying over MgSO₄, the volatiles were removed in vacuo and the material was purified by SiO₂ chromatography (25-50-100% EtOAc/hexanes) to yield (Z)-2-(5-bromo-6-methyl-2-(tosylimino)pyridin-1(2H)-yl)acetamide (561 mg, 5%). LC/MS (m/z): 398.0 (MH⁺), Rₜ: 2.09 min; HPLC Rₜ: 2.62 min. The isomeric alkylation product 2-(N-(5-bromo-6-methylpyridin-2-yl)-4-methylphenylsulfonamido)acetamide was also obtained (3.22 g, 31%). LC/MS (m/z): 398.0 (MH⁺), Rₜ: 2.63 min; HPLC Rₜ: 3.65 min.

### Step 3: N-(6-bromo-5-methylimidazo[1,2-a]pyridin-2-yl)-2,2,2-trifluoroacetamide

To a solution of (Z)-2-(5-bromo-6-methyl-2-(tosylimino)pyridin-1(2H)-yl)acetamide (500 mg, 1.26 mmol) in CH₂Cl₂ (30 mL) was added trifluoroacetic anhydride (10 mL). The resulting solution was refluxed in a 50° C. oil bath for 7 hours. Upon cooling, the volatiles were removed in vacuo and theresidue was partitioned between ethyl acetate (200 mL) and NaHCO_{3(sat.)} (50 mL). The two phases were separated, the organic phase was washed further with NaCl_{(sat.)} dried over Na₂SO₄, filtered and concentrated under reduced pressure to yield N-(6-bromo-5-methylimidazo[1,2-a]pyridin-2-yl)-2,2,2-trifluoroacetamide (420 mg, 99%) which was used without further purification. LC/MS (m/z): 321.9 (MH⁺), Rₜ: 2.30 min; HPLC Rₜ: 3.23 min.

According to Method 27, following compounds were prepared from the corresponding 2-aminopyridines:

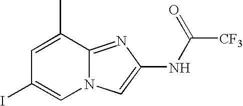

2,2,2-Trifluoro-N-(6-iodo-8-methylimidazo[1,2-a]pyridin-2-yl)acetamide from 5-iodo-3-methylpyridin-2-amine. LC/MS (m/z): 369.8 (MH⁺), Rₜ: 1.91 min; HPLC Rₜ: 2.07 min.

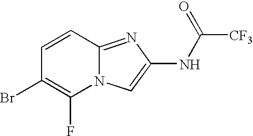

N-(6-Bromo-5-fluoroimidazo[1,2-a]pyridin-2-yl)-2,2,2-trifluoroacetamide from 5-bromo-6-fluoropyridin-2-amine. LC/MS (m/z): 327.9 (MH⁺), Rₜ: 2.03 min; HPLC Rₜ: 2.29 min.

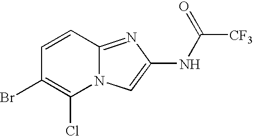

N-(6-Bromo-5-chloroimidazo[1,2-a]pyridin-2-yl)-2,2,2-trifluoroacetamide from 5-bromo-6-chloropyridin-2-amine. LC/MS (m/z): 343.9 (MH⁺), Rₜ: 2.13 min; HPLC Rₜ: 2.44 min.

### Method 28

### Preparation of N-(6-bromo-5-methoxyimidazo[1,2-a]pyridin-2-yl)-2,2,2-trifluoroacetamide

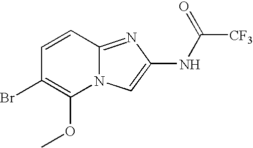

A mixture of N-(6-bromo-5-fluoroimidazo[1,2-a]pyridin-2-yl)-2,2,2-trifluoroacetamide (159 mg, 0.49 mmol) and potassium carbonate (202 mg, 1.5 mmol) in methanol (1 mL) was heated under microwave irradiation at 120° C. for 20 min. The reaction mixture was diluted with methanol (5 mL), filtered, and concentrated in vacuo. The residue was dissolved in EtOAc (30 mL) and washed with water (20 mL). The water wash was extracted twice with EtOAc (50 mL) and the organic phases were combined, washed with 40 mL sat. NaCl and dried over sodium sulfate. Concentration in vacuo gave a crude brown oil (47 mg), which was used in the next step without further purification. LC/MS (m/z): 340.0 (MH⁺), Rₜ: 1.89 min; HPLC Rₜ: 1.98 min.

### Method 29

### Preparation of N-(6-bromo-5-methylimidazo[1,2-a]pyridin-2-yl)acetamide

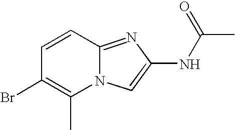

A solution of N-(6-bromo-5-methylimidazo[1,2-a]pyridin-2-yl)-2,2,2-trifluoroacetamide (420 mg, 1.30 mmol) and K₂CO₃ (1.8 g, 13.0 mmol) in 30 mL of 1:1:1 (MeOH, THF, H₂O) was heated at 80° C. for 16 hours. Upon cooling the layers were separated, the organic layer was dried over Na₂SO₄, was filtered and the volatiles were removed in vacuo. Dichloromethane (10 mL), DMAP (63 mg, 0.52 mmol), DIEA (0.45 mL, 2.6 mmol) and acetic anhydride (0.245 mL, 2.6 mmol) were added and the solution was stirred for 24 hours. Upon removal of volatiles in vacuo the material was purified by preparative HPLC. The product fractions were added to ethyl acetate (500 mL) and solid Na₂CO₃ (3 g) was added. The organic layer was separated, washed with NaCl_{(sat.)}(50 mL), was dried over MgSO₄, filtered and the volatiles were removed in vacuo to yield N-(6-bromo-5-methylimidazo[1,2-a]pyridin-2-yl)acetamide. LC/MS (m/z): 267.9 (MH⁺), Rₜ: 1.42 min; HPLC Rₜ: 1.73 min.

According to Method 29, following compounds were prepared from the corresponding trifluoroacetamides:

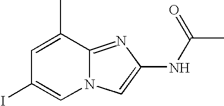

N-(6-iodo-8-methylimidazo[1,2-a]pyridin-2-yl)acetamide from 2,2,2-trifluoro-N-(6-iodo-8-methylimidazo[1,2-a]pyridin-2-yl)acetamide. LC/MS (m/z): 316.0 (MH⁺), Rₜ: 1.53 min;

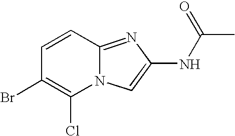

N-(6-bromo-5-chloroimidazo[1,2-a]pyridin-2-yl)acetamide from N-(6-bromo-5-chloroimidazo[1,2-a]pyridin-2-yl)-2,2,2-trifluoroacetamide. LC/MS (m/z): 289.9 (MH⁺), Rₜ: 1.29 min; HPLC Rₜ: 1.07 min.

### Method 30

### Preparation of N-(6-bromo-8-fluoroimidazo[1,2-a]pyridin-2-yl)acetamide

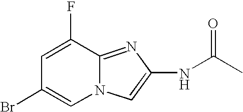

A solution of 5-bromo-3-fluoropyridin-2-amine (1.0 g, 5.24 mmol) and N-acetyl-2-bromoacetamide (1.4 g, 7.85 mmol) in hexamethylphosphoramide (5 mL) was heated at 100° C. overnight. After cooling to RT, water (35 mL) was added. The solid was filtered from the mixture and dried under a flow of air for 4 h to yield a brown powder (616 mg, 43%). LC/MS (m/z): 273.9 (MH⁺), Rₜ: 2.01 min; HPLC Rₜ: 2.34 min.

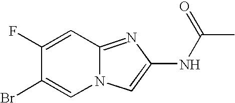

N-(6-bromo-7-fluoroH-imidazo[1,2-a]pyridin-2-yl)acetamide was prepared according to Method 30. LC/MS (m/z): 273.0 (MH⁺), Rₜ: 1.74 min.

### Method 31

### Preparation of tert-butyl 4-(5-(6-iodoimidazo[1,2-a]pyridin-2-ylcarbamoyl)pyridin-2-yl)piperazine-1-carboxylate

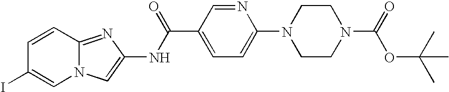

According to Method 11, 6-fluoro-N-(6-iodoimidazo[1,2-a]pyridin-2-yl)nicotinamide was prepared from 6-iodoimidazo[1,2-a]pyridin-2-amine and 6-fluoronicotinic acid. LC/MS (m/z): 383.0 (MH⁺), Rₜ: 2.11 min; HPLC Rₜ: 2.41 min.

A solution of 6-fluoro-N-(6-iodoimidazo[1,2-a]pyridin-2-yl)nicotinamide (35 mg, 0.092 mmol) and tert-butyl piperazine-1-carboxylate (64 mg, 0.34 mmol) in acetonitrile (1 mL) was stirred for 2 days at rt. The crude was concentrated and used in the next step without further purification. LC/MS (m/z): 549.1 (MH⁺), Rₜ: 2.42 min; HPLC Rₜ: 2.83 min.

### Method 32

### Preparation of tert-butyl 2-(3-(6-chloroimidazo[1,2-b]pyridazin-2-ylamino)-3-oxopropyl)piperidine-1-carboxylate

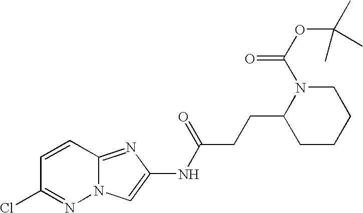

To a solution of 6-chloroimidazo[1,2-b]pyridazin-2-amine (250 mg, 1.48 mmol) and 3-(1-(tert-butoxycarbonyl)piperidin-2-yl)propanoic acid (571 mg, 2.22 mmol) in 30 mL DCM was added HATU (620 mg, 1.63 mmol) and DIEA (0.772 mL, 4.44 mmol). After stirring overnight, DCM (50 mL) was added and the solution was washed with water (2×60 mL), sat. sodium bicarbonate (40 mL), and brine (40 mL). The solution was dried over sodium sulfate, concentrated under vacuum and used in the next step without further purification (509 mg, 84%). LC/MS (m/z): 408.2 (MH⁺), Rₜ: 3.06 min; HPLC Rₜ: 4.07 min.

### Method 33

### Preparation of N-(6-chloroimidazo[1,2-b]pyridazin-2-yl)-3-(1-ethylpiperidin-2-yl)propanamide

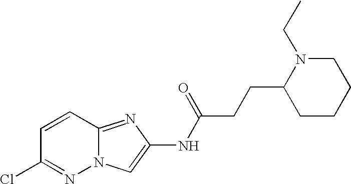

N-(6-chloroimidazo[1,2-b]pyridazin-2-yl)-3-(piperidin-2-yl)propanamide was prepared from tert-butyl 2-(3-(6-chloroimidazo[1,2-b]pyridazin-2-ylamino)-3-oxopropyl)piperidine-1-carboxylate using TFA/DCM. LC/MS (m/z): 308.0 (MH⁺), Rₜ: 1.81 min; HPLC Rₜ: 1.86 min.

N-(6-chloroimidazo[1,2-b]pyridazin-2-yl)-3-(piperidin-2-yl)propanamide was treated with acetic acid and acetaldehyde in methanol, followed by sodium cyanoborohydride to give N-(6-chloroimidazo[1,2-b]pyridazin-2-yl)-3-(1-ethylpiperidin-2-yl)propanamide. LC/MS (m/z): 336.1 (MH⁺), Rₜ: 1.88 min; HPLC Rₜ: 1.98 min.

### Method 34

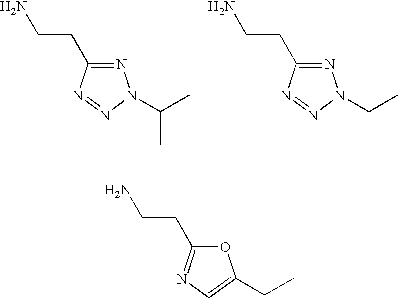

These compounds, namely:
2-(2-Isopropyl-2H-tetrazol-5-yl)-ethylamine,
2-(2-Ethyl-2H-tetrazol-5-yl)-ethylamine and
2-(5-Ethyl-oxazol-2-yl)-ethylamine were prepared according to Bloomfield, Graham Charles; Bruce, Ian; Hayler, Judy; Leblanc, Catherine; Le Grand, Darren Mark; McCarthy, Clive. Preparation of phenylthiazolylureas as inhibitors of phosphatidylinositol 3-kinase. PCT Int. Appl. (2005), 88 pp. WO 2005021519.

### Method 35

### Synthesis of 2-(5-cyclopropyl-tetrazol-2-yl)-ethylamine

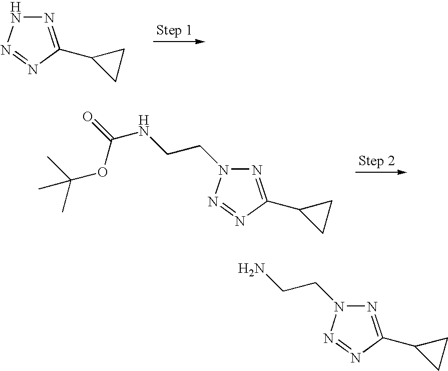

### Step 1: [2-(5-Cyclopropyl-tetrazol-2-yl)-ethyl]-carbamic acid tert-butyl ester

5-Cyclopropyl-2H-tetrazole (0.5 g, 4.5 mmol) was dissolved in dry acetonitrile (7 ml) and triethylamine (9.5 ml, 68 mmol). The reaction mixture was stirred for 10 minutes at room temperature then 2-(Boc-amino)ethyl bromide was added and the mixture was heated to reflux 3 hours. The reaction mixture was partitioned between water and EtOAc and the organic extract was dried (MgSO₄) and concentrated in vacuo. Purification by column chromatography on a 100 g Jones silica cartridge eluting with 50% EtOAc: iso-hexanes afforded the title compound as a colourless oil.

### Step 2: 2-(5-Cyclopropyl-tetrazol-2-yl)-ethylamine

[2-(5-Cyclopropyl-tetrazol-2-yl)-ethyl]-carbamic acid tert-butyl ester (0.42 g, 1.65 mmol) was dissolved in CH₂Cl₂ (3 mL) and 4M HCl in 1,4-dioxane (2 mL) was added. The reaction mixture was stirred at room temperature overnight. The resulting precipitate was filtered and dried under vacuum overnight at 30° C. to afford the title compound as the HCl salt.

### Method 36

### Synthesis of 2-(5-Ethyl-tetrazol-2-yl)-ethylamine

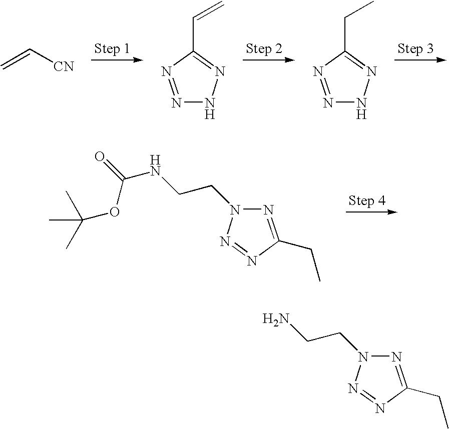

### Step 1: 5-Vinyl-2H-tetrazole

AlCl₃ (3.3 g, 25 mmol) was placed in an oven-dried flask under an atmosphere of Argon. 50 mL of dry THF was slowly added followed by the slow addition of NaN₃ (6.4 g, 99 mmol) and finally acrylonitrile (1.32 g, 25 mmol). The reaction mixture was heated at reflux for 2 hours, allowed to cool to room temperature and then treated with 15% HCl (40 mL) whilst Argon was bubbled through the solution for 5 minutes. The reaction mixture was partitioned between EtOAc and water, the organic portion was washed with brine, dried (MgSO₄), and concentrated in vacuo. Purification by recrystallisation (CHCl₃) afforded the title compound.

### Step 2: 5-Ethyl-2H-tetrazole

A solution of 5-vinyl-2H-tetrazole (1.2 g, 12.5 mmol) in MeOH under an atmosphere of Argon was treated with a catalytic amount of 10% palladium on carbon and the flask was purged with Hydrogen. The reaction mixture was stirred at room temperature for 1 hour and then filtered through a celite (filter agent) plug. The solvent was removed in vacuo to afford the title compound.

### Step 3: [2-(5-Ethyl-tetrazol-2-yl)-ethyl]-carbamic acid tert-butyl ester

This compound was prepared analogously to [2-(5-cyclopropyl-tetrazol-2-yl)-ethyl]-carbamic acid tert-butyl ester (Method 35 step 1) by replacing 5-cyclopropyl-2H-tetrazole with 5-ethyl-2H-tetrazole. Purification by column chromatography on a 100 g Jones silica cartridge eluting with 0 to 4% MeOH:CH₂Cl₂ afforded the title compound as a colourless oil.

### Step 4: 2-(5-Ethyl-tetrazol-2-yl)-ethylamine

This compound was prepared analogously to 2-(5-cyclopropyl-tetrazol-2-yl)-ethylamine (Method 35 step 2) by replacing [2-(5-cyclopropyl-tetrazol-2-yl)-ethyl]-carbamic acid tert-butyl ester with [2-(5-ethyl-tetrazol-2-yl)-ethyl]-carbamic acid tert-butyl ester to afford the title compound as the HCl salt.

### Method 37

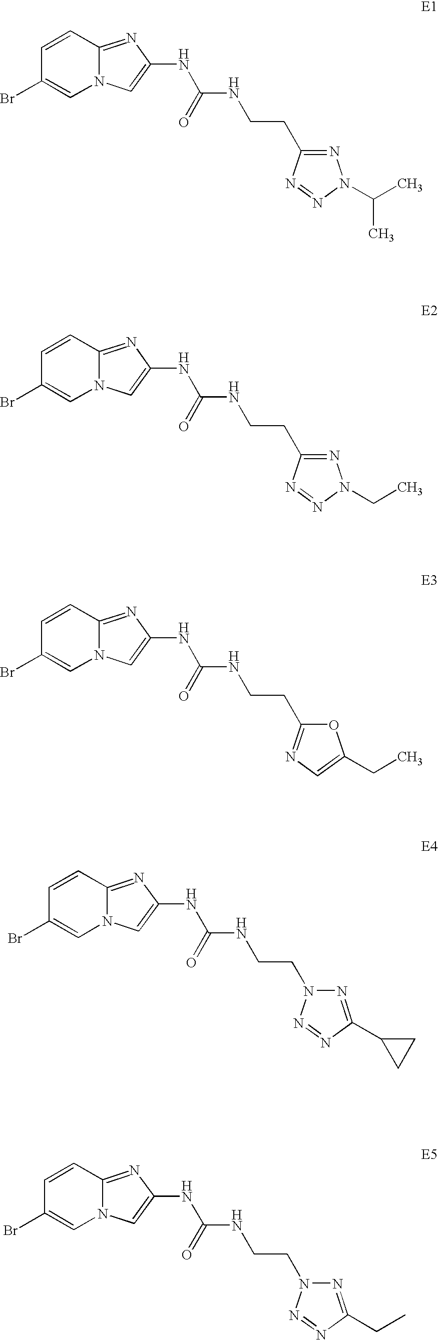

### Intermediate E1

### 1-(6-Bromo-imidazo[1,2-a]pyridin-2-yl)-3-[2-(2-isopropyl-2H-tetrazol-5-yl)-ethyl]-urea

Triethylamine (0.15 ml, 1.1 mmol) was added to a stirred mixture of (6-bromo-imidazo[1,2-a]pyridin-2-yl)-carbamic acid phenyl ester (Method 8) (0.30 g, 0.90 mmol) and 2-(2-isopropyl-2H-tetrazol-5-yl)-ethylamine hydrochloride (Method 34) (0.207 g, 1.1 mmol) in NMP (3 ml). The reaction was stirred at 80° C. for 2 hours. The cooled mixture was diluted with water (100 ml) and the resulting suspension was filtered and dried in a vacuum oven to afford the title compound. LC/MS (m/z): 395.1 (MH⁺).

### Intermediates E2-E5

Theses intermediates namely,

E2 1-(6-Bromo-imidazo[1,2-a]pyridin-2-yl)-3-[2-(2-ethyl-2H-tetrazol-5-yl)-ethyl]-urea,
E3: 1-(6-Bromo-imidazo[1,2-a]pyridin-2-yl)-3-{2-[2-(2-fluoro-ethyl)-2H-tetrazol-5-yl]-ethyl}-urea,
E4: 1-(6-Bromo-imidazo[1,2-a]pyridin-2-yl)-3-[2-(5-cyclopropyl-tetrazol-2-yl)-ethyl]-urea, and
E5: 1-(6-Bromo-imidazo[1,2-a]pyridin-2-yl)-3-[2-(5-ethyl-tetrazol-2-yl)-ethyl]-urea were prepared analogously to Intermediate E1 by replacing 2-(2-isopropyl-2H-tetrazol-5-yl)-ethylamine hydrochloride (Method 34) with the appropriate tetrazole or oxazole.

### Method 38

### Preparation of (S)-2-cyano-N-(6-iodoH-imidazo[1,2-a]pyridin-2-yl)pyrrolidine-1-carboxamide

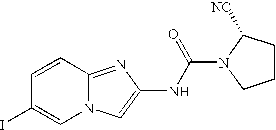

To a solution of 6-iodo-H-imidazo[1,2-a]pyridin-2-amine (260 mg, 1.0 mmol) in THF (10 mL) at 0° C. was added CDI (243 mg, 1.5 mmol). The resulting solution became non-homogeneous and was stirred for 2 h while warming to room temperature. To aid with solubilization DMF was added (1.5 mL), followed by (S)-pyrrolidine-2-carbonitrile hydrochloride (318 mg, 2.4 mmol) and DIEA (0.357 mL, 2 mmol). The reaction mixture was maintained at room temperature for 16 h. The crude reaction mixture was diluted with EtOAc (100 mL) and H₂O (50 mL). The organic layer was separated, and the aqueous phase was extracted with EtOAc (2×75 mL). The combined organic portions were washed with water (2×100 mL) and brine (100 mL), dried over anhydrous Na₂SO₄, filtered, concentrated and dried in vacuo to give (S)-2-cyano-N-(6-iodoH-imidazo[1,2-a]pyridin-2-yl)pyrrolidine-1-carboxamide as a brown solid. The crude product was used for the next step without further purification. LC/MS (m/z): 382.0 (MH⁺), Rₜ: 1.85 min.

### Method 39

### Preparation of methyl 1-(6-iodo-H-imidazo[1,2-a]pyridin-2-ylcarbamoyl)pyrrolidine-3-carboxylate

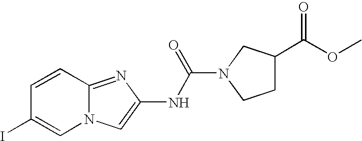

To a solution of 6-iodo-H-imidazo[1,2-a]pyridin-2-amine (260 mg, 1.0 mmol) in THF (10 mL) at 0° C. was added CDI (243 mg, 1.5 mmol). The resulting solution became non-homogeneous and was stirred for 2 h while warming to room temperature. To aid with solubilization DMF was added (1.5 mL), followed by methylpyrrolidine-3-carboxylate hydrochloride (400 mg, 2.4 mmol) and iPr₂NEt (0.357 mL, 2 mmol). The reaction mixture was maintained at room temperature for 16 h. The crude reaction mixture was diluted with EtOAc (100 mL) and H₂O (50 mL). The organic layer was separated, and the aqueous phase was extracted with EtOAc (2×75 mL). The combined organic portions were washed with water (2×100 mL) and brine (100 mL), dried over anhydrous Na₂SO₄, filtered, concentrated and dried in vacuo to give methyl 1-(6-iodoH-imidazo[1,2-a]pyridin-2-ylcarbamoyl)pyrrolidine-3-carboxylate as a brown solid. The crude product was used for the next step without further purification. LC/MS (m/z): 415.0 (MH⁺), Rₜ: 1.89 min.

### Method 40

### Preparation of (S)-benzyl 1-(6-iodoH-imidazo[1,2-a]pyridin-2-ylcarbamoyl)azetidine-2-carboxylate

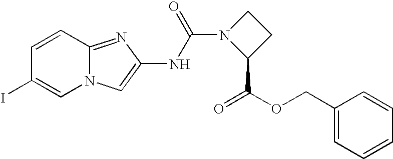

(S)-benzyl azetidine-2-carboxylate: p-Toluenesulfonic acid (228 mg, 1.2 mmol) was added to a stirring mixture of (S)-azetidine-2-carboxylic acid (101 mg, 1.0 mmol) and benzyl alcohol (0.518 mL, 5.0 mmol) in toluene (5 mL). The reaction flask was sealed, then heated for 4 h in an oil bath at 80° C. After cooling to room temperature the crude reaction mixture was used as is in the next step.

To a solution of 6-iodo-H-imidazo[1,2-a]pyridin-2-amine (260 mg, 1.0 mmol) in THF (10 mL) at 0° C. was added CDI (243 mg, 1.5 mmol). The resulting solution became non-homogeneous and was stirred for 2 h while warming to rt. To aid with solubilization, DMF was added (1.5 mL), followed by crude (S)-benzyl azetidine-2-carboxylate and iPr₂NEt (0.446 mL, 2.5 mmol). The reaction mixture was maintained at room temperature for 16 h. The crude reaction mixture was diluted with EtOAc (100 mL) and H₂O (50 mL). The organic layer was separated, and the aqueous phase was extracted with EtOAc (2×75 mL). The combined organic portions were washed with water (3×100 mL) and brine (100 mL), dried over anhydrous Na₂SO₄, filtered, concentrated and dried in vacuo. The crude product was purified by silica gel chromatography, eluting with a gradient of 1:1 hexanes/EtOAc (1×250 mL), 1:2 hexanes/EtOAc (1×250 mL) to give (S)-benzyl 1-(6-iodoH-imidazo[1,2-a]pyridin-2-ylcarbamoyl)azetidine-2-carboxylate. LC/MS (m/z): 477.1 (MH⁺), Rₜ: 2.31 min.

### Method 41

### Preparation of (S)-methyl 1-(6-iodoH-imidazo[1,2-a]pyridin-2-ylcarbamoyl)pyrrolidine-2-carboxylate

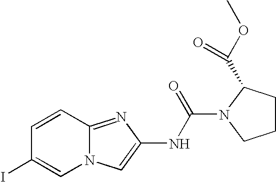

To a solution of 6-iodo-H-imidazo[1,2-a]pyridin-2-amine (260 mg, 1.0 mmol) in THF (10 mL) at 0° C. was added CDI (243 mg, 1.5 mmol). The resulting solution became non-homogeneous and was stirred for 2 h while warming to room temperature. To aid with solubilization, 1.5 mL of DMF were added, followed by (S)-methylpyrrolidine-2-carboxylate hydrochloride (397 mg, 2.4 mmol) and iPr₂NEt (0.357 mL, 2 mmol). The reaction mixture was maintained at room temperature for 16 h. The crude reaction mixture was diluted with EtOAc (100 mL) and H₂O (50 mL). The organic layer was separated, and the aqueous phase was extracted with EtOAc (2×75 mL). The combined organic portions were washed with water (2×100 mL) and brine (100 mL), dried over anhydrous Na₂SO₄, filtered, concentrated and dried in vacuo to give (S)-methyl 1-(6-iodoH-imidazo[1,2-a]pyridin-2-ylcarbamoyl)pyrrolidine-2-carboxylate as a brown solid. The crude product was used for the next step without further purification. LC/MS (m/z): 382.0 (MH⁺), Rₜ: 1.85 min.

### Method 42

### Preparation of (S)-tert-butyl 2-(6-iodoH-imidazo[1,2-a]pyridin-2-ylcarbamoyl)azetidine-1-carboxylate

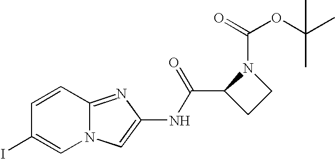

DIC (0.172 mL, 1.1 mmol) was added to a stirring solution of 6-iodo-H-imidazo[1,2-a]pyridin-2-amine (260 mg, 1.0 mmol) and (S)-1-(tert-butoxycarbonyl)azetidine-2-carboxylic acid (201 mg, 1 mmol) in CH₂Cl₂. The reaction was maintained at room temperature for 16 h. The crude reaction mixture was diluted with CH₂Cl₂ (50 mL) and H₂O (30 mL). The organic layer was separated, and the aqueous phase was extracted with CH₂Cl₂ (2×50 mL). The combined organic extracts were washed with brine (80 mL), dried over anhydrous Na₂SO₄, filtered, concentrated and dried in vacuo to give (S)-tert-butyl 2-(6-iodoH-imidazo[1,2-a]pyridin-2-ylcarbamoyl)azetidine-1-carboxylate as a brown solid. The crude product was used for the next step without further purification. LC/MS (m/z): 443.0 (MH⁺), Rₜ: 2.33 min.

### Method 43

### Preparation of (S)-tert-butyl 2-(6-iodoH-imidazo[1,2-a]pyridin-2-ylcarbamoyl)piperidine-1-carboxylate

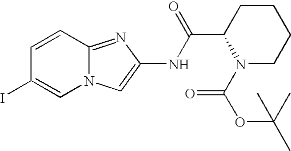

DIC (0.172 mL, 1.1 mmol) was added to a stirring solution of 6-iodo-H-imidazo[1,2-a]pyridin-2-amine (260 mg, 1.0 mmol) and (S)-1-(tert-butoxycarbonyl)piperidine-2-carboxylic acid (254 mg, 1.1 mmol) in CH₂Cl₂. The reaction mixture was maintained at room temperature for 16 h. The crude reaction mixture was diluted with CH₂Cl₂ (50 mL) and H₂O (30 mL). The organic layer was separated, and the aqueous phase was extracted with CH₂Cl₂ (2×50 mL). The combined organic extracts were washed with brine (80 mL), dried over anhydrous Na₂SO₄, filtered, concentrated and dried in vacuo to give (S)-tert-butyl 2-(6-iodoH-imidazo[1,2-a]pyridin-2-ylcarbamoyl)piperidine-1-carboxylate as a brown solid. The crude product was used for the next step without further purification. LC/MS (m/z): 471.1 (MH⁺), Rₜ: 2.68 min.

### Method 44

### Preparation of N-(6-iodoimidazo[1,2-a]pyridin-2-yl)-2-(2-methoxyphenyl)pyrrolidine-1-carboxamide

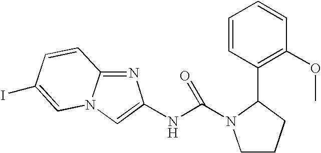

A solution of 6-iodoimidazo[1,2-a]pyridin-2-amine (0.05 g, 0.19 mmol), DIEA (0.50 mL, 0.29 mmol), and CDI (60 mg, 0.31 mmol) in THF (3 mL) was stirred overnight, then 2-(2-methoxyphenyl)pyrrolidine (34 mg, 0.19 mmol) was added. After stirring for 5 h at room temperature, the reaction mixture was concentrated to yield N-(6-iodoimidazo[1,2-a]pyridin-2-yl)-2-(2-methoxyphenyl)pyrrolidine-1-carboxamide (LC/MS (m/z): 462.9 (MH⁺), Rₜ: 2.38 min. The crude product was used for next step without further purification.

According to Method 44, the following compounds were prepared from 6-iodoimidazo[1,2-a]pyridin-2-amine and the corresponding amines:

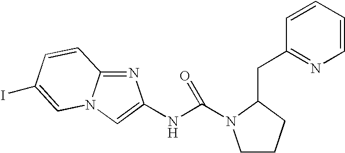

N-(6-iodoimidazo[1,2-a]pyridin-2-yl)-2-(pyridin-2-ylmethyl)pyrrolidine-1-carboxamide from 2-(pyrrolidin-2-ylmethyl)pyridine. (LC/MS (m/z): 448.0 (MH⁺), Rₜ: 1.65 min.

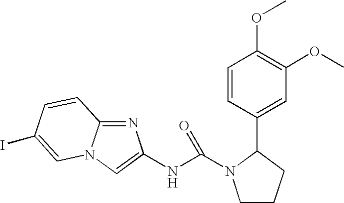

2-(3,4-dimethoxyphenyl)-N-(6-iodoimidazo[1,2-a]pyridin-2-yl)pyrrolidine-1-carboxamide from 2-(3,4-dimethoxyphenyl)pyrrolidine. (LC/MS (m/z): 493.0 (MH⁺), Rₜ: 2.18 min.

### Method 45

### Preparation of tert-butyl 6-iodo-H-imidazo[1,2-a]pyridin-2-ylcarbamate

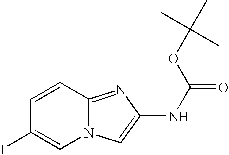

A flame dried round-bottom flask under nitrogen at room temperature was charged with 6-iodoimidazo[1,2-a]pyridine-2-amine (1.43 g, 5.52 mmol), di-tert-butyldicarbonate (0.84 g, 3.86 mmol) and THF (60 mL). The resulting reaction mixture was refluxed overnight. The reaction mixture was cooled to room temperature, quenched with water and extracted with EtOAc. The organic extracts were washed with brine, dried over sodium sulfate, filtered and concentrated to afford tert-butyl 6-iodo-H-imidazo[1,2-a]pyridin-2-ylcarbamate as an orange oil. LC/MS (m/z): 360.1 (MH⁺).

### Method 46

### Preparation of tert-butyl 6-(6-amino-5-(trifluoromethyl)pyridin-3-yl)H-imidazo[1,2-a]pyridin-2-ylcarbamate

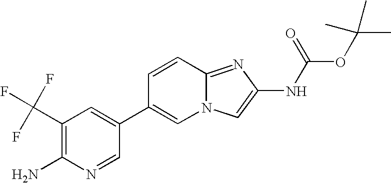

A glass pressure vessel was charged with tert-butyl 6-iodo-H-imidazo[1,2-a]pyridin-2-ylcarbamate (930 mg, 2.59 mmol), 3-(trifluoromethyl)-5-(4,4,5,5-tetramethyl-1,3,2-dioxaborolan-2-yl)pyridin-2-amine (821 mg, 2.85 mmol), sodium carbonate (1.09 g, 10.36 mmol), DME (10 mL), water (5 mL), and Pd(dppf)Cl₂-DCM (106 mg, 0.13 mmol). The reaction mixture was degassed with nitrogen for 10 minutes and the vessel sealed. The reaction mixture was then heated for 15 minutes at 110° C. in an oil bath. The reaction mixture was then cooled to room temperature and water and EtOAc were added. The two phases were separated and the organic phase was washed with water, brine, dried with sodium sulfate, filtered and concentrated to yield tert-butyl 6-(6-amino-5-(trifluoromethyl)pyridin-3-yl)H-imidazo[1,2-a]pyridin-2-ylcarbamate as a dark oil. LC/MS (m/z): 394.1 (MH⁺).

### Method 47

### Preparation of tert-butyl 6-(6-amino-5-(trifluoromethyl)pyridin-3-yl)-3-bromo-H-imidazo[1,2-a]pyridin-2-ylcarbamate

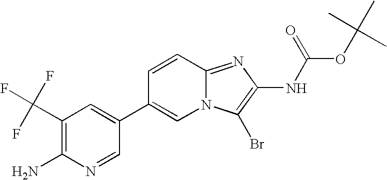

A round-bottom flask was charged with tert-butyl 6-(6-amino-5-(trifluoromethyl)pyridin-3-yl)H-imidazo[1,2-a]pyridin-2-ylcarbamate (768 mg, 1.95 mmol) and acetonitrile (30 mL). A drying tube was placed on the top of the round-bottom flask and the reaction mixture was cooled to 0° C. in an ice bath. To the cold reaction mixture N-bromosuccinimide (416 mg, 2.34 mmol) was added portion wise over two minutes. After stirring for 10 minutes at 0° C., water was added, followed by EtOAc. The two phases were separated and the organic phase was washed with brine, dried with sodium sulfate, filtered and concentrated to a red residue. The residue was then purified via flash chromatography on SiO₂ (acetone/hexanes) to afford tert-butyl 6-(6-amino-5-(trifluoromethyl)pyridin-3-yl)-3-bromoH-imidazo[1,2-a]pyridin-2-ylcarbamate. LC/MS (m/z): 472.1 (MH⁺).

### Method 48

### Preparation of 2-bromo-N-(6-chloroimidazo[1,2-b]pyridazin-2-yl)acetamide

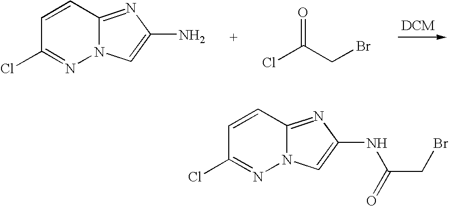

6-Chloroimidazo[1,2-b]pyridazin-2-amine (500 mg, 2.9 mmol) was suspended in DCM (20 mL) and the mixture was cooled down to 0° C. 2-Bromoacetyl chloride (0.27 mL, 3.3 mmol) was then added dropwise under vigorous stirring. The reaction mixture was warmed to room temperature and stirred overnight. Water was added, followed by additional DCM. The two phases were separated and the solvent removed under reduced pressure. The crude product thus obtained was used in the next step without further purification. LCMS (m/z): 290.9 (MH⁺), Rₜ: 2.17 min. ¹H NMR (DMSO-D₆, 300 MHz): δ 11.4 (1H, bs, NH), 8.28 (1H, s), 8.02 (1H, d, J=8.9 Hz), 7.34 (1H, d, J=8.9 Hz), 4.32 (2H, s).

### Method 49

### Preparation of (S)-tert-butyl3-(2-(6-chloroimidazo[1,2-b]pyridazin-2-ylamino)-2-oxoethoxy)pyrrolidine-1-carboxylate

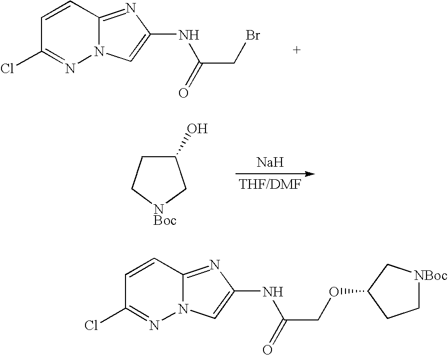

A suspension of NaH (22 mg, 0.55 mmol) in THF (2 mL), in a round bottom flask flame dried and under N₂, was cooled down to 0° C. A mixture of (S)-tert-butyl 3-hydroxypyrrolidine-1-carboxylate (62 mg, 0.33 mmol) and 2-bromo-N-(6-chloroimidazo[1,2-b]pyridazin-2-yl)acetamide (80 mg, 0.28 mmol) in DMF/THF (1:1, 2 mL) was added dropwise. The reaction mixture turned yellow and then dark orange. The reaction mixture was stirred at room temperature then carefully quenched with water, followed by 1N HCl, dropwise, until neutral pH. EtOAc was added, the two phases were separated and the aqueous phase was extracted with EtOAc. The organic extracts were combined, washed with water (1×), brine (1×) and dried (Na₂SO₄). The solvent was removed under reduced pressure and the crude product thus obtained was used in the next step without further purification. LC/MS (m/z): 396.1 (MH⁺), Rₜ: 3.70 min.

The following compound was prepared according to Method 49:

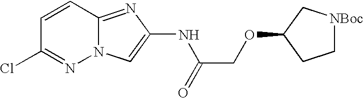

### (R)-tert-Butyl3-(2-(6-chloroimidazo[1,2-b]pyridazin-2-ylamino)-2-oxoethoxy)pyrrolidine-1-carboxylate. LC/MS (m/z): 396.1 (MH⁺), Rₜ: 3.70 min.

### Method 50

### Preparation of (R)-tert-butyl 2-(6-chloroimidazo[1,2-b]pyridazin-2-ylcarbamoyl)pyrrolidine-1-carboxylate

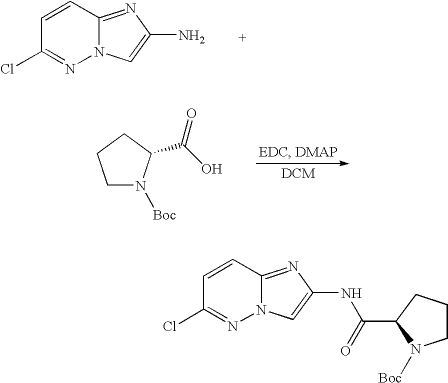

EDC (114 mg, 0.6 mmol) was added to a mixture of 6-chloroimidazo[1,2-b]pyridazin-2-amine (50 mg, 0.3 mmol), (R)-1-(tert-butoxycarbonyl)pyrrolidine-2-carboxylic acid (77 mg, 0.36 mmol) and DMAP (4 mg, 0.03 mmol) in DCM (2 mL)). The reaction mixture was stirred at room temperature overnight. Water was added, the mixture was diluted with additional DCM and the two phases were separated. The organic extracts were dried (Na₂SO₄) and the solvent was removed under reduced pressure. The crude product thus obtained was used in the next step without further purification. LC/MS (m/z): 366.0 (MH⁺), Rₜ: 2.58 min.

The following compound was prepared according to Method 50:

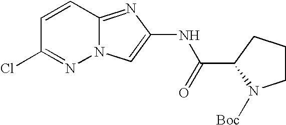

(S)-tert-butyl 2-(6-chloroimidazo[1,2-b]pyridazin-2-ylcarbamoyl)pyrrolidine-1-carboxylate. LC/MS (m/z): 366.0 (MH⁺), Rₜ: 2.58 min

### Method 51

### Preparation of N-(6-(4,4,5,5-tetramethyl-1,3,2-dioxaborolan-2-yl)imidazo[1,2-a]pyridin-2-yl)acetamide

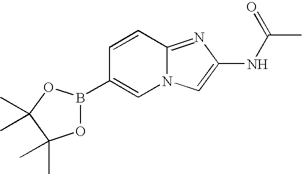

[N-(6-(4,4,5,5-tetramethyl-1,3,2-dioxaborolan-2-yl)imidazo[1,2-a]pyridin-2-yl)acetamide was prepared according to Method 7 from N-(6-iodoimidazo[1,2-a]pyridin-2-yl)acetamide. The crude product was used for the next step without further purification. LC/MS (m/z): 301.9 (MH⁺), Rₜ: 1.64 min.

### Method 52

### Preparation of tert-butyl acetyl-6-iodoH-imidazo[1,2-a]pyridin-2-ylcarbamate

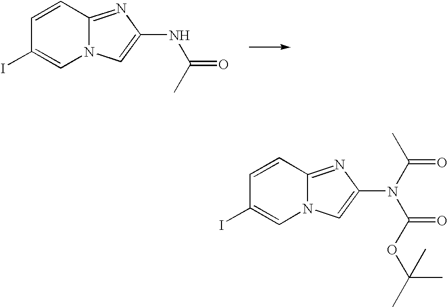

A flame dried round-bottom flask equipped with a stir bar under nitrogen was charged with N-(6-iodoH-imidazo[1,2-a]pyridin-2-yl)acetamide (968 mg, 3.22 mmol), Di(tert)-butyl dicarbonate (1.05 g, 4.82 mmol), 4-(dimethylamine)pyridine (39 mg, 0.322 mmol) and THF (30 mL). The reaction mixture was heated to reflux for 15 minutes, cooled to room temperature and quenched with water. The aqueous mixture was extracted with EtOAc, the organic phases were combined and washed with brine, dried with sodium sulfate, filtered and concentrated to a yellow foam. The crude material was purified by flash chromatography on silica gel (acetone:hexanes) to give tert-butyl-acetyl-6-iodoH-imidazo[1,2-a]pyridin-2-ylcarbamate as a yellow solid. LC/MS (m/z): 402.2 (MH⁺).

### Method 53

### tert-Butyl acetyl(6-(4,4,5,5-tetramethyl-1,3,2-dioxaborolan-2-yl)imidazo[1,2-a]pyridin-2-yl)carbamate

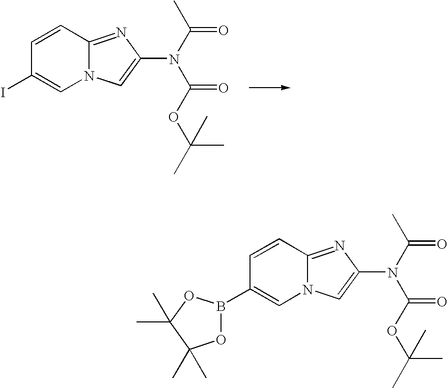

A mixture of tert-butyl acetyl(6-iodoimidazo[1,2-a]pyridin-2-yl)carbamate (2.34 g, 5.8 mmol), bis(pinacolato)diboron (2.17 g, 8.54 mmol), potassium acetate (1.75 g), tricyclohexylphosphine (158 mg, 10 mole %), bis(palladium)tris(dibenzylidene acetone) (261 mg, 5 mole %) and 1,4-dioxane (25 mL), was subjected to four cycles of freeze/pump/thaw to 0.1 mmHg° then sealed in vacuo and immersed into a pre-equilibrated bath at 110° C. for 24 hrs. The system was then cooled to RT. The mixture was diluted (EtOAc) filtered on Celite and concentrated to a red oil (4.5 g). Purification by flash column chromatography on silica gel (100% dichloromethane to 25% acetonitrile in dichloromethane) afforded the desired product (1.7 g, 73%). LC/MS (m/z): 220 (MH⁺), Rₜ: 1.81 min.

### Method 54

### 2-Acetamidoimidazo[1,2-a]pyridin-6-ylboronic acid

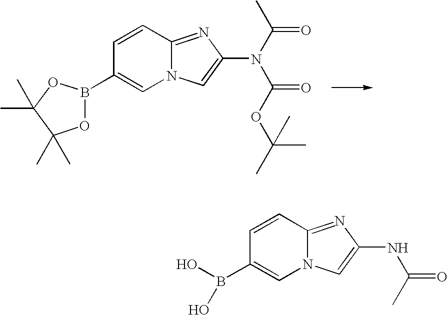

tert-butyl acetyl(6-(4,4,5,5-tetramethyl-1,3,2-dioxaborolan-2-yl)imidazo[1,2-a]pyridin-2-yl)carbamate (1.64 g, 4.0 mmol) was dissolved in trifluoroacetic acid (10 mL) at room temperature. After 25 minutes, the reaction mixture was diluted with anhydrous diethyl ether (100 mL) and cooled to 0° C. The solid thus formed was collected, washed (ether) and air-dried, obtaining the TFA salt of the desired product as white crystals (937 mg, 70%). LC/MS (m/z): 220 (MH⁺).

Boronic Acids/Boronate Esters: Aryl and heteroaryl boronic acids/boronate esters are commercially available or prepared from the corresponding aryl or heteroaryl bromides following general procedures for preparing boronic acids/boronate esters from aryl or heteroaryl halides.

### Method 55

### Synthesis of 5-(4,4,5,5-Tetramethyl-[1,3,2]dioxaborolan-2-yl)-3-trifluoromethyl-pyridin-2-ylamine

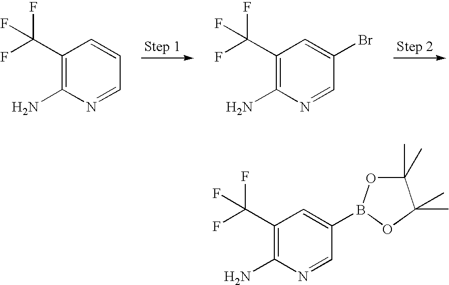

### Step 1: 5-Bromo-3-trifluoromethyl-pyridin-2-ylamine

A solution of 2-amino-3-trifluoromethylpyridine (0.980 g, 5.92 mmol) in CHCl₃ (7 ml) and AcOH (5 ml) was cooled to 0-10° C. (ice-bath) and a solution of bromine in CHCl₃ (0.424 ml, 8.3 mmol) was added carefully dropwise. The reaction was stirred at this temperature for 1 hour then allowed to warm room temperature. The solvent was removed in vacuo and the residue was dissolved in EtOAc. The solution was washed with saturated NaHCO₃, dried over MgSO₄, filtered and concentrated to afford the title compound.

### Step 2: 5-(4,4,5,5-Tetramethyl-[1,3,2]dioxaborolan-2-yl)-3-trifluoromethyl-pyridin-2-ylamine

A mixture comprising 5-bromo-3-trifluoromethyl-pyridin-2-ylamine (step 1) (1.0 g, 4.14 mmol), bis(pinacolato) diboron (1.26 g, 4.98 mmol), Pd(dppf)₂Cl₂ (0.90 g, 0.12 mmol) and potassium acetate (1.14 g, 11.6 mmol) in dry DMF (20 ml) was flushed with Argon and heated using microwave irradiation for 2 hours at 150° C. After cooling to room temperature, the mixture was filtered through celite (filter agent) and concentrated in vacuo to afford a black residue. The residue was dissolved in EtOAc, loaded onto a SCX column (silica based cation exchange sorbent) and washed with an EtOAc (200 ml) 0.35M NH₃ in methanol (200 ml) and concentrated to afford the title compound.

### Method 56

### Preparation of 5-bromo-4-(trifluoromethyl)-2-pyridylamine

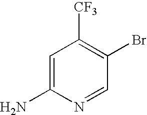

To a solution of 2-amino-4-trifluoromethylpyridine (10.0 g, 62.1 mmol) in chloroform (200 mL) was added NBS (12.0 g, 67.4 mmol). The solution was stirred in the dark for 2 hours, at which time it was added to CH₂Cl₂ (200 mL) and 1N NaOH (200 mL). The layers were separated and the organic layer was washed with NaCl_{(sat.)} (100 mL), dried over Na₂SO₄, filtered and concentrated. The crude material was purified by chromatography on silica gel (0-5% EtOAc/CH₂Cl₂) yielding 12.0 g (80%) of 5-bromo-4-(trifluoromethyl)-2-pyridylamine. LC/MS (m/z): 241/243 (MH⁺); ¹H NMR (CDCl₃, 300 MHz): δ 8.28(s, 1H), 6.77 (s, 1H), 4.78 (bs, 2H).

### Method 57

### Preparation of 5-bromo-3-(trifluoromethyl)-2-pyridylamine

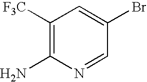

To a solution of 2-amino-3-trifluoromethylpyridine (15.4 g, 95 mmol) in ACN (300 mL) was added NBS (18.6 g, 104 mmol). The solution was stirred in the dark for 6 hours. The solvent was removed and ethyl acetate (500 mL) and water were added. The two phases were separated and the organic layer was washed with NaCl_{(sat.)} (200 mL), dried over Na₂SO₄, filtered and concentrated, yielding 22.8 g (99%) of 5-bromo-3-(trifluoromethyl)-2-pyridylamine which was used in the next step without further purification. LC/MS (m/z): 241/243 (MH⁺); ¹H NMR (CDCl₃, 300 MHz): δ 8.28 (s, 1H), 6.77 (s, 1H), 4.78 (bs, 2H).

According to Method 57, the following bromides were prepared from bromination of the corresponding 2-aminopyridines or 2-aminopyrazines:

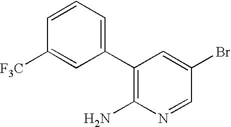

5-Bromo-3-(3-(trifluoromethyl)phenyl)pyridin-2-amine was prepared from 3-(3-(trifluoromethyl)phenyl)pyridin-2-amine. The crude product was used for the next step without further purification. LC/MS (m/z): 318.9 (MH⁺), Rₜ: 2.43 min.

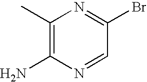

5-Bromo-3-methylpyrazin-2-amine was prepared from crude 3-methylpyrazin-2-amine. The crude product was used for the next step without further purification. LC/MS (m/z): 187.8 (MH⁺), Rₜ: 1.34 min.

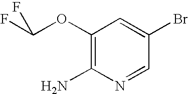

5-Bromo-3-(difluoromethoxy)pyridin-2-amine was prepared from 3-(difluoromethoxy)pyridin-2-amine. The crude product was used for the next step without further purification. LC/MS (m/z): 238.8 (MH⁺), Rₜ: 1.50 min.

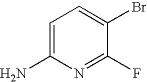

5-Bromo-6-fluoropyridin-2-amine was prepared from 6-fluoropyridin-2-amine. LC/MS (m/z): 190.9 (MH⁺), Rₜ: 2.13 min; HPLC Rₜ: 2.71 min.

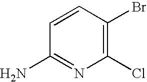

5-Bromo-6-chloropyridin-2-amine was prepared from 6-chloropyridin-2-amine. LC/MS (m/z): 208.9 (MH⁺), Rₜ: 2.26 min; HPLC Rₜ: 2.88 min.

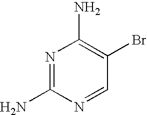

5-Bromopyrimidine-2,4-diamine was prepared from 2,4-diaminopyrimidine. LCMS (m/z): 189/191 (MH⁺). ¹H NMR (DMSO-d₆): δ 7.78 (s, 1H), 6.58 (bs, 2H), 6.08 (bs, 2H).

### Method 58

### Preparation of 5-(4,4,5,5-tetramethyl(1,3,2-dioxaborolan-2-yl))-4-(trifluoromethyl)-2-pyridylamine

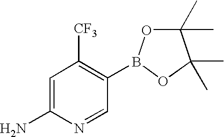

To a dry 500 mL flask was added 5-bromo-4-(trifluoromethyl)-2-pyridylamine (11.8 g, 49.0 mmol), potassium acetate (14.4 g, 146.9 mmol), 4,4,5,5-tetramethyl-2-(4,4,5,5-tetramethyl-1,3,2-dioxaborolan-2-yl)-1,3,2-dioxaborolane (13.6 g, 53.9 mmol) and dioxane (300 mL). Argon was bubbled through the solution for 15 minutes, at which time 1,1′-bis(diphenylphosphino)ferrocene palladium(II) chloride dichloromethane adduct (2.0 g, 2.45 mmol) was added. The reaction was refluxed in a 115° C. oil bath for 8 hours under argon. After cooling to room temperature, the dioxane was removed in vacuo. EtOAc (500 mL) was added, and the resulting slurry was sonicated and filtered. Additional EtOAc (500 mL) was used to wash the solid. The combined organic extracts were concentrated and the crude material was partially purified by SiO₂ chromatography (30-40% EtOAc/Hexanes). Upon removal of solvent, hexanes (75 mL) was added; after sonication, the resulting solid was filtered and dried on a high vacuum for 3 days yielding 2.4 g of an off-white solid. By ¹H NMR the material was a 5:1 mixture of boronate ester and 2-amino-4-trifluoromethylpyridine byproduct. The material was used as is in subsequent Suzuki reactions. LC/MS (m/z): 207 (MH⁺ of boronic acid, deriving from in situ product hydrolysis on LC); ¹H NMR (CDCl₃, 300 MHz): δ 8.50 (s, 1H), 6.72 (s, 1H), 4.80 (bs, 2H), 1.34 (s, 12H).

### Method 59

### Preparation of 5-bromo-4-(trifluoromethyl)pyrimidin-2-amine

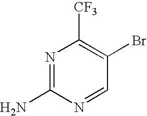

To a solution of 2-amino-4-trifluoromethylpyrimidine (8.0 g, 49.1 mmol) in chloroform (300 mL) was added N-bromosuccinimide (8.9 g, 50 mmol). The solution was stirred in the dark for 16 hours, at which time additional N-bromosuccinimide (4.0 g, 22.5 mmol) was added. After stirring for an additional 4 hours the solution was added to CH₂Cl₂ (200 mL) and 1N NaOH (200 mL). The layers were separated and the organic layer was washed with NaCl_{(sat.)} (100 mL), dried over Na₂SO₄, filtered and concentrated, yielding 10.9 g (82%) of 5-bromo-4-(trifluoromethyl)-2-pyrimidylamine. LC/MS (m/z): 242/244 (MH⁺); ¹H NMR (CDCl₃, 300 MHz): [symbol] 8.52 (s, 1H), 5.38 (bs, 2H).

### Method 60

### Preparation of 5-(4,4,5,5-tetramethyl(1,3,2-dioxaborolan-2-yl))-4-(trifluoromethyl)pyrimidine-2-ylamine

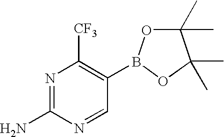

To a dry 500 mL flask was added 5-bromo-4-(trifluoromethyl)-2-pyrimidylamine (10.1 g, 41.7 mmol), potassium acetate (12.3 g, 125.2 mmol), 4,4,5,5-tetramethyl-2-(4,4,5,5-tetramethyl-1,3,2-dioxaborolan-2-yl)-1,3,2-dioxaborolane (11.6 g, 45.9 mmol) and dioxane (150 mL). Argon was bubbled through the solution for 15 minutes, at which time 1,1′-bis(diphenylphosphino)ferrocene palladium (II) chloride (1.7 g, 2.1 mmol) was added. The reaction was refluxed in a 115° C. oil bath for 6 hours under argon. After cooling to room temperature, the dioxane was removed in vacuo. EtOAc (500 mL) was added and the resulting slurry was sonicated and filtered. Additional EtOAc (500 mL) was used to wash the solid. The combined organic extracts were concentrated and the crude material was purified by chromatography on silicagel (30-40% EtOAc/hexanes) yielding 4.40 g of an off white solid. By ¹H NMR the material was a 1:1 mixture of boronate ester and 2-amino-4-trifluoromethylpyrimidine byproduct. The material was used as is in subsequent Suzuki reactions. LC/MS (m/z): 208 (MH⁺ of boronic acid, deriving from in situ product hydrolysis on LC); ¹H NMR (CDCl₃, 300 MHz): δ 8.72 (s, 1H), 5.50 (bs, 2H), 1.34 (s, 12H).

According to Method 60, the following boronic ester was prepared from the corresponding bromide:

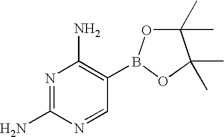

5-(4,4,5,5-Tetramethyl-1,3,2-dioxaborolan-2-yl)pyrimidine-2,4-diamine. LCMS (m/z): 155 (MH⁺ of boronic acid). ¹H NMR (CDCl₃+CD₃OD): δ 8.16 (s, 1H), 1.34 (s, 12H).

### Method 61

### Preparation of 5-bromo-3-methoxy-2-pyridylamine

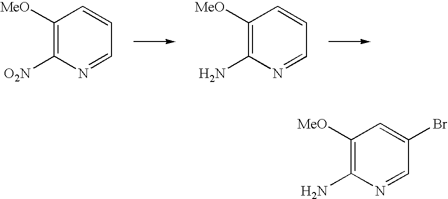

To an argon purged solution of 3-methoxy-2-nitropyridine (462 mg, 3.0 mmol) in ethanol (15 mL) was added 10% Pd on carbon (4.0 mmol). The reaction vessel was placed under slight vacuum and then filled with hydrogen. After stirring overnight, the mixture was purged with argon, filtered and concentrated to give 3-methoxypyridin-2-amine (330 mg, 88%). LC/MS (m/z): 125.0 (MH⁺), Rₜ: 0.33 min.

NBS (8.6 g, 47 mmol) was added to a solution of 2-amino-3-methoxypyridine (6.0 g, 47 mmol) in ACN (200 mL). The solution was stirred in the dark for 6 hours. The solvent was removed and EtOAc (400 mL) and water were added. The two phases were separated and the organic layer was washed with brine (200 mL), dried over Na₂SO₄, filtered and concentrated, yielding 4.5 g (46%) of 5-bromo-3-methoxy-2-pyridylamine. LC/MS (m/z): 203/205 (MH⁺); ¹HNMR (CDCl₃, 300 MHz): δ 8.28 (s, 1H), 6.77 (s, 1H), 4.78 (bs, 2H).

According to Method 61, the following amines were prepared from the corresponding 2-nitropyridines:

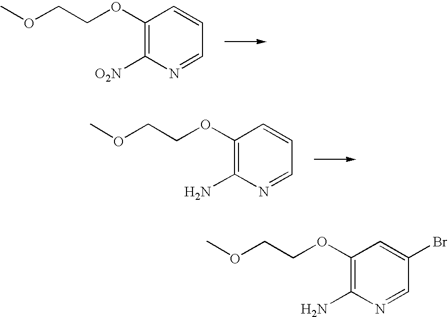

5-Bromo-3-(2-methoxyethoxy)pyridin-2-amine: LC/MS (m/z): 246.9 (MH⁺), Rₜ: 1.26 min.

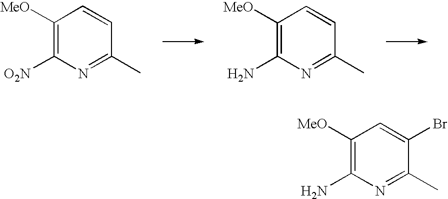

5-Bromo-3-methoxy-6-methylpyridin-2-amine: LC/MS (m/z): 216.9 (MH⁺), Rₜ: 1.30 min.

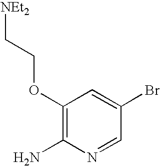

5-Bromo-3-(2-(diethylamino)ethoxy)pyridin-2-amine. LC/MS (m/z): 288.1 (MH⁺), Rₜ: 0.79 min.

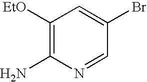

### 5-Bromo-3-ethoxypyridin-2-amine LC/MS (m/z): 216.0/218.0 (MH⁺), Rₜ: 1.51 min

### Method 62

### Alternate Preparation of 3-(2-methoxyethoxy)-2-nitropyridine

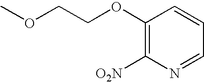

Anhydrous potassium carbonate (2.76 g, 20 mmol) was added to a solution of 2-nitropyridin-3-ol (1.8 g, 13.0 mmol) and 1-bromo-2-methoxyethane (1.47 mL, 16 mmol) in DMF (4 mL) in a microwave reaction vessel. The reaction mixture was then placed to a microwave reactor and heated to 90° C. for 1200 seconds. The reaction mixture was extracted with EtOAc (20 mL). The organic extracts were washed with H₂O (3×20 mL) and brine. The combined organic layers were dried over anhydrous sodium sulfate, filtered, concentrated, and dried in vacuo to give 5-bromo-3-morpholinopyrazin-2-amine as a dark brown oil (540 mg, 21%). The crude product was used for the next step without further purification. LC/MS (m/z): 198.9 (MH⁺), Rₜ: 1.69 min; HPLC Rₜ: 2.26 min.

According to Method 62, the following compound was prepared from a commercially available alkyl halide:

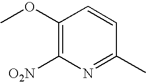

3-Methoxy-6-methyl-2-nitropyridine was prepared from 6-methyl-2-nitropyridin-3-ol and methyl iodide. LC/MS (m/z): 168.9 (MH⁺), Rₜ: 1.80 min.

### Method 63

### Preparation of 5-(4,4,5,5-tetramethyl(1,3,2-dioxaborolan-2-yl))-3-methoxy-2-pyridylamine

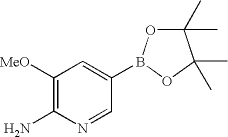

A dry 1 L round bottom flask was charged with 5-bromo-3-methoxy-2-pyridylamine (4 g, 19.7 mmol), potassium acetate (5.8 g, 59 mmol), 4,4,5,5-tetramethyl-2-(4,4,5,5-tetramethyl-1,3,2-dioxaborolan-2-yl)-1,3,2-dioxaborolane (6.5 g, 25.6 mmol) and dioxane (200 mL). Argon was bubbled through the solution for 15 minutes, and 1,1′-bis(diphenylphosphino)ferrocene palladium (II) chloride dichloromethane adduct (0.48 g, 5.9 mmol) was added. The reaction was refluxed at 115° C. for 8 hours under Ar. After cooling to room temperature, the reaction was filtered. EtOAc (400 mL) was used to wash the solid. The combined organics were concentrated and the crude material was purified by silica gel chromatography (50-100% EtOAc in dichloromethane with 0.1% TEA). Upon removal of the solvent, the residue was treated with chloroform (2 mL) and hexanes (150 mL) stirred and sonicated for 30 minutes. The resulting solid was filtered to give the desired boronate ester (1.5 g, 35%). LC/MS (m/z): 167 (MH⁺ of in situ hydrolysis to the acid on LC); ¹H NMR (CDCl₃, 300 MHz): δ 8.55 (s, 1H), 8.07 (s, 1H), 5.24 (bs, 2H), 1.33 (s. 12H). This material contains a UV active byproduct derived from the boronate ester, which can be identified by its CH₃ resonance in the ¹H NMR spectrum (δ=1.26 ppm). This impurity does not affect the subsequent reaction step. The material is therefore used without further purification.

### Method 64

### Preparation of 5-bromo-4-fluoropyridin-2-amine

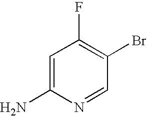

NBS (126 mg, 0.71 mmol) was added to a solution of 4-fluoropyridin-2-amine TFA salt (162 mg, 0.72 mmol) in acetonitrile (4 mL) in an aluminum foil-wrapped flask in a darkened hood. The reaction solution was stirred at room temperature in darkness for 2 hours. After evaporation of the solvent, the crude product was purified on a silica gel column eluting with EtOAc to give 5-bromo-4-fluoropyridin-2-amine as an ivory solid (92 mg, 67%). LC/MS (m/z): 190.9/192.9 (MH⁺), Rₜ: 1.02 minutes.

### Method 65

### Preparation of 4-fluoro-5-(4,4,5,5-tetramethyl-1,3,2-dioxaborolan-2-yl)-pyridin-2-amine

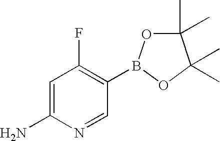

In a sealable Pyrex pressure vessel, a mixture of 5-bromo-4-fluoropyridin-2-amine (25 mg, 0.13 mmol), 4,4,5,5-tetramethyl-2-(4,4,5,5-tetramethyl-1,3,2-dioxaborolan-2-yl)-1,3,2-dioxaborolane (40 mg, 0.16 mmol), potassium acetate (51 mg, 0.52 mmol) and dichloro[1,1′-bis(diphenylphosphino)ferrocene]palladium(II)-dichloromethane adduct (16 mg, 0.019 mmol) was suspended in dioxane (1.7 mL) under argon. The pressure vessel was sealed and the reaction mixture was stirred at 110° C. for 2 hours. After the reaction was complete as judged by LC/MS, the reaction mixture was cooled to room temperature and the 4-fluoro-5-(4,4,5,5-tetramethyl-1,3,2-dioxaborolan-2-yl)pyridin-2-amine was used in subsequent reactions without further purification, assuming a quantitative yield (0.13 mmol). LC/MS (m/z): 157.0 (MH⁺ of the boronic acid formed by product hydrolysis on LC), Rₜ: 0.34 minutes.

### Method 66

### Preparation of 3-fluoro-5-(4,4,5,5-tetramethyl-1,3,2-dioxaborolan-2-yl)pyridin-2-amine

### Synthesis of N-allyl-3-fluoropyridin-2-amine

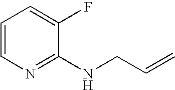

To a preformed bright-yellow complex of Pd(dppf)Cl₂ CH₂Cl₂ (41 mg, 0.05 mmol), dppf (83 mg, 0.15 mmol) and NaOt-Bu (1.4 g, 15 mmol) in THF (20 mL) was added 2-chloro-3-fluoropyridine (1.32 g, 10 mmol) and allylamine (1.2 mL, 15 mmol). The mixture was sparged with nitrogen and the pressure vessel was capped and sealed. The reaction was heated at 65-70° C. for 16 hours. The cooled reaction was filtered through a plug of Celite and the pad was washed with EtOAc (30 mL). The solvent was removed under reduced pressure to give a brown thick oil. The crude product was purified by silica gel chromatography eluting with 5% MeOH in EtOAc. The product-containing fractions were diluted with EtOAc (100 mL) and extracted with 1 M HCl (2×50 mL). The aqueous acidic solution was lyophilized to a light brown solid giving N-allyl-3-fluoropyridin-2-amine as an HCl salt (1.6 g, 85%). LC/MS (m/z): 153.1 (MH⁺), Rₜ 0.5 minutes.

### Synthesis of 3-fluoropyridin-2-amine

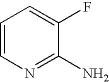

10% Pd/C (1.23 g) was added to a solution of N-allyl-3-fluoropyridin-2-amine (1.62 g, 7.18 mmol) and BF₃.Et₂O (0.9 mL, 7.18 mmol) in EtOH (20 mL) at RT under nitrogen in one portion. After stirring at 80° C. for 2 days, the reaction mixture was filtered through a plug of Celite and the pad was washed with EtOH (20 mL). 6 N HCl was added to the light yellow filtrate until the solution was acidic. The HCl salt of 3-fluoropyridin-2-amine is much less volatile than the free base. The filtrate was concentrated under reduced pressure. The salt residue was dried in vacuo to give 3-fluoropyridin-2-amine as a light yellow glassy solid (1.66 g, quant. yield). LC/MS (m/z): 113.0 (MH⁺), Rₜ 0.41 minutes.

### Synthesis of 5-bromo-3-fluoropyridin-2-amine and 3-fluoro-5-(4,4,5,5-tetramethyl-1,3,2-dioxaborolan-2-yl)pyridin-2-amine

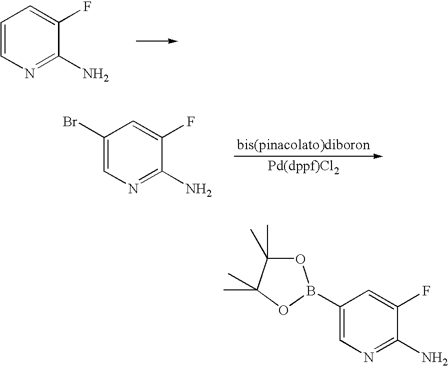

Solid NBS (750 mg, 4.2 mmol) was added to a solution of 3-fluoropyridin-2-amine HCl salt (1.66 g, 7.18 mmol) in ACN (30 mL) at RT with stirring. The reaction was shielded from light and stirred under nitrogen. After 1 h, an additional amount of NBS (250 mg, 1.4 mmol) was added to the reaction. After 1 h, the solvent was removed under reduced pressure and the residue purified by silica gel flash chromatography eluting with 70% EtOAc/hexane followed by 100% EtOAc to afford 5-bromo-3-fluoropyridin-2-amine as a yellow-brown solid (1.26 g, 92% yield). LC/MS (m/z): 191.0/193.0 (MH⁺), Rₜ 1.18 minutes.

The bromide was converted to the pinacolborane ester under conditions described in Method 65. LC/MS (m/z): 157.0 (MH⁺), Rₜ 0.36 minutes.

### Method 67

### Synthesis of 4-fluoro-5-(4,4,5,5-tetramethyl-1,3,2-dioxaborolan-2-yl)pyridin-2-amine N-allyl-4-fluoropyridin-2-amine

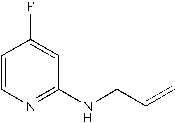

To a red-brown complex of Pd(dppf)Cl₂ (817 mg, 1.0 mmol), dppf (1.66 g, 3.0 mmol) and NaOtBu (2.9 g, 30 mmol) in toluene (30 mL) was added 2-chloro-4-fluoropyridine (2.16 g, 20 mmol) and allylamine (1.2 mL, 15 mmol). The mixture was sparged with nitrogen and the pressure vessel was capped and sealed. The reaction was heated at 120-125° C. for 18 hours. The cooled dark brown reaction was filtered through a plug of Celite and the pad was washed with EtOAc (60 mL). The solvent was gently removed under reduced pressure to give a brown thick oil which can sublime under vacuum. The crude mixture was acidified with 6 N HCl (10 mL) and lyophilized to dryness to give a brown powder as the HCl salt. The crude product was partitioned between EtOAc (100 mL) and sat. NaHCO₃ (80 mL). The layers were separated and the aqueous layer was extracted again with EtOAc (100 mL). The combined organic layers are washed with brine (100 mL), dried over sodium sulfate, filtered and concentrated under reduced pressure to give a brown solid N-allyl-4-fluoropyridin-2-amine (690 mg, 25%). LC/MS (m/z): 153.0 (MH⁺), Rₜ 1.13 minutes.

### Synthesis of 4-fluoropyridin-2-amine

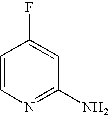

10% Pd/C (552 mg) was added to a solution of N-allyl-4-fluoropyridin-2-amine (690 mg, 3.07 mmol) and BF₃.Et₂O (0.386 mL, 3.07 mmol) in abs. EtOH (12 mL) at RT under nitrogen in one portion. After stirring at 80° C. for 24 h, reaction mixture was filtered through a plug of Celite and the pad was washed with MeOH (100 mL). 6 N HCl (2 mL) was added to the dark filtrate until the solution was acidic. The HCl salt of 4-fluoropyridin-2-amine is much less volatile than the free base. The filtrate was concentrated under reduced pressure and dried in vacuo. The crude product was purified by preparative HPLC to give 4-fluoropyridin-2-amine TFA salt as a brown powder (162 mg, 23%). LC/MS (m/z): 113.0 (MH⁺), Rₜ 0.40 minutes.

### Synthesis of 5-bromo-4-fluoropyridin-2-amine and 4-fluoro-5-(4,4,5,5-tetramethyl-1,3,2-dioxaborolan-2-yl)pyridin-2-amine

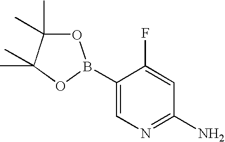

Solid NBS (78 mg, 0.43 mmol) was added to a solution of 3-fluoropyridin-2-amine HCl salt (162 mg, 0.72 mmol) in ACN (4 mL) at RT with stirring. The reaction was shielded from light and stirred under nitrogen. After 1.5 h, an additional amount of NBS (15 mg, 0.084 mmol) was added to the reaction. Checking the reaction again after 1.5 h, an additional amount of NBS (15 mg, 0.084 mmol) was added to the reaction until the starting material had been consumed by LC/MS. After 1 h, the solvent was removed under reduced pressure and the residue purified by silica flash chromatography eluting with 50% ethyl acetate/hexane to afford 5-bromo-4-fluoropyridin-2-amine as a ivory solid (92 mg, 68%). LC/MS (m/z): 190.9/192.9 (MH⁺), Rₜ 1.02 minutes.

The bromide was converted to the pinacolborane under conditions described in Method 65. LC/MS (m/z): 157.0 (MH⁺), Rₜ 0.34 minutes.

### Method 68

### Preparation of 5-bromo-3-fluoropyridin-2-amine

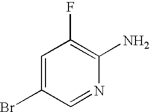

To crude 3-fluoropyridin-2-amine (2.17 g, 19.4 mmol) in acetonitrile (75 mL) was added N-bromosuccinimide (1.38 g, 7.8 mmol). After stirring overnight, the reaction mixture was concentrated to give a residue, which was dissolved in DCM (20 mL). This solution was chilled in the freezer overnight and filtered to yield white crystals (1.0 g, 27%). LC/MS (m/z): 193.0 (MH⁺), Rₜ: 1.33 min.

### Method 69

### Preparation of 3-cyanopyridine-2-amine

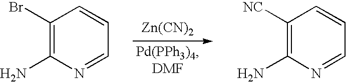

3-Bromopyridine-2-amine (300 mg, 1.73 mmol) was dissolved in DMF (2.5 mL) in a microwave safe vessel, and Zn(CN)₂ (203 mg, 1.73 mmol) was added in one portion. The reaction mixture was purged with N₂ for 5 minutes, then Pd(PPh₃)₄ (100 mg, 0.086 mmol) was added. The vessel was sealed and the reaction mixture was submitted to microwave irradiation at 120° C. for 20 minutes. Water and EtOAc were added to the reaction mixture. The two phases were separated and the aqueous phase was extracted with EtOAc. The organic extracts were combined and washed with water (1×), brine (1×) and dried (Na₂SO₄). The solvent was removed under reduced pressure and the resulting crude 3-cyanopyridine-2-amine was used in the next step without further purification. LC/MS (m/z): 119 (MH⁺), Rₜ: 0.35 min.

### Method 70

### Preparation of 5-bromo-3-cyanopyridine-2-amine

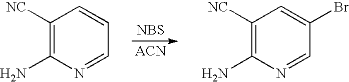

The desired compound was obtained according to the bromination procedure in Method 57 from 3-cyanopyridin-2-amine. Purification by column chromatography on silica gel (20% EtOAc/Hexanes to 50% EtOAc/Hexanes) afforded 5-bromo-3-cyanopyridine-2-amine. LCMS (m/z): 199.0/201.0 (MH⁺), Rₜ: 1.90 min.

### Method 71

### Preparation of 3-(2-methoxyethoxy)-5-bromopyrazin-2-amine

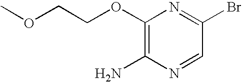

A 100 mL round bottom flask was flame dried under N₂ and cooled to room temperature, then charged with a suspension of 95% NaH (235 mg, 10.3 mmol) in dry THF (40 mL). The mixture was cooled to 0° C. in an ice-water bath and 2-methoxyethanol (0.750 mL, 9.5 mmol) was added dropwise. After stirring at 0° C. for 30 min, 3,5-dibromopyrazin-2-amine (2 g, 7.9 mmol) was added and the reaction stirred while warming to room temperature. The flask was then sealed and heated in a 50° C. oil bath for 16 h. The crude mixture was quenched with water and diluted with EtOAc. The organic layer was separated, and the aqueous phase was extracted with EtOAc (2×100 mL). The combined organic extracts were washed with brine (2000 mL), dried over anhydrous Na₂SO₄, filtered, concentrated and dried in vacuo to give 3-(2-methoxyethoxy)-5-bromopyrazin-2-amine. The crude product was used for the next step without further purification. LC/MS (m/z): 250.0 (MH⁺), Rₜ: 1.98 min.

According to Method 71, the following compounds were prepared from commercially available alcohols and 3,5-dibromopyrazin-2-amine:

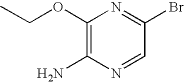

5-Bromo-3-ethoxypyrazin-2-amine was prepared from ethanol. LC/MS (m/z): 217.8 (MH⁺), Rₜ: 1.94 min.

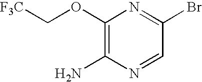

3-(2,2,2-Trifluoroethoxy)-5-bromopyrazin-2-amine from 2,2,2-trifluoroethanol, LC/MS (m/z): 274.0 (MH⁺), Rₜ: 2.64 min.

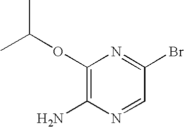

5-Bromo-3-isopropoxypyrazin-2-amine was prepared from isopropanol. LC/MS (m/z): 231.9 (MH⁺), Rₜ: 2.29 min.

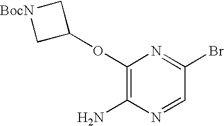

tert-Butyl 3-(3-amino-6-bromopyrazin-2-yloxy)azetidine-1-carboxylate was prepared from tert-butyl 3-hydroxyazetidine-1-carboxylate. LC/MS (m/z): 344.7 (MH⁺), Rₜ: 2.58 min.

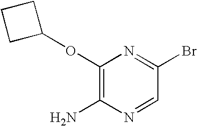

5-Bromo-3-cyclobutoxypyrazin-2-amine was prepared from cyclobutanol. LC/MS (m/z): 244.0 (MH⁺), Rₜ: 2.52 min.

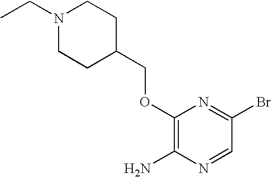

### 5-Bromo-3-((1-ethylpiperidin-4-yl)methoxy)pyrazin-2-amine from 3,5-dibromopyrazin-2-amine and (1-ethylpiperidin-4-yl)methanol. LC/MS (m/z): 381.1 (MH⁺), Rₜ: 2.00 min; HPLC Rₜ: 2.23 min.

### Method 72

### Preparation of 2-bromo-3-(difluoromethoxy)pyridine

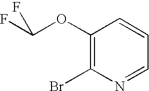

Anhydrous potassium carbonate (1.7 g, 12 mmol) was added to a solution of 2-bromopyridin-3-ol (1.74 g, 10.0 mmol) and sodium difluorochloroacetate (3.0 g, 20 mmol) in DMF (18 mL) and H₂O (2 mL). The reaction mixture was then heated to 100° C. for 2 h, allowed to cool to room temperature and extracted with EtOAc (100 mL). The organic extracts were washed with H₂O (100 mL×3) and brine. The combined organic layer was dried over anhydrous sodium sulfate, filtered, concentrated, and dried in vacuo. The crude was purified by column chromatography to give 2-bromo-3-(difluoromethoxy)pyridine in 44% yields (980 mg). LC/MS (m/z): 223.8 (MH⁺), Rₜ: 2.14 min.

### Method 73

### Preparation of 3-(difluoromethoxy)pyridin-2-amine

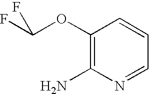

2-Bromo-3-(difluoromethoxy)pyridine (980 mg, 0.044 mol) was suspended in a saturated NH₄OH solution and placed in a high pressure vessel. The reaction mixture was heated to 150° C. (240 psi) for 2 days. The volatile materials were evaporated and the residue dried in vacuo to give crude 3-(difluoromethoxy)pyridin-2-amine (415 mg) containing NH₄Br salt. The crude product was used for the next step without further purification. LC/MS (m/z): 160.9 (MH⁺), Rₜ: 2.16 min.

### Method 74

### Preparation of 3-methylpyrazin-2-amine

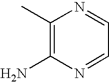

The 2-chloro-3-methylpyrazine (1.5 g, 0.012 mol) was suspended in a saturated NH₄OH solution and placed in a high-pressure vessel. The reaction mixture was heated to 150° C. (200 psi) for 3 days. The white solid was filtered, washed with excess amount of water, and dried in vacuo to give crude 3-methylpyrazin-2-amine in 66% yield (0.84 g). The crude product was used for the next step without further purification. LC/MS (m/z): 110.0 (MH⁺), Rₜ: 0.43 min.

### Method 75

### Preparation of 5-bromo-3-morpholinopyrazin-2-amine

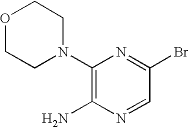

To a solution of 3,5-dibromopyrazin-2-amine (0.5 g, 2.0 mmol) in NMP (6 mL) was added anhydrous cesium carbonate (1.5 g, 5.0 mmol). The reaction mixture was then heated to 85° C. for 15 h. The reaction mixture was extracted with EtOAc (20 mL) and the combined organic extacts were washed with H₂O (20 mL×3) brine, dried over anhydrous sodium sulfate, filtered, concentrated, and dried in vacuo to give crude 5-bromo-3-morpholinopyrazin-2-amine as a brown solid (370 mg, 71%). LC/MS (m/z): 259.0 (MH⁺), Rₜ: 1.89 min.

According to Method 75, the following compound was prepared from commercially available amine:

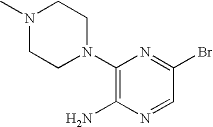

### 5-Bromo-3-(4-methylpiperazin-1-yl)pyrazin-2-amine was prepared 1-methylpiperazine. LC/MS (m/z): 271.6 (MH⁺), Rₜ: 1.25 min.

### Method 76

### Preparation of 3-(azetidin-3-yloxy)-5-bromopyrazin-2-amine

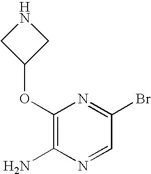

30% TFA in DCM (4 mL) was added to tert-butyl 3-(3-amino-6-bromopyrazin-2-yloxy)azetidine-1-carboxylate (169 mg, 0.49 mmol, prepared as in Method 71). After 45 min, the solution was concentrated in vacuo resulting in an amber oil. LC/MS (m/z): 247.0 (MH⁺), Rₜ: 1.28 min; HPLC Rₜ: 1.23 min.

### Method 77

### Preparation of (3-(3-amino-6-bromopyrazin-2-yloxy)azetidin-1-yl)(phenyl)methanone

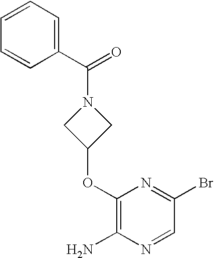

Benzoic anhydride (600 mg, 2.25 mmol) was added to a solution of 3-(azetidin-3-yloxy)-5-bromopyrazin-2-amine (60 mg, 0.25 mmol) in DCM (50 mL). After stirring overnight, the reaction mixture was concentrated in vacuo and dissolved in EtOAc (30 mL). The Organic solution was washed with sat. sodium bicarbonate (20 mL), extracted with 1M HCl (2×20 mL). The acidic aqueous extracts were collected, basified with sodium bicarbonate and extracted with EtOAc (2×20 mL). The organic solution was washed with brine, dried over sodium sulfate and concentrated in vacuo yielding an off white solid (87 mg, 99%). LC/MS (m/z): 349.1 (MH⁺), Rₜ: 2.43 min; HPLC Rₜ: 3.02 min.

### Method 78

### Preparation of tert-butyl 4-(3-amino-6-bromopyrazin-2-yloxy)piperidine-1-carboxylate

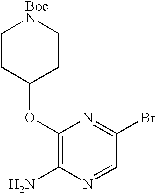

Sodium hydride in mineral oil (60%, 180 mg, 4.5 mmol) was suspended in THF (10 mL). Tert-butyl 4-hydroxypiperidine-1-carboxylate (754 mg, 3.75 mmol) was added. The reaction mixture was stirred for 1 h at room temperature, then 3,5-dibromopyrazin-2-amine (949 mg, 3.75 mmol) was added. After stirring for 5 days and the addition of more sodium hydride (180 mg, 4.5 mmol) in two portions, the reaction mixture was concentrated, cooled to 0° C., diluted with EtOAc (40 mL), quenched and washed with water (2×30 mL). The organic layers were separated and washed with sat. NaCl (30 mL), dried over sodium sulfate and concentrated in vacuo to give a dark oil. Purification by column chromatography on silica gel (15-40% EtOAc in Hexane) yielded the title compound (318 mg, 23%). LC/MS (m/z): 375.1 (MH⁺), Rₜ: 3.02 min.

### Method 79

### Preparation of 5-bromo-3-phenoxypyrazin-2-amine

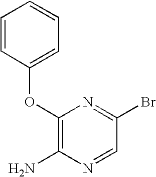

A mixture of 3,5-dibromopyrazin-2-amine (200 mg, 0.79 mmol), phenol (89 mg, 0.95 mmol) and potassium carbonate (273 mg, 1.98 mmol) in NMP (2 mL) was heated at 150° C. for 10 min in a microwave reactor. The reaction mixture was filtered, purified by reverse phase preparative HPLC and then lyophilized to give the desired product (130 mg, 62%). LC/MS (m/z): 268.0 (MH⁺), Rₜ: 2.66 min.

According to Method 79, the following compounds were prepared from commercially available substituted phenols:

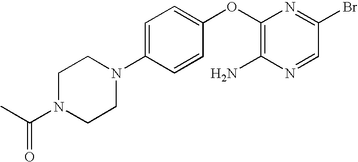

1-(4-(4-(3-Amino-6-bromopyrazin-2-yloxy)phenyl)piperazin-1-yl)ethanone from 1-(4-(4-hydroxyphenyl)piperazin-1-yl)ethanone. LC/MS (m/z): 392.1 (MH⁺), Rₜ: 2.16 min.

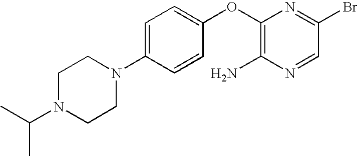

5-Bromo-3-(4-(4-isopropylpiperazin-1-yl)phenoxy)pyrazin-2-amine from 4-(4-isopropylpiperazin-1-yl)phenol. LC/MS (m/z): 394.1 (MH⁺), Rₜ: 2.01 min.

### Method 80

### Preparation of 3-((dimethylamino)methyl)pyridin-2-amine

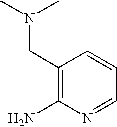

To 2-aminopyridine-3-carbaldehyde (500 mg, 4.1 mmol) in dichloromethane (6 mL) was added dimethylamine (4.1 mL, 2M in ethanol, 8.2 mmol), followed by glacial acetic acid (3 mL). After stirring for 30 min, borane-pyridine (0.414 mL, 4.1 mmol) was added. After five hours at room temperature, the reaction mixture was treated with a saturated sodium bicarbonate solution. The aqueous layer was extracted with dichloromethane (10 mL). The combined organic layers were washed with brine, dried with sodium sulfate and concentrated to give crude 3-((dimethylamino)methyl)pyridin-2-amine, which was used for the next step without further purification. LC/MS (m/z): 152.1 (MH⁺), Rₜ: 0.33 min.

### Method 81

### Preparation of 5-bromo-3-((dimethylamino)methyl)pyridin-2-amine

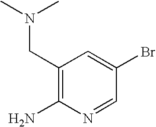

5-Bromo-3-((dimethylamino)methyl)pyridin-2-amine was prepared from 3-((dimethyl-amino)methyl)pyridin-2-amine by NBS bromination according to the procedure outlined in Method 6. LC/MS (m/z): 232.0 (MH⁺), Rₜ: 0.43 min.

### Method 82

### Preparation of 5-bromo-3-(pyrrolidin-1-ylmethyl)pyridin-2-amine

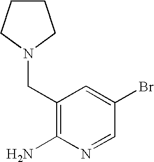

To a solution of pyrrolidine (0.317 mL, 3.8 mmol) in methanol (2 mL) was added acetic acid (0.04 mL), 2-amino-5-bromonicotinaldehyde (500 mg, 2.5 mmol) and sodium cyanoborohydride (138 mg, 2.2 mmol). After stirring overnight, the reaction mixture was concentrated in vacuo, mixed with 10 mL water and extracted twice with 15 mL of EtOAc. The organic phase was extracted twice with 10 mL of 1 M HCl. The combined organic layers were basified with 6 N NaOH, and extracted with EtOAc (2×10 mL). The organic phase was washed with sat NaCl, dried over sodium sulfate, concentrated in vacuo, and used in the next step without further purification. LC/MS (m/z): 255.9 (MH⁺), Rₜ: 0.67 min; HPLC Rₜ: 0.85 min.

### Method 83

### Preparation of methyl 2-amino-5-bromopyridine-3-carboxylate

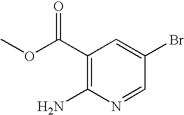

A suspension of 2-amino-5-bromopyridine-3-carboxylic acid (500 mg, 2.3 mmol) in THF (10 mL) was cooled to 0° C. in an ice-water bath. Et₃N (1.92 mL, 13.8 mmol) was added, followed by Me₂SO₄ (0.878 mL, 9.2 mmol). The reaction mixture was maintained at 0° C. for 1 h, allowed to warm to room temperature and stirred for 16 h. The crude reaction mixture was concentrated then diluted with EtOAc (100 mL) and H₂O (50 mL). The organic layer was separated, and the aqueous phase was extracted with EtOAc (2×75 mL). The combined organic extracts were washed with water (2×100 mL) and brine (100 mL), dried over anhydrous Na₂SO₄, filtered, concentrated and dried in vacuo to give methyl 2-amino-5-bromopyridine-3-carboxylate. The crude product was used for the next step without further purification. LC/MS (m/z): 230.9 (MH⁺), Rₜ: 2.03 min.

### Method 84

### Preparation of 2-amino-5-bromo-N-(2-(pyrrolidin-1-yl)ethyl)pyridine-3-carboxamide

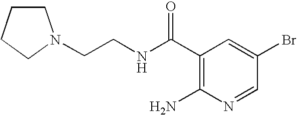

1-(2-Aminoethyl)pyrrolidine (0.264 mL, 2.1 mmol) was added to a stirring solution of 2-amino-5-bromopyridine-3-carboxylic acid (325 mg, 1.5 mmol), iPr₂NEt (0.536 mL, 3.0 mmol), EDC (345 mg, 1.8 mmol), and HOBt (243 mg, 1.8 mmol) in DMF (0.030 mL). The reaction mixture was stirred at room temperature for 16 h then diluted with EtOAc (100 mL) and H₂O (50 mL). The organic layer was separated, and the aqueous phase was extracted with EtOAc (2×75 mL). The combined organic extracts were washed with brine (100 mL), dried over anhydrous Na₂SO₄, filtered, concentrated and dried in vacuo to give 2-amino-5-bromo-N-(2-(pyrrolidin-1-yl)ethyl)pyridine-3-carboxamide. The crude product was used for the next step without further purification. LC/MS (m/z): 315.0 (MH⁺), Rₜ: 0.91 min.

According to Method 84, the following compounds were prepared from the corresponding amines:

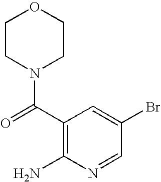

(2-Amino-5-bromopyridin-3-yl)(morpholino)methanone from morpholine. LC/MS (m/z): 285.9, 287.9 (MH⁺), Rₜ: 1.35 min.

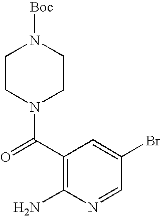

tert-Butyl 4-(2-amino-5-bromonicotinoyl)piperazine-1-carboxylate from Boc-piperazine. LC/MS (m/z): 387.0 (MH⁺), Rₜ: 2.25 min.

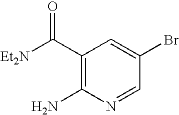

2-Amino-5-bromo-N,N-diethylnicotinamide from dimethylamine. LC/MS (m/z %): 272/274 (M+H), Rₜ: 1.74 min.

### Method 85

### Preparation of 3-(2-methoxyethoxy)-5-(4,4,5,5-tetramethyl-1,3,2-dioxaborolan-2-yl)pyrazin-2-amine

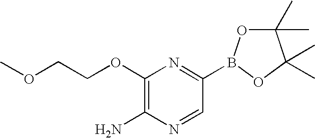

To a solution of 3-(2-methoxyethoxy)-5-bromopyrazin-2-amine (310 mg, 1.25 mmol) in dioxane (5 mL) in a microwave reaction vessel was added bis(pinacolato)diboron (635 mg, 2.5 mmol), Pd(dba)₂ (58 mg, 0.063 mmol), PCy₃ (26 mg, 0.094 mmol) and KOAc (368 mg, 3.75 mmol). The reaction mixture was then heated twice in a microwave reactor at 110° C. for 600 sec. The crude product was used for the next step without workup or further purification. LC/MS (m/z): 214.1/296.1 (MH⁺), Rₜ: 0.70 min.

According to Method 85, the following compounds were prepared from the corresponding bromides:

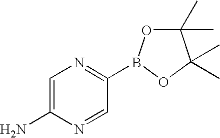

5-(4,4,5,5-tetramethyl-1,3,2-dioxaborolan-2-yl)pyrazin-2-amine. LC/MS (m/z): 140.1 (MH⁺), Rₜ: 0.37 min.

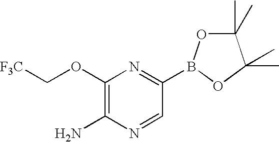

3-(2,2,2-Trifluoroethoxy)-5-(4,4,5,5-tetramethyl-1,3,2-dioxaborolan-2-yl)pyrazin-2-amine LC/MS (m/z): 238.1 (MH⁺), Rₜ: 0.92 min.

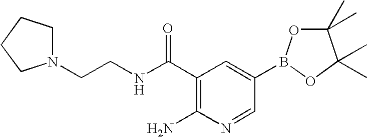

2-Amino-5-(4,4,5,5-tetramethyl-1,3,2-dioxaborolan-2-yl)-N-(2-(pyrrolidin-1-yl)ethyl)pyridine-3-carboxamide. LC/MS (m/z): 279.2 (MH⁺), Rₜ: 0.31 min.

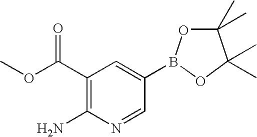

Methyl 2-amino-5-(4,4,5,5-tetramethyl-1,3,2-dioxaborolan-2-yl)pyridine-3-carboxylate. LC/MS (m/z): 197.1 (MH⁺), Rₜ: 0.46 min.

### Method 86

### Preparation of 2-amino-5-bromopyridine-3-sulfonyl chloride

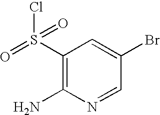

2-Amino-5-bromopyridine-3-sulfonyl chloride (Dorogov, M. V. et. al. Russian patent, RU2263667, (2005)): Chlorosulfonic acid (30 mL) was cooled to −30° C. under nitrogen. 2-Amino-5-bromopyridine (6.0 g, 34.68 mmol) was added slowly under nitrogen flow over 5 minutes. The resulting suspension was refluxed at 200° C. for 4 hr and cooled to room temperature. The reaction mixture was cautiously dripped onto ice/HCl with stirring. The solid was collected, washed with water, air-dried, and dried in-vacuo to give 2-amino-5-bromopyridine-3-sulfonyl chloride (3.36 g, 35.7%).

### Method 87

### Preparation of 3-(4-benzylpiperidin-1-ylsulfonyl)-5-bromopyridin-2-amine

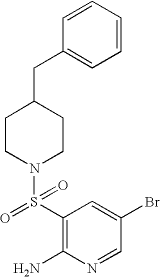

To a mixture of 4-benzyl piperidine (0.25 g, 1.43 mmol) and 2-amino-5-bromopyridine-3-sulfonyl chloride (0.25 g, 0.92 mmol) in pyridine (2 mL) was added DIEA (0.5 mL). The suspension was shaken at room temperature for 14 hr. NaHCO₃ (sat. aq. 1 mL) and ethyl acetate (4 mL) were added and the crystalline product was collected, washed with ether, and air-dried to give 3-(4-benzylpiperidin-1-ylsulfonyl)-5-bromopyridin-2-amine (0.38 g, 60%).

### Method 88

### 3-amino-N-(pyridin-2-yl)propanamide hydroiodide

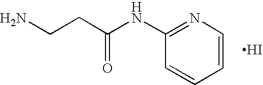

Step 1: [2-(Pyridin-2-ylcarbamoyl)-ethyl]-carbamic acid tert-butyl ester. TEA (2.2 ml, 16 mmol) is added to a stirred solution of BOC-á-alanine (2.4 g, 12.7 mmol), HOAt (0.68 g, 5.0 mmol), EDCI.HCl (2.43 g, 12.7 mmol) in DCM and stirred at room temperature. After 1 hour, 2-aminopyridine (1.0 g, 10.6 mmol) is added and the mixture is stirred at room temperature for a further 3 hours. The mixture is then diluted with DCM (200 ml) and washed with 0.1 M HCl followed by 1M NaOH. The organic portion is dried (MgSO₄) and concentrated in vacuo to afford the title compound as a white crystalline solid (1.78 g, 63%).

Step 2: 3-Amino-N-pyridin-2-yl-propionamide hydroiodide: To a stirred suspension of [2-(pyridin-2-ylcarbamoyl)-ethyl]-carbamic acid tert-butyl ester (1.0 g, 3.8 mmol) in MeCN (20 ml) is added dropwise TMSI (0.65 ml, 4.5 mmol). After 30 minutes, MeOH (1 ml) is added and stirring continued for a further 20 minutes whereupon the product, a yellow crystalline solid, precipitates (1.06 g, 95%).

### Compounds of Formula II

### Example 1

### Preparation of N-(6-(6-amino-5-(trifluoromethyl)pyridin-3-yl)imidazo[1,2-a]pyridin-2-yl)acetamide

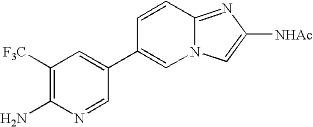

N-(6-iodoimidazo[1,2-a]pyridin-2-yl)acetamide (30.1 mg, 0.1 mmol) and 5-(4,4,5,5-tetramethyl-1,3,2-dioxaborolan-2-yl)-3-(trifluoromethyl)pyridin-2-amine (86.4 mg, 0.3 mmol) was mixed with 2 mL of DME and 2 M Na₂CO₃ aqueous solution (3:1) in the microwave reaction vessel. The reaction mixture was degassed by anhydrous N₂ stream for 15 min followed by the addition of Pd(dppf)C₁₂-DCM (12.2 mg, 0.015 mmol). The reaction mixture was then heated in a microwave reactor at 100° C. for 600 sec. Excess amount of anhydrous Na₂SO₄ was added and the reaction mixture was diluted with EtOAc (3 mL). The organic layer was filtered, concentrated, and dried in vacuo. The crude solid was purified by a preparative HPLC to give N-(6-(6-amino-5-(trifluoromethyl)pyridin-3-yl)imidazo[1,2-a]pyridin-2-yl)acetamide as its TFA salt (7.2 mg, 21%). LC/MS (m/z): 336.1 (MH⁺), Rₜ: 1.59 min; HPLC Rₜ: 1.69 min; ¹H NMR its (free base, DMSO-1,300 MHz) δ 8.90 (m, 1H), 8.55 (d, 1H, J=2.7 Hz), 8.06 (1H, s), 8.02 (d, 1H, J=2.1 Hz), 7.57 (dd, 1H, J=1.8 and 9.3 Hz), 7.54 (d, 1H, J=9.3 Hz), 6.63 (2H, s), 2.08 (s, 3H); ¹³C NMR (free base, DMSO-d⁶, 75 MHz) 167.6, 154.9, 150.1, 142.2, 140.0, 133.0 (2C), 123.6, 122.9, 121.2, 120.5, 119.5, 115.2, 100.7, 22.9.

According to Example 1, the following compounds were prepared from the corresponding commercially available boronic acids or esters:

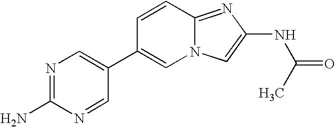

N-(6-(6-aminopyridin-3-yl)imidazo[1,2-a]pyridin-2-yl)acetamide, TFA salt (5.0% yield). LC/MS (m/z): 268.1 (MH⁺), Rₜ: 1.16 min; HPLC Rₜ: 1.16 min

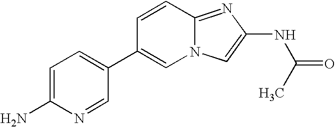

N-(6-(6-fluoropyridin-3-yl)imidazo[1,2-a]pyridin-2-yl)acetamide, TFA salt (9.2% yield). LC/MS (m/z): 271.0 (MH⁺), Rₜ: 1.46 min; HPLC Rₜ: 1.73 min; ¹H NMR (CD₃OD, 300 MHz) δ 8.78 (m, 1H), 8.25 (dd, 1H, J=2.4 and 9.3 Hz), 8.158 (d, 1H, J=2.1 Hz), 7.665 (dd, 1H, J=1.8 and 9.6 Hz), 7.61 (d, 1H, J=9.3 Hz), 7.125 (dd, 1H, J=0.6 and 9.3 Hz), 2.19 (s, 3H).

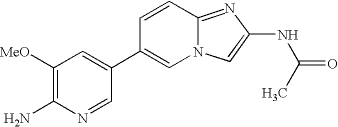

N-(6-(6-amino-5-methoxypyridin-3-yl)imidazo[1,2-a]pyridin-2-yl)acetamide, TFA salt (24.0% yield). LC/MS (m/z): 298.2 (MH⁺), Rₜ: 1.25 min; HPLC Rₜ: 1.33 min; ¹H NMR (DMSO-d⁶, 300 MHz) δ 9.08 (s, 1H), 8.20 (bs, 2H), 8.11 (s, 1H), 7.92 (s, 1H), 7.78 (s, 1H), 7.73 (d, 1H), 7.63 (d, 1H), 4.05 (s, 3H), 2.11 (s, 3H)

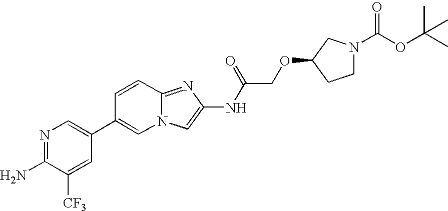

(R)-tert-butyl 3-(2-(6-(6-amino-5-(trifluoromethyl)pyridin-3-yl)imidazo[1,2-a]pyridin-2-ylamino)-2-oxoethoxy)pyrrolidine-1-carboxylate from (R)-tert-butyl 3-(2-(6-iodoimidazo[1,2-a]pyridin-2-ylamino)-2-oxoethoxy)pyrrolidine-1-carboxylate. LC/MS (m/z): 521.2 (MH⁺), Rₜ: 2.36 min; HPLC Rₜ: 2.76 min.

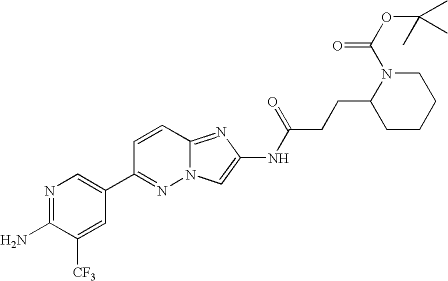

tert-Butyl 2-(3-(6-(6-amino-5-(trifluoromethyl)pyridin-3-yl)imidazo[1,2-b]pyridazin-2-ylamino)-3-oxopropyl)piperidine-1-carboxylate. LC/MS (m/z): 534.1 (MH⁺), Rₜ: 2.89 min; HPLC Rₜ: 3.74 min.

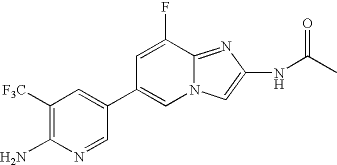

N-(6-(6-amino-5-(trifluoromethyl)pyridin-3-yl)-8-fluoroimidazo[1,2-a]pyridin-2-yl)acetamide. LC/MS (m/z): 354.1 (MH⁺), Rₜ: 1.93 min; HPLC Rₜ: 2.13 min.

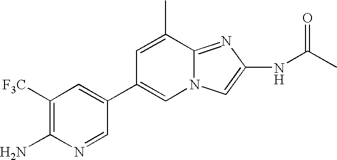

N-(6-(6-amino-5-(trifluoromethyl)pyridin-3-yl)-8-methylimidazo[1,2-a]pyridin-2-yl)acetamide. LC/MS (m/z): 361.2 (MH⁺), Rₜ: 1.98 min; HPLC Rₜ: 2.15 min.

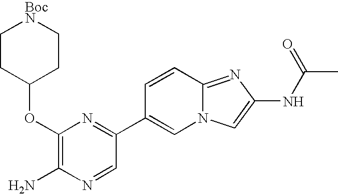

tert-Butyl 4-(6-(2-acetamidoimidazo[1,2-a]pyridin-6-yl)-3-aminopyrazin-2-yloxy)piperidine-1-carboxylate. LC/MS (m/z): 468.3 (MH⁺), Rₜ: 2.16 min; HPLC Rₜ: 2.43 min.

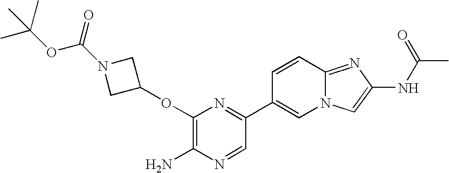

tert-Butyl 3-(6-(2-acetamidoimidazo[1,2-a]pyridin-6-yl)-3-aminopyrazin-2-yloxy)azetidine-1-carboxylate. LC/MS (m/z): 440.1 (MH⁺), Rₜ: 1.81 min; HPLC Rₜ: 1.30 min.

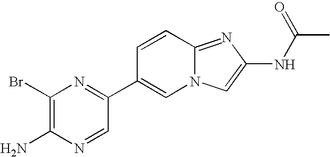

N-(6-(5-amino-6-bromopyrazin-2-yl)imidazo[1,2-a]pyridin-2-yl)acetamide TFA salt was prepared from the reaction of 3,5-dibromopyrazin-2-amine with N-(6-(4,4,5,5-tetramethyl-1,3,2-dioxaborolan-2-yl)imidazo[1,2-a]pyridin-2-yl)acetamide. LC/MS (m/z): 346.7 (MH⁺), Rₜ: 1.56 min; HPLC Rₜ: 1.89 min.

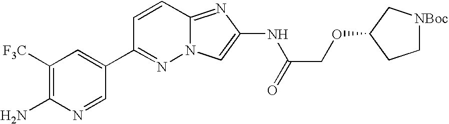

(S)-tert-Butyl 3-(2-(6-(6-amino-5-(trifluoromethyl)pyridin-3-yl)imidazo[1,2-b]pyridazin-2-ylamino)-2-oxoethoxy)pyrrolidine-1-carboxylate. LC/MS (m/z): 522.1 (MH⁺), Rₜ: 2.64 min.

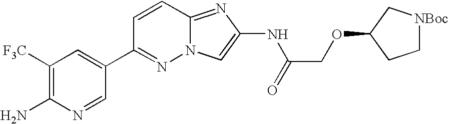

(R)-tert-Butyl 3-(2-(6-(6-amino-5-(trifluoromethyl)pyridin-3-yl)imidazo[1,2-b]pyridazin-2-ylamino)-2-oxoethoxy)pyrrolidine-1-carboxylate. LC/MS (m/z): 522.1 (MH⁺), Rₜ: 2.64 min.

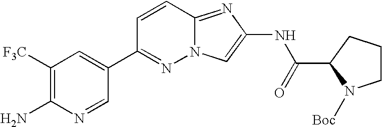

(R)-tert-butyl 2-(6-(6-amino-5-(trifluoromethyl)pyridin-3-yl)imidazo[1,2-b]pyridazin-2-ylcarbamoyl)pyrrolidine-1-carboxylate (40% yield). LC/MS (m/z): 492.1 (MH⁺), Rₜ: 2.51 min.

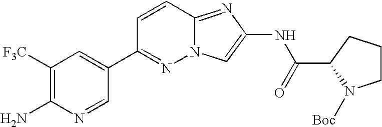

(S)-tert-butyl 2-(6-(6-amino-5-(trifluoromethyl)pyridin-3-yl)imidazo[1,2-b]pyridazin-2-ylcarbamoyl)pyrrolidine-1-carboxylate. LC/MS (m/z): 492.1 (MH⁺), Rₜ: 2.51 min.

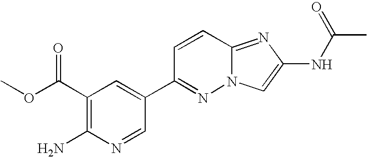

5-(2-Acetamidoimidazo[1,2-b]pyridazin-6-yl)-2-aminopyridine-3-carboxylate. LC/MS (m/z): 327.1 (MH⁺), Rₜ: 1.74 min

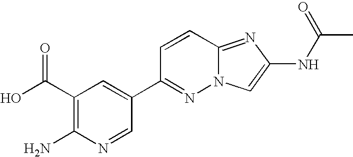

5-(2-Acetamidoimidazo[1,2-b]pyridazin-6-yl)-2-aminopyridine-3-carboxylic acid. LC/MS (m/z): 313.1 (MH⁺), Rₜ: 1.46 min.

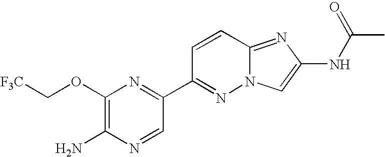

N-(6-(6-(2,2,2-trifluoroethoxy)-5-aminopyrazin-2-yl)imidazo[1,2-b]pyridazin-2-yl)acetamide. LC/MS (m/z): 368.1 (MH⁺), Rₜ: 2.09 min.

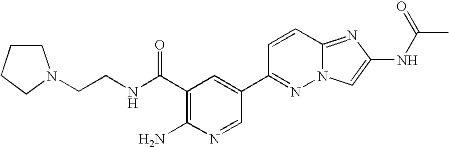

5-(2-acetamidoimidazo[1,2-b]pyridazin-6-yl)-2-amino-N-(2-(pyrrolidin-1-yl)ethyl)pyridine-3-carboxamide. LC/MS (m/z): 409.2 (MH⁺), Rₜ: 1.53 min.

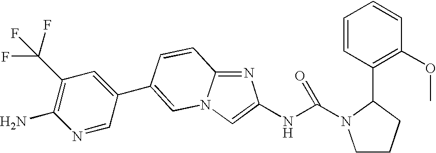

N-(6-(6-Amino-5-(trifluoromethyl)pyridin-3-yl)imidazo[1,2-a]pyridin-2-yl)-2-(2-methoxyphenyl)pyrrolidine-1-carboxamide was prepared as its TFA salt (4.9 mg, 4%). LC/MS (m/z): 497.0 (MH⁺), Rₜ: 2.24 min; HPLC Rₜ: 2.90 min

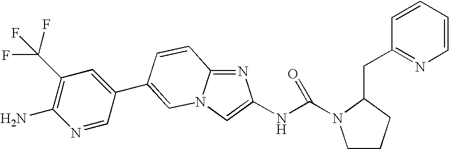

N-(6-(6-Amino-5-(trifluoromethyl)pyridin-3-yl)imidazo[1,2-a]pyridin-2-yl)-2-(pyridin-2-ylmethyl)pyrrolidine-1-carboxamide was prepared as its TFA salt (17%). LC/MS (m/z): 482.0 (MH⁺), Rₜ: 1.53 min, HPLC Rₜ: 1.75 min.

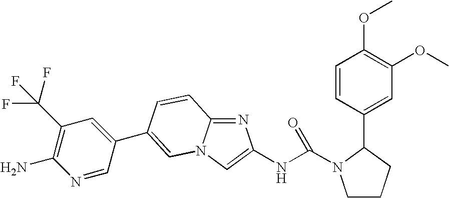

N-(6-(6-Amino-5-(trifluoromethyl)pyridin-3-yl)imidazo[1,2-a]pyridin-2-yl)-2-(3,4-dimethoxyphenyl)pyrrolidine-1-carboxamide was prepared as its TFA salt (8%). LC/MS (m/z): 527.0 (MH⁺), Rₜ: 2.04 min, HPLC Rₜ: 2.55 min.

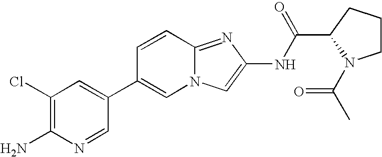

(S)-1-acetyl-N-(6-(6-amino-5-chloropyridin-3-yl)imidazo[1,2-a]pyridin-2-yl)pyrrolidine-2-carboxamide was prepared as its TFA salt (12%). LC/MS (m/z): 399.1 (MH⁺), Rₜ: 1.48 min.

### Example 2

### Preparation of N-(6-(6-amino-5-(trifluoromethyl)pyridin-3-yl)imidazo[1,2-b]pyridazin-2-yl)acetamide

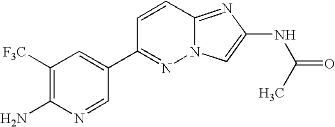

According to Example 1 (microwave: 125° C., 10 min), N-(6-(6-amino-5-(trifluoromethyl)pyridin-3-yl)imidazo[1,2-b]pyridazin-2-yl)acetamide TFA salt was prepared in 6.0% yield from the reaction of N-(6-chloroimidazo[1,2-b]pyridazin-2-yl)acetamide, with 5-(4,4,5,5-tetramethyl(1,3,2-dioxaborolan-2-yl))-3-(trifluoromethyl)-2-pyridylamine. LC/MS (m/z): 337.0 (MH⁺), Rₜ: 1.79 min; HPLC Rₜ: 2.15 min.

### Example 3

### Preparation of methyl 6-(6-amino-5-(trifluoromethyl)pyridin-3-yl)imidazo[1,2-a]pyridin-2-ylcarbamate

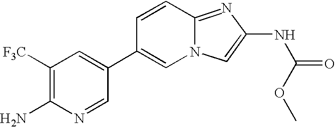

According to Example 1, methyl 6-(6-amino-5-(trifluoromethyl)pyridin-3-yl)imidazo[1,2-a]pyridin-2-ylcarbamate TFA salt was prepared in 8.0% yield from the reaction of a mixture of 6-iodoimidazo[1,2-a]pyridin-2-ylcarbamate and 1,3-bis(6-iodoimidazo[1,2-a]pyridin-2-yl)urea with 5-(4,4,5,5-tetramethyl(1,3,2-dioxaborolan-2-yl))-3-(trifluoromethyl)-2-pyridylamine. LC/MS (m/z): 352.0 (MH⁺), Rₜ: 1.68 min; HPLC Rₜ: 1.86 min.

### Example 4

### Preparation of N-(6-(6-amino-5-(trifluoromethyl)pyridin-3-yl)-3-bromo-imidazo[1,2-a]pyridin-2-yl)acetamide

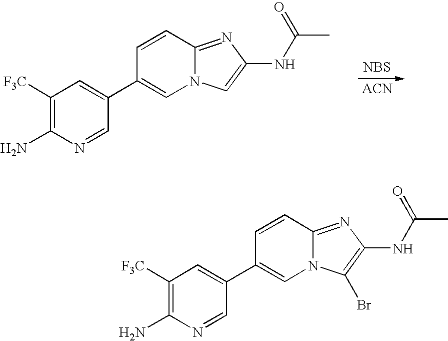

N-(6-(6-amino-5-(trifluoromethyl)pyridin-3-yl)-imidazo[1,2-a]pyridin-2-yl)-acetamide (50 mg, 0.15 mmol) was dissolved in ACN (3 ml) in a round bottom flask. NBS (26.5 mg, 0.15 mmol) was added at 0° C. and the solution was stirred for 5 minutes. A very small amount of Na₂S₂O₃ was added to the reaction. To the mixture was added water (10 mL) and ethyl acetate (10 mL), and the layers were separated. The organic layer was washed with brine (10 mL), dried over NaSO₄ and evaporated to afford N-(6-(6-amino-5-(trifluoromethyl)pyridin-3-yl)-3-bromo-imidazo[1,2-a]pyridin-2-yl)acetamide (61.5 mg, 78%). LC/MS (m/z): 414.0 (MH⁺), Rₜ: 1.71 min; HPLC Rₜ: 1.79 min.

The following compound was prepared according to Example 4:

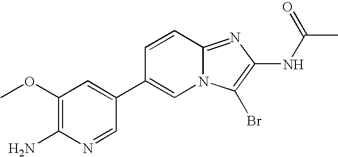

N-(6-(6-amino-5-methoxypyridin-3-yl)-3-bromo-imidazo[1,2-a]pyridin-2-yl)acetamide TFA salt was prepared from the reaction of N-(6-(6-amino-5-methoxypyridin-3-yl)imidazo[1,2-a]pyridin-2-yl)acetamide with NBS in 12% yield: LC/MS (m/z): 377.9 (MH⁺), Rₜ: 1.42 min; HPLC Rₜ: 1.31 min.

### Example 5

### Preparation of N-(6-(6-aminopyridin-3-yl)-3-bromo-imidazo[1,2-a]pyridin-2-yl)acetamide and N-(6-(6-amino-5-bromopyridin-3-yl)-3-bromo-imidazo[1,2-a]pyridin-2-yl)acetamide

According to Example 4, the reaction of N-(6-(6-aminopyridin-3-yl)imidazo[1,2-a]pyridin-2-yl)acetamide with NBS gave N-(6-(6-aminopyridin-3-yl)-3-bromo-imidazo[1,2-a]pyridin-2-yl)acetamide and N-(6-(6-amino-5-bromopyridin-3-yl)-3-bromo-imidazo[1,2-a]pyridin-2-yl)acetamide. The two compounds were separated by reverse phase preparative HPLC.

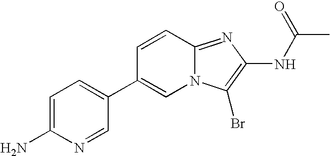

N-(6-(6-aminopyridin-3-yl)-3-bromo-imidazo[1,2-a]pyridin-2-yl)acetamide, TFA salt (yield 8.6%). LC/MS (m/z): 346.0 (MH⁺), Rₜ: 1.28 min; HPLC Rₜ: 1.18 min.

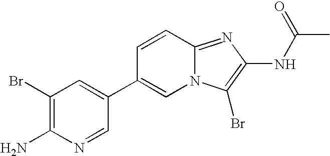

N-(6-(6-amino-5-bromopyridin-3-yl)-3-bromo-imidazo[1,2-a]pyridin-2-yl)acetamide, TFA salt (yield 2.7%). LC/MS (m/z): 423.9.0 (MH⁺), Rₜ: 1.46 min; HPLC Rₜ: 1.44 min.

### Example 6

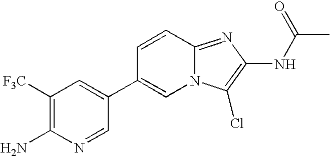

According to Example 4, the reaction of N-(6-(6-amino-5-(trifluoromethyl)pyridin-3-yl)imidazo[1,2-a]pyridin-2-yl)acetamide with NCS (30 min, room temperature) gave N-(6-(6-amino-5-(trifluoromethyl)pyridin-3-yl)-3-chloro-imidazo[1,2-a]pyridin-2-yl)acetamide as the TFA salt (22.4%). LC/MS (m/z): 370.0 (MH⁺), Rₜ: 1.63 min; HPLC Rₜ: 1.79 min; ¹H NMR (CD₃OD, 300 MHz) δ 8.62 (s, 1H), 8.55 (s, 1H), 8.23 (s, 1H), 7.85 (d, 1H, J=5.1 Hz), 7.73 (d, 2H, J=4.8 Hz), 2.24 (s, 3H).

### Example 7

### Preparation of 4-(2-acetamido-6-(6-amino-5-(trifluoromethyl)pyridin-3-yl)-imidazo[1,2-a]pyridin-3-yl)benzamide

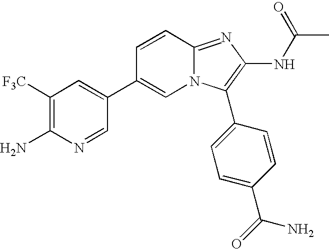

A mixture of N-(6-(6-amino-5-(trifluoromethyl)pyridin-3-yl)-3-bromo-imidazo[1,2-a]pyridin-2-yl)acetamide (20.7 mg, 0.05 mmol) and 4-carbamoylphenylboronic acid (24.8 mg, 0.15 mmol) in DME (0.750 mL) and aqueous Na₂CO₃ solution (2M, 0.250 mL) was purged with nitrogen for 5 minutes. To the mixture was added 1,1′-bis(diphenylphosphino)ferrocene palladium(II) chloride-DCM (6.1 mg, 0.0075 mmol). The vial was capped and heated at 100° C. for 600 seconds. Excess amount of anhydrous Na₂SO₄ was added and the reaction mixture was diluted with EtOAc (3 mL). The organic layer was filtered, concentrated, and dried in vacuo. The crude solid was purified by a preparative HPLC to give 4-(2-acetamido-6-(6-amino-5-(trifluoromethyl)pyridin-3-yl)-imidazo[1,2-a]pyridin-3-yl)benzamide (7.8 mg, 27.4%). LC/MS (m/z) 455.2 (MH⁺), 1.58 min; HPLC Rₜ: 1.71 min.

According to Example 7, the following compounds were prepared from the corresponding boronic acids or esters.

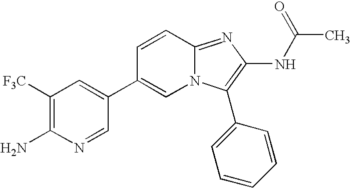

N-(6-(6-amino-5-(trifluoromethyl)pyridin-3-yl)-3-phenylimidazo[1,2-a]pyridin-2-yl)acetamide, TFA salt (yield 18.7%). LC/MS (m/z) 412.1 (MH⁺), Rₜ: 1.90 min; HPLC Rₜ: 2.14 min.

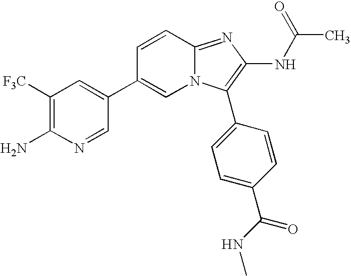

4-(2-acetamido-6-(6-amino-5-(trifluoromethyl)pyridin-3-yl)imidazo[1,2-a]pyridin-3-yl)-N-methylbenzamide, TFA salt (yield 11.5%). LC/MS (m/z) 469. 1 (MH⁺), Rₜ: 1.64 min; HPLC Rₜ: 1.80 min

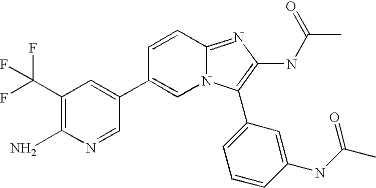

N-(3-(2-acetamido-6-(6-amino-5-(trifluoromethyl)pyridin-3-yl)imidazo[1,2-a]pyridin-3-yl)phenyl)acetamide (yield 32%). LC/MS (m/z) 469.1 (MH⁺), Rₜ: 1.89 min; HPLC Rₜ: 1.89 min.

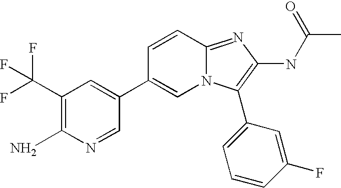

N-(6-(6-amino-5-(trifluoromethyl)pyridin-3-yl)-3-(3-fluorophenyl)-imidazo[1,2-a]pyridin-2-yl)acetamide (yield 39%). LC/MS (m/z) 430.1 (MH⁺), Rₜ: 2.06 min; HPLC Rₜ: 2.21 min.

### Example 8

### Preparation of N-(6-(6-amino-5-(trifluoromethyl)pyridin-3-yl)-3-(3-(diethylamino)prop-1-ynyl)imidazo[1,2-a]pyridin-2-yl)acetamide

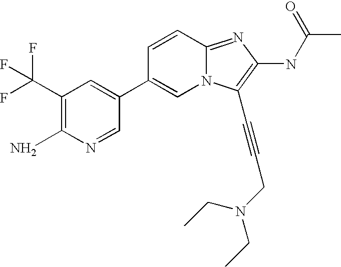

To a mixture of N-(6-(6-amino-5-(trifluoromethyl)pyridin-3-yl)-3-bromo-imidazo[1,2-a]pyridin-2-yl)acetamide (25 mg, 0.06 mmol), N,N-diethylprop-2-yn-1-amine (13 mg, 0.12 mmol) and a few drops of DMF in triethylamine (0.200 mL) was added copper(I) iodide (1 mg, 0.006 mmol). The solution purged with nitrogen for five minutes. Tetrakis(triphenylphosphine)palladium(0) (3.5 mg, 0.003 mmol) was added. The mixture was heated at 65° C. for five hours, treated with ethyl acetate and water. The organic layer was washed with brine, dried over sodium sulfated and evaporated to give the brown crude material. Purification by silica gel column chromatography using 2% methanol in dichloromethane afforded N-(6-(6-amino-5-(trifluoromethyl)pyridin-3-yl)-3-(3-(diethylamino)prop-1-ynyl)imidazo[1,2-a]pyridin-2-yl)acetamide as a white solid. LC/MS m/z 445.1 (MH⁺), Rₜ: 1.69 min.

According to Example 8, the following compounds were prepared from the corresponding aryl alkynes.

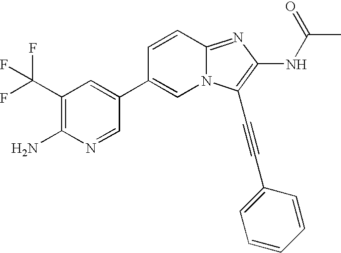

N-(6-(6-amino-5-(trifluoromethyl)pyridin-3-yl)-3-(phenylethynyl)imidazo[1,2-a]pyridin-2-yl)acetamide. LC/MS (m/z) 436.2 (MH⁺), Rₜ: 2.37 min.

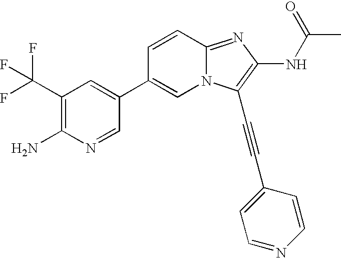

N-(6-(6-amino-5-(trifluoromethyl)pyridin-3-yl)-3-(pyridin-4-ylethynyl)imidazo[1,2-a]pyridin-2-yl)acetamide. LC/MS (m/z) 437.0 (MH⁺), Rₜ: 1.49 min; HPLC Rₜ: 1.84 min.

### Example 9

### Preparation of (R)—N-(6-(6-amino-5-(trifluoromethyl)pyridin-3-yl)imidazo[1,2-a]pyridin-2-yl)-2-(pyrrolidin-3-yloxy)acetamide

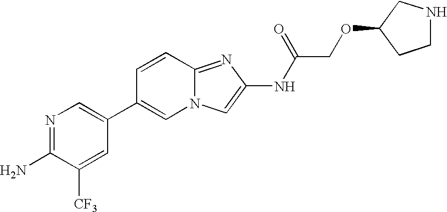

To a solution of (R)-tert-butyl 3-(2-(6-(6-amino-5-(trifluoromethyl)pyridin-3-yl)imidazo[1,2-a]pyridin-2-ylamino)-2-oxoethoxy)pyrrolidine-1-carboxylate (38 mg, 0.09 mmol prepared as in Example 1 and Method 49) in DCM (2 mL) were added 3 drops of TFA and 1 drop of water. After 4 h, the reaction mix was concentrated, purified on a reverse phase column, and lyophilized to give the desired product (12 mg, 43%). LC/MS (m/z): 421.1 (MH⁺), Rₜ: 1.65 min; HPLC Rₜ: 1.58 min.

### Example 10

### Preparation of N-(6-(6-amino-5-(trifluoromethyl)pyridin-3-yl)imidazo[1,2-b]pyridazin-2-yl)-3-(piperidin-2-yl)propanamide

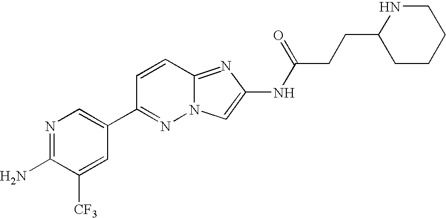

To tert-butyl 2-(3-(6-(6-amino-5-(trifluoromethyl)pyridin-3-yl)imidazo[1,2-b]pyridazin-2-ylamino)-3-oxopropyl)piperidine-1-carboxylate (10 mg, 0.02 mmol) was added 1 mL 4N HCl in dioxane. After 1 h, the solution was reduced in volume, purified on a reverse phase column and then lyophilized to give the desired product (3.5 mg, 40%). LC/MS (m/z): 434.1 (MH⁺), Rₜ: 1.88 min; HPLC Rₜ: 1.94 min.

### Example 11

### Preparation of N-(6-(5-amino-6-(piperidin-4-yloxy)pyrazin-2-yl)imidazo[1,2-a]pyridin-2-yl)acetamide

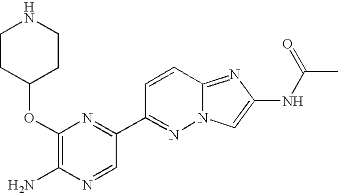

To tert-butyl 4-(6-(2-acetamidoimidazo[1,2-a]pyridin-6-yl)-3-aminopyrazin-2-yloxy)piperidine-1-carboxylate (10 mg, 0.02 mmol) was added 20% TFA in DCM (1 mL). After 15 min, the solution was concentrated in vacuo, purified on a reverse phase column and then lypholized to give the desired product (2 mg, 27%). LC/MS (m/z): 368.2 (MH⁺), Rₜ: 1.43 min; HPLC Rₜ: 1.28 min.

### Example 12

### Preparation of N-(6-(6-amino-5-(trifluoromethyl)pyridin-3-yl)imidazo[1,2-a]pyridin-2-yl)-2-(1-ethylpiperidin-4-yloxy)acetamide

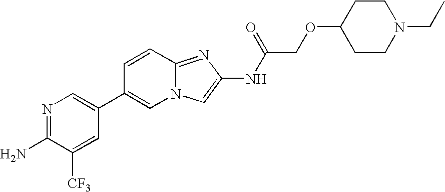

To a solution of N-(6-(6-amino-5-(trifluoromethyl)pyridin-3-yl)imidazo[1,2-a]pyridin-2-yl)-2-(piperidin-4-yloxy)acetamide (8 mg, 0.018 mmol) in methanol (0.300 mL) was added acetic acid (0.002 mL), acetaldehyde (0.003 mL, 0.06 mmol) and sodium cyanoborohydride (1.5 mg, 0.02 mmol). After stirring overnight, the reaction mixture was concentrated, purified by reverse phase preparative HPLC and lyophilized to give the desired product (1.9 mg, 24%). LC/MS (m/z): 463.2 (MH⁺), Rₜ: 1.75 min; HPLC Rₜ: 1.68 min.

According to Example 12, the following compounds were prepared from the reductive alkylation of an amine:

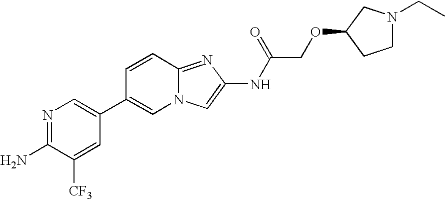

(R)—N-(6-(6-amino-5-(trifluoromethyl)pyridin-3-yl)imidazo[1,2-a]pyridin-2-yl)-2-(1-ethylpyrrolidin-3-yloxy)acetamide. LC/MS (m/z): 449.2 (MH⁺), Rₜ: 1.72 min; HPLC Rₜ: 1.63 min.

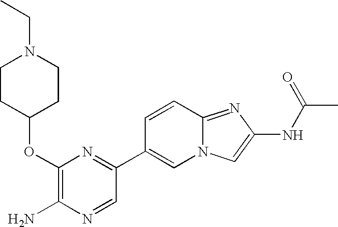

N-(6-(5-amino-6-(1-ethylpiperidin-4-yloxy)pyrazin-2-yl)imidazo[1,2-a]pyridin-2-yl)acetamide. LC/MS (m/z): 396.2 (MH⁺), Rₜ: 1.50 min; HPLC Rₜ: 1.39 min.

### Example 13

### Preparation of N-(6-(6-amino-5-(trifluoromethyl)pyridin-3-yl)-5-hydroxyimidazo[1,2-a]pyridin-2-yl)-2,2,2-trifluoroacetamide

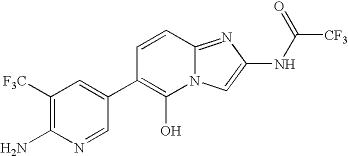

Pd(dppf)₂Cl₂-DCM (50 mg, 0.06 mmol) was added to a mixture of N-(6-bromo-5-fluoroimidazo[1,2-a]pyridin-2-yl)-2,2,2-trifluoroacetamide (40 mg, 0.12 mmol) 5-(4,4,5,5-tetramethyl-1,3,2-dioxaborolan-2-yl)-3-(trifluoromethyl)pyridin-2-amine (71 mg, 0.25 mmol) and sodium carbonate (2M, 0.5 mL) in DME (1.3 mL) which was previously flushed with nitrogen. After microwave heating at 105° C. for 10 min the organic layer was decanted, concentrated in vacuo, purified on a reverse phase column and then lyophilized to give N-(6-(6-amino-5-(trifluoromethyl)pyridin-3-yl)-5-hydroxyimidazo[1,2-a]pyridin-2-yl)-2,2,2-trifluoroacetamide (2 mg, 4%). LC/MS (m/z): 406.0 (MH⁺), Rₜ: 2.20 min; HPLC Rₜ: 2.55 min.

### Example 14

### Preparation of N-(6-(6-amino-5-formylpyridin-3-yl)imidazo[1,2-a]pyridin-2-yl)acetamide

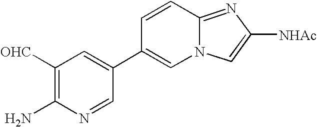

To a solution of 2-amino-5-bromonicotinaldehyde (503 mg, 2.5 mmol) in dioxane (10 mL) in a microwave reaction vessel was added bispinacolatodiboron (762 mg, 3.0 mmol), Pd(dppf)Cl₂-DCM (204 mg, 0.25 mmol), and anhydrous KOAc (368 mg, 3.75 mmol). The reaction mixture was then heated twice in a microwave reactor at 95° C. for 1200 sec. After the solid residue was removed, the crude 2-amino-5-(4,4,5,5-tetramethyl-1,3,2-dioxaborolan-2-yl)nicotinaldehyde in dioxane was added to the solution of N-(6-iodoimidazo[1,2-a]pyridin-2-yl)acetamide (600 mg, 2.0 mmol) in 20 mL of DME and 2 M Na₂CO₃ aqueous solution (3:1) in a sealed reaction vessel. The reaction mixture was degassed by anhydrous N₂ stream for 15 min followed by the addition of Pd(dppf)Cl₂-DCM (163 mg, 0.2 mmol). The reaction mixture was then heated to 100° C. for 15 h. To the reaction mixture was added excess amount of anhydrous Na₂SO₄ and diluted with EtOAc (3 mL). The organic layer was filtered, concentrated, and dried in vacuo. The crude solid was purified by a preparative HPLC to give N-(6-(6-amino-5-formylpyridin-3-yl)imidazo[1,2-a]pyridin-2-yl)acetamide as its TFA salt, which was treated with saturated NaHCO₃ (200 mL) solution and extracted with EtOAc (300 mL), dried over anhydrous Na₂SO₄, filtered and dried in vacuo to give the free amine (88 mg, 15%). LC/MS (m/z): 296.0 (MH⁺), Rₜ: 1.16 min; HPLC Rₜ: 1.26 min.

### Example 15

### Preparation of N-(6-(6-amino-5-((2,2-dimethylhydrazono)methyl)pyridin-3-yl)imidazo[1,2-a]pyridin-2-yl)acetamide

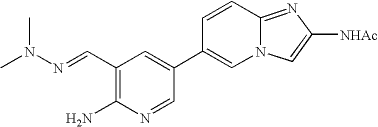

To a solution of N-(6-(6-amino-5-formylpyridin-3-yl)imidazo[1,2-a]pyridin-2-yl)acetamide (16.3 mg, 0.06 mmol) and dimethylhydrazine (16.6 mg, 0.28 mmol) in EtOH (0.7 mL) in a microwave reaction vessel was added piperidine (23 mg, 0.28 mmol). The reaction mixture was then heated in a microwave reactor at 150° C. for 1800 sec. After the volatile material was evaporated, the crude compound was purified by a preparative HPLC to give N-(6-(6-amino-5-((2,2-dimethylhydrazono)methyl)pyridin-3-yl)imidazo[1,2-a]pyridin-2-yl)acetamide as its TFA salt (5.1 mg, 43%). LC/MS (m/z): 338.1 (MH⁺), Rₜ: 1.39 min; HPLC Rₜ: 1.67 min.

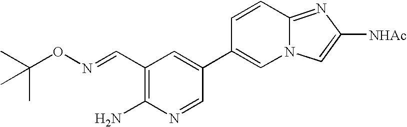

According to Example 15, N-(6-(6-amino-5-((tert-butoxyimino)methyl)pyridin-3-yl)imidazo[1,2-a]pyridin-2-yl)acetamide was prepared from N-(6-(6-amino-5-formylpyridin-3-yl)imidazo[1,2-a]pyridin-2-yl)acetamide and the corresponding commercially available oximes as its TFA salt (4.0% yield). LC/MS (m/z): 367.1 (MH⁺), Rₜ: 1.68 min; HPLC Rₜ: 2.11 min.

### Example 16

### Preparation of N-(6-(6-amino-5-(trifluoromethyl)pyridin-3-yl)-3-phenethylimidazo[1,2-a]pyridin-2-yl)acetamide

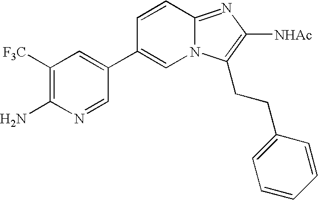

To a solution of N-(6-(6-amino-5-(trifluoromethyl)pyridin-3-yl)-3-(phenylethynyl)imidazo[1,2-a]pyridin-2-yl)acetamide trifluoroacetic acid salt (10 mg, 0.018 mmol, prepared as in example 8) in methanol (1 mL) was added palladium-on-charcoal (5 mg, 50% wt/wt). The reaction was charged with a hydrogen balloon, and stirred at room temperature for 5 h. After the palladium catalyst was removed through Celite pad, the organic layer was concentrated and the crude product was purified a preparative HPLC to give N-(6-(6-amino-5-(trifluoromethyl)pyridin-3-yl)-3-phenethylimidazo[1,2-a]pyridin-2-yl)acetamide as its TFA salt (1.9 mg, 20%). LC/MS (m/z): 440.1 (MH⁺), Rₜ: 1.90 min; HPLC Rₜ: 2.45 min.

The following compound was prepared according to Example 16.

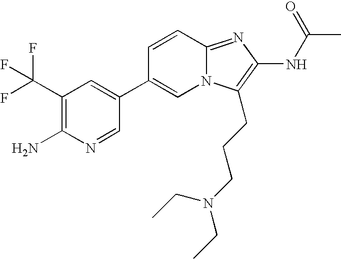

N-(6-(6-amino-5-(trifluoromethyl)pyridin-3-yl)-3-(3-(diethylamino)propyl)imidazo[1,2-a]pyridin-2-yl)acetamide. LC/MS (m/z): 225.1 (MH⁺), Rₜ: 1.51 min.

### Example 17

### Preparation of N-(6-(6-amino-5-(2-phenoxyphenyl)pyridin-3-yl)imidazo[1,2-b]pyridazin-2-yl)acetamide

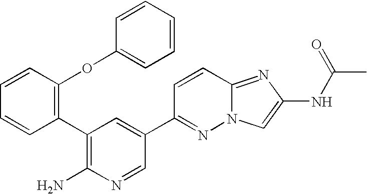

A mixture of N-(6-(6-amino-5-chloropyridin-3-yl)imidazo[1,2-b]pyridazin-2-yl)acetamide (15 mg, 0.05 mmol), 2-phenoxyphenylboronic acid (32 mg, 0.15 mmol) and 1,1-bis(diphenylphosphino)ferrocene palladium (II) chloride-dichloromethane complex (40 mg, 0.05 mmol) in 0.5 mL solution of DME and 2 M aq. sodium carbonate (3:1) was heated in the microwave at 125° C. for 900 seconds. The crude product was purified by reverse phase prep HPLC to give N-(6-(6-amino-5-(2-phenoxyphenyl)pyridin-3-yl)imidazo[1,2-b]pyridazin-2-yl)acetamide. LC/MS (m/z): 437.1 (MH⁺), Rₜ: 1.98 min; HPLC Rₜ: 2.61 min.

According to Example 17, the following compounds were prepared from the corresponding boronic esters and N-(6-(6-amino-5-chloropyridin-3-yl)imidazo[1,2-b]pyridazin-2-yl)acetamide:

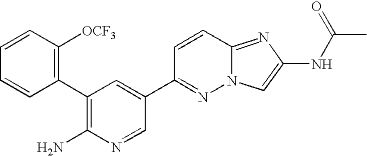

N-(6-(6-amino-5-(2-(trifluoromethoxy)phenyl)pyridin-3-yl)imidazo[1,2-b]pyridazin-2-yl)acetamide. LC/MS (m/z): 429.1 (MH⁺), Rₜ: 1.84 min; HPLC Rₜ: 2.28 min.

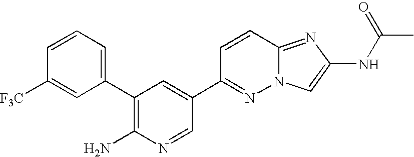

N-(6-(6-amino-5-(3-(trifluoromethyl)phenyl)pyridin-3-yl)imidazo[1,2-b]pyridazin-2-yl)acetamide (microwave: 125° C., 10 min). LC/MS (m/z): 412.9 (MH⁺), Rₜ: 1.90 min; HPLC Rₜ: 2.46 min.

### Example 18

### Preparation of N-(6-(6-amino-5-(4-(trifluoromethyl)phenyl)pyridin-3-yl)imidazo[1,2-a]pyridin-2-yl)acetamide

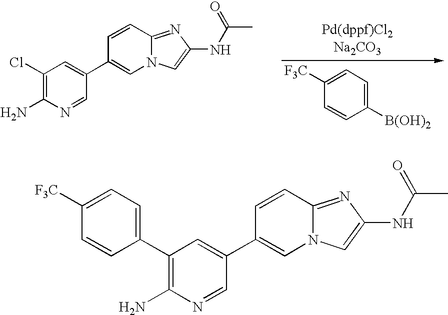

A mixture of N-(6-(6-amino-5-chloropyridin-3-yl)imidazo[1,2-a]pyridin-2-yl)acetamide (15 mg, 0.050 mmol), 4-(trifluoromethyl)phenylboronic acid (95 mg, 0.50 mmol) and 1,1-bis(diphenylphosphino)ferrocene palladium (II) chloride (20 mg, 0.025 mmol) in 1,4-dioxane (2 mL) and 0.25 mL of 2 M aq. sodium carbonate was heated in the microwave at 125° C. for 1500 seconds. The crude product was purified by reverse phase prep HPLC to give the title compound. LC/MS (m/z): 412.4 (MH⁺), Rₜ: 2.02 min; HPLC Rₜ: 2.225 min.

### Example 19

### Preparation of N-(6-(5-amino-6-(azetidin-3-yloxy)pyrazin-2-yl)imidazo[1,2-a]pyridin-2-yl)acetamide

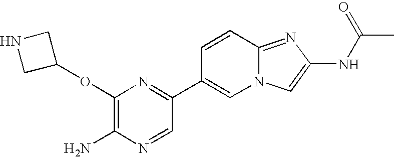

To a solution of tert-butyl 3-(6-(2-acetamidoimidazo[1,2-a]pyridin-6-yl)-3-aminopyrazin-2-yloxy)azetidine-1-carboxylate TFA salt (5 mg, 0.01 mmol) in CH₂Cl₂ (1 mL) was added trifluoroacetic acid (0.5 mL). The reaction mixture was stirred for 30 min at room temperature. The volatile materials were evaporated and the crude material was purified by prep HPLC to give N-(6-(5-amino-6-(azetidin-3-yloxy)pyrazin-2-yl)imidazo[1,2-a]pyridin-2-yl)acetamide TFA salt. LC/MS (m/z): 340.1 (MH⁺), Rₜ: 1.14 min; HPLC Rₜ: 1.21 min.

### Example 20

### Preparation of methyl 1-(6-(6-amino-5-(trifluoromethyl)pyridin-3-yl)H-imidazo[1,2-a]pyridin-2-ylcarbamoyl)pyrrolidine-3-carboxylate

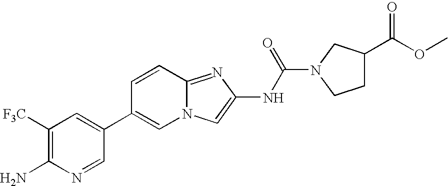

Methyl 1-(6-iodoH-imidazo[1,2-a]pyridin-2-ylcarbamoyl)pyrrolidine-3-carboxylate (414 mg, 1.0 mmol) and 5-(4,4,5,5-tetramethyl-1,3,2-dioxaborolan-2-yl)-3-(trifluoromethyl)pyridin-2-amine (345 mg, 1.2 mmol) was mixed with DME (5 mL) and 2 M Na₂CO₃ aqueous solution (3:1) in the microwave reaction vessel. The reaction mixture was degassed by anhydrous N₂ stream for 5 min followed by the addition of Pd(dppf)Cl₂-DCM (81 mg, 0.1 mmol). The reaction mixture was then heated in a microwave reactor at 110° C. for 600 sec. To the reaction mixture was added excess amount of anhydrous Na₂SO₄ and diluted with EtOAc (3 mL). The organic layer was filtered, concentrated, and dried in vacuo. The crude solid was purified by a preparative HPLC to give methyl 1-(6-(6-amino-5-(trifluoromethyl)pyridin-3-yl)H-imidazo[1,2-a]pyridin-2-ylcarbamoyl)pyrrolidine-3-carboxylate as its TFA salt. LC/MS (m/z): 449.2 (MH⁺), Rₜ: 1.94 min.

### Example 21

### Preparation of 1-(6-(6-amino-5-(trifluoromethyl)pyridin-3-yl)H-imidazo[1,2-a]pyridin-2-ylcarbamoyl)pyrrolidine-3-carboxylic acid

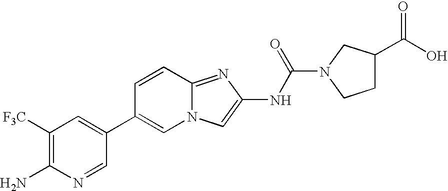

To a stirring suspension of methyl 1-(6-(6-amino-5-(trifluoromethyl)pyridin-3-yl)H-imidazo[1,2-a]pyridin-2-ylcarbamoyl)pyrrolidine-3-carboxylate (200 mg, 0.45 mmol) in THF (4 mL) was added a 1.0 M LiOH solution (0.5 mL). After 2 h the crude reaction mixture was concentrated and the residue was purified by preparative HPLC to give 1-(6-(6-amino-5-(trifluoromethyl)pyridin-3-yl)H-imidazo[1,2-a]pyridin-2-ylcarbamoyl)pyrrolidine-3-carboxylic acid as its TFA salt. LC/MS (m/z): 435.1 (MH⁺), Rₜ: 1.77 min.

### Example 22

### Preparation of N¹-(6-(6-amino-5-(trifluoromethyl)pyridin-3-yl)H-imidazo[1,2-a]pyridin-2-yl)-N³-methylpyrrolidine-1,3-dicarboxamide

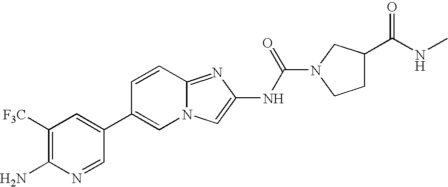

To a stirring suspension of 1-(6-(6-amino-5-(trifluoromethyl)pyridin-3-yl)H-imidazo[1,2-a]pyridin-2-ylcarbamoyl)pyrrolidine-3-carboxylic acid (30 mg, 0.07 mmol) in DMF (1 mL) was added iPr₂NEt (0.1 mL, 0.56 mmol), followed shortly by EDC (67 mg, 0.35 mmol) and HOBt (47 mg, 0.35 mmol). After stirring for 2 h at rt, a 2.0 M solution of methylamine in THF (0.2 mL) was added and the reaction was maintained at room temperature for 16 h. The crude reaction mixture was diluted with EtOAc (50 mL) and saturated aqueous NaHCO₃ solution (30 mL). The organic layer was separated, and the aqueous phase was extracted with EtOAc (2×30 mL). The combined organic portions were washed with brine (50 mL), dried over anhydrous Na₂SO₄, filtered, concentrated and dried in vacuo. The crude solid was purified by preparative HPLC to give N¹-(6-(6-amino-5-(trifluoromethyl)pyridin-3-yl)H-imidazo[1,2-a]pyridin-2-yl)-N³-methylpyrrolidine-1,3-dicarboxamide as its TFA salt. LC/MS (m/z): 448.2 (MH⁺), Rₜ: 1.70 min.

### Example 23

### Preparation of (S)-1-(6-(6-amino-5-(trifluoromethyl)pyridin-3-yl)H-imidazo[1,2-a]pyridin-2-ylcarbamoyl)azetidine-2-carboxylic acid

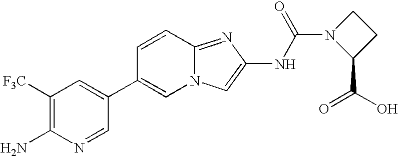

(S)-benzyl 1-(6-iodoH-imidazo[1,2-a]pyridin-2-ylcarbamoyl)azetidine-2-carboxylate (20 mg, 0.042 mmol) and 5-(4,4,5,5-tetramethyl-1,3,2-dioxaborolan-2-yl)-3-(trifluoromethyl)pyridin-2-amine (18 mg, 0.063 mmol) were mixed with 1 mL of DME and 2 M Na₂CO₃ aqueous solution (3:1) in a microwave reaction vessel. The reaction mixture was degassed by anhydrous N₂ stream for 15 min and Pd(dppf)₂Cl₂-DCM (5 mg, 0.004 mmol) was added. The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by a preparative HPLC to give (S)-1-(6-(6-amino-5-(trifluoromethyl)pyridin-3-yl)H-imidazo[1,2-a]pyridin-2-ylcarbamoyl)azetidine-2-carboxylic acid as its TFA salt. LC/MS (m/z): 421.1 (MH⁺), Rₜ: 1.72 min.

### Example 24

### Preparation of (S)—N¹-(6-(6-amino-5-(trifluoromethyl)pyridin-3-yl)H-imidazo[1,2-a]pyridin-2-yl)-N²-methylazetidine-1,2-dicarboxamide

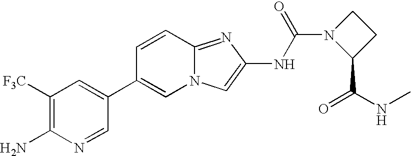

To a stirring suspension of (S)-1-(6-(6-amino-5-(trifluoromethyl)pyridin-3-yl)H-imidazo[1,2-a]pyridin-2-ylcarbamoyl)azetidine-2-carboxylic acid (17 mg, 0.07 mmol) in THF (0.3 mL) was added iPr₂NEt (0.015 mL, 0.56 mmol), followed shortly by EDC (67 mg, 0.35 mmol), HOBt (47 mg, 0.08 mmol), and a 2.0 M solution of methylamine in THF (0.030 mL). The reaction mixture was maintained at room temperature for 16 h. The crude reaction mixture was concentrated in vacuo, the residue dissolved in DMSO and purified by preparative HPLC to give (S)—N¹-(6-(6-amino-5-(trifluoromethyl)pyridin-3-yl)H-imidazo[1,2-a]pyridin-2-yl)-N²-methylazetidine-1,2-dicarboxamide as its TFA salt. LC/MS (m/z): 434.1 (MH⁺), Rₜ: 1.68 min.

### Example 25

### Preparation of (S)-1-(6-(6-amino-5-(trifluoromethyl)pyridin-3-yl)H-imidazo[1,2-a]pyridin-2-ylcarbamoyl)pyrrolidine-2-carboxylic acid

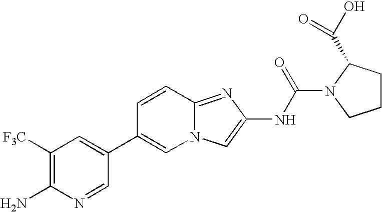

(S)-methyl 1-(6-iodoH-imidazo[1,2-a]pyridin-2-ylcarbamoyl)pyrrolidine-2-carboxylate (212 mg, 0.51 mmol) and 5-(4,4,5,5-tetramethyl-1,3,2-dioxaborolan-2-yl)-3-(trifluoromethyl)pyridin-2-amine (221 mg, 0.77 mmol) was mixed with 3 mL of DME and 2 M Na₂CO₃ aqueous solution (3:1) in the microwave reaction vessel. The reaction mixture was degassed by anhydrous N₂ stream for 15 min followed by the addition of Pd(dppf)Cl₂-DCM (63 mg, 0.077 mmol). The reaction mixture was then heated in a microwave reactor at 110° C. for 600 sec. To the reaction mixture was added excess amount of anhydrous Na₂SO₄ and diluted with EtOAc (3 mL). The organic layer was filtered, concentrated, and dried in vacuo. The crude solid was purified by preparative HPLC to give (S)-1-(6-(6-amino-5-(trifluoromethyl)pyridin-3-yl)H-imidazo[1,2-a]pyridin-2-ylcarbamoyl)pyrrolidine-2-carboxylic acid as its TFA salt. LC/MS (m/z): 435.1 (MH⁺), Rₜ: 1.79 min.

### Example 26

### Preparation of (S)—N¹-(6-(6-amino-5-(trifluoromethyl)pyridin-3-yl)H-imidazo[1,2-a]pyridin-2-yl)pyrrolidine-1,2-dicarboxamide

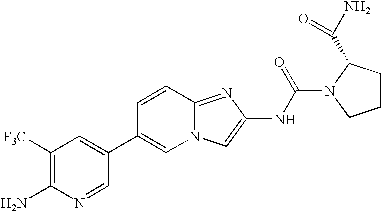

CDI (24 mg, 0.15 mmol) was added to a solution of (S)-1-(6-(6-amino-5-(trifluoromethyl)pyridin-3-yl)H-imidazo[1,2-a]pyridin-2-yl)carbamoyl)pyrrolidine-2-carboxylic acid (43 mg, 0.1 mmol) in DMF (0.3 mL). The resulting mixture was heated in an oil bath at 40° C. for 30 min. After cooling to room temperature a solution of NH₄OH (0.035 mL) in DMF (0.065 mL) was added and the reaction mixture was heated in an oil bath at 80° C. for 16 h. The crude mixture was dissolved in DMSO and purified by reverse phase preparative HPLC to give (S)-N,N-(6-(6-amino-5-(trifluoromethyl)pyridin-3-yl)H-imidazo[1,2-a]pyridin-2-yl)pyrrolidine-1,2-dicarboxamide as its TFA salt. LC/MS (m/z): 434.2 (MH⁺), Rₜ: 1.68 min.

### Example 27

### Preparation of (S)—N¹-(6-(6-amino-5-(trifluoromethyl)pyridin-3-yl)H-imidazo[1,2-a]pyridin-2-yl)-N²-methylpyrrolidine-1,2-dicarboxamide

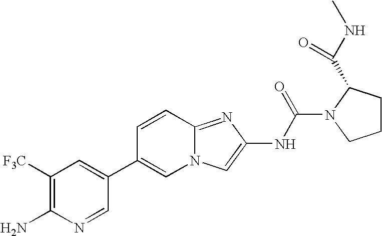

To a solution of (S)-1-(6-(6-amino-5-(trifluoromethyl)pyridin-3-yl)H-imidazo[1,2-a]pyridin-2-yl)carbamoyl)pyrrolidine-2-carboxylic acid (23 mg, 0.05 mmol) in THF (0.300 mL) was added DIEA (0.019 mL, 0.1 mmol), followed shortly by EDC (13 mg, 0.065 mmol), HOBt (9 mg, 0.065 mmol), and a 2.0 M solution of methylamine in THF (0.040 mL). After stirring for 3 h at room temperature, DMF (0.5 mL) was added to aid solubilization and the reaction was maintained at room temperature for 16 h. The crude mixture was diluted with DMSO and purified by reverse phase preparative HPLC to give (S)—N¹-(6-(6-amino-5-(trifluoromethyl)pyridin-3-yl)H-imidazo[1,2-a]pyridin-2-yl)-N²-methylpyrrolidine-1,2-dicarboxamide as its TFA salt. LC/MS (m/z): 448.2 (MH⁺), Rₜ: 1.72 min.

### Example 28

### Preparation of (S)-tert-butyl 2-(6-(6-amino-5-(trifluoromethyl)pyridin-3-yl)H-imidazo[1,2-a]pyridin-2-ylcarbamoyl)azetidine-1-carboxylate

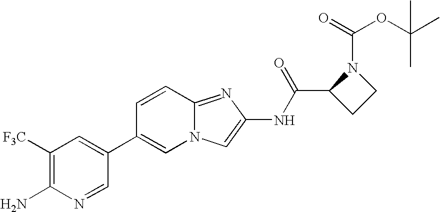

(S)-tert-butyl 2-(6-iodoH-imidazo[1,2-a]pyridin-2-ylcarbamoyl)azetidine-1-carboxylate (442 mg, 1 mmol) and 5-(4,4,5,5-tetramethyl-1,3,2-dioxaborolan-2-yl)-3-(trifluoromethyl)pyridin-2-amine (345 mg, 1.2 mmol) was mixed with 5 mL of DME and 2 M Na₂CO₃ aqueous solution (3:1) in the microwave reaction vessel. The reaction mixture was degassed by anhydrous N₂ stream for 15 min followed by the addition of Pd(dppf)Cl₂-DCM (81 mg, 0.1 mmol). The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by preparative HPLC to give (S)-tert-butyl 2-(6-(6-amino-5-(trifluoromethyl)pyridin-3-yl)H-imidazo[1,2-a]pyridin-2-ylcarbamoyl)azetidine-1-carboxylate as its TFA salt. LC/MS (m/z): 477.2 (MH⁺), Rₜ: 2.26 min.

### Example 29

### Preparation of (S)—N-(6-(6-amino-5-(trifluoromethyl)pyridin-3-yl)H-imidazo[1,2-a]pyridin-2-yl)azetidine-2-carboxamide

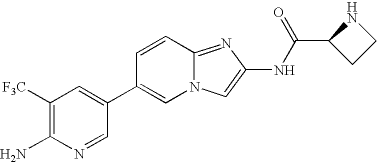

TFA (0.750 mL) was added to a stirring solution of (S)-tert-butyl 2-(6-(6-amino-5-(trifluoromethyl)pyridin-3-yl)H-imidazo[1,2-a]pyridin-2-ylcarbamoyl)azetidine-1-carboxylate (250 mg, 0.52 mmol) in CH₂Cl₂ (3 mL). The reaction mixture was maintained at room temperature for 16 h. The crude mixture was neutralized with saturated aqueous sodium carbonate (5 mL) then diluted with CH₂Cl₂ (10 mL) and H₂O (10 mL). The organic layer was separated, and the aqueous phase was extracted with CH₂Cl₂ (2×20 mL). The combined organic portions were washed with brine (40 mL), dried over anhydrous Na₂SO₄, filtered, concentrated and dried in vacuo to give (S)—N-(6-(6-amino-5-(trifluoromethyl)pyridin-3-yl)H-imidazo[1,2-a]pyridin-2-yl)azetidine-2-carboxamide as a brown solid. The crude product was used for the next step without further purification. LC/MS (m/z): 377.1 (MH⁺), Rₜ: 1.61 min.

### Example 30

### Preparation of (S)—N²-(6-(6-amino-5-(trifluoromethyl)pyridin-3-yl)H-imidazo[1,2-a]pyridin-2-yl)azetidine-1,2-dicarboxamide

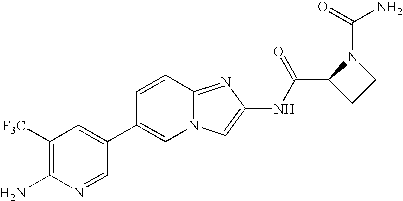

KCNO (60 mg, 0.72 mmol) was added to a stirring solution of (S)—N-(6-(6-amino-5-(trifluoromethyl)pyridin-3-yl)H-imidazo[1,2-a]pyridin-2-yl)azetidine-2-carboxamide (30 mg, 0.08 mmol) in DMF (0.3 mL) in a microwave reaction vessel. The reaction mixture was then heated in a microwave reactor at 100° C. for 1200 sec. The crude reaction mixture was diluted with DMSO and purified by reverse phase preparative HPLC to give (S)—N²-(6-(6-amino-5-(trifluoromethyl)pyridin-3-yl)H-imidazo[1,2-a]pyridin-2-yl)azetidine-1,2-dicarboxamide as its TFA salt. LC/MS (m/z): 420.1 (MH⁺), Rₜ: 1.70 min.

### Example 31

### Preparation of tert-butyl 2-(6-(6-(2-methoxyethoxy)-5-aminopyrazin-2-yl)H-imidazo[1,2-a]pyridin-2-ylcarbamoyl)azetidine-1-carboxylate

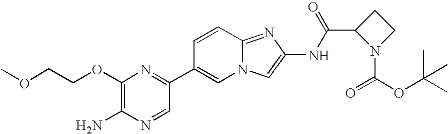

tert-Butyl 2-(6-iodoH-imidazo[1,2-a]pyridin-2-ylcarbamoyl)azetidine-1-carboxylate (442 mg, 1 mmol) and crude 3-(2-methoxyethoxy)-5-(4,4,5,5-tetramethyl-1,3,2-dioxaborolan-2-yl)pyrazin-2-amine (1.25 mmol) was mixed with 2 M Na₂CO₃ aqueous solution (1 mL) and DME (3 mL) in the microwave reaction vessel. The reaction mixture was degassed by anhydrous N₂ stream for 15 min and Pd(dppf)₂Cl₂-DCM (81 mg, 0.1 mmol) was added. The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by reverse phase preparative HPLC to give tert-butyl 2-(6-(6-(2-methoxyethoxy)-5-aminopyrazin-2-yl)H-imidazo[1,2-a]pyridin-2-ylcarbamoyl)azetidine-1-carboxylate as its TFA salt. LC/MS (m/z): 484.2 (MH⁺), Rₜ: 2.09 min.

### Example 32

### Preparation of N-(6-(6-(2-methoxyethoxy)-5-aminopyrazin-2-yl)H-imidazo[1,2-a]pyridin-2-yl)azetidine-2-carboxamide

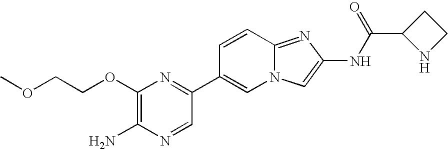

TFA (0.2 mL) was added to a stirring solution of tert-butyl 2-(6-(6-(2-methoxyethoxy)-5-aminopyrazin-2-yl)H-imidazo[1,2-a]pyridin-2-ylcarbamoyl)azetidine-1-carboxylate (64 mg, 0.133 mmol) in CH₂Cl₂ (0.8 mL). The reaction mixture was maintained at rt for 16 h. The crude mixture was neutralized with saturated aqueous sodium carbonate (5 mL) then diluted with CH₂Cl₂ (10 mL) and H₂O (10 mL). The organic layer was separated, and the aqueous phase was extracted with CH₂Cl₂ (2×20 mL). The combined organic portions were washed with brine (40 mL), dried over anhydrous Na₂SO₄, filtered, concentrated and dried in vacuo to give N-(6-(6-(2-methoxyethoxy)-5-aminopyrazin-2-yl)H-imidazo[1,2-a]pyridin-2-yl)azetidine-2-carboxamide as a brown solid. The crude product was used for the next step without further purification. LC/MS (m/z): 384.2 (MH⁺), Rₜ: 1.44 min.

### Example 33

### Preparation of N-(6-(6-(2-methoxyethoxy)-5-aminopyrazin-2-yl)H-imidazo[1,2-a]pyridin-2-yl)-1-acetylazetidine-2-carboxamide

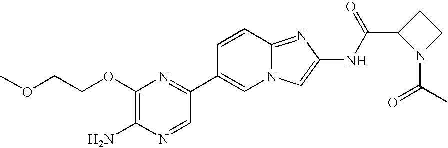

Et₃N (0.010 mL) was added to a stirring solution of N-(6-(6-(2-methoxyethoxy)-5-aminopyrazin-2-yl)H-imidazo[1,2-a]pyridin-2-yl)azetidine-2-carboxamide (20 mg, 0.052 mmol) in CH₂Cl₂ (0.5 mL) followed shortly by acetic anhydride (0.006 mL, 0.063 mmol). The reaction mixture was maintained at rt for 2 h. The reaction mixture was concentrated, the crude residue was dissolved in DMSO and purified by reverse phase preparative HPLC to give N-(6-(6-(2-methoxyethoxy)-5-aminopyrazin-2-yl)H-imidazo[1,2-a]pyridin-2-yl)-1-acetylazetidine-2-carboxamide as its TFA salt. LC/MS (m/z): 426.2 (MH⁺), Rₜ: 1.61 min.

### Example 34

### Preparation of (S)-tert-butyl 2-(6-(6-amino-5-(trifluoromethyl)pyridin-3-yl)H-imidazo[1,2-a]pyridin-2-ylcarbamoyl)piperidine-1-carboxylate

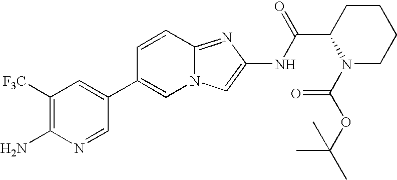

(S)-tert-butyl 2-(6-iodoH-imidazo[1,2-a]pyridin-2-ylcarbamoyl)piperidine-1-carboxylate (470 mg, 1 mmol) and 5-(4,4,5,5-tetramethyl-1,3,2-dioxaborolan-2-yl)-3-(trifluoromethyl)pyridin-2-amine (432 mg, 1.5 mmol) was mixed with DME (5 mL) and 2 M Na₂CO₃ aqueous solution (3:1) in the microwave reaction vessel. The reaction mixture was degassed by anhydrous N₂ stream for 15 min followed by the addition of Pd(dppf)₂Cl₂-DCM 81 mg, 0.1 mmol. The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by preparative HPLC to give (S)-tert-butyl 2-(6-(6-amino-5-(trifluoromethyl)pyridin-3-yl)H-imidazo[1,2-a]pyridin-2-ylcarbamoyl)piperidine-1-carboxylate as its TFA salt. LC/MS (m/z): 505.2 (MH⁺), Rₜ: 2.51 min.

### Example 35

### Preparation of (S)—N-(6-(6-amino-5-(trifluoromethyl)pyridin-3-yl)H-imidazo[1,2-a]pyridin-2-yl)piperidine-2-carboxamide

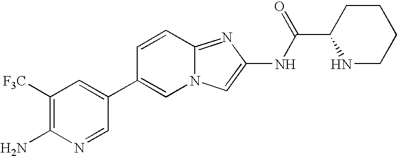

TFA (0.3 mL) was added to a stirring solution of (S)-tert-butyl 2-(6-(6-amino-5-(trifluoromethyl)pyridin-3-yl)H-imidazo[1,2-a]pyridin-2-ylcarbamoyl)piperidine-1-carboxylate (112 mg, 0.22 mmol) in CH₂Cl₂ (1 mL). The reaction mixture was maintained at rt for 1 h. The crude mixture was neutralized with saturated aqueous sodium carbonate (5 mL) then diluted with CH₂Cl₂ (10 mL) and H₂O (10 mL). The organic layer was separated, and the aqueous phase was extracted with CH₂Cl₂ (2×20 mL). The combined organic portions were washed with brine (40 mL), dried over anhydrous Na₂SO₄, filtered, concentrated and dried in vacuo to give (S)—N-(6-(6-amino-5-(trifluoromethyl)pyridin-3-yl)H-imidazo[1,2-a]pyridin-2-yl)piperidine-2-carboxamide as a brown solid. The crude product was used for the next step without further purification. LC/MS (m/z): 405.2 (MH⁺), Rₜ: 1.68 min.

### Example 36

### Preparation of (S)—N-(6-(6-amino-5-(trifluoromethyl)pyridin-3-yl)H-imidazo[1,2-a]pyridin-2-yl)-1-benzylpiperidine-2-carboxamide

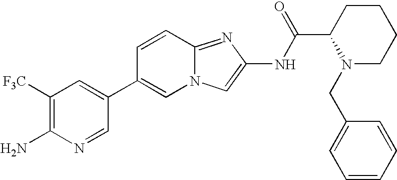

Benzyl bromide (0.010 mL, 0.068 mmol) was added to a stirring solution of (S)—N-(6-(6-amino-5-(trifluoromethyl)pyridin-3-yl)H-imidazo[1,2-a]pyridin-2-yl)piperidine-2-carboxamide (25 mg, 0.062 mmol) and Et₃N (0.010 mL, 0.075 mmol) in CH₂Cl₂ (0.5 mL). The reaction mixture was maintained at rt for 16 h. The reaction mixture was concentrated and the crude residue was dissolved in DMSO then purified by preparative HPLC to give (S)—N-(6-(6-amino-5-(trifluoromethyl)pyridin-3-yl)H-imidazo[1,2-a]pyridin-2-yl)-1-benzylpiperidine-2-carboxamide as its TFA salt. LC/MS (m/z): 495.2 (MH⁺), Rₜ: 2.02 min.

### Example 37

### Preparation of (3Z)-1-(3-(6-(6-amino-5-(trifluoromethyl)pyridin-3-yl)imidazo[1,2-b]pyridazin-2-ylcarbamoyl)propyl)-3-cyano-2-phenylisourea

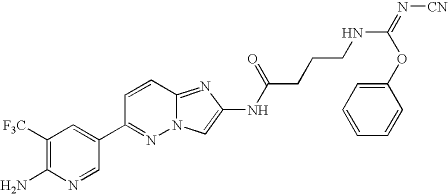

Diphenyl cyanocarbonimidate (63 mg, 0.26 mmol) was added to a stirring solution of 4-amino-N-(6-(6-amino-5-(trifluoromethyl)pyridin-3-yl)imidazo[1,2-b]pyridazin-2-yl)butanamide (100 mg, 0.26 mmol) in MeOH (5 mL). The reaction was heated for 2 h in a 60° C. oil bath. After cooling to rt, CH₂Cl₂ (10 mL) was added to the crude reaction mixture to form a precipitate. The liquor was decanted off and concentrated down to give (3Z)-1-(3-(6-(6-amino-5-(trifluoromethyl)pyridin-3-yl)imidazo[1,2-b]pyridazin-2-ylcarbamoyl)propyl)-3-cyano-2-phenylisourea as a pale solid. The crude product was used for the next step without further purification. LC/MS (m/z): 524.1 (MH⁺), Rₜ: 2.44 min.

### Example 38

### Preparation of (2E)-(3-(6-(6-amino-5-(trifluoromethyl)pyridin-3-yl)imidazo[1,2-b]pyridazin-2-ylcarbamoyl)propyl)-2-cyanoguanidine

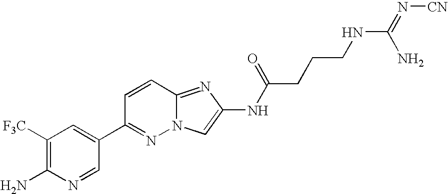

A mixture of (3Z)-1-(3-(6-(6-amino-5-(trifluoromethyl)pyridin-3-yl)imidazo[1,2-b]pyridazin-2-ylcarbamoyl)propyl)-3-cyano-2-phenylisourea (40 mg, 0.076 mmol) and NH₄OH (1.2 mL) in EtOH (0.4 mL) was heated for 1 h in a 60° C. oil bath. The reaction mixture was concentrated down and the crude residue was dissolved in DMSO then purified by preparative HPLC to give (2)-(3-(6-(6-amino-5-(trifluoromethyl)pyridin-3-yl)imidazo[1,2-b]pyridazin-2-ylcarbamoyl)propyl)-2-cyanoguanidine as its TFA salt. LC/MS (m/z): 447.2 (MH⁺), Rₜ: 1.92 min.

### Example 39

### Preparation of N-(6-(6-amino-5-(trifluoromethyl)pyridin-3-yl)-7-fluoroH-imidazo[1,2-a]pyridin-2-yl)acetamide

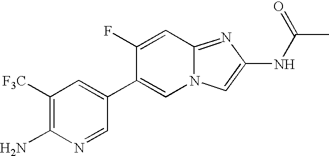

N-(6-bromo-7-fluoroH-imidazo[1,2-a]pyridin-2-yl)acetamide (32 mg, 0.11 mmol) and 5-(4,4,5,5-tetramethyl-1,3,2-dioxaborolan-2-yl)-3-(trifluoromethyl)pyridin-2-amine (63 mg, 0.22 mmol) was mixed with DME and 2 M Na₂CO₃ aqueous solution (3:1, 1.2 mL) in a microwave reaction vessel. The reaction mixture was degassed by anhydrous N₂ stream for 15 min followed by the addition of Pd(dppf)C₁₂-DCM (10 mg, 0.011 mmol). The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by a preparative HPLC to give N-(6-(6-amino-5-(trifluoromethyl)pyridin-3-yl)-7-fluoroH-imidazo[1,2-a]pyridin-2-yl)acetamide as its TFA salt. LC/MS (m/z): 354.0 (MH⁺), Rₜ: 1.83 min.

### Example 40

### Preparation of N-(6-(5-aminopyrazin-2-yl)imidazo[1,2-b]pyridazin-2-yl)acetamide

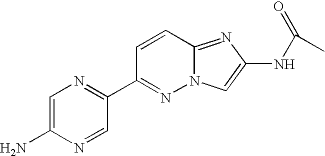

N-(6-chloroimidazo[1,2-b]pyridazin-2-yl)acetamide (32 mg, 0.15 mmol) and crude 5-(4,4,5,5-tetramethyl-1,3,2-dioxaborolan-2-yl)pyrazin-2-amine (0.2 mmol) was mixed with 2 M Na₂CO₃ aqueous solution (0.5 mL) in the microwave reaction vessel. The reaction mixture was degassed by anhydrous N₂ stream for 15 min followed by the addition of Pd(dppf)Cl₂-DCM (12 mg, 0.015 mmol). The reaction mixture was then heated in a microwave reactor at 110° C. for 600 sec. Excess amount of anhydrous Na₂SO₄ was added and the reaction mixture was diluted with EtOAc (3 mL). The organic layer was filtered, concentrated, and dried in vacuo. The crude solid was purified by preparative HPLC to give N-(6-(5-aminopyrazin-2-yl)imidazo[1,2-b]pyridazin-2-yl)acetamide as its TFA salt. LC/MS (m/z): 270.1 (MH⁺), Rₜ: 1.57 min.

### Example 41

### Preparation of N-(4-(2-acetamido-6-(6-amino-5-(trifluoromethyl)pyridin-3-yl)imidazo[1,2-a]pyridin-3-yl)phenyl)acetamide

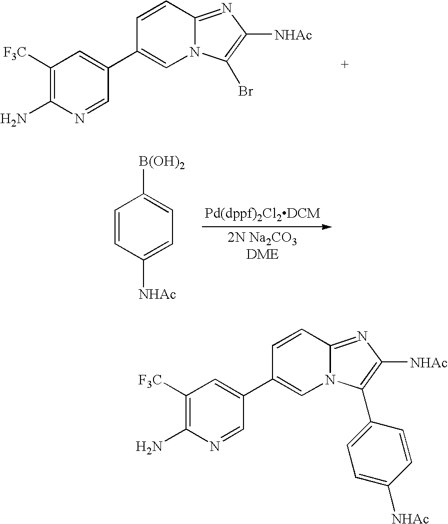

N-(6-(6-Amino-5-(trifluoromethyl)pyridin-3-yl)-3-bromoimidazo[1,2-a]pyridin-2-yl)acetamide (18 mg, 0.043 mmol) was dissolved in DME (1 mL). 4-Acetamidophenylboronic acid (0.087 mmol) was added, followed by 2 M aq. Na₂CO₃ (0.3 mL). The reaction mixture was purged with N₂ for 2 min, then Pd(dppf)₂Cl₂ dichloromethane adduct (2 mg, 0.002 mmol) was added. The reaction mixture was stirred at 95° C. for 3 h. Water and EtOAc were added to the reaction mixture. The two phases were separated and the aqueous phase was extracted with EtOAc. The organic extracts were combined and washed with water (1×), brine (1×) and dried (Na₂SO₄). The solvent was removed under reduced pressure and the residue was dissolved in DMSO and purified by reverse phase preparatory HPLC to give N-(4-(2-acetamido-6-(6-amino-5-(trifluoromethyl)pyridin-3-yl)imidazo[1,2-a]pyridin-3-yl)phenyl)acetamide as the TFA salt (5.7 mg, 23%). LC/MS (m/z): 469.1 (MH⁺), Rₜ: 1.85 min.

### Example 42

### Preparation of N-(6-(6-amino-5-(trifluoromethyl)pyridin-3-yl)-3-cyanoimidazo[1,2-a]pyridin-2-yl)acetamide and N-(6-(6-amino-5-(trifluoromethyl)pyridin-3-yl)-3-(aminooxycarbonyl)imidazo[1,2-a]pyridin-2-yl)acetamide

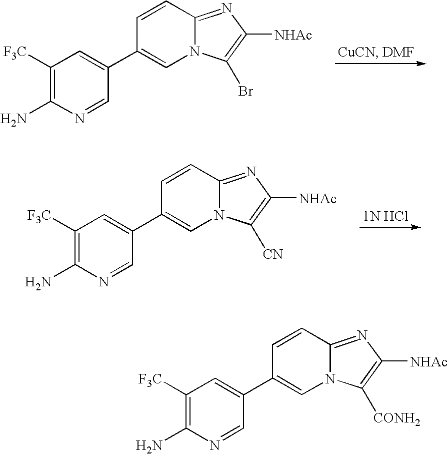

N-(6-(6-Amino-5-(trifluoromethyl)pyridin-3-yl)-3-bromoimidazo[1,2-a]pyridin-2-yl)acetamide (130 mg, 0.31 mmo, 1 equiv.) was dissolved in DMF (3 mL) and CuCN (56 mg, 0.62 mmol, 2 equiv.) was added. The reaction mixture was heated under microwave irradiation at 200° C. for 5 min. The DMF was concentrated under reduced pressure, the residue was triturated with water and purified with reverse phase preparative HPLC to obtain N-(6-(6-amino-5-(trifluoromethyl)pyridin-3-yl)-3-cyanoimidazo[1,2-a]pyridin-2-yl)acetamide TFA salt. LC/MS (m/z): 361.0 (MH⁺), Rₜ: 1.88 min.

The nitrile was treated with a 1.5 mL of ACN/H₂O/1N HCl (1:1:1) and lyophilized to give N-(6-(6-amino-5-(trifluoromethyl)pyridin-3-yl)-3-(aminooxycarbonyl)imidazo[1,2-a]pyridin-2-yl)acetamide (6% yield). LC/MS (m/z): 379.0 (MH⁺), Rₜ: 1.50 min. (Note: the nitrile hydrolizes to the amide even in the presence of traces of TFA)

### Example 43

### Preparation of (S)—N-(6-(6-amino-5-(trifluoromethyl)pyridin-3-yl)imidazo[1,2-b]pyridazin-2-yl)-2-(pyrrolidin-3-yloxy)acetamide

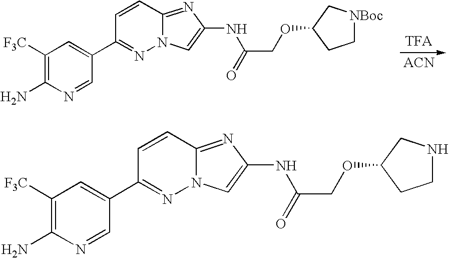

(S)-tert-Butyl 3-(2-(6-(6-amino-5-(trifluoromethyl)pyridin-3-yl)imidazo[1,2-b]pyridazin-2-ylamino)-2-oxoethoxy)pyrrolidine-1-carboxylate (4.5 mg, 0.008 mmol) was suspended in CAN (0.30 mL) and trifluoroacetic acid (0.1 mL) was added. The reaction mixture was stirred at room temperature overnight. Water was added (0.2 mL) and the mixture directly lyophilized to obtain the desired product as a TFA salt (quant., 99% purity). LC/MS (m/z): 422.1 (MH⁺), Rₜ: 1.81 min.

The following compounds were prepared according to Example 43 from the corresponding Boc-protected amine:

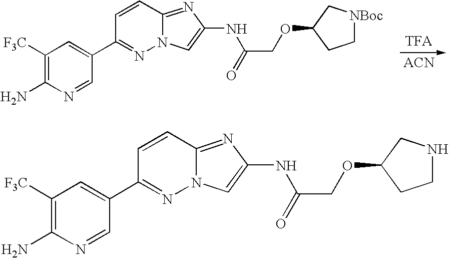

(R)—N-(6-(6-Amino-5-(trifluoromethyl)pyridin-3-yl)imidazo[1,2-b]pyridazin-2-yl)-2-(pyrrolidin-3-yloxy)acetamide. LC/MS (m/z.): 422.1 (MH⁺), Rₜ: 1.81 min

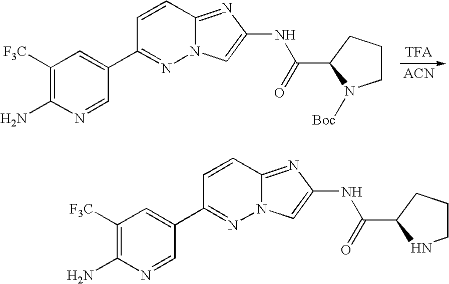

(R)—N-(6-(6-Amino-5-(trifluoromethyl)pyridin-3-yl)imidazo[1,2-b]pyridazin-2-yl)pyrrolidine-2-carboxamide LC/MS (m/z): 392.1 (MH⁺), Rₜ: 1.76 min

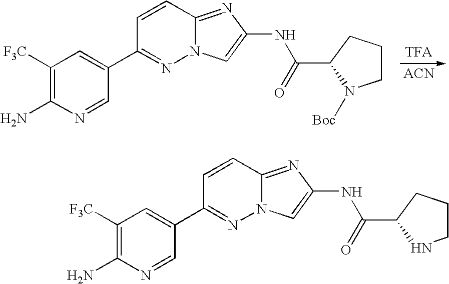

(S)—N-(6-(6-Amino-5-(trifluoromethyl)pyridin-3-yl)imidazo[1,2-b]pyridazin-2-yl)pyrrolidine-2-carboxamide. LC/MS (m/z): 392.2 (MH⁺), Rₜ: 1.81 min.

### Example 44

### N-(6-(6-amino-5-(4-benzylpiperazin-1-ylsulfonyl)pyridin-3-yl)imidazo[1,2-a]pyridin-2-yl)acetamide

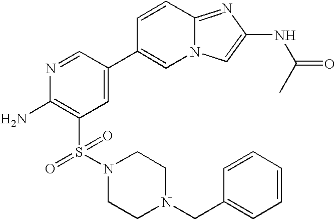

A mixture of 2-acetamidoimidazo[1,2-a]pyridin-6-ylboronic acid (25.4 mg, 0.07 mmol), 3-(4-benzylpiperazin-1-ylsulfonyl)-5-bromopyridin-2-amine (21 mg, 0.05 mmol), PdCl₂(dppf)-CH₂Cl₂ (4 mg, 10 mole %), 2M aq. Na₂CO₃ (0.3 mL) in 1,2-dimethoxyethane (1 mL) was degassed briefly with nitrogen, sealed and subjected to microwave irradiation at 110° C. for 600 seconds. The mixture was diluted with ethyl acetate, and the two phases were separated. The organic phase was dried (Na₂SO₄), filtered and concentrated. The crude material was purified on reverse phase preparative HPLC, affording the desired product as a TFA salt. LC/MS (m/z): 506.1 (MH⁺), Rₜ: 1.68 min.

### Example 45

### Preparation of N-(3-acetyl-6-(6-amino-5-(trifluoromethyl)pyridin-3-yl)imidazo[1,2-a]pyridin-2-yl)acetamide

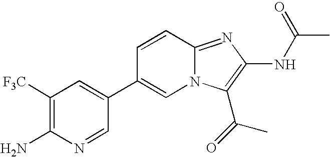

Copper(I) iodide (0.8 mg, 0.004 mmol) and dichloro(bis-triphenylphosphine)palladium (2.8 mg, 0.004 mmol) were added to a mixture of N-(6-(6-amino-5-(trifluoromethyl)pyridin-3-yl)-3-bromo-imidazo[1,2-a]pyridin-2-yl)acetamide (35 mg, 0.084 mmol) and trimethylsilylacetylene (0.024 ml, 0.17 mmol) in triethylamine (0.08 mL) and DMF (0.16 mμL). The mixture was heated at 80° C. for 15 h then partitioned between ethyl acetate and water. The organic layer was washed with brine, dried over sodium sulfate and evaporated to give the crude material. N-(3-acetyl-6-(6-amino-5-(trifluoromethyl)pyridin-3-yl)imidazo[1,2-a]pyridin-2-yl)acetamide TFA salt was obtained after reverse phase preparative HPLC. LC/MS m/z 378.0 (MH⁺), Rₜ: 1.71 min, HPLC Rₜ: 1.83 min.

### Example 46

### Preparation of N-(6-(6-amino-5-(trifluoromethyl)pyridin-3-yl)-3-vinylimidazo[1,2-a]pyridin-2-yl)acetamide

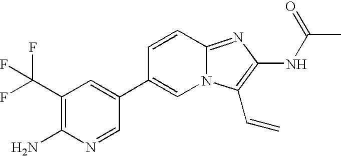

N-(6-(6-amino-5-(trifluoromethyl)pyridin-3-yl)-3-vinylimidazo[1,2-a]pyridin-2-yl)acetamide was prepared from a Suzuki coupling reaction of N-(6-(6-amino-5-(trifluoromethyl)pyridin-3-yl)-3-bromo-imidazo[1,2-a]pyridin-2-yl)acetamide (See Example 4) with commercially available vinylboronic acid pinacolester.

According to Example 46, the following compounds were prepared:

N-(6-(6-amino-5-(trifluoromethyl)pyridin-3-yl)-3-vinylimidazo[1,2-a]pyridin-2-yl)acetamide. LC/MS (m/z) 362.0 (MH⁺), Rₜ: 1.47 min; HPLC Rₜ: 1.74 min.

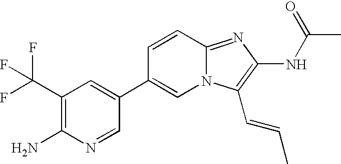

(E)-N-(6-(6-amino-5-(trifluoromethyl)pyridin-3-yl)-3-(prop-1-enyl)imidazo[1,2-a]pyridin-2-yl)acetamide. LC/MS (m/z) 376.0 (MH⁺), Rₜ: 1.60 min; HPLC Rₜ: 1.96 min.

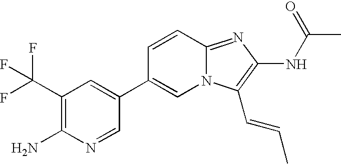

(Z)-N-(6-(6-amino-5-(trifluoromethyl)pyridin-3-yl)-3-(prop-1-enyl)imidazo[1,2-a]pyridin-2-yl)acetamide. LC/MS (m/z) 376.0 (MH⁺), Rₜ: 1.53 min; HPLC Rₜ: 1.82 min.

The compounds in Table 1 were synthesized according to the Examples provided above. PI3K inhibitory (IC₅₀) values of the compounds were determined according to the various assays described in Biological Methods 1-3. In Tables 1, 2, and 3, a “+” indicates that the compound has an IC₅₀ or EC₅₀ value of greater than or equal to 25 μM, a “++” indicates that the compound has an IC₅₀ or EC₅₀ value of lower than 25 μM, a “+++” indicates that the compound has an IC₅₀ or EC₅₀ value of lower than 10 μM, a “++++” indicates that the compound has an IC₅₀ or EC₅₀ value of lower than 1 μM, and N/D indicates that the activity was not determined for the assay indicated.

TABLE 1

| | | | A2780 | A2780 | | LC/MS |
|---|---|---|---|---|---|---|
| Com- | | PI3K | pAKT | Cell | | (m/z, |
| pound | | Alpha | 473 | prolif. | | Rt) |
| # | Structure | IC50 | EC50 | EC50 | Name | min |
| 1 | 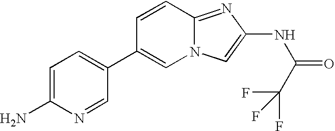 | ++++ | +++ | ++ | N-[6-(6- Amino- pyridin-3-yl)- imidazo[1,2- a]pyridin-2- yl]-2,2,2- trifluoro- acetamide | 322.1, 1.67 |
| 2 | 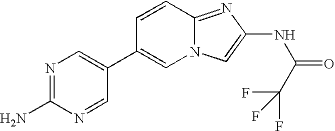 | ++++ | ++ | ++ | N-[6-(2- Amino- pyrimidin-5- yl)- imidazo[1,2- a]pyridin-2- yl]-2,2,2- trifluoro- acetamide | 323.2, 1.48 |
| 3 | 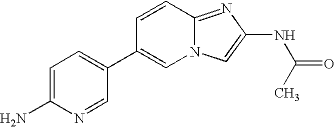 | ++++ | ++++ | +++ | N-[6-(6- Amino- pyridin-3-yl)- imidazo[1,2- a]pyridin-2- yl]-acetamide | 271.0, 1.46 |
| 4 | 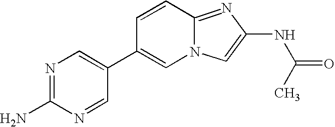 | ++++ | +++ | ++ | N-[6-(2- Amino- pyrimidin-5- yl)- imidazo[1,2- a]pyridin-2- yl]-acetamide | 268.1, 1.16 |
| 5 | 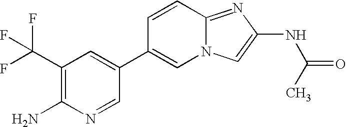 | ++++ | ++++ | +++ | N-[6-(6- Amino-5- trifluoromethyl- pyridin-3-yl)- imidazo[1,2- a]pyridin-2- yl]-acetamide | 336.1, 1.59 |
| 6 | 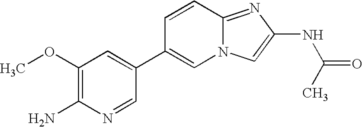 | ++++ | ++++ | +++ | N-[6-(6- Amino-5- methoxy- pyridin-3-yl)- imidazo[1,2- a]pyridin-2- yl]-acetamide | 298.2, 1.25 |
| 7 | 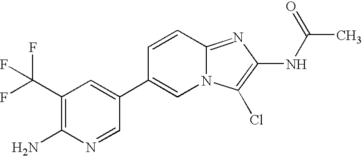 | ++++ | +++ | +++ | N-[6-(6- Amino-5- trifluoromethyl- pyridin-3-yl)- 3-chloro- imidazo[1,2- a]pyridin-2- yl]-acetamide | 370.0, 1.63 |
| 8 | 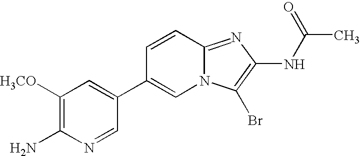 | ++++ | ++++ | +++ | N-[6-(6- Amino-5- methoxy- pyridin-3-yl)- 3-bromo- imidazo[1,2- a]pyridin-2- yl]-acetamide | 375.9/ 377.9, 1.31 |
| 9 | 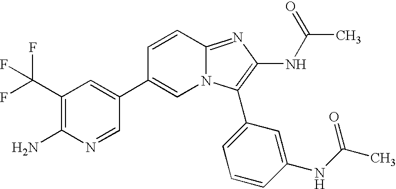 | ++++ | N/D | ++ | N-[3-(3- Acetylamino- phenyl)-6-(6- amino-5- trifluoromethyl- pyridin-3-yl)- imidazo[1,2- a]pyridin-2- yl]-acetamide | 469.1, 1.93 |
| 10 | 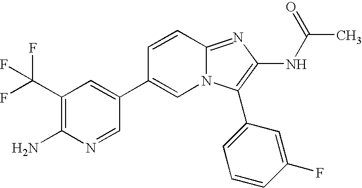 | ++++ | N/D | ++ | N-[6-(6- Amino-5- trifluoromethyl- pyridin-3-yl)- 3-(3-fluoro- phenyl)- imidazo[1,2- a]pyridin-2- yl]-acetamide | 430.1, 2.21 |
| 11 | 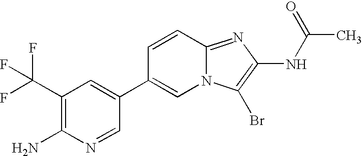 | ++++ | +++ | ++ | N-[6-(6- Amino-5- trifluoromethyl- pyridin-3-yl)- 3-bromo- imidazo[1,2- a]pyridin-2- yl]-acetamide | 414.0, 1.71 |
| 12 | 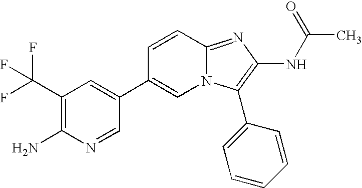 | ++++ | +++ | ++ | N-[6-(6- Amino-5- trifluoromethyl- pyridin-3-yl)- 3-phenyl- imidazo[1,2- a]pyridin-2- yl]-acetamide | 412.1, 1.90 |
| 13 | 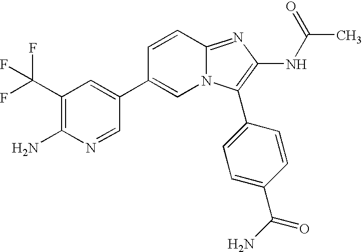 | ++++ | ++ | ++ | 4-[2- Acetylamino- 6-(6-amino-5- trifluoromethyl- pyridin-3-yl)- imidazo[1,2- a]pyridin-3- yl]-benzamide | 455.2, 1.58 |
| 14 | 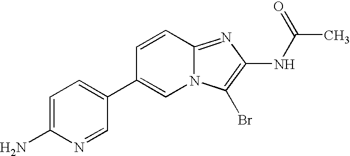 | +++ | N/D | N/D | N-[6-(6- Amino- pyridin-3-yl)- 3-bromo- imidazo[1,2- a]pyridin-2- yl]-acetamide | 346.0, 1.28 |
| 15 | 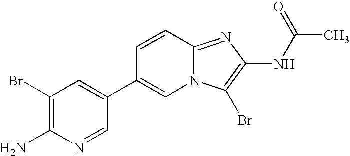 | ++++ | N/D | +++ | N-[6-(6- Amino-5- bromo- pyridin-3-yl)- 3-bromo- imidazo[1,2- a]pyridin-2- yl]-acetamide | 423.9, 1.46 |
| 16 | 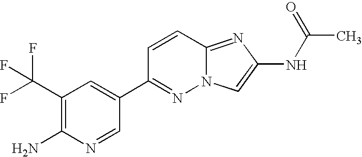 | ++++ | ++++ | ++++ | N-[6-(6- Amino-5- trifluoromethyl- pyridin-3-yl)- imidazo[1,2- b]pyridazin-2- yl]-acetamide | 337.0, 1.79 |
| 17 | 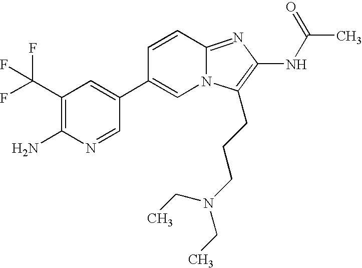 | +++ | N/D | N/D | N-[6-(6- Amino-5- trifluoromethyl- pyridin-3-yl)- 3-(3- diethylamino- propyl)- imidazo[1,2- a]pyridin-2- yl]-acetamide | 225.1, 1.51 |
| 18 | 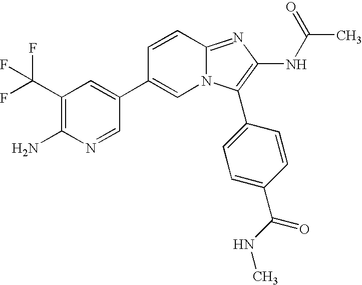 | ++++ | +++ | ++ | 4-[2- Acetylamino- 6-(6-amino-5- trifluoromethyl- pyridin-3-yl)- imidazo[1,2- a]pyridin-3- yl]-N-methyl- benzamide | 469.1, 1.64 |
| 19 | 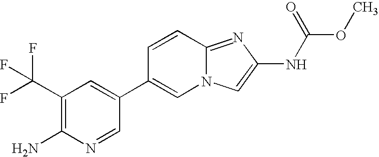 | ++++ | ++++ | +++ | [6-(6-Amino- 5-trifluoromethyl- pyridin-3-yl)- imidazo[1,2- a]pyridin-2- yl]-carbamic acid methyl ester | 352.0, 1.68 |
| 20 | 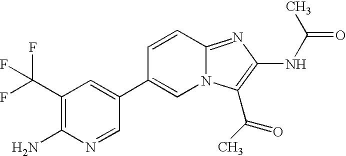 | +++ | N/D | N/D | N-[3-Acetyl-6- (6-amino-5- trifluoromethyl- pyridin-3-yl)- imidazo[1,2- a]pyridin-2- yl]-acetamide | 378.0, 1.71 |
| 21 | 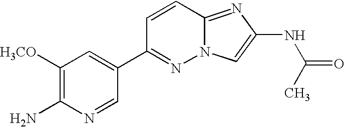 | ++++ | ++++ | +++ | N-[6-(6- Amino-5- methoxy- pyridin-3-yl)- imidazo[1,2- b]pyridazin-2- yl]-acetamide | 299.1, 1.67 |
| 22 | 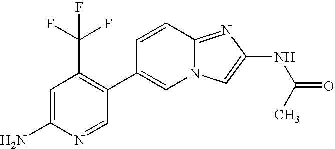 | +++ | N/D | N/D | N-[6-(6- Amino-4- trifluoromethyl- pyridin-3-yl)- imidazo[1,2- a]pyridin-2- yl]-acetamide | 336.1, 1.63 |
| 23 | 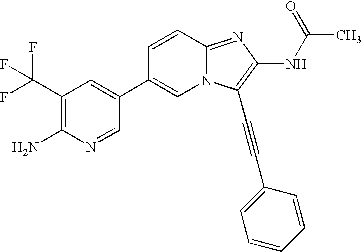 | ++++ | ++++ | +++ | N-[6-(6- Amino-5- trifluoromethyl- pyridin-3-yl)- 3-phenylethynyl- imidazo[1,2- a]pyridin-2- yl]-acetamide | 436.2, 2.37 |
| 24 | 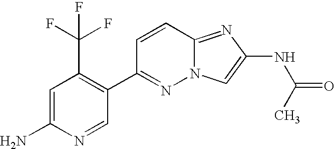 | ++++ | N/D | ++ | N-[6-(6- Amino-4- trifluoromethyl- pyridin-3-yl)- imidazo[1,2- b]pyridazin-2- yl]-acetamide | 337.2, 1.63 |
| 25 | 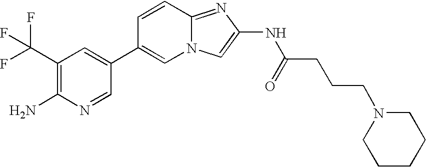 | ++++ | ++++ | +++ | N-[6-(6- Amino-5- trifluoromethyl- pyridin-3-yl)- imidazo[1,2- a]pyridin-2- yl]-4- piperidin-1-yl- butyramide | 447.1, 1.74 |
| 26 | 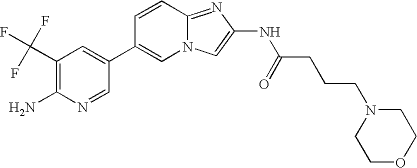 | ++++ | ++++ | +++ | N-[6-(6- Amino-5- trifluoromethyl- pyridin-3-yl)- imidazo[1,2- a]pyridin-2- yl]-4- morpholin-4- yl-butyramide | 449.1, 1.63 |
| 27 | 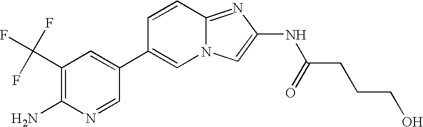 | ++++ | ++++ | +++ | N-[6-(6- Amino-5- trifluoromethyl- pyridin-3-yl)- imidazo[1,2- a]pyridin-2- yl]-4-hydroxy- butyramide | 380.1, 1.70 |
| 28 | 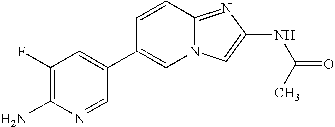 | ++++ | ++++ | ++ | N-[6-(6- Amino-5- fluoro-pyridin- 3-yl)- imidazo[1,2- a]pyridin-2- yl]-acetamide | 286.0, 1.26 |
| 29 | 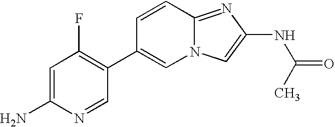 | ++++ | +++ | ++ | N-[6-(6- Amino-4- fluoro-pyridin- 3-yl)- imidazo[1,2- a]pyridin-2- yl]-acetamide | 286.0, 121 |
| 30 | 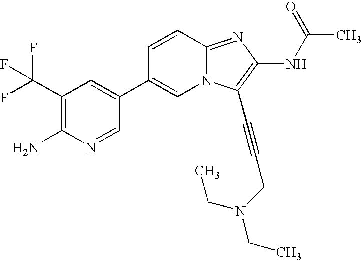 | ++++ | ++++ | +++ | N-[6-(6- Amino-5- trifluoromethyl- pyridin-3-yl)- 3-(3- diethylamino- prop-1-ynyl)- imidazo[1,2- a]pyridin-2- yl]-acetamide | 445.1, 1.66 |
| 31 | 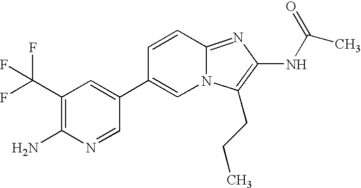 | ++++ | N/D | ++ | N-[6-(6- Amino-5- trifluoromethyl- pyridin-3-yl)- 3-propyl- imidazo[1,2- a]pyridin-2- yl]-acetamide | 378.0, 1.97 |
| 32 | 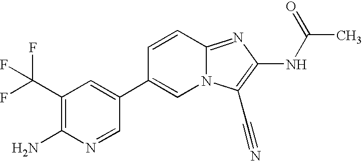 | ++++ | ++++ | ++++ | N-[6-(6- Amino-5- trifluoromethyl- pyridin-3-yl)- 3-cyano- imidazo[1,2- a]pyridin-2- yl]-acetamide | 361.0, 2.06 |
| 33 | 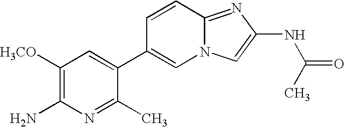 | + | ++ | +++ | N-(6-(6- amino-5- methoxy-2- methylpyridin- 3-yl)imidazo[1,2- a]pyridin-2- yl)acetamide | 312.0, 1.13 |
| 34 | 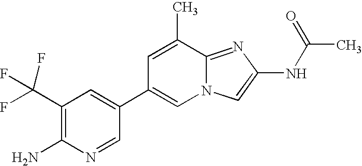 | + | N/D | N/D | N-(6-(6- amino-5- (trifluoromethyl) pyridin-3- yl)-8- methylimidazo[1,2- a]pyridin-2- yl)acetamide | 350.1, 1.64 |
| 35 | 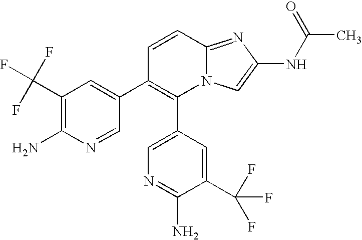 | + | N/D | N/D | N-(5,6-bis(6- amino-5- (trifluoromethyl) pyridin-3- yl)imidazo[1,2- a]pyridin-2- yl)acetamide | 496.0, 1.77 |
| 36 | 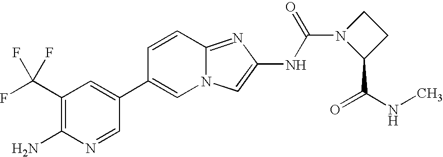 | ++ | N/D | ++ | (S)-N1-(6-(6- amino-5- (trifluoromethyl) pyridin-3- yl)imidazo[1,2- a]pyridin-2- yl)-N2- methylazetidine- 1,2-dicarboxamide | 434.2, 1.64 |
| 37 | 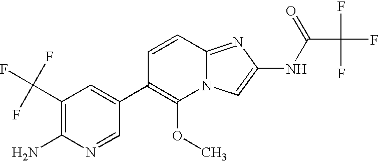 | ++ | N/D | ++ | N-(6-(6- amino-5- (trifluoromethyl) pyridin-3- yl)-5- methoxy- imidazo[1,2- a]pyridin-2- yl)-2,2,2- trifluoroacetamide | 420.1, 2.03 |
| 38 | 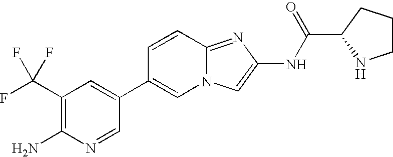 | ++ | N/D | +++ | (S)-N-(6-(6- amino-5- (trifluoromethyl) pyridin-3- yl)imidazo[1,2- a]pyridin-2- yl)pyrrolidine- 2-carboxamide | 391.1, 1.66 |
| 39 | 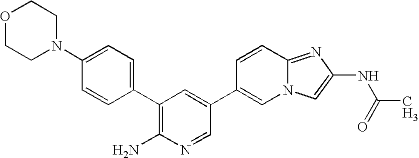 | +++ | +++ | +++ | N-(6-(6- amino-5-(4- morpholino- phenyl)pyridin- 3-yl)imidazo[1,2- a]pyridin-2- yl)acetamide | 429.2, 1.76 |
| 40 | 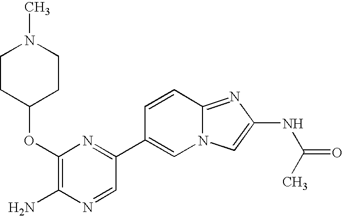 | +++ | N/D | ++ | N-(6-(5- amino-6-(1- methylpiperidin-4- yloxy)pyrazin-2- yl)imidazo[1,2- a]pyridin-2- yl)acetamide | 382.1, 1.49 |
| 41 | 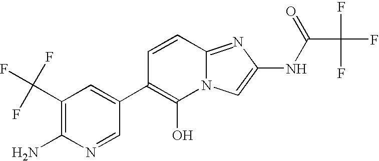 | +++ | N/D | ++ | N-(6-(6- amino-5- (trifluoromethyl) pyridin-3- yl)-5- hydroxyimi- dazo[1,2- a]pyridin-2- yl)-2,2,2- trifluoroacetamide | 406.0, 2.20 |
| 42 | 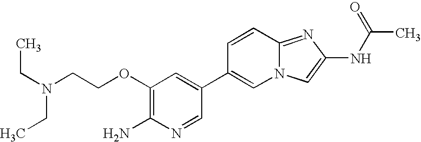 | +++ | N/D | ++ | N-(6-(6- amino-5-(2- (diethylamino) ethoxy)pyridin- 3-yl)imidazo[1,2- a]pyridin-2- yl)acetamide | 383.2, 1.31 |
| 43 | 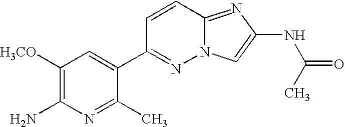 | +++ | N/D | ++ | N-(6-(6- amino-5- methoxy-2- methylpyridin- 3-yl)imidazo[1,2- b]pyridazin- 2-yl)acetamide | 313.0, 1.27 |
| 44 | 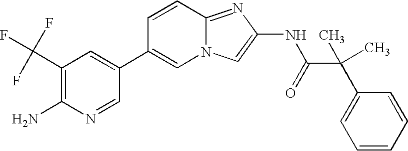 | +++ | N/D | ++ | N-(6-(6- amino-5- (trifluoromethyl) pyridin-3- yl)imidazo[1,2- a]pyridin-2- yl)-2-methyl-2- phenylpropan amide | 440.1, 2.42 |
| 45 | 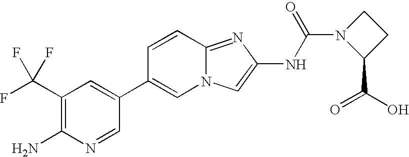 | +++ | N/D | ++ | (S)-1-(6-(6- amino-5- (trifluoromethyl) pyridin-3- yl)imidazo[1,2- a]pyridin-2- ylcarbamoyl) azetidine-2- carboxylic acid | 421.1, 1.72 |
| 46 | 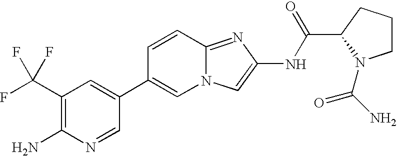 | +++ | N/D | +++ | (S)-N2-(6-(6- amino-5- (trifluoromethyl) pyridin-3- yl)imidazo[1,2- a]pyridin-2- yl)pyrrolidine- 1,2-dicarboxamide | 434.2, 1.76 |
| 47 | 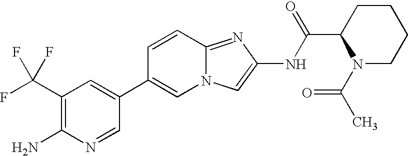 | +++ | N/D | N/D | (R)-1-acetyl- N-(6-(6- amino-5- (trifluoromethyl) pyridin-3- yl)imidazo[1,2- a]pyridin-2- yl)piperidine- 2-carboxamide | 447.1, 1.94 |
| 48 | 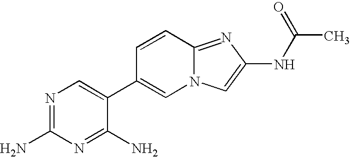 | +++ | N/D | N/D | N-(6-(2,4- diaminopyrimidin-5- yl)imidazo[1,2- a]pyridin-2- yl)acetamide | 284.0, 0.83 |
| 49 | 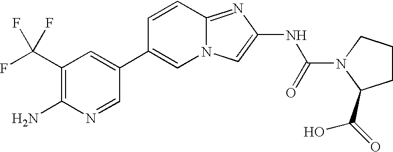 | +++ | N/D | N/D | (S)-1-(6-(6- amino-5- (trifluoromethyl) pyridin-3- yl)imidazo[1,2- a]pyridin-2- ylcarbamoyl) pyrrolidine-2- carboxylic acid | 435.1, 1.79 |
| 50 | 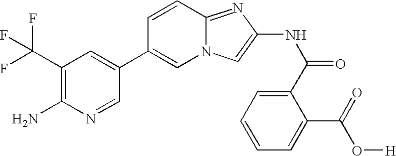 | +++ | N/D | N/D | 2-(6-(6- amino-5- (trifluoromethyl) pyridin-3- yl)imidazo[1,2- a]pyridin-2- ylcarbamoyl) benzoic acid | 442.0, 1.92 |
| 51 | 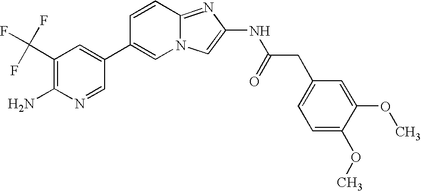 | ++++ | ++++ | +++ | N-(6-(6- amino-5- (trifluoromethyl) pyridin-3- yl)imidazo[1,2- a]pyridin-2- yl)-2-(3,4- dimethoxyphenyl) acetamide | 472.1, 2.16 |
| 52 | 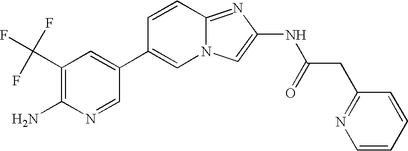 | ++++ | N/D | +++ | N-(6-(6- amino-5- (trifluoromethyl) pyridin-3- yl)imidazo[1,2- a]pyridin-2- yl)-2-(pyridin- 2-yl)acetamide | 413.1, 1.70 |
| 53 | 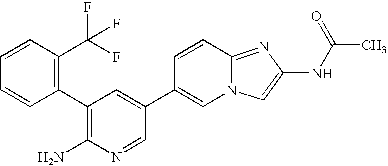 | ++++ | ++++ | ++++ | N-(6-(6- amino-5-(2- (trifluoromethyl) phenyl)pyridin-3- yl)imidazo[1,2- b]pyridazin- 2-yl)acetamide | 413.1, 1.67 |
| 54 | 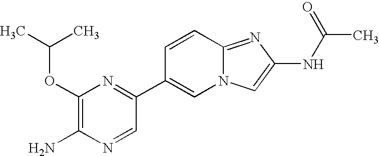 | ++++ | ++++ | +++ | N-(6-(5- amino-6- isopropoxy- pyrazin-2- yl)imidazo[1,2- a]pyridin-2- yl)acetamide | 327.1, 1.46 |
| 55 | 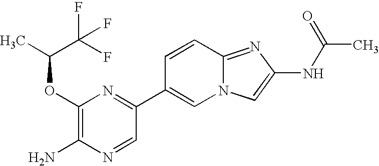 | ++++ | ++++ | ++++ | (S)-N-(6-(5- amino-6- (1,1,1- trifluoropropan-2- yloxy)pyrazin- 2-yl)imidazo[1,2- a]pyridin-2- yl)acetamide | 381.1, 2.04 |
| 56 | 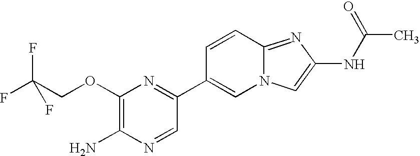 | ++++ | ++++ | ++++ | N-(6-(5- amino-6- (2,2,2- trifluoroethoxy) pyrazin-2- yl)imidazo[1,2- a]pyridin-2- yl)acetamide | 367.1, 1.92 |
| 57 | 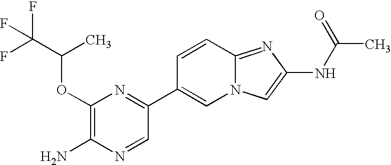 | ++++ | ++++ | ++++ | N-(6-(5- amino-6- (1,1,1- trifluoropropan-2- yloxy)pyrazin-2- yl)imidazo[1,2- a]pyridin-2- yl)acetamide | 381.1, 1.76 |
| 58 | 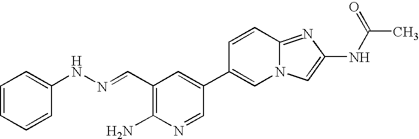 | ++++ | +++ | ++++ | (E)-N-(6-(6- amino-5-((2- phenylhydrazono) methyl)pyridin-3- yl)imidazo[1,2- a]pyridin-2- yl)acetamide | 385.9, 1.75 |
| 59 | 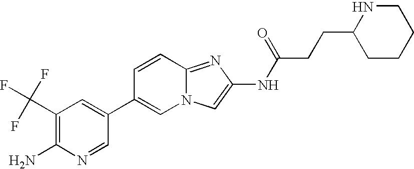 | ++++ | ++++ | ++++ | N-(6-(6- amino-5- (trifluoromethyl) pyridin-3- yl)imidazo[1,2- b]pyridazin- 2-yl)-3- (piperidin-2- yl)propanamide | 434.1, 1.88 |
| 60 | 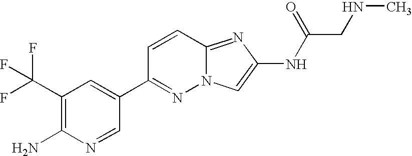 | ++++ | ++++ | ++++ | N-(6-(6- amino-5- (trifluoromethyl) pyridin-3- yl)imidazo[1,2- b]pyridazin- 2-yl)-2- (methylamino) acetamide | 365.9, 1.39 |
| 61 | 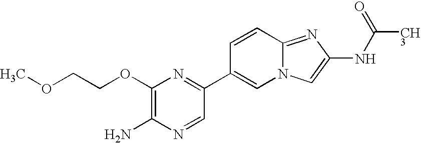 | ++++ | ++++ | ++++ | N-(6-(5- amino-6-(2- methoxyethoxy) pyrazin-2- yl)imidazo[1,2- a]pyridin-2- yl)acetamide | 343.1, 1.63 |
| 62 | 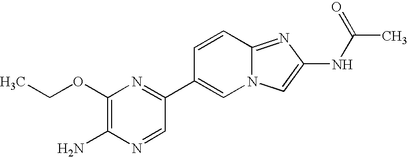 | ++++ | ++++ | ++++ | N-(6-(5- amino-6- ethoxypyrazin-2- yl)imidazo[1,2- a]pyridin-2- yl)acetamide | 313.0, 1.37 |
| 63 | 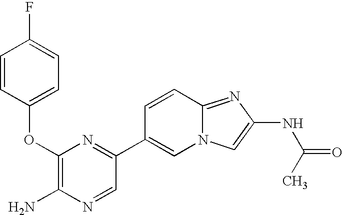 | ++++ | ++++ | ++++ | N-(6-(5- amino-6-(4- fluorophenoxy) pyrazin-2- yl)imidazo[1,2- a]pyridin-2- yl)acetamide | 379.1, 2.05 |
| 64 | 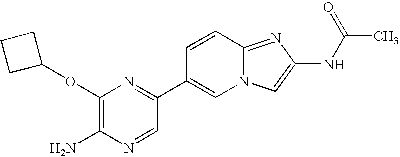 | ++++ | ++++ | ++++ | N-(6-(5- amino-6- cyclobutoxy- pyrazin-2- yl)imidazo[1,2- a]pyridin-2- yl)acetamide | 339.1, 1.57 |
| 65 | 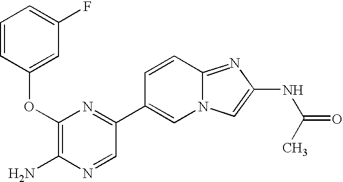 | ++++ | ++++ | ++++ | N-(6-(5- amino-6-(3- fluorophenoxy) pyrazin-2- yl)imidazo[1,2- a]pyridin-2- yl)acetamide | 379.1, 2.04 |
| 66 | 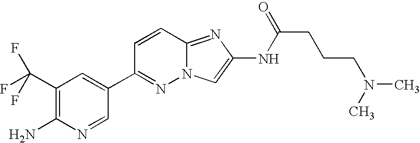 | ++++ | ++++ | ++++ | N-(6-(6- amino-5- (trifluoromethyl) pyridin-3- yl)imidazo[1,2- b]pyridazin- 2-yl)-4- (dimethylamino) butanamide | 408.1, 1.79 |
| 67 | 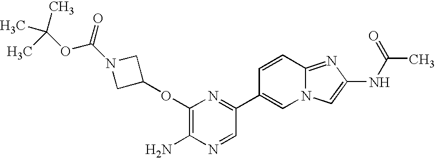 | ++++ | ++++ | ++++ | tert-butyl 3-(6- (2-acetamido- imidazo[1,2- a]pyridin-6-yl)-3- aminopyrazin- 2-yloxy)axetidine- 1-carboxylate | 440.1, 1.81 |
| 68 | 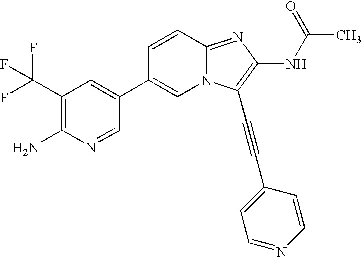 | ++++ | ++++ | ++++ | N-(6-(6- amino-5- (trifluoromethyl) pyridin-3- 4-ylethynyl)imi- dazo[1,2- a]pyridin-2- yl)acetamide | 437.0, 1.49 |
| 69 | 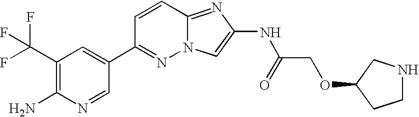 | ++++ | ++++ | ++++ | (R)-N-(6-(6- amino-5- (trifluoromethyl) pyridin-3- yl)imidazo[1,2- b]pyridazin- 2-yl)-2- (pyrrolidin-3- yloxy)acetamide | 469, 1.81 |
| 70 | 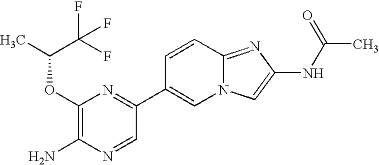 | ++++ | ++++ | ++++ | (R)-N-(6-(5- amino-6- (1,1,1- trifluoropropan-2- yloxy)pyrazin-2- a]pyridin-2- yl)acetamide | 381.1, 2.01 |
| 71 | 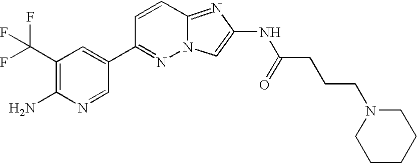 | ++++ | ++++ | ++++ | N-(6-(6- amino-5- (trifluoromethyl) pyridin-3- yl)imidazo[1,2- b]pyridazin- 2-yl)-4- (piperidin-1- yl)butanamide | 448.2, 1.60 |
| 72 | 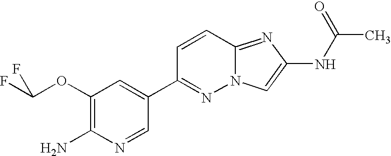 | ++++ | ++++ | ++++ | N-(6-(6- amino-5- (difluoromethoxy) pyridin-3- yl)imidazo[1,2- b]pyridazin- 2-yl)acetamide | 335.0, 1.42 |
| 73 | 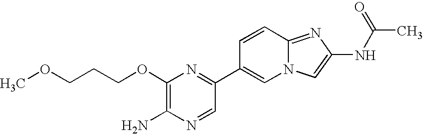 | ++++ | ++++ | ++++ | N-(6-(5- amino-6-(3- methoxypropoxy) pyrazin-2- yl)imidazo[1,2- a pyridin-2- yl)acetamide | 357.1, 1.42 |
| 74 | 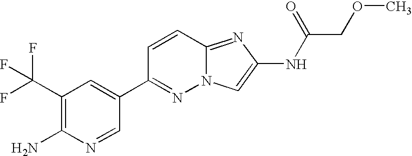 | ++++ | ++++ | ++++ | n-(6-(6- amino-5- (trifluoromethyl) pyridin-3- yl)imidazo[1,2- b]pyridazin- 2-yl)-2- methoxyacetamide | 366.9, 1.75 |
| 75 | 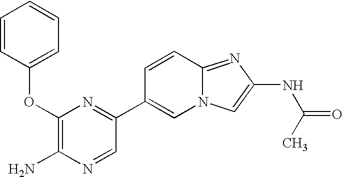 | ++++ | ++++ | ++++ | N-(6-(5- amino-6- phenoxypyrazin-2- yl)imidazo[1,2- a]pyridin-2- yl)acetamide | 361.1, 1.98 |
| 76 | 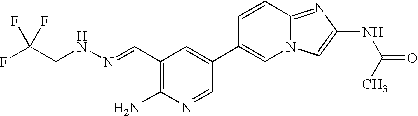 | ++++ | ++++ | ++++ | (E)-N-(6-(6- amino-5-((2- (2,2,2- trifluoroethyl) hydrazono) methyl)pyridin-3- yl)imidazo[1,2- a]pyridin-2- yl)acetamide | 391.8, 1.46 |
| 77 | 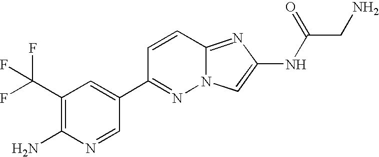 | ++++ | ++++ | ++++ | 2-amino-N-(6- (6-amino-5- (trifluoromethyl) pyridin-3- yl)imidazo[1,2- b]pyridazin- 2-yl)acetamide | 352.0, 1.33 |
| 78 | 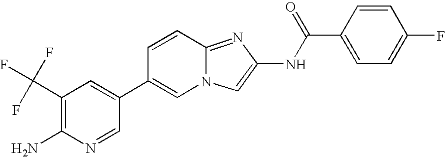 | ++++ | ++++ | ++++ | N-(6-(6- amino-5- (trifluoromethyl) pyridin-3- yl)imidazo[1,2- a]pyridin-2- yl)-4- fluorobenzamide | 416.1, 2.13 |
| 79 | 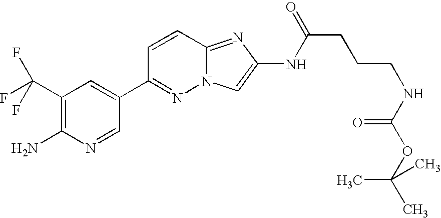 | ++++ | +++ | ++++ | tert-butyl 4-(6- (6-amino-5- (trifluoromethyl) pyridin-3- yl)imidazo[1,2- b]pyridazin- 2-ylamino)-4- oxobutylcarbamate | 480.1, 2.46 |
| 80 | 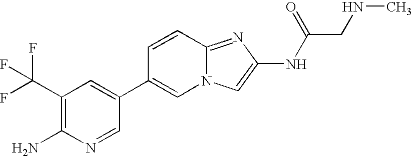 | ++++ | ++++ | ++++ | N-(6-(6- amino-5- (trifluoromethyl) pyridin-3- yl)imidazo[1,2- a]pyridin-2- yl)-2- (methylamino) acetamide | 365.0, 1.26 |
| 81 | 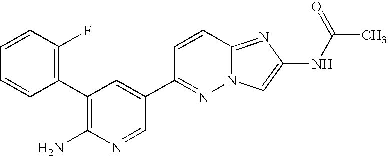 | ++++ | ++++ | ++++ | N-(6-(6- amino-5-(2- fluorophenyl) pyridin-3- yl)imidazo[1,2- b]pyridazin-2- yl)acetamide | 363.1, 1.63 |
| 82 | 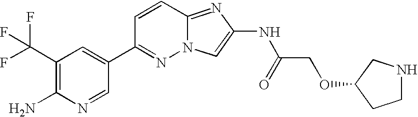 | ++++ | ++++ | ++++ | (S)-N-(6-(6- amino-5- (trifluoromethyl) pyridin-3- yl)imidazo[1,2- b]pyridazin- 2-yl)-2- (pyrrolidin-3- yloxy)acetamide | 469, 1.81 |
| 83 | 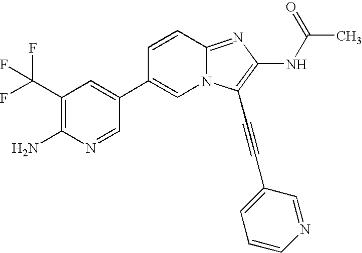 | ++++ | ++++ | ++++ | N-(6-(6- amino-5- (trifluoromethyl) pyridin-3- yl)-3-(pyridin-3- ylethynyl)imi- dazo[1,2- a]pyridin-2- yl)acetamide | 437.0, 1.53 |
| 84 | 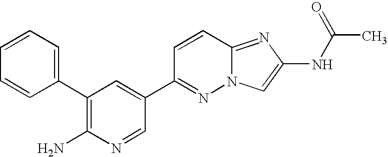 | ++++ | ++++ | ++++ | N-(6-(6- amino-5- phenylpyridin-3- yl)imidazo[1,2- b]pyridazin- 2-yl)acetamide | 345.1, 1.58 |
| 85 | 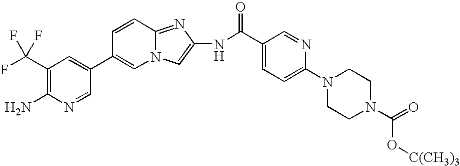 | ++++ | ++++ | ++++ | tert-butyl 4-(5- (6-(6-amino-5- (trifluoromethyl) pyridin-3- yl)imidazo[1,2- a]pyridin-2- ylcarbamoyl) pyridin-2- yl)piperazine- 1-carboxylate | 583.2, 2.36 |
| 86 | 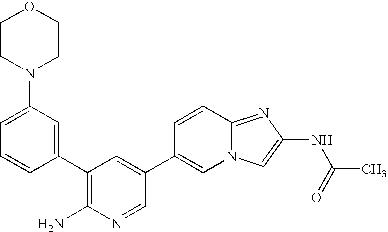 | ++++ | ++++ | ++++ | N-(6-(6- amino-5-(3- morpholino- phenyl)pyridin-3- yl)imidazo[1,2- a]pyridin-2- yl)acetamide | 429.2, 1.76 |
| 87 | 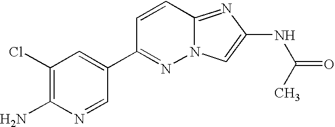 | ++++ | ++++ | ++++ | N-(6-(6- amino-5- chloropyridin- 3-yl)imidazo[1,2- b]pyridazin-2- yl)acetamide | 302.9, 1.37 |
| 88 | 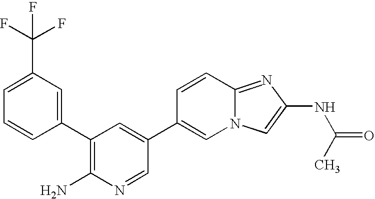 | ++++ | ++++ | ++++ | N-(6-(6- amino-5-(3- (trifluoromethyl) phenyl)pyridin-3- yl)imidazo[1,2- a]pyridin-2- yl)acetamide | 412.2, 1.92 |
| 89 | 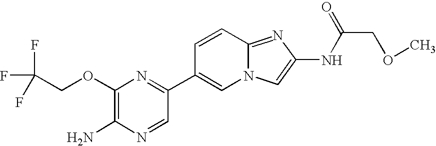 | ++++ | ++++ | ++++ | N-(6-(5- amino-6- (2,2,2- trifluoroethoxy) pyrazin-2- yl)imidazo[1,2- a]pyridin-2- methoxyacetamide | 397.0, 1.75 |
| 90 | 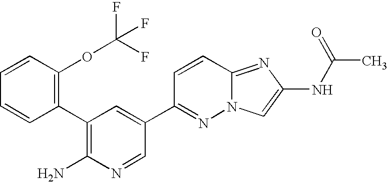 | ++++ | ++++ | ++++ | N-(6-(6- amino-5-(2- (trifluoromethoxy) phenyl)pyridin-3- yl)imidazo[1,2- b]pyridazin- 2-yl)acetamide | 429.1, 1.83 |
| 91 | 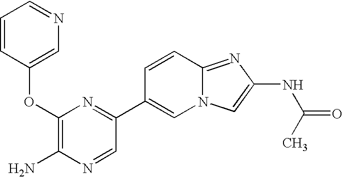 | ++++ | ++++ | ++++ | N-(6-(5- amino-6- (pyridin-3- yloxy)pyrazin- 2-yl)imidazo[1,2- a]pyridin-2- yl)acetamide | 362.2, 1.51 |
| 92 | 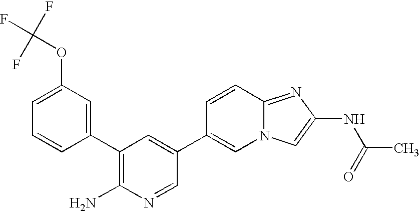 | ++++ | +++ | ++++ | N-(6-(6- amino-5-(3- (trifluoromethoxy) phenyl)pyridin-3- yl)imidazo[1,2- a]pyridin-2- yl)acetamide | 428.1, 2.05 |
| 93 | 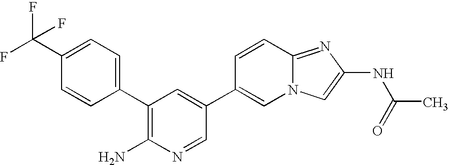 | ++++ | ++++ | ++++ | N-(6-(6- amino-5-(4- (trifluoromethyl) phenyl)pyridin-3- yl)imidazo[1,2- a]pyridin-2- yl)acetamide | 412.4, 2.02 |
| 94 | 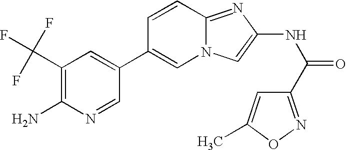 | ++++ | ++++ | ++++ | N-(6-(6- amino-5- (trifluoromethyl) pyridin-3- yl)imidazo[1,2- a]pyridin-2- yl)-5- methylisoxazole-3- carboxamide | 403.0, 2.04 |
| 95 | 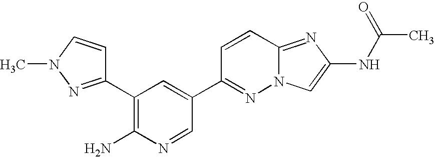 | ++++ | ++++ | ++++ | N-(6-(6- amino-5-(1- methyl-1H- pyrazol-3- yl)pyridin-3- yl)imidazo[1,2- b]pyridazin- 2-yl)acetamide | 349.1, 1.54 |
| 96 | 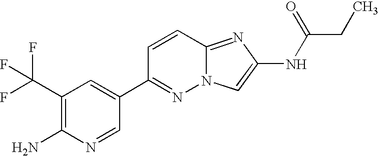 | ++++ | ++++ | ++++ | N-(6-(6- amino-5- (trifluoromethyl) pyridin-3- yl)imidazo[1,2- b]pyridazin- 2-yl)propionamide | 351.0, 1.80 |
| 97 | 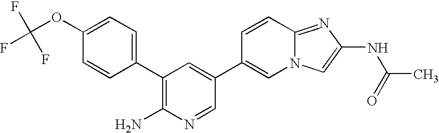 | ++++ | +++ | ++++ | N-(6-(6- amino-5-(4- (trifluoromethoxy) phenyl)pyridin-3- yl)imidazo[1,2- a]pyridin-2- yl)acetamide | 428.1, 2.07 |
| 98 | 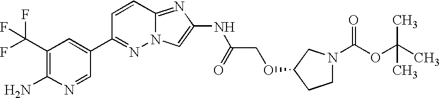 | ++++ | ++++ | ++++ | (S)-tert-butyl 3-(2-(6-(6- amino-5- (trifluoromethyl) pyridin-3- yl)imidazo[1,2- b]pyridazin- 2-ylamino)-2- oxoethoxy) pyrrolidine-1- carboxylate | 522.1 2.64 |
| 99 | 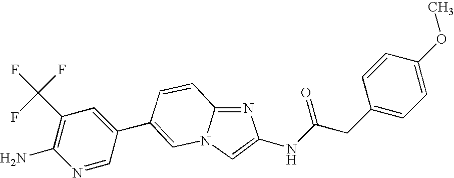 | ++++ | ++++ | ++++ | N-(6-(6- amino-5- (trifluoromethyl) pyridin-3- yl)imidazo[1,2- a]pyridin-2- yl)-2-(4- methoxyphenyl) acetamide | 442.2, 2.18 |
| 100 | 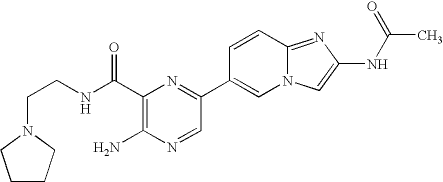 | ++++ | ++++ | ++++ | 6-(2-acetamido- imidazo[1,2- a]pyridin-6- yl)-3-amino- N-(2- (pyrrolidin-1- yl)ethyl) pyrazine-2- carboxamide | 409.2, 1.63 |
| 101 | 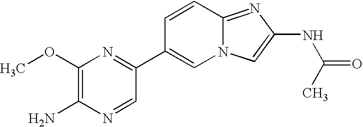 | ++++ | ++++ | ++++ | N-(6-(5- amino-6- methoxypyrazin-2- yl)imidazo[1,2- a]pyridin-2- yl)acetamide | 299.0, 1.25 |
| 102 | 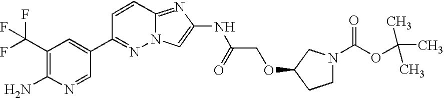 | ++++ | ++++ | ++++ | (R)-tert-butyl 3-(2-(6-(6- amino-5- (trifluoromethyl) pyridin-3- yl)imidazo[1,2- b]pyridazin- 2-ylamino)-2- oxoethoxy) pyrrolidine-1- carboxylate | 522.1 2.64 |
| 103 | 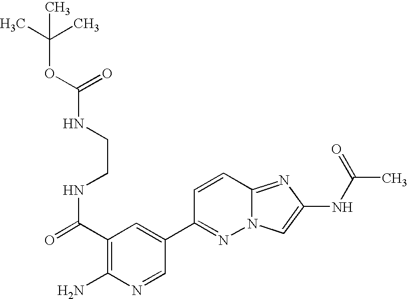 | ++++ | ++++ | ++++ | tert-butyl 2-(5- (2-acetamido- imidazo[1,2- b]pyridazin-6- yl)-2- aminonicotin- amido)ethyl- carbamate | 455.2, 1.94 |
| 104 | 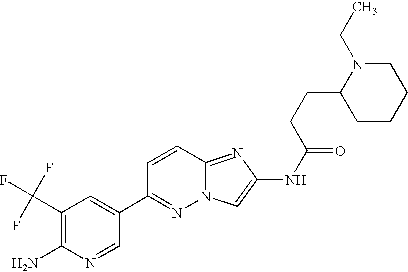 | ++++ | ++++ | ++++ | N-(6-(6- amino-5- (trifluoromethyl) pyridin-3- yl)imidazo[1,2- b]pyridazin- 2-yl)-3-(1- ethylpiperidin- 2-yl)propanamide | 462.1, 1.90 |
| 105 | 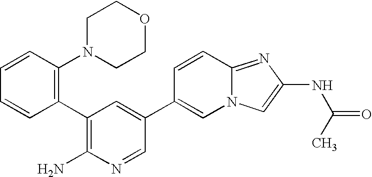 | ++++ | ++++ | ++++ | N-(6-(6- amino-5-(2- morpholino- phenyl)pyridin- 3-yl)imidazo[1,2- a]pyridin-2- yl)acetamide | 429.2, 1.83 |
| 106 | 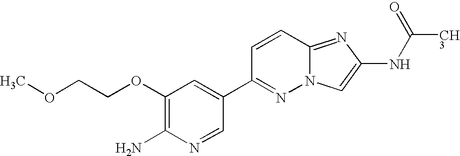 | ++++ | ++++ | ++++ | N-(6-(6- amino-5-(2- methoxyethoxy) pyridin-3- yl)imidazo[1,2- b]pyridazin- 2-yl)acetamide | 343.0, 1.38 |
| 107 | 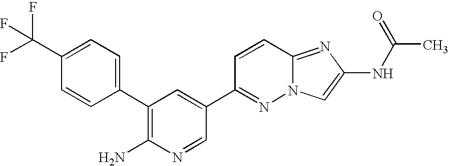 | ++++ | ++++ | ++++ | N-(6-(6- amino-5-(4- (trifluoromethyl) phenyl)pyridin-3- yl)imidazo[1,2- b]pyridazin- 2-yl)acetamide | 413.1, 1.94 |
| 108 | 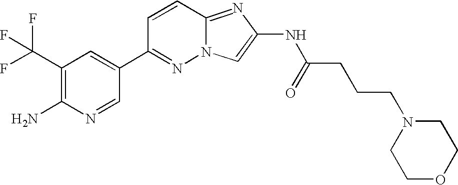 | ++++ | ++++ | ++++ | N-(6-(6- amino-5- (trifluoromethyl) pyridin-3- yl)imidazo[1,2- b]pyridazin- 2-yl)-4- morpholino- butanamide | 450.1, 1.49 |
| 109 | 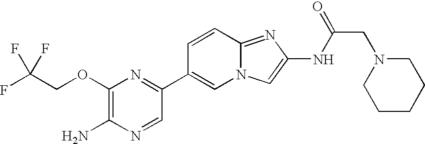 | ++++ | N/D | ++++ | N-(6-(5- amino-6- (2,2,2- trifluoroethoxy) pyrazin-2- yl)imidazo[1,2- a]pyridin-2- yl)-2- (piperidin-1- yl)acetamide | 450.2, 1.87 |
| 110 | 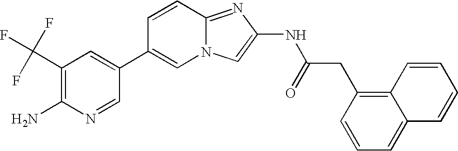 | ++++ | ++++ | ++++ | N-(6-(6- amino-5- (trifluoromethyl) pyridin-3- yl)imidazo[1,2- a]pyridin-2- yl)-2- (naphthalen-1- yl)acetamide | 462.1, 2.44 |
| 111 | 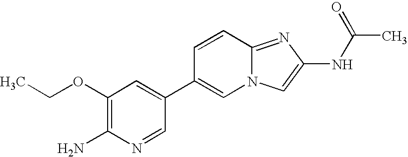 | ++++ | ++++ | +++ | N-(6-(6- amino-5- ethoxypyridin-3- yl)imidazo[1,2- a]pyridin-2- yl)acetamide | 312.1, 1.57 |
| 112 | 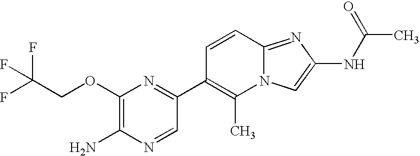 | ++++ | N/D | +++ | N-(6-(5- amino-6- (2,2,2- trifluoroethoxy) pyrazin-2-yl)- 5-methylimidazo [1,2-a]pyridin-2- yl)acetamide | 381.1, 1.94 |
| 113 | 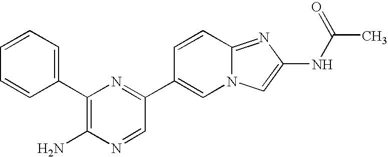 | ++++ | ++++ | +++ | N-(6-(5- amino-6- phenylpyrazin-2- yl)imidazo[1,2- a]pyridin-2- yl)acetamide | 345.1, 1.70 |
| 114 | 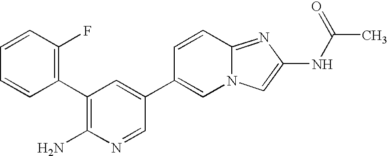 | ++++ | ++++ | +++ | N-(6-(6- amino-5-(2- fluorophenyl) pyridin-3- yl)imidazo[1,2- a]pyridin-2- yl)acetamide | 362.2, 1.74 |
| 115 | 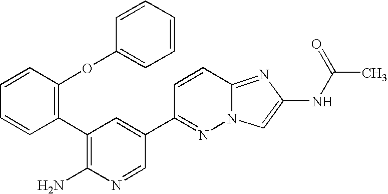 | ++++ | ++++ | +++ | N-(6-(6- amino-5-(2- phenoxyphenyl) pyridin-3- yl)imidazo[1,2- b]pyridazin-2- yl)acetamide | 437.1, 1.98 |
| 116 | 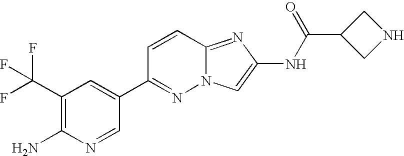 | ++++ | ++++ | +++ | N-(6-(6- amino-5- (trifluoromethyl) pyridin-3- yl)imidazo[1,2 b]pyridazin- 2-yl)azetidine- 3-carboxamide | 378.0, 1.44 |
| 117 | 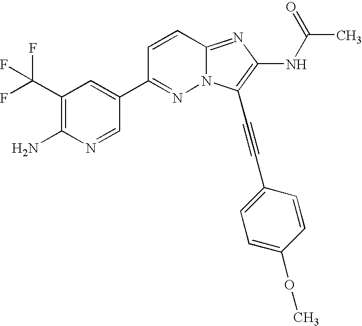 | ++++ | ++++ | +++ | N-(6-(6- amino-5- (trifluoromethyl) pyridin-3- yl)-3-((4- methoxyphenyl) ethynyl) imidazo[1,2- a]pyridin-2- yl)acetamide | 466.1, 2.07 |
| 118 | 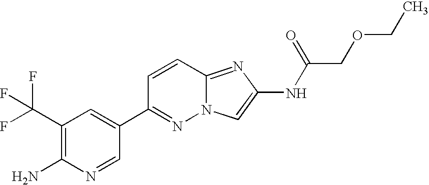 | ++++ | ++++ | +++ | N-(6-(6- amino-5- (trifluoromethyl) pyridin-3- yl)imidazo[1,2- b]pyridazin- 2-yl)-2- ethoxyacetamide | 381, 1.92 |
| 119 | 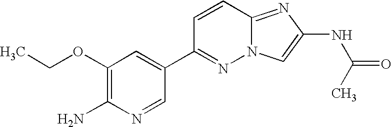 | ++++ | ++++ | +++ | N-(6-(6- amino-5- ethoxypyridin- 3-yl)imidazo[1,2- b]pyridazin- 2-yl)acetamide | 313.0, 1.41 |
| 120 | 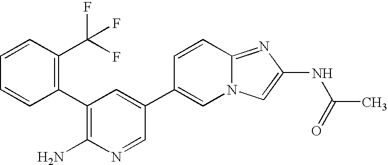 | ++++ | ++++ | +++ | N-(6-(6- amino-5-(2- (trifluoromethyl) phenyl)pyridin-3- yl)imidazo[1,2- a]pyridin-2- yl)acetamide | 412.2, 1.83 |
| 121 | 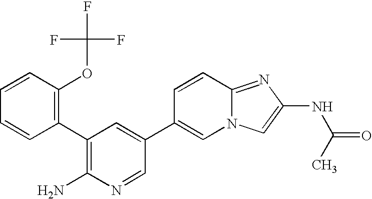 | ++++ | ++++ | +++ | N-(6-(6- amino-5-(2- (trifluoromethoxy) phenyl)pyridin-3- yl)imidazo[1,2- a]pyridin-2- yl)acetamide | 428.2, 1.87 |
| 122 | 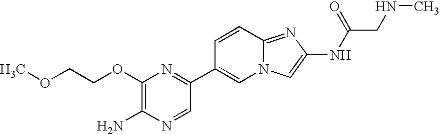 | ++++ | ++++ | +++ | N-(6-(5- amino-6-(2- methoxyethoxy) pyrazin-2- yl)imidazo[1,2- a]pyridin-2- yl)-2- (methylamino) acetamide | 372.0, 1.17 |
| 123 | 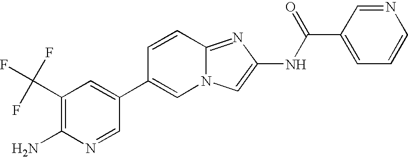 | ++++ | ++++ | +++ | N-(6-(6- amino-5- (trifluoromethyl) pyridin-3- yl)imidazo[1,2- a]pyridin-2- yl)nicotinamide | 399.1, 1.75 |
| 124 | 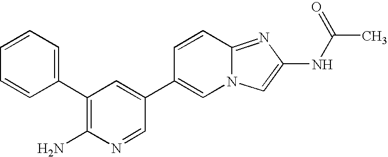 | ++++ | ++++ | +++ | N-(6-(6- amino-5- phenylpyridin- 3-yl)imidazo[1,2- a]pyridin-2- yl)acetamide | 344.1, 1.51 |
| 125 | 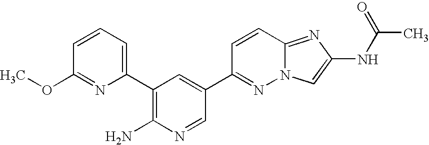 | ++++ | ++++ | +++ | N-(6-(2′- amino-6- methoxy-2,3′- bipyridin-5- yl)imidazo[1,2- b]pyridazin-2- yl)acetamide | 376.1, 1.74 |
| 126 | 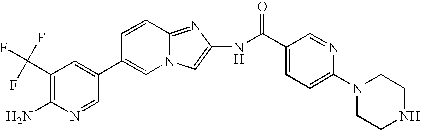 | ++++ | ++++ | +++ | N-(6-(6- amino-5- (trifluoromethyl) pyridin-3- yl)imidazo[1,2- a]pyridin-2- yl)-6- (piperazin-1- yl)nicotinamide | 483.2, 1.79 |
| 127 | 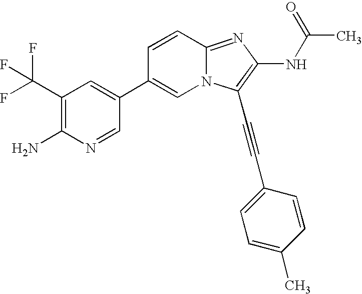 | ++++ | ++++ | +++ | N-(6-(6- amino-5- (trifluoromethyl) pyridin-3- yl)-3-(p- tolylethynyl) imidazo[1,2- a]pyridin-2- yl)acetamide | 450.1, 2.19 |
| 128 | 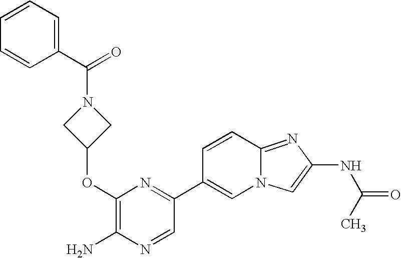 | ++++ | ++++ | +++ | N-(6-(5- amino-6-(1- benzoylazetidin-3- yloxy)pyrazin-2- yl)imidazo[1,2- a]pyridin-2- yl)acetamide | 444.1, 1.93 |
| 129 | 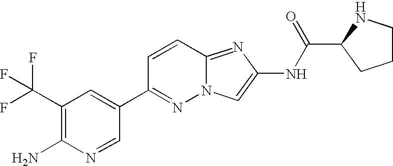 | ++++ | N/D | +++ | (S)-N-(6-(6- amino-5- (trifluoromethyl) pyridin-3- yl)imidazo[1,2- b]pyridazin- 2-yl)pyrrolidine- 2-carboxamide | 392.2, 1.81 |
| 130 | 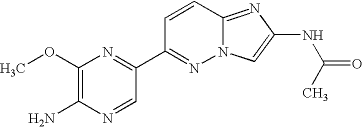 | ++++ | ++++ | +++ | N-(6-(5- amino-6- methoxypyrazin-2- yl)imidazo[1,2- b]pyridazin- 2-yl)acetamide | 300.0, 1.36 |
| 131 | 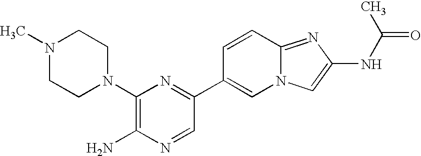 | ++++ | ++++ | +++ | N-(6-(5- amino-6-(4- methylpiperazin-1- yl)pyrazin-2- yl)imidazo[1,2- a]pyridin-2- yl)acetamide | 367.1, 1.16 |
| 132 | 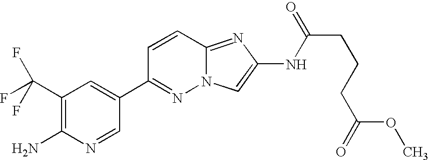 | ++++ | ++++ | +++ | methyl 5-(6- (6-amino-5- (trifluoromethyl) pyridin-3- yl)imidazo[1,2- b]pyridazin- 2-ylamino)-5- oxopentanoate | 423.1, 2.16 |
| 133 | 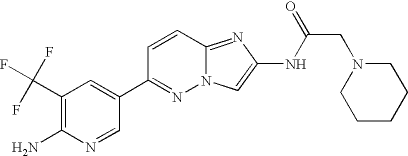 | ++++ | ++++ | +++ | N-(6-(6- amino-5- (trifluoromethyl) pyridin-3- yl)imidazo[1,2- b]pyridazin- 2-yl)-2- (piperidin-1- yl)acetamide | 420.2, 1.87 |
| 134 | 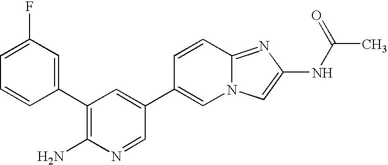 | ++++ | N/D | +++ | N-(6-(6- amino-5-(3- fluorophenyl) pyridin-3- yl)imidazo[1,2- a]pyridin-2- yl)acetamide | 362.2, 1.77 |
| 135 | 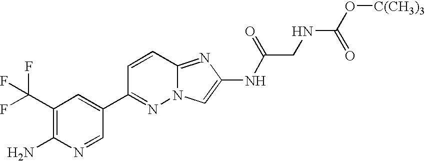 | ++++ | ++++ | +++ | tert-butyl 2-(6- (6-amino-5- (trifluoromethyl) pyridin-3- yl)imidazo[1,2- b]pyridazin- 2-ylamino)-2- oxoethylcarbamate | 452.1, 2.08 |
| 136 | 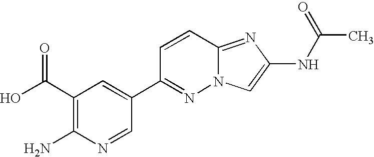 | ++++ | ++ | ++ | 5-(2- acetamido- imidazo[1,2- b]pyridazin-6- yl)-2- aminonicotinic acid | 313.0, 1.46 |
| 137 | 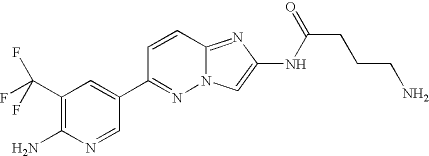 | ++++ | N/D | ++ | 4-amino-N-(6- (6-amino-5- (trifluoromethyl) pyridin-3- yl)imidazo[1,2- b]pyridazin- 2-yl)butanamide | 380.0, 1.68 |
| 138 | 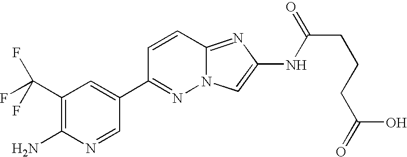 | ++++ | N/D | ++ | 5-(6-(6- amino-5- (trifluoromethyl) pyridin-3- yl)imidazo[1,2- b]pyridazin- 2-ylamino)-5- oxopentanoic acid | 409.0, 1.98 |
| 139 | 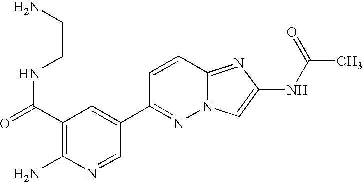 | ++++ | N/D | ++ | 5-(2- acetamido- imidazo[1,2- b]pyridazin-6- yl)-2-amino- N-(2- aminoethyl) nicotinamide | 355.1, 1.39 |
| 140 | 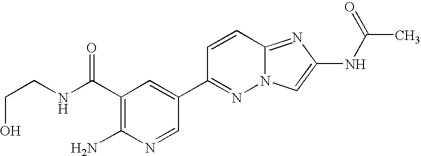 | ++++ | N/D | ++ | 5-(2- acetamido- imidazo[1,2- b]pyridazin-6- yl)-2-amino- N-(2- hydroxyethyl) nicotinamide | 356.1, 1.44 |
| 141 | 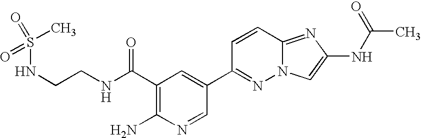 | ++++ | N/D | ++ | 5-(2- acetamido- imidazo[1,2- b]pyridazin-6- yl)-2-amino- N-(2- (methylsulfon amido)ethyl) nicotinamide | 433.1, 1.57 |
| 142 | 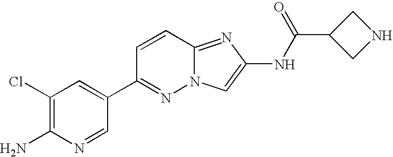 | ++++ | N/D | ++ | N-(6-(6- amino-5- chloropyridin-3- yl)imidazo[1,2- b]pyridazin- 2-yl)azetidine- 3-carboxamide | 343.7, 1.21 |
| 143 | 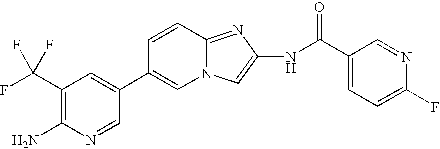 | ++++ | N/D | ++ | N-(6-(6- amino-5- (trifluoromethyl) pyridin-3- yl)imidazo[1,2- a]pyridin-2- yl)-6- fluoronicotinamide | 417.1, 2.07 |
| 144 | 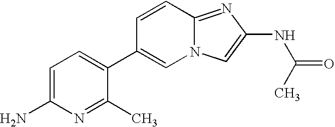 | ++++ | N/D | ++ | N-(6-(6- amino-2- methylpyridin-3- yl)imidazo[1, 2-a]pyridin-2- yl)acetamide | 282.1, 1.19 |
| 145 | 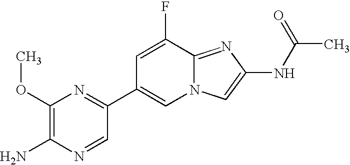 | ++++ | N/D | ++ | N-(6-(5- amino-6- methoxypyrazin- 2-yl)-8- fluoroimidazo [1,2-a]pyridin- 2-yl)acetamide | 317.1, 1.70 |
| 146 | 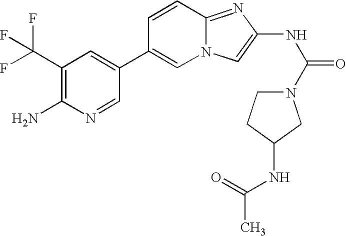 | ++++ | N/D | ++ | 3-acetamido- N-(6-(6- amino-5- (trifluoromethyl) pyridin-3- yl)imidazo[1, 2-a]pyridin-2- yl)pyrrolidine- 1-carboxamide | 448.2, 1.71 |
| 147 | 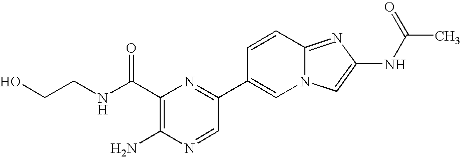 | ++++ | N/D | ++ | 6-(2- acetamido- imidazo[1,2- a]pyridin-6- yl)-3-amino- N-(2- hydroxyethyl) pyrazine-2- carboxamide | 356.2, 1.63 |
| 148 | 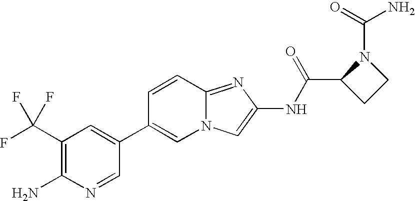 | ++++ | N/D | ++ | (S)-N2-(6-(6- amino-5- (trifluoromethyl) pyridin-3- yl)imidazo[1, 2-a]pyridin-2- yl)azetidine- 1,2-dicarboxamide | 420.1, 1.70 |
| 149 | 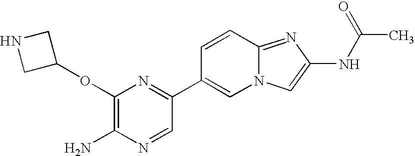 | ++++ | N/D | ++ | N-(6-(5- amino-6- (azetidin-3- yloxy)pyrazin- 2-yl)imidazo[1, 2-a]pyridin-2- yl)acetamide | 340.1, 1.14 |
| 150 | 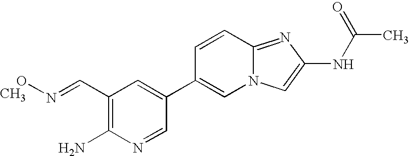 | ++++ | N/D | ++ | (E)-N-(6-(6- amino-5- ((methoxy- imino)methyl) pyridin-3- yl)imidazo[1, 2-a]pyridin-2- yl)acetamide | 325.0, 1.34 |
| 151 | 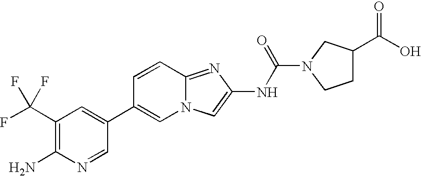 | ++++ | N/D | ++ | 1-(6-(6- amino-5- (trifluoromethyl) yl)pyridin-3- yl)imidazo[1, 2-a]pyridin-2- ylcarbamoyl) pyrrolidine-3- carboxylic acid | 435.1, 1.77 |
| 152 | 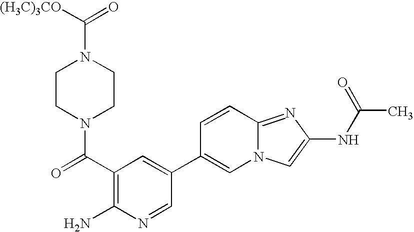 | ++++ | N/D | ++ | tert-butyl 4- (5-(2- acetamido- imidazo[1,2- a]pyridin-6- yl)-2- aminonicotinoyl) piperazine- 1-carboxylate | 380.2, 1.90 |
| 153 | 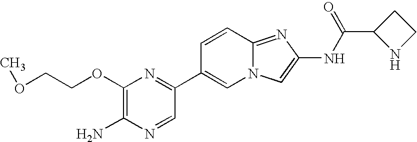 | ++++ | N/D | ++ | N-(6-(5- amino-6-(2- methoxyethoxy) pyrazin-2- yl)imidazo[1, 2-a]pyridin-2- yl)azetidine- 2-carboxamide | 384.2, 1.44 |
| 154 | 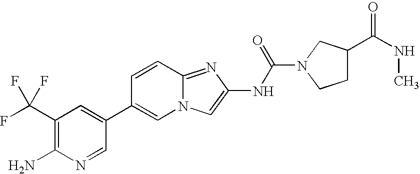 | ++++ | N/D | ++ | N1-(6-(6- amino-5- (trifluoromethyl) pyridin-3- yl)imidazo[1, 2-a]pyridin-2- yl)-N3- methylpyrrolidine- 1,3-dicarboxamide | 448.1, 1.70 |
| 155 | 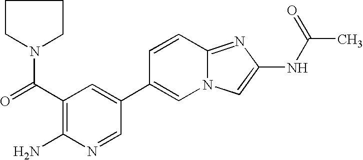 | ++++ | N/D | ++ | N-(6-(6- amino-5- (pyrrolidine-1- carbonyl)pyridin-3- yl)imidazo[1, 2-a]pyridin-2- yl)acetamide | 365.1, 1.48 |
| 156 | 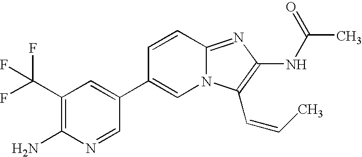 | ++++ | N/D | ++ | (Z)-N-(6-(6- amino-5- (trifluoromethyl) pyridin-3- yl)-3-(prop-1- enyl)imidazo [1,2-a]pyridin- 2-yl)acetamide | 376.0, 1.53 |
| 157 | 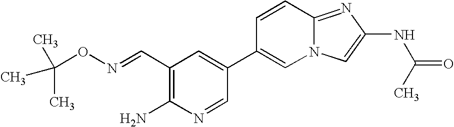 | ++++ | N/D | ++ | (E)-N-(6-(6- amino-5- ((tert- butoxyimino) methyl)pyridin-3- yl)imidazo[1, 2-a]pyridin-2- yl)acetamide | 367.1, 1.68 |
| 158 | 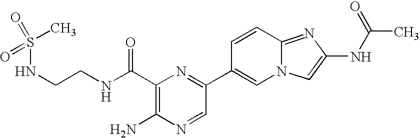 | ++++ | N/D | ++ | 6-(2- acetamido- imidazo[1,2- a]pyridin-6- yl)-3-amino- N-(2- (methylsulfon amido)ethyl) pyrazine-2- carboxamide | 433.2, 1.72 |
| 159 | 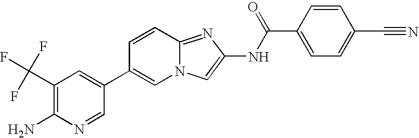 | ++++ | N/D | ++ | N-(6-(6- amino-5- (trifluoromethyl) pyridin-3- yl)imidazo[1, 2-a]pyridin-2- yl)-4- cyanobenzamide | 423.2, 2.21 |
| 160 | 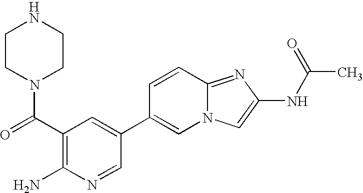 | ++++ | N/D | ++ | N-(6-(6- amino-5- (piperazine-1- carbonyl)pyridin-3- yl)imidazo[1, 2-a]pyridin-2- yl)acetamide | 380.1, 1.06 |
| 161 | 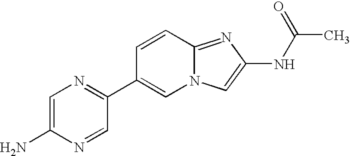 | ++++ | N/D | ++ | N-(6-(5- aminopyrazin-2- yl)imidazo[1, 2-a]pyridin-2- yl)acetamide | 270.1, 1.57 |
| 162 | 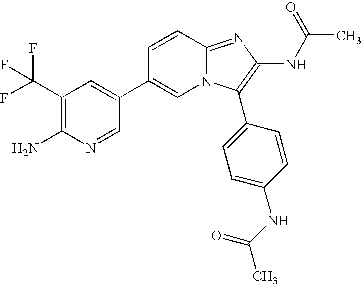 | ++++ | N/D | ++ | N-(4-(2- acetamido-6- (6-amino-5- (trifluoromethyl) yl)pyridin-3- yl)imidazo[1, 2-a]pyridin-3- yl)phenyl) acetamide | 469.0, 1.83 |
| 163 | 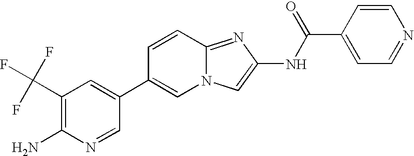 | ++++ | N/D | ++ | N-(6-(6- amino-5- (trifluoromethyl) yl)pyridin-3- yl)imidazo[1, 2-a]pyridin-2- yl)isonicotinamide | 399.1, 1.74 |
| 164 | 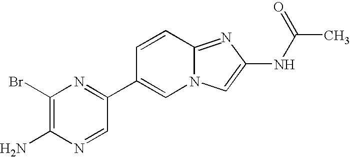 | ++++ | N/D | ++ | N-(6-(5- amino-6- bromopyrazin-2- yl)imidazo[1, 2-a]pyridin-2- yl)acetamide | 346.7, 1.56 |
| 165 | 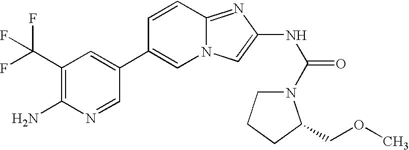 | ++++ | N/D | ++ | (S)-N-(6-(6- amino-5- (trifluoromethyl) pyridin-3- yl)imidazo[1, 2-a]pyridin-2- yl)-2- (methoxymethyl) pyrrolidine- 1-carboxamide | 435.1, 2.13 |
| 166 | 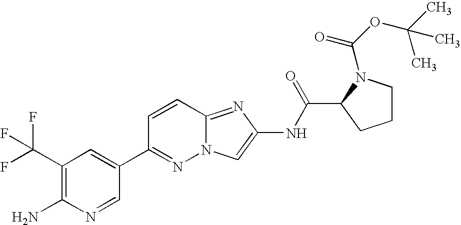 | ++++ | N/D | ++ | (S)-tert-butyl 2-(6-(6- amino-5- (trifluoromethyl) pyridin-3- yl)imidazo[1, 2-b]pyridazin- 2-ylcarbamoyl) pyrrolidine-1- carboxylate | 492.1, 2.50 |
| 167 | 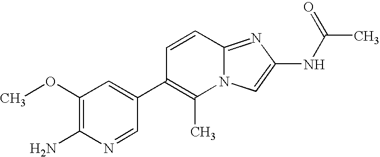 | ++++ | N/D | ++ | N-(6-(6- amino-5- methoxypyridin- 3-yl)-5- methylimidazo[1,2- a]pyridin-2- yl)acetamide | 312.0, 1.12 |
| 168 | 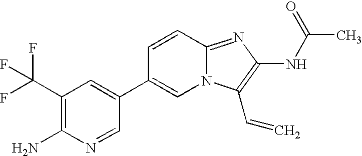 | ++++ | N/D | ++ | N-(6-(6- amino-5- (trifluoromethyl) pyridin-3- yl)-3- vinylimidazo[1,2- a]pyridin-2- yl)acetamide | 362.0, 1.47 |
| 169 | 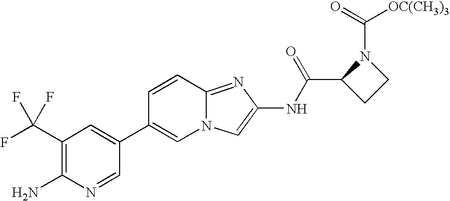 | ++++ | N/D | ++ | (S)-tert-butyl 2-(6-(6- amino-5- (trifluoromethyl) pyridin-3- yl)imidazo[1, 2-a]pyridin-2- ylcarbamoyl) azetidine-1- carboxylate | 477.2, 2.20 |
| 170 | 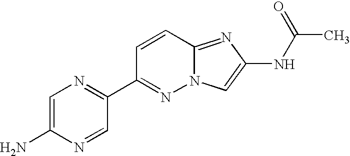 | ++++ | N/D | ++ | N-(6-(5- aminopyrazin-2- yl)imidazo[1, 2-bipyridazin-2- yl)acetamide | 270.1, 1.57 |
| 171 | 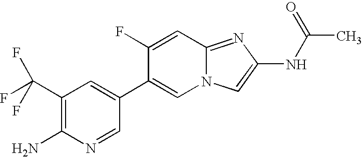 | ++++ | N/D | ++ | N-(6-(6- amino-5- (trifluoromethyl) pyridin-3- yl)-7- fluoroimidazo [1,2-a]pyridin- 2-yl)acetamide | 354.0, 1.83 |
| 172 | 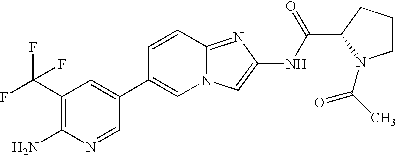 | ++++ | N/D | ++ | (S)-1-acetyl- N-(6-(6- amino-5- (trifluoromethyl) pyridin-3- yl)imidazo[1, 2-a]pyridin-2- yl)pyrrolidine- 2-carboxamide | 433.2, 1.83 |
| 173 | 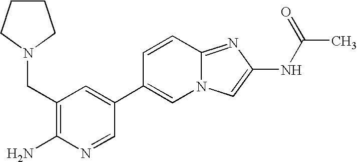 | ++++ | N/D | ++ | N-(6-(6- amino-5- (pyrrolidin-1- ylmethyl)pyridin-3- yl)imidazo[1, 2-a]pyridin-2- yl)acetamide | 351.1, 1.26 |
| 174 | 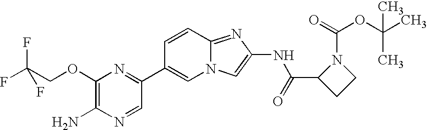 | ++++ | N/D | ++ | tert-butyl 2- (6-(5-amino- 6-(2,2,2- trifluoroethoxy) pyrazin-2- yl)imidazo[1, 2-a]pyridin-2- ylcarbamoyl) azetidine-1- carboxylate | 508.2, 2.42 |
| 175 | 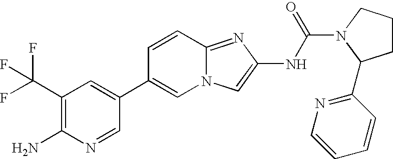 | ++++ | N/D | ++ | N-(6-(6- amino-5- (trifluoromethyl) pyridin-3- yl)imidazo[1, 2-a]pyridin-2- yl)-2-(pyridin-2- yl)pyrrolidine-1- carboxamide | 468.2, 1.72 |
| 176 | 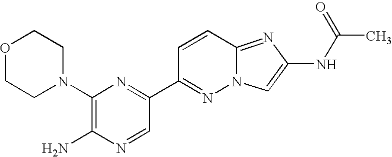 | ++++ | N/D | ++ | N-(6-(5- amino-6- morpholino- pyrazin-2- yl)imidazo[1, 2-b]pyridazin-2- yl)acetamide | 355.1, 1.75 |
| 177 | 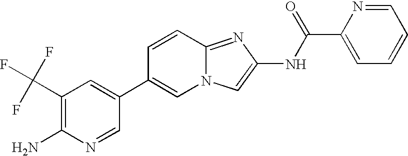 | ++++ | N/D | ++ | N-(6-(6- amino-5- (trifluoromethyl) yl)pyridin-3- yl)imidazo[1, 2-a]pyridin-2- yl)picolinamide | 399.1, 2.05 |
| 178 | 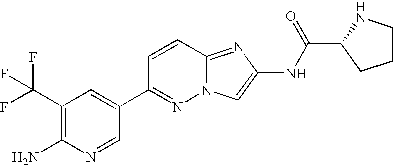 | ++++ | N/D | ++ | (R)-N-(6-(6- amino-5- (trifluoromethyl) pyridin-3- yl)imidazo[1, 2-b]pyridazin- 2-yl)pyrrolidine- 2-carboxamide | 392.1, 1.76 |
| 179 | 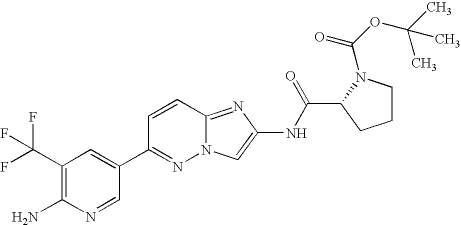 | ++++ | N/D | ++ | (R)-tert-butyl amino-5- (trifluoromethyl) pyridin-3- yl)imidazo[1, 2-b]pyridazin- 2-ylcarbamoyl) pyrrolidine-1- carboxylate | 492.1, 2.50 |
| 180 | 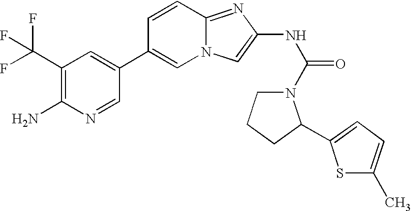 | ++++ | N/D | ++ | N-(6-(6- amino-5- (trifluoromethyl) pyridin-3- yl)imidazo[1, 2-a]pyridin-2- yl)-2-(5- methyithiophen- 2-yl)pyrrolidine-1- carboxamide | 487.1, 2.42 |
| 181 | 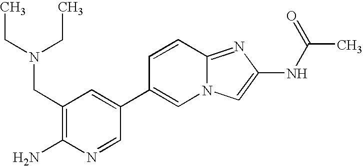 | ++++ | N/D | ++ | N-(6-(6- amino-5- ((diethylamino) methyl)pyridin-3- yl)imidazo[1, 2-a]pyridin-2- yl)acetamide | 353.2, 1.30 |
| 182 | 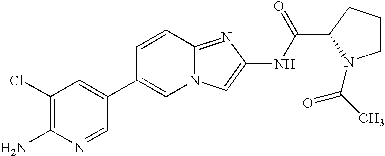 | ++++ | N/D | ++ | (S)-1-acetyl- N-(6-(6- amino-5- chloropyridin-3- yl)imidazo[1, 2-a]pyridin-2- yl)pyrrolidine-2- carboxamide | 399.1, 1.48 |
| 183 | 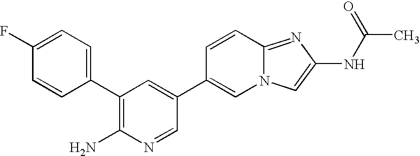 | ++++ | N/D | ++ | N-(6-(6- amino-5-(4- fluorophenyl) pyridin-3- yl)imidazo[1, 2-a]pyridin-2- yl)acetamide | 362.2, 1.79 |
| 184 | 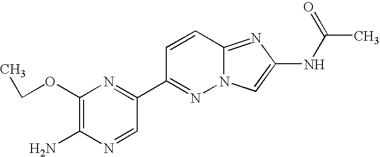 | ++++ | ++++ | +++ | N-(6-(5- amino-6- ethoxypyrazin-2- yl)imidazo[1, 2-b]pyridazin- 2-yl)acetamide | 314.0, 1.50 |
| 185 | 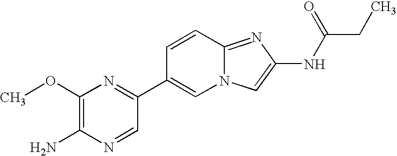 | ++++ | N/D | +++ | N-(6-(5- amino-6- methoxypyrazin-2- yl)imidazo[1, 2-a]pyridin-2- yl)propionamide | 312.7, 1.40 |
| 186 | 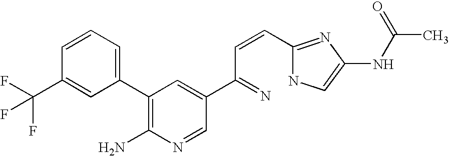 | ++++ | N/D | +++ | N-(6-(6- amino-5-(3- (trifluoromethyl) phenyl)pyridin-3- yl)imidazo[1, 2-b]pyridazin- 2-yl)acetamide | 412.9, 1.90 |
| 187 | 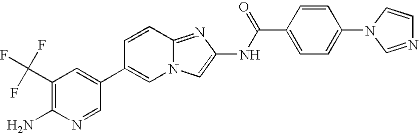 | ++++ | ++++ | +++ | N-(6-(6- amino-5- (trifluoromethyl) pyridin-3- yl)imidazo[1, 2-a]pyridin-2- yl)-4-(1H- imidazol-1- yl)benzamide | 464.1, 1.81 |
| 188 | 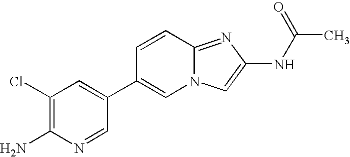 | ++++ | ++++ | +++ | N-(6-(6- amino-5- chloropyridin-3- yl)imidazo[1, 2-a]pyridin-2- yl)acetamide | 302.0, 1.50 |
| 189 | 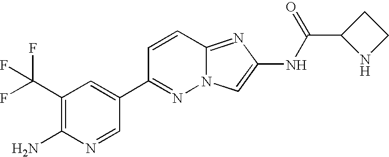 | ++++ | N/D | +++ | N-(6-(6- amino-5- (trifluoromethyl) imidazo[1, 2-b]pyridazin- 2-yl)azetidine 2-carboxamide | 377.9, 1.42 |
| 190 | 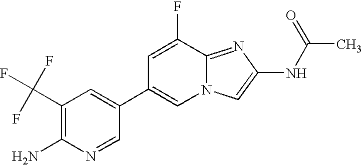 | ++++ | ++++ | +++ | N-(6-(6- amino-5- (trifluoromethyl) pyridin-3- yl)-8- fluoroimidazo [1,2-a]pyridin- 2-yl)acetamide | 354.0, 1.94 |
| 191 | 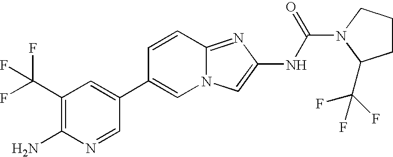 | ++++ | N/D | +++ | N-(6-(6- amino-5- (trifluoromethyl) pyridin-3- yl)imidazo[1, 2-a]pyridin-2- yl)-2- (trifluoromethyl) yl)pyrrolidine- 1-carboxamide | 459.1, 2.22 |
| 192 | 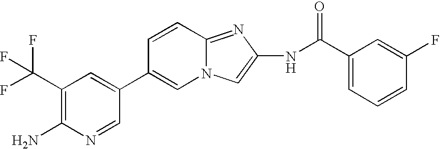 | ++++ | N/D | +++ | N-(6-(6- amino-5- (trifluoromethyl) pyridin-3- yl)imidazo[1, 2-a]pyridin-2- yl)-3- fluorobenzamide | 416.1, 2.17 |
| 193 | 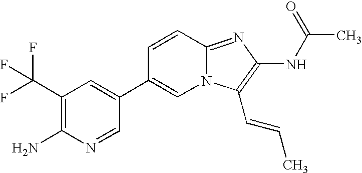 | ++++ | +++ | +++ | (E)-N-(6-(6- amino-5- (trifluoromethyl) pyridin-3- yl)-3-(prop-1- enyl)imidazo[1,2- a]pyridin-2- yl)acetamide | 376.0, 1.60 |
| 194 | 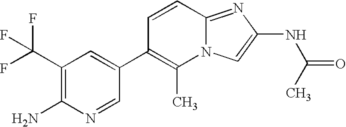 | ++++ | +++ | +++ | N-(6-(6- amino-5- (trifluoromethyl) pyridin-3- yl)-5- methylimidazo[1,2- a]pyridin-2- yl)acetamide | 350.1, 1.41 |
| 195 | 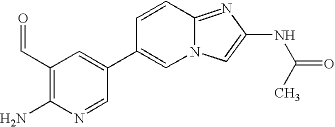 | ++++ | ++++ | +++ | N-(6-(6- amino-5- formylpyridin-3- yl)imidazo[1, 2-a]pyridin-2- yl)acetamide | 296.0, 1.16 |
| 196 | 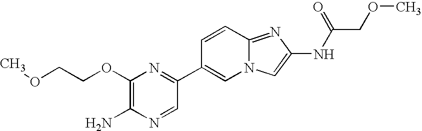 | ++++ | ++++ | +++ | N-(6-(5- amino-6-(2- methoxyethoxy) pyrazin-2- yl)imidazo[1, 2-a]pyridin-2- yl)-2- methoxyacetamide | 373.0, 1.40 |
| 197 | 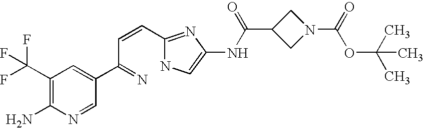 | ++++ | N/D | +++ | tert-butyl 3- (6-(6-amino- 5-(trifluoromethyl) pyridin-3- yl)imidazo[1, 2-b]pyridazin- 2-ylcarbamoyl) azetidine-1- carboxylate | 478.1, 2.50 |
| 198 | 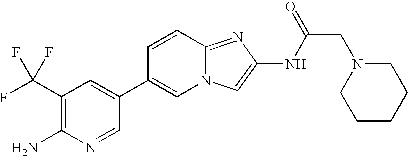 | ++++ | N/D | +++ | N-(6-(6- amino-5- (trifluoromethyl) pyridin-3- yl)imidazo[1, 2-a]pyridin-2- yl)-2- (piperidin-1- yl)acetamide | 419.2, 1.74 |
| 199 | 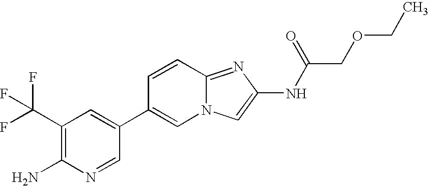 | ++++ | N/D | +++ | N-(6-(6- amino-5- (trifluoromethyl) pyridin-3- yl)imidazo[1, 2-a]pyridin-2- yl)-2- ethoxyacetamide | 379.9, 1.62 |
| 200 | 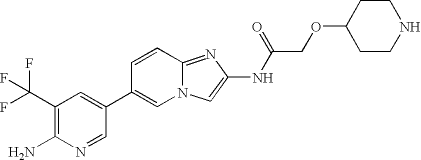 | ++++ | N/D | +++ | N-(6-(6- amino-5- (trifluoromethyl) pyridin-3- yl)imidazo[1, 2-a]pyridin-2- yl)-2- (piperidin-4- yloxy)acetamide | 435.2, 1.66 |
| 201 | 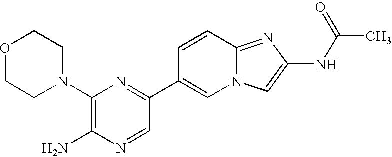 | ++++ | N/D | +++ | N-(6-(5- amino-6- morpholino- pyrazin-2- yl)imidazo[1, 2-a]pyridin-2- yl)acetamide | 354.1, 1.65 |
| 202 | 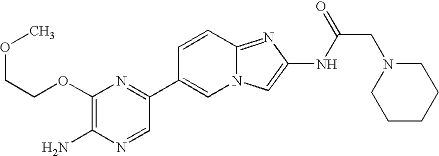 | ++++ | N/D | +++ | N-(6-(5- amino-6-(2- methoxyethoxy) pyrazin-2- yl)imidazo[1, 2-a]pyridin-2- yl)-2- (piperidin-1- yl)acetamide | 426.2, 1.63 |
| 203 | 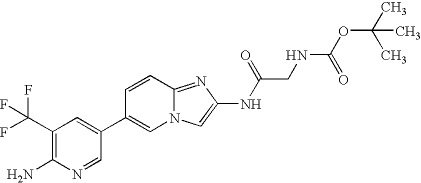 | ++++ | N/D | +++ | tert-butyl 2- (6-(6-amino- 5-(trifluoromethyl) pyridin-3- yl)imidazo[1, 2-a]pyridin-2- ylamino)-2- oxoethylcarbamate | 451.1, 1.88 |
| 204 | 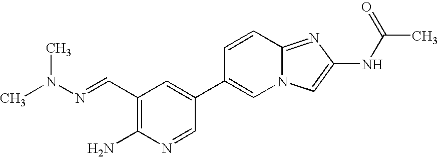 | ++++ | ++++ | +++ | (E)-N-(6-(6- amino-5- ((2,2- dimethylhy- drazono)methyl) pyridin-3- yl)imidazo[1, 2-a]pyridin-2- yl)acetamide | 338.1, 1.39 |
| 205 | 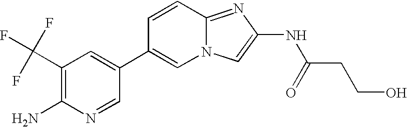 | ++++ | ++++ | +++ | N-(6-(6- amino-5- (trifluoromethyl) pyridin-3- yl)imidazo[1, 2-a]pyridin-2- yl)-3- hydroxypropanamide | 366.1, 1.64 |
| 206 | 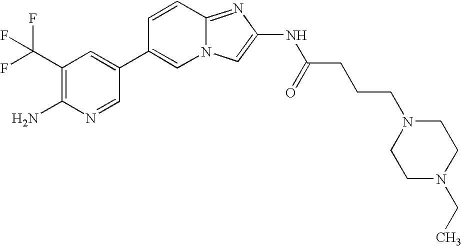 | ++++ | ++++ | +++ | N-(6-(6- amino-5- (trifluoromethyl) pyridin-3- yl)imidazo[1, 2-a]pyridin-2- yl)-4-(4- ethylpiperazin-1- yl)butanamide | 476.1, 1.62 |
| 207 | 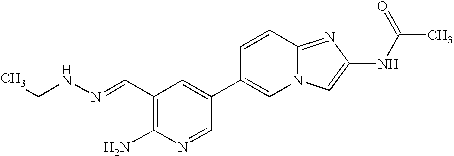 | ++++ | N/D | +++ | (E)-N-(6-(6- amino-5-((2- ethylhydrazono) methyl)pyridin-3- yl)imidazo[1, 2-a]pyridin-2- yl)acetamide | 337.8, 1.38 |
| 208 | 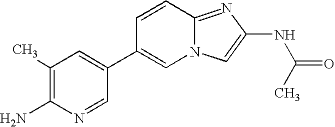 | ++++ | ++++ | +++ | N-(6-(6- amino-5- methylpyridin-3- yl)imidazo[1, 2-a]pyridin-2- yl)acetamide | 282.1, 1.44 |
| 209 | 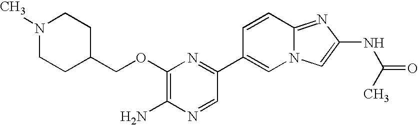 | ++++ | N/D | +++ | N-(6-(5- amino-6-((1- methylpiperidin-4- yl)methoxy) pyrazin-2- yl)imidazo[1, 2-a]pyridin-2- yl)acetamide | 396.2, 1.47 |
| 210 | 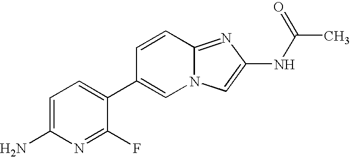 | ++++ | N/D | +++ | N-(6-(6- amino-2- fluoropyridin-3- yl)imidazo[1, 2-a]pyridin-2- yl)acetamide | 285.9, 1.35 |
| 211 | 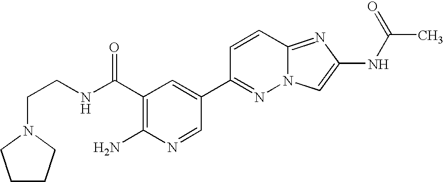 | ++++ | N/D | +++ | 5-(2- acetamido- imidazo[1,2- b]pyridazin-6- yl)-2-amino- N-(2- (pyrrolidin-1- yl)ethyl) nicotinamide | 409.1, 1.48 |
| 212 | 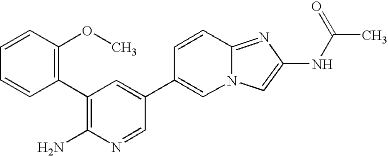 | ++++ | N/D | +++ | N-(6-(6- amino-5-(2- methoxyphenyl) pyridin-3- yl)imidazo[1, 2-b]pyridazin- 2-yl)acetamide | 374.8, 1.67 |
| 213 | 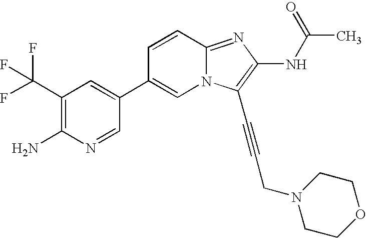 | ++++ | +++ | +++ | N-(6-(6- amino-5- (trifluoromethyl) pyridin-3- yl)-3-(3- morpholino- prop-1- ynyl)imidazo[1, 2-a]pyridin- 2-yl)acetamide | 459.2, 1.43 |
| 214 | 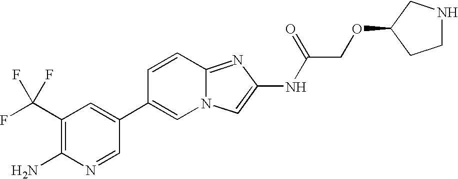 | ++++ | N/D | +++ | (R)-N-(6-(6- amino-5- (trifluoromethyl) pyridin-3- yl)imidazo[1, 2-a]pyridin-2- yl)-2- (pyrrolidin-3- yloxy)acetamide | 421.1, 1.65 |
| 215 | 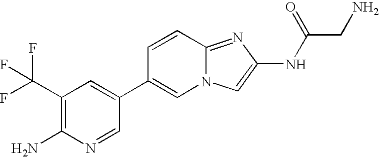 | ++++ | +++ | +++ | 2-amino-N- (6-(6-amino-5- (trifluoromethyl) pyridin-3- yl)imidazo[1, 2-a]pyridin-2- yl)acetamide | 351.0, 1.19 |
| 216 | 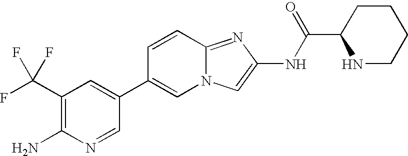 | ++++ | N/D | +++ | (R)-N-(6-(6- amino-5- (trifluoromethyl) pyridin-3- yl)imidazo[1, 2-a]pyridin-2- yl)piperidine- 2-carboxamide | 405.1, 1.64 |
| 217 | 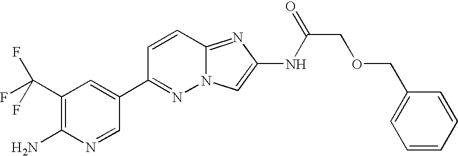 | ++++ | N/D | +++ | N-(6-(6- amino-5- (trifluoromethyl) pyridin-3- yl)imidazo[1, 2-b]pyridazin- 2-yl)-2- (benzyloxy) acetamide | 443.0, 2.62 |
| 218 | 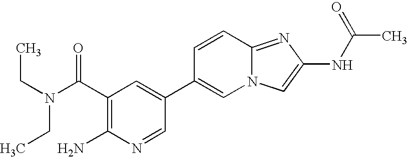 | ++++ | ++++ | +++ | 5-(2- acetamido- imidazo[1,2- a]pyridin-6- yl)-2-amino- N,N-diethyl- nicotinamide | 367.1, 1.59 |
| 219 | 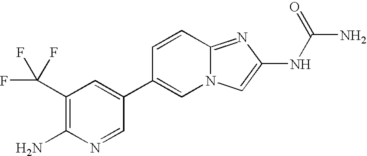 | ++++ | N/D | +++ | 1-(6-(6- amino-5- (trifluoromethyl) pyridin-3- yl)imidazo[1, 2-a]pyridin-2- yl)urea | 337.1, 1.61 |
| 220 | 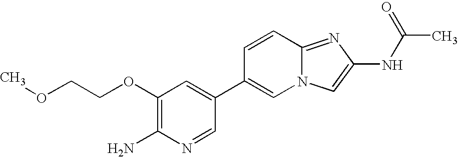 | ++++ | N/D | +++ | N-(6-(6- amino-5-(2- methoxyethoxy) pyridin-3- yl)imidazo[1, 2-a]pyridin-2- yl)acetamide | 342.0, 1.24 |
| 221 | 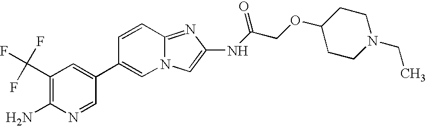 | ++++ | N/D | +++ | N-(6-(6- amino-5- (trifluoromethyl) yl)imidazo[1, 2-a]pyridin-2- yl)-2-(1- ethylpiperidin-4- yloxy)acetamide | 463.2, 1.73 |
| 222 | 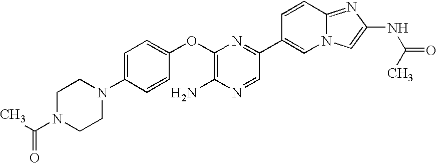 | ++++ | N/D | +++ | N-(6-(6-(4-(4- acetylpiperazin-1- yl)phenoxy)-5- aminopyrazin-2- yl)imidazo[1, 2-a]pyridin-2- yl)acetamide | 487.2, 1.78 |
| 223 | 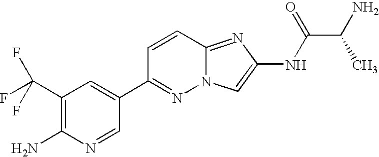 | ++++ | N/D | +++ | (R)-2-amino- N-(6-(6- amino-5- (trifluoromethyl) pyridin-3- yl)imidazo[1, 2-b]pyridazin- 2-yl)propanamide | 366.1, 1.70 |
| 224 | 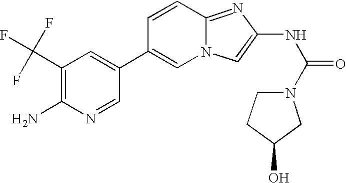 | ++++ | N/D | +++ | (S)-N-(6-(6- amino-5- (trifluoromethyl) pyridin-3- yl)imidazo[1, 2-a]pyridin-2- yl)-3- hydroxy- pyrrolidine- 1-carboxamide | 407.1, 1.68 |
| 225 | 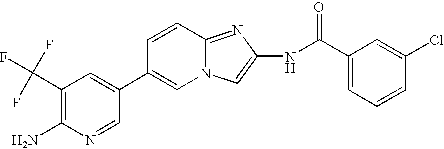 | ++++ | N/D | +++ | N-(6-(6- amino-5- (trifluoromethyl) pyridin-3- yl)imidazo[1, 2-a]pyridin-2- yl)-3- chlorobenzamide | 432.1, 2.45 |
| 226 | 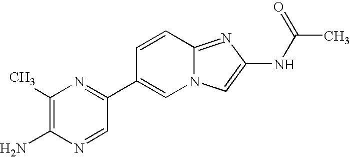 | ++++ | N/D | +++ | N-(6-(5- amino-6- methylpyrazin-2- yl)imidazo[1, 2-a]pyridin-2- yl)acetamide | 282.9, 1.23 |
| 227 | 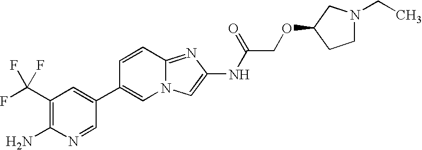 | ++++ | N/D | +++ | (R)-N-(6-(6- amino-5- (trifluoromethyl) pyridin-3- yl)imidazo[1, 2-a]pyridin-2- yl)-2-(1- ethylpyrrolidn-3- yloxy)acetamide | 449.2, 1.72 |
| 228 | 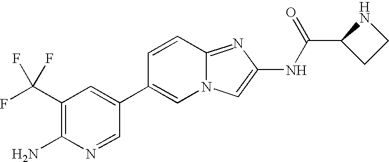 | ++++ | N/D | +++ | (S)-N-(6-(6- amino-5- (trifluoromethyl) pyridin-3- yl)imidazo[1, 2-a]pyridin-2- yl)azetidine- 2-carboxamide | 377.0, 1.59 |
| 229 | 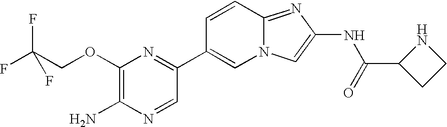 | ++++ | N/D | +++ | N-(6-(5- amino-6- (2,2,2- trifluoroethoxy) pyrazin-2- yl)imidazo[1, 2-a]pyridin-2- yl)azetidine- 2-carboxamide | 408.1, 1.76 |
| 230 | 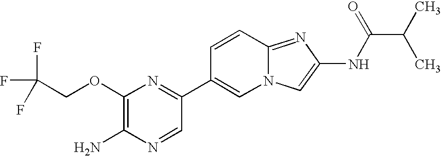 | ++++ | N/D | +++ | N-(6-(5- amino-6- (2,2,2- trifluoroethoxy) pyrazin-2- yl)imidazo[1, 2-a]pyridin-2- yl)isobutyramide | 395.1, 1.97 |
| 231 | 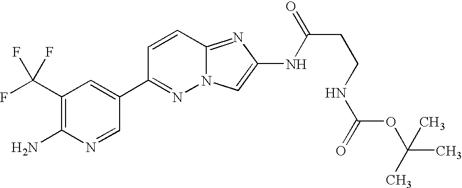 | ++++ | N/D | +++ | tert-butyl 3- (6-(6-amino-5- (trifluoromethyl) pyridin-3- yl)imidazo[1, 2-b]pyridazin- 2-ylamino)-3- oxopropyl- carbamate | 466.1, 2.40 |
| 232 | 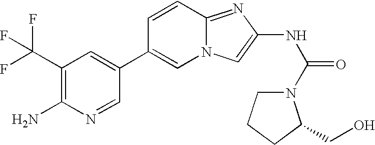 | ++++ | N/D | +++ | (S)-N-(6-(6- amino-5- (trifluoromethyl) pyridin-3- yl)imidazo[1, 2-a]pyridin-2- yl)-2- (hydroxymethyl) pyrrolidine-1- carboxamide | 421.1, 1.82 |
| 233 | 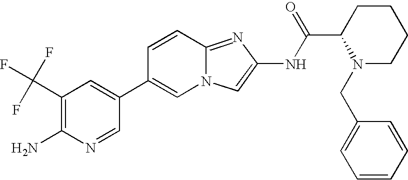 | ++++ | N/D | +++ | (S)-N-(6-(6- amino-5- (trifluoromethyl) pyridin-3- yl)imidazo[1, 2-a]pyridin-2- benzylpiperidine-2- carboxamide | 495.2, 1.96 |
| 234 | 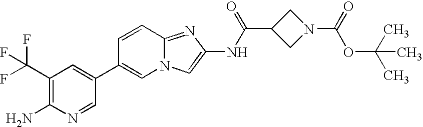 | ++++ | N/D | +++ | tert-butyl 3- (6-(6-amino-5- (trifluoromethyl) pyridin-3- yl)imidazo[1, 2-a]pyridin-2- ylcarbamoyl) azetidine-1- carboxylate | 477.1, 2.24 |
| 235 | 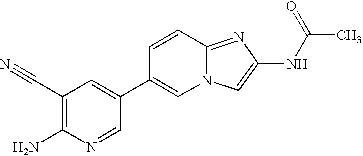 | ++++ | +++ | +++ | N-(6-(6- amino-5- cyanopyridin-3- yl)imidazo[1, 2-a]pyridin-2- yl)acetamide | 293.1, 1.59 |
| 236 | 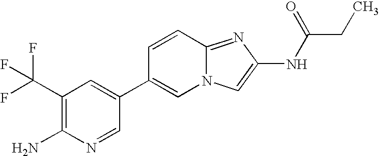 | ++++ | N/D | +++ | N-(6-(6- amino-5- (trifluoromethyl) pyridin-3- yl)imidazo[1, 2-a]pyridin-2- yl)propionamide | 350.0, 1.55 |
| 237 | 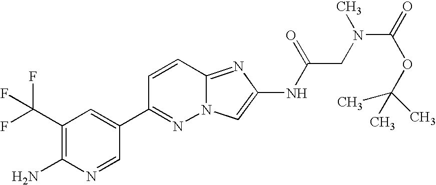 | ++++ | N/D | +++ | tert-butyl 2- (6-(6-amino-5- (trifluoromethyl) pyridin-3- yl)imidazo[1, 2-b]pyridazin- 2-ylamino)-2- oxoethyl(methyl) carbamate | 466.0, 2.19 |
| 238 | 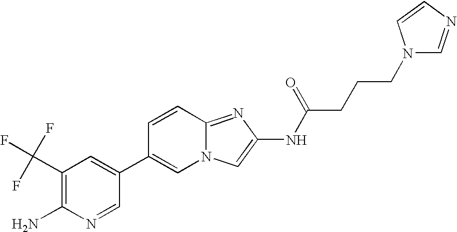 | ++++ | N/D | +++ | N-(6-(6- amino-5- (trifluoromethyl) pyridin-3- yl)imidazo[1, 2-a]pyridin-2- yl)-4-(1H- imidazol-1- yl)butanamide | 430.1, 1.63 |
| 239 | 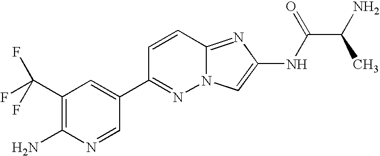 | ++++ | N/D | +++ | (S)-2-amino- N-(6-(6- amino-5- (trifluoromethyl) pyridin-3- yl)imidazo[1, 2-b]pyridazin-2- yl)propanamide | 366.1, 1.66 |
| 240 | 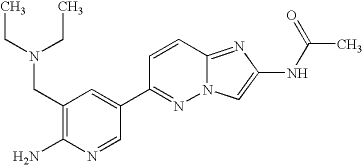 | ++++ | N/D | +++ | N-(6-(6- amino-5- ((diethylamino) methyl)pyridin-3- yl)imidazo[1, 2-b]pyridazin- 2-yl)acetamide | 354.1, 1.43 |
| 241 | 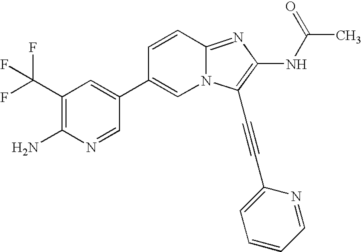 | ++++ | +++ | +++ | N-(6-(6- amino-5- (trifluoromethyl) pyridin-3- yl)-3-(pyridin-2- ylethnyl)imi- dazo[1,2- a]pyridin-2- yl)acetamide | 437.1, 1.67 |
| 242 | 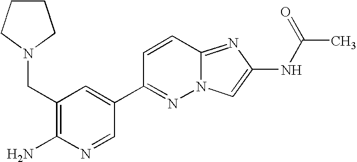 | ++++ | N/D | +++ | N-(6-(6- amino-5- (pyrrolidin-1- ylmethyl)pyridin-3- yl)imidazo[1, 2-b]pyridazin- 2-yl)acetamide | 352.1, 1.35 |
| 243 | 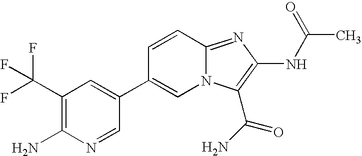 | ++++ | N/D | +++ | 2-acetamido- 6-(6-amino-5- (trifluoromethyl) pyridin-3- yl)imidazo[1, 2-a]pyridine- 3-carboxamide | 379.0, 1.50 |
| 244 | 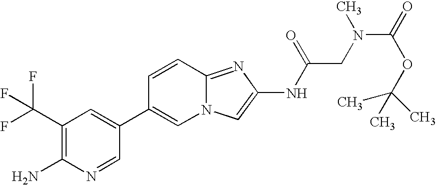 | ++++ | N/D | +++ | tert-butyl 2- (6-(6-amino-5- (trifluoromethyl) pyridin-3- yl)imidazo[1, 2-a]pyridin-2- ylamino)-2- oxoethyl(methyl) carbamate | 465.0, 1.93 |
| 245 | 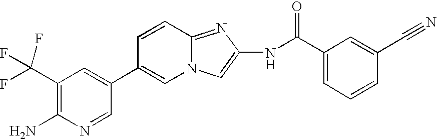 | ++++ | N/D | +++ | N-(6-(6- amino-5- (trifluoromethyl) pyridin-3- yl)imidazo[1, 2-a]pyridin-2- yl)-3- cyanobenzamide | 423.1, 2.19 |
| 246 | 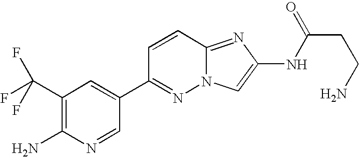 | ++++ | N/D | +++ | 3-amino-N- (6-(6-amino-5- (trifluoromethyl) pyridin-3- yl)imidazo[1, 2-b]pyridazin-2- yl)propanamide | 366.1, 1.68 |
| 247 | 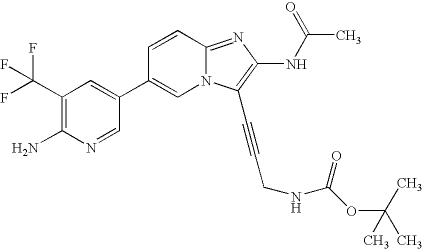 | ++++ | N/D | +++ | tert-butyl 3-(2- acetamido-6- (6-amino-5- (trifluoromethyl) pyridin-3- yl)imidazo[1, 2-a]pyridin-3- yl)prop-2- ynylcarbamate | 289.2, 1.99 |
| 248 | 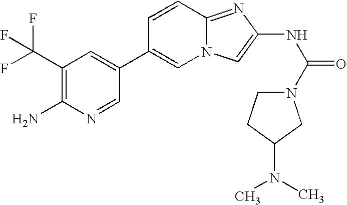 | ++++ | N/D | +++ | N-(6-(6- amino-5- (trifluoromethyl) pyridin-3- yl)imidazo[1, 2-a]pyridin-2- yl)-3- (dimethylamino) pyrrolidine-1- carboxamide | 434.2, 1.59 |
| 249 | 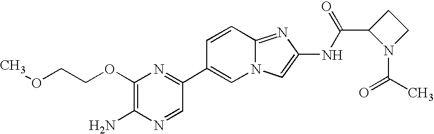 | ++++ | N/D | +++ | 1-acetyl-N-(6- (5-amino-6-(2- methoxyethoxy) pyrazin-2- yl)imidazo[1, 2-a]pyridin-2- yl)azetidine- 2-carboxamide | 426.2, 1.61 |
| 250 | 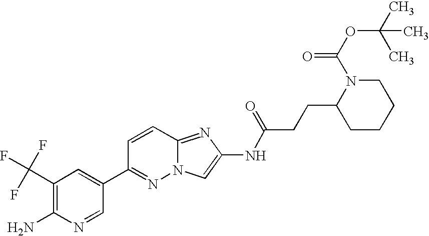 | ++++ | N/D | +++ | tert-butyl 2- (3-(6-(6- amino-5- (trifluoromethyl) pyridin-3- yl)imidazo[1, 2-b]pyridazin- 2-ylamino)-3- oxopropyl) piperidine-1- carboxylate | 534.1, 2.89 |
| 251 | 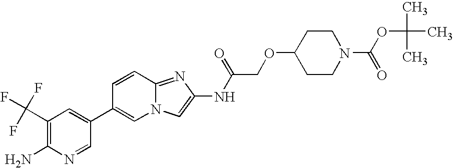 | ++++ | N/D | +++ | tert-butyl 4- (2-(6-(6- amino-5- (trifluoromethyl) pyridin-3- yl)imidazo[1, 2-a]pyridin-2- ylamino)-2- oxoethoxy) piperidine-1- carboxylate | 535.2, 2.48 |
| 252 | 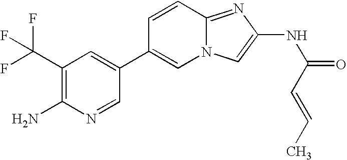 | ++++ | N/D | +++ | (E)-N-(6-(6- amino-5- (trifluoromethyl) pyridin-3- yl)imidazo[1, 2-a]pyridin-2- yl)but-2- enamide | 362.0, 1.80 |
| 253 | 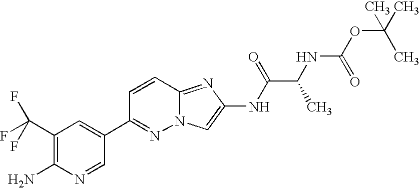 | ++++ | N/D | +++ | (R)-tert-butyl 1-(6-(6- amino-5- (trifluoromethyl) pyridin-3- yl)imidazo[1, 2-b]pyridazin- 2-ylamino)-1- oxopropan-2- ylcarbamate | 466.1, 2.46 |
| 254 | 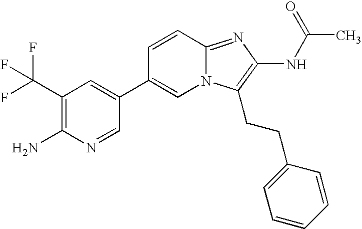 | ++++ | N/D | +++ | N-(6-(6- amino-5- (trifluoromethyl) pyridin-3- yl)-3- phenethyl- imidazo[1,2- a]pyridin-2- yl)acetamide | 440.1, 1.90 |
| 255 | 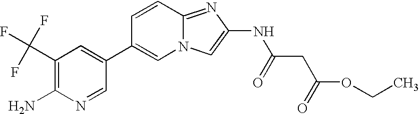 | ++++ | N/D | +++ | ethyl 3-(6-(6- amino-5- (trifluoromethyl) pyridin-3- yl)imidazo[1, 2-a]pyridin-2- ylamino)-3- oxopropanoate | 408.1, 1.96 |
| 256 | 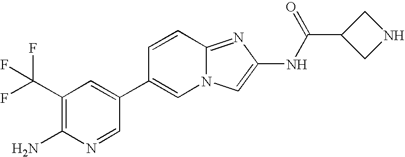 | ++++ | N/D | +++ | N-(6-(6- amino-5- (trifluoromethyl) pyridin-3- yl)imidazo[1, 2-a]pyridin-2- yl)azetidine- 3-carboxamide | 376.9, 1.30 |
| 257 | 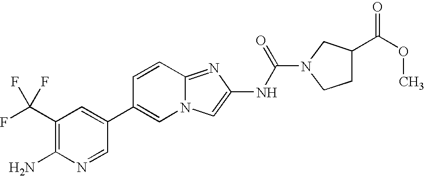 | ++++ | N/D | +++ | methyl 1-(6- (6-amino-5- (trifluoromethyl) pyridin-3- yl)imidazo[1, 2-a]pyridin-2- ylcarbamoyl) pyrrolidine-3- carboxylate | 449.5, 1.92 |
| 258 | 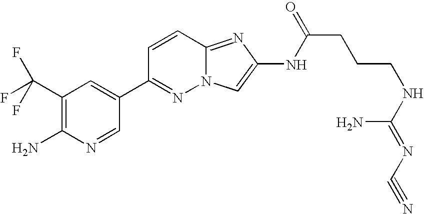 | ++++ | N/D | N/D | (E)-N-(6-(6- amino-5- (trifluoromethyl) pyridin-3- yl)imidazo[1, 2-b]pyridazin- 2-yl)-4-(2- cyanoguanidino) butanamide | 447.2, 1.92 |
| 259 | 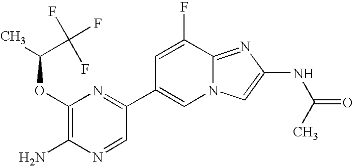 | ++++ | N/D | N/D | (S)-N-(6-(5- amino-6- (1,1,1- trifluoropropan-2- yloxy)pyrazin- 2-yl)-8- fluoroimidazo [1,2-a]pyridin- 2-yl)acetamide | 399.1, 2.27 |
| 260 | 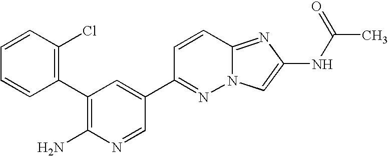 | ++++ | N/D | N/D | N-(6-(6- amino-5-(2- chlorophenyl) pyridin-3- yl)imidazo[1, 2-b]pyridazin- 2-yl)acetamide | 379.0, 1.72 |
| 261 | 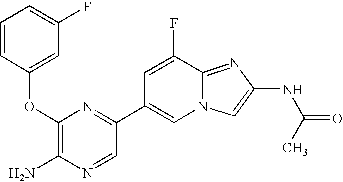 | ++++ | N/D | N/D | N-(6-(5- amino-6-(3- fluorophenoxy) pyrazin-2-yl)-8- fluoroimidazo [1,2-a]pyridin- 2-yl)acetamide | 397.1, 2.31 |
| 262 | 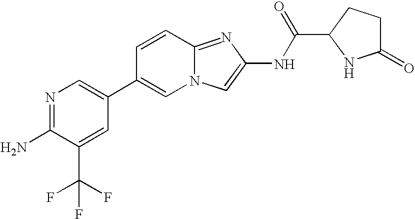 | ++++ | N/D | N/D | N-(6-(6- amino-5- (trifluoromethyl) pyridin-3- yl)imidazo[1, 2-a]pyridin-2- yl)-5- oxopyrrolidine-2- carboxamide | 405.1, 1.66 |
| 263 | 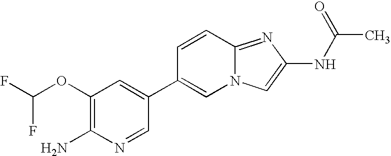 | ++++ | N/D | N/D | N-(6-(6- amino-5- (difluoromethoxy) pyridin-3- yl)imidazo[1, 2-a]pyridin-2- yl)acetamide | 333.8, 1.26 |
| 264 | 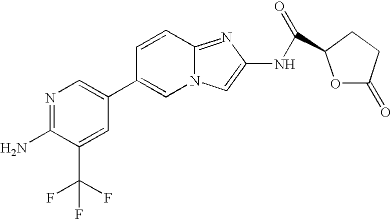 | ++++ | N/D | N/D | (R)-N-(6-(6- amino-5- (trifluoromethyl) pyridin-3- yl)imidazo[1, 2-a]pyridin-2- yl)-5- oxotetrahydro furan-2- carboxamide | 406.0, 1.79 |
| 265 | 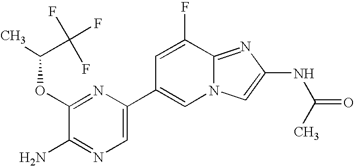 | ++++ | N/D | N/D | (R)-N-(6-(5- amino-6- (1,1,1- trifluoropropan-2- yloxy)pyrazin- 2-yl)-8- fluoroimidazo [1,2-a]pyridin-2- yl)acetamide | 399.1, 2.25 |
| 266 | 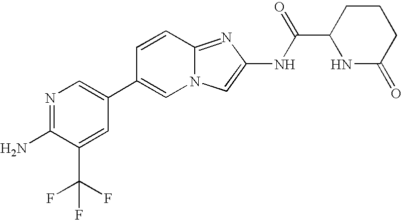 | ++++ | N/D | N/D | N-(6-(6- amino-5- (trifluoromethyl) pyridin-3- yl)imidazo[1, 2-a]pyridin-2- yl)-6-oxo- piperidine- 2-carboxamide | 419.0, 1.74 |
| 267 | 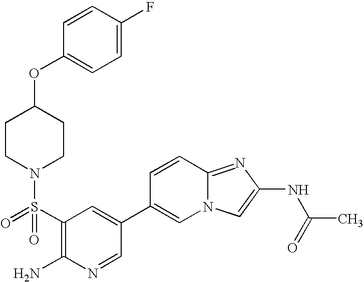 | ++++ | N/D | N/D | N-(6-(6- amino-5-(4-(4- fluorophenoxy) piperidin-1- ylsulfonyl) pyridin-3- yl)imidazo[1, 2-a]pyridin-2- yl)acetamide | 394.1 |
| 268 | 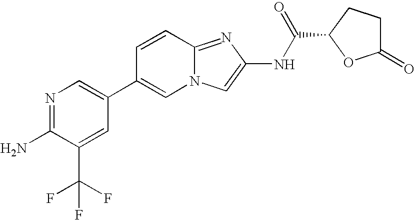 | ++++ | N/D | N/D | (S)-N-(6-(6- amino-5- (trifluoromethyl) imidazo[1, 2-a]pyridin-2- yl)-5-oxotetrahydro furan-2- carboxamide | 406.1, 1.75 |
| 269 | 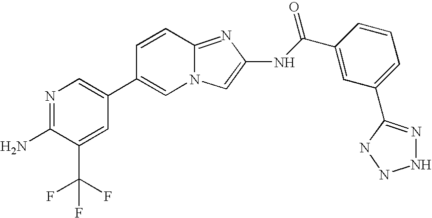 | ++++ | N/D | N/D | N-(6-(6- amino-5- (trifluoromethyl) pyridin-3- yl)imidazo[1, 2-a]pyridin-2- yl)-3-(2H- tetrazol-5- yl)benzamide | 466.1, 2.09 |
| 270 | 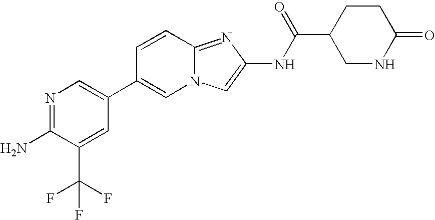 | ++++ | N/D | N/D | N-(6-(6- amino-5- (trifluoromethyl) pyridin-3- yl)imidazo[1, 2-a]pyridin-2- yl)-6- oxopiperidine-3- carboxamide | 419.1, 1.68 |
| 271 | 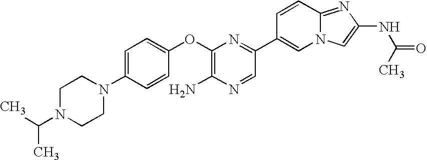 | ++++ | N/D | N/D | N-(6-(5- amino-6-(4- (4-isopropyl- piperazin-2- yl)phenoxy) pyrazin-2- yl)imidazo[1, 2-a]pyridin-2- yl)acetamide | 487.2, 1.74 |
| 272 | 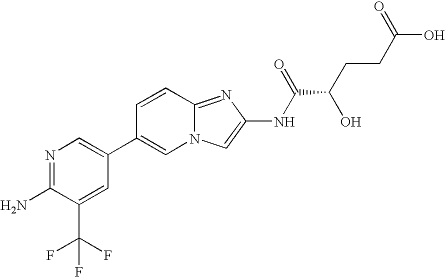 | ++++ | N/D | N/D | (S)-5-(6-(6- amino-5- (trifluoromethyl) pyridin-3- yl)imidazo[1, 2-a]pyridin-2- ylamino)-4- hydroxy-5- oxopentanoic acid | 424.2, 1.68 |
| 273 | 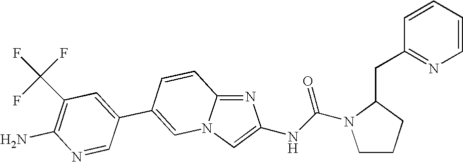 | ++++ | N/D | N/D | N-(6-(6- amino-5- (trifluoromethyl) pyridin-3- yl)imidazo[1, 2-a]pyridin-2- yl)-2-(pyridin-2- ylmethyl) pyrrolidine-1- carboxamide | 482.0, 1.53 |
| 274 | 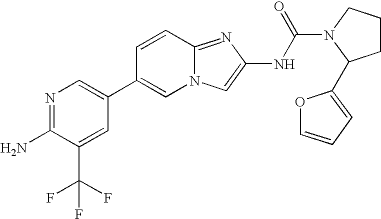 | ++++ | N/D | N/D | N-(6-(6- amino-5- (trifluoromethyl) pyridin-3- yl)imidazo[1, 2-a]pyridin-2- yl)-2-(furan-2- yl)pyrrolidine- 1-carboxamide | 457.1, 2.19 |
| 275 | 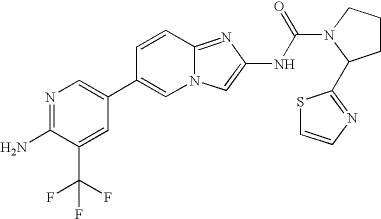 | ++++ | N/D | N/D | N-(6-(6- amino-5- (trifluoromethyl) pyridin-3- yl)imidazo[1, 2-a]pyridin-2- yl)-2-(thiazol-2- yl)pyrrolidine- 1-carboxamide | 474.0, 1.98 |
| 276 | 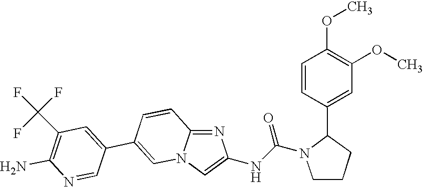 | ++++ | N/D | N/D | N-(6-(6- amino-5- (trifluoromethyl) pyridin-3- yl)imidazo[1, 2-a]pyridin-2- yl)-2-(3,4- dimethoxyphenyl) pyrrolidine-1- carboxamide | 527.0, 2.04 |
| 277 | 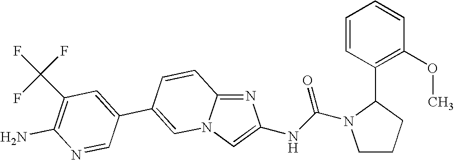 | ++++ | N/D | N/D | N-(6-(6- amino-5- (trifluoromethyl) pyridin-3- yl)imidazo[1, 2-a]pyridin-2- yl)-2-(2- methoxyphenyl) pyrrolidine- 1-carboxamide | 497.0, 2.24 |
| 278 | 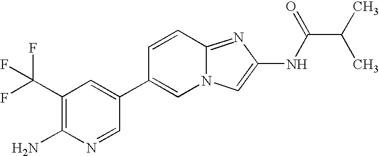 | N/D | N/D | ++ | N-(6-(6- amino-5- (trifluoromethyl) pyridin-3- yl)imidazo[1, 2-a]pyridin-2- yl)isobutyramide | 363.9, 1.70 |
| 279 | 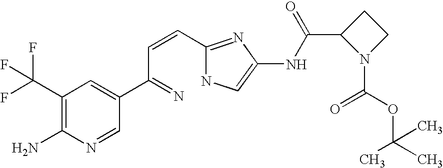 | N/D | N/D | ++ | tert-butyl 2- (6-(6-amino-5- (trifluoromethyl) pyridin-3- yl)imidazo[1, 2-b]pyridazin- 2-ylcarbamoyl) azetidine-1- carboxylate | 478.0, 2.26 |
| 280 | 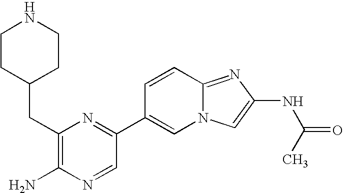 | N/D | N/D | ++ | N-(6-(5- amino-6- (piperidin-4- yloxy)pyrazin-2- yl)imidazo[1, 2-a]pyridin-2- yl)acetamide | 368.2, 1.43 |
| 281 | 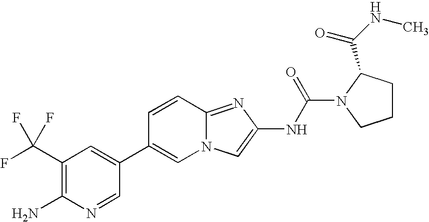 | N/D | N/D | ++ | (S)-N1-(6-(6- amino-5- (trifluoromethyl) pyridin-3- yl)imidazo[1, 2-a]pyridin-2- yl)-N2- methylpyrrolidine- 1,2-dicarboxamide | 448.2, 1.70 |
| 282 | 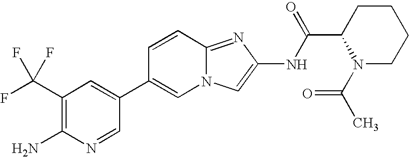 | N/D | N/D | ++ | (S)-1-acetyl- N-(6-(6- amino-5- (trifluoromethyl) pyridin-3- yl)imidazo[1, 2-a]pyridin-2- yl)piperidine- 2-carboxamide | 447.2, 1.96 |
| 283 | 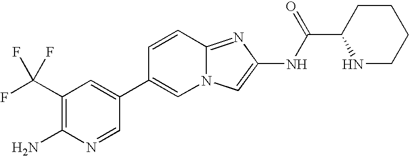 | N/D | ++++ | +++ | (S)-N-(6-(6- amino-5- (trifluoromethyl) pyridin-3- yl)imidazo[1, 2-a]pyridin-2- yl)piperidine- 2-carboxamide | 405.2, 1.64 |
| 284 | 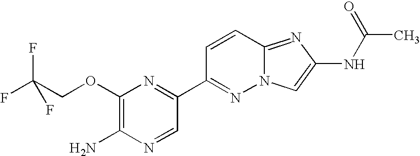 | N/D | N/D | +++ | N-(6-(5- amino-6- (2,2,2- trifluoroethoxy) pyrazin-2- yl)imidazo[1, 2-b]pyridazin- 2-yl)acetamide | 368.1, 2.05 |
| 285 | 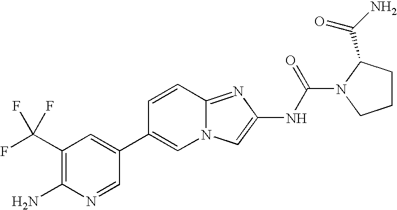 | N/D | N/D | +++ | (S)-N1-(6-(6- amino-5- (trifluoromethyl) pyridin-3- yl)imidazo[1, 2-a]pyridin-2- yl)pyrrolidine- 1,2-dicarboxamide | 434.2, 1.63 |
| 286 | 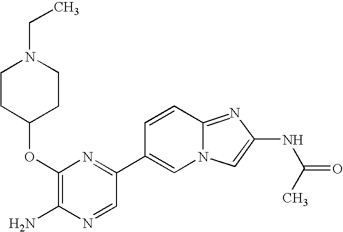 | N/D | N/D | +++ | N-(6-(5- amino-6-(1- ethylpiperidin-4- yloxy)pyrazin-2- yl)imidazo[1, 2-a]pyridin-2- yl)acetamide | 396.2, 1.50 |
| 287 | 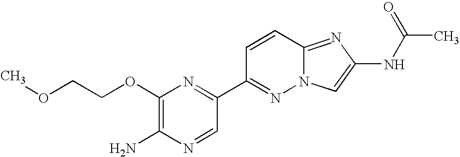 | N/D | N/D | +++ | N-(6-(5- amino-6-(2- methoxyethoxy) pyrazin-2- yl)imidazo[1, 2-b]pyridazin- 2-yl)acetamide | 344.1, 1.68 |
| 288 | 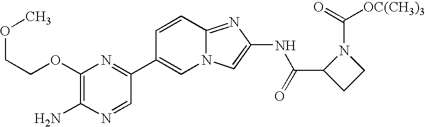 | N/D | N/D | N/D | tert-butyl 2- (6-(5-amino- 6-(2-methoxy- ethoxy)pyrazin-2- yl)imidazo[1, 2-a]pyridin-2- ylcarbamoyl) azetidine-1- carboxylate | 484.2, 2.05 |

Each of the compounds screened in Table 1 exhibited an IC₅₀ value of less than about 10 μM with respect to inhibition of PI3K. Many of the examples of Table 1 exhibited IC₅₀ values of less than about 1 μM and even less than about 0.1 μM with respect to inhibition of PI3K. For this reason, each of the compounds is individually preferred and preferred as a member of a group.

### Compounds of Formula III

### Example 47

### Preparation of N-(6-(2-aminopyrimidin-5-yl)benzo[d]thiazol-2-yl)acetamide

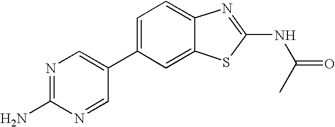

To a mixture of N-(6-bromobenzo[d]thiazol-2-yl)acetamide (15 mg, 0.06 mmol) and 5-(4,4,5,5-tetramethyl-1,3,2-dioxaborolan-2-yl)pyrimidin-2-amine (26 mg, 0.11 mmol) in DME (1.5 mL) and aqueous sodium carbonate (2M, 0.6 mL) was added Pd(dppf)Cl₂-DCM (23 mg, 0.03 mmol). This mixture was heated in a microwave at 120° C. for 800 sec. The two phases were separated and the organic layer was concentrated, dissolved in DMSO, filtered and purified by preparative HPLC to give N-(6-(2-aminopyrimidin-5-yl)benzo[d]thiazol-2-yl)acetamide as a TFA salt. LC/MS (m/z) 286.0 (MH⁺), Rₜ: 1.63 min.

### Example 48

### Preparation of N-(6-(6-amino-5-methoxypyridin-3-yl)benzo[d]thiazol-2-yl)acetamide

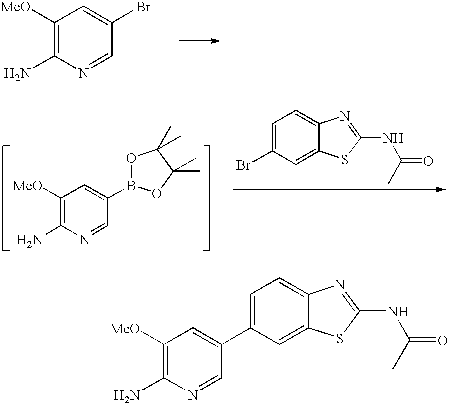

To a nitrogen sparged mixture of 5-bromo-3-methoxypyridin-2-amine (118 mg, 0.58 mmol), bis(pinacolato)diboron (160 mg, 0.63 mmol), and potassium acetate (169 mg, 1.73 mmol) in dioxane (1 mL) was added 1,1′-bis(diphenylphosphino)ferrocene]-dichloro palladium(II) complex with dichloromethane (1:1) (24 mg, 0.03 mmol). The solution was heated for four hours at 115° C. and cooled to room temperature. The boronic ester intermediate was used for next step without further purification.

Half of this solution (500 μL, 0.3 mmol) was added to N-(6-bromobenzo[d]thiazol-2-yl)acetamide (24 mg, 0.09 mmol) in 1.3 mL DME. After flushing with nitrogen, [1,1′-bis(diphenylphosphino)ferrocene]dichloro palladium(II) complex with dichloromethane (1:1) (37 mg, 0.05 mmol) was added, and the mixture was heated in a microwave at 115° C. for 700 sec. The organic layer was then reduced in volume, dissolved in DMSO, filtered and purified by preparative HPLC to give N-(6-(6-amino-5-methoxypyridin-3-yl)benzo[d]thiazol-2-yl)acetamide as its TFA salt. LC/MS (m/z): 315.1 (MH⁺); HPLC Rₜ: 1.87 min.

The following compounds were prepared as their TFA salts from the corresponding boronic esters (commercially available or prepared according to Methods 7 or 8, or prepared from the bromides in situ) according to Example 48.

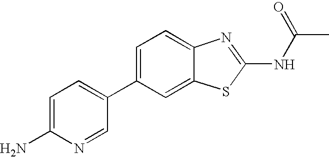

N-(6-(6-aminopyridin-3-yl)benzo[d]thiazol-2-yl)acetamide. LC/MS (m/z) 285.0 (MH⁺), Rₜ: 1.73 min; HPLC Rₜ: 1.69 min.

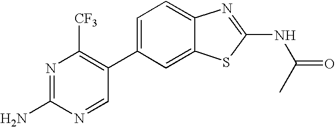

N-(6-(2-amino-4-(trifluoromethyl)pyrimidin-5-yl)benzo[d]thiazol-2-yl)acetamide: LC/MS (m/z) 354.0 (MH⁺), Rₜ: 2.23 min; HPLC Rₜ: 2.76 min.

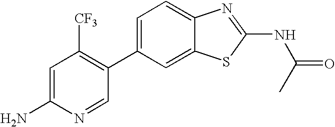

N-(6-(6-amino-4-(trifluoromethyl)pyridin-3-yl)benzo[d]thiazol-2-yl)acetamide. LC/MS (m/z) 352.9 (MH⁺), Rₜ: 1.68 min; HPLC Rₜ: 2.09 min.

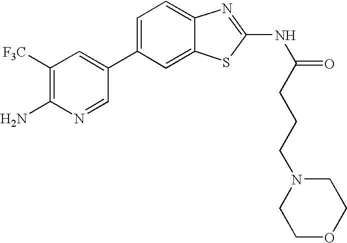

N-(6-(6-amino-5-(trifluoromethyl)pyridin-3-yl)benzo[d]thiazol-2-yl)-4-morpholinobutanamide. LC/MS (m/z) 466.0 (MH⁺), Rₜ: 1.87 min; HPLC Rₜ: 1.92 min.

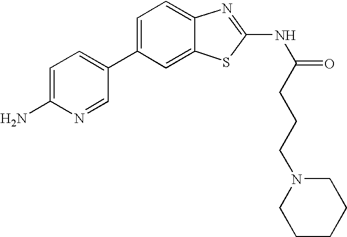

N-(6-(6-aminopyridin-3-yl)benzo[d]thiazol-2-yl)-4-(piperidin-1-yl)butanamide. LC/MS (m/z) 396.1 (MH⁺), Rₜ: 1.67 min; HPLC Rₜ: 1.56 min.

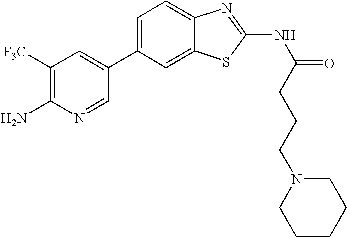

N-(6-(6-amino-5-(trifluoromethyl)pyridin-3-yl)benzo[d]thiazol-2-yl)-4-(piperidin-1-yl)butanamide. LC/MS (m/z) 464.0 (MH⁺), Rₜ: 1.98 min; HPLC Rₜ: 2.13 min.

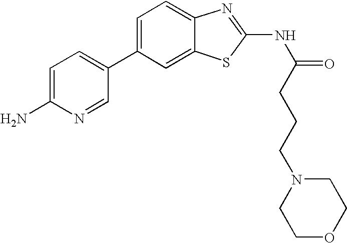

N-(6-(6-aminopyridin-3-yl)benzo[d]thiazol-2-yl)-4-morpholinobutanamide. LC/MS (m/z) 398.1 (MH⁺), Rₜ: 1.58 min; HPLC Rₜ: 1.49 min.

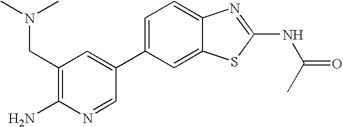

N-(6-(6-amino-5-((dimethylamino)methyl)pyridin-3-yl)benzo[d]thiazol-2-yl)acetamide. LC/MS (m/z) 342.1 (MH⁺), Rₜ: 1.52 min; HPLC Rₜ: 1.41 min.

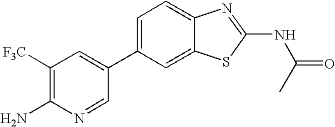

N-(6-(6-amino-5-(trifluoromethyl)pyridin-3-yl)benzo[d]thiazol-2-yl)acetamide: LC/MS (m/z) 353.0 (MH⁺), Rₜ: 2.10 min; HPLC Rₜ: 2.36 min.

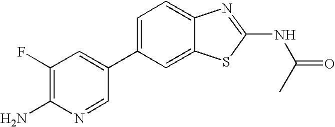

N-(6-(6-amino-5-fluoropyridin-3-yl)benzo[d]thiazol-2-yl)acetamide. LC/MS (m/z) 303.0 (MH⁺), Rₜ: 1.76 min; HPLC Rₜ: 1.78 min.

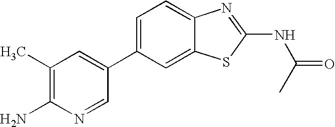

N-(6-(6-amino-5-methylpyridin-3-yl)benzo[d]thiazol-2-yl)acetamide. LC/MS (m/z) 299.1 (MH⁺), 1.80 min; HPLC Rₜ: 1.89 min.

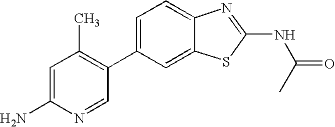

N-(6-(6-amino-4-methylpyridin-3-yl)benzo[d]thiazol-2-yl)acetamide. LC/MS (m/z) 299.1 (MH⁺), Rₜ: 1.78 min; HPLC Rₜ: 1.89 min.

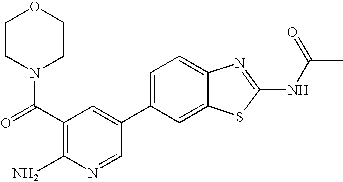

N-(6-(6-amino-5-(morpholine-4-carbonyl)pyridin-3-yl)benzo[d]thiazol-2-yl)acetamide. LC/MS (m/z) (MH⁺), Rₜ: 1.72 min; HPLC Rₜ: 1.65 min.

### Example 49

### Preparation of N-(6-(6-(propylamino)pyridin-3-yl)benzo[d]thiazol-2-yl)acetamide

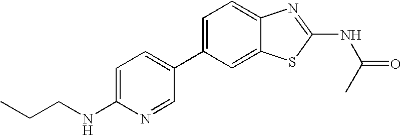

To a solution of N-(6-(6-fluoropyridin-3-yl)benzo[d]thiazol-2-yl)acetamide (6 mg, 0.02 mmol) and propylamine (0.14 mL, 1.73 mmol) in NMP (0.35 mL) was added potassium carbonate (29 mg, 0.21 mmol). This mixture was heated in an oil bath at 120° C. for 2 days, filtered and purified on a reverse-phase preparatory HPLC obtaining the desired compound as the TFA salt. LC/MS (m/z) 327.1 (MH⁺); HPLC Rₜ: 2.17 min.

The following compound was prepared according to Example 47:

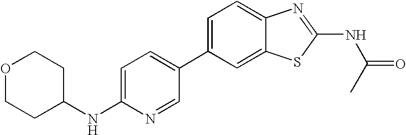

N-(6-(6-(tetrahydro-2H-pyran-4-ylamino)pyridin-3-yl)benzo[d]thiazol-2-yl)acetamide (TFA salt). LC/MS (m/z) 369.1 (MH⁺), Rₜ: 1.82 min; HPLC Rₜ: 2.04 min.

### Example 50

### Preparation of N-(6-(6-(piperidin-4-ylamino)pyridin-3-yl)benzo[d]thiazol-2-yl)acetamide

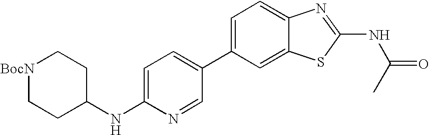

t-Butyl 4-(5-(2-acetamidobenzo[d]thiazol-6-yl)pyridin-2-ylamino)piperidine-1-carboxylate was prepared according to Example 11. LC/MS (m/z) 468.1 (MH⁺), Rₜ: 2.36 min.

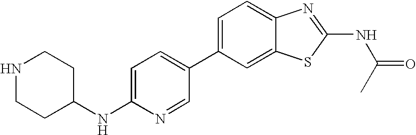

To t-butyl 4-(5-(2-acetamidobenzo[d]thiazol-6-yl)pyridin-2-ylamino)piperi-dine-1-carboxylate (4 mg, 0.009 mmol) was added HCl in dioxane (4N, 1 mL). After 3 hours, the reaction mixture was concentrated, the residue was dissolved in 1 mL acetonitrile/water (1:1) and lyophilized to give N-(6-(6-piperidin-4-ylamino)pyridin-3-yl)benzo[d]thiazol-2-yl)acetamide (1.9 mg). LC/MS (m/z) 368.1 (MH⁺), Rₜ: 1.66 min; HPLC Rₜ: 1.56 min.

### Example 51

### Preparation of N-(6-(6-acetamido-5-(trifluoromethyl)pyridin-3-yl)benzo[d]thiazol-2-yl)acetamide

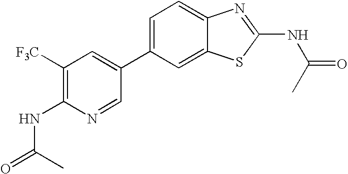

To N-(6-(6-amino-5-(trifluoromethyl)pyridin-3-yl)benzo[d]thiazol-2-yl)acetamide (17 mg, 0.05 mmol) in DMA (0.5 mL) was added acetic anhydride (0.2 mL, 2.12 mmol) and diisopropylethylamine (0.250 mL, 1.43 mmol). This solution was heated at 100° C. for 1 day, filtered and purified on a reverse-phase preparatory HPLC obtaining the desired product (1.9 mg). LC/MS (m/z) 395.0 (MH⁺), Rₜ: 2.16 min; HPLC Rₜ: 2.53 min.

Additionally, benzoxazole compounds of Formula III are synthesized according to the benzothiazole Examples and Methods using Suzuki coupling on 5-halo-2-amidobenzoxazole, as provided in Kalcheva V. et al. Khimiya Geterotsiklicheskikh Soedinenii (1984), 11, 1467-71.

The compounds in Table 2 were synthesized according to the examples provided above. PI3K inhibitory (IC₅₀) values of the compounds were determined according to Biological Method 1.

TABLE 2

| | | | | A2780 | | LC/MS |
|---|---|---|---|---|---|---|
| Com- | | PI3 K | A2780 | Cell | | (m/z, |
| pound | | Alpha | pAKT473 | prolif. | | Rt) |
| # | Structure | IC50 | EC50 | EC50 | Name | min |
| 2-1 | 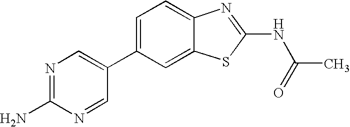 | ++++ | ++++ | +++ | N-[6-(2-Amino- pyrimidin-5-yl)- benzothiazol-2- yl]-acetamide | 286.0, 1.63 min |
| 2-2 | 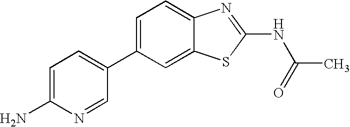 | ++++ | ++++ | +++ | N-[6-(6-Amino- pyridin-3-yl)- benzothiazol-2- yl]-acetamide | 285.0, 1.73 |
| 2-3 | 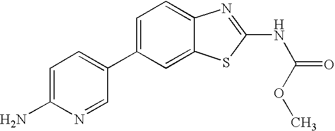 | ++++ | ++++ | +++ | [6-(6-Amino- pyridin-3-yl)- benzothiazol-2- yl]-carbamic acid methyl ester | 301.1, 1.81 |
| 2-4 | 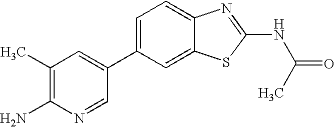 | ++++ | ++++ | +++ | N-[6-(6-Amino-5- methyl-pyridin-3- yl)-benzothiazol- 2-yl]-acetamide | 299.1, 1.80 |
| 2-5 | 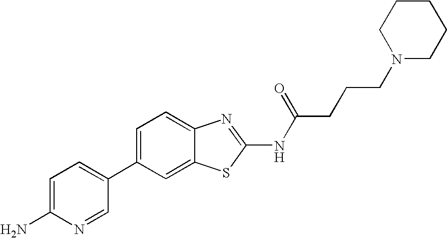 | ++++ | ++++ | +++ | N-[6-(6-Amino- pyridin-3-yl)- benzothiazol-2- yl]-4-piperidin-1- yl-butyramide | 396.1, 1.67 |
| 2-6 | 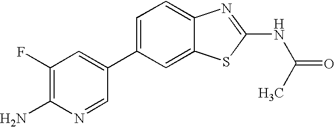 | ++++ | ++++ | +++ | N-[6-(6-Amino-5- fluoro-pyridin-3- yl)-benzothiazol- 2-yl]-acetamide | 303.0, 1.76 |
| 2-7 | 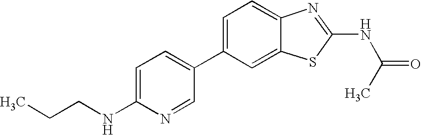 | ++++ | ++++ | ++ | N-[6-(6- Propylamino- pyridin-3-yl)- benzothiazol-2- yl]-acetamide | 327.1, 1.99 |
| 2-8 | 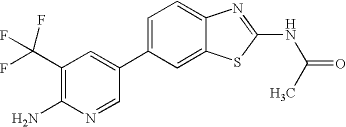 | ++++ | ++++ | ++++ | N-[6-(6-Amino-5- trifluoromethyl- pyridin-3-yl)- benzothiazol-2- yl]-acetamide | 353.0, 2.10 |
| 2-9 | 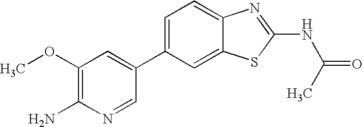 | ++++ | ++++ | ++++ | N-[6-(6-Amino-5- methoxy-pyridin- 3-yl)- benzothiazol-2- yl]-acetamide | 315.1, 1.78 |
| 2-10 | 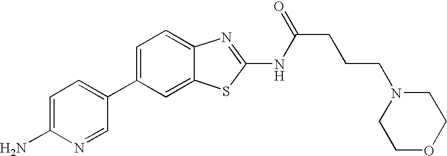 | ++++ | ++++ | ++ | N-[6-(6-Amino- pyridin-3-yl)- benzothiazol-2- yl]-4-morpholin-4- yl-butyramide | 398.1, 1.58 |
| 2-11 | 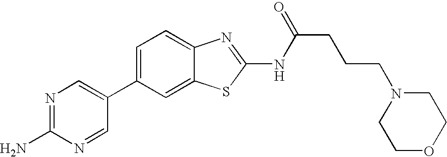 | ++++ | ++++ | +++ | N-[6-(2-Amino- pyrimidin-5-yl)- benzothiazol-2- yl]-4-morpholin-4- yl-butyramide | 399.1, 1.55 |
| 2-12 | 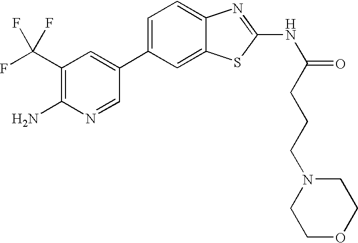 | ++++ | ++++ | +++ | N-[6-(6-Amino-5- trifluoromethyl- pyridin-3-yl)- benzothiazol-2- yl]-4-morpholin-4- yl-butyram ide | 466.0, 1.87 |
| 2-13 | 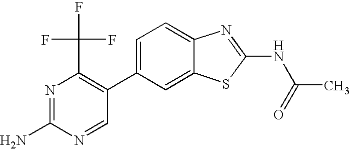 | ++++ | N/D | ++ | N-[6-(2-Amino-4- trifluoromethyl- pyrimidin-5-yl)- benzothiazol-2- yl]-acetamide | 354.0, 2.23 |
| 2-14 | 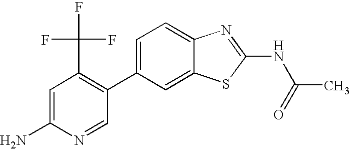 | ++++ | N/D | N/D | N-[6-(6-Amino-4- trifluoromethyl- pyridin-3-yl)- benzothiazol-2- yl]-acetamide | 352.9, 1.68 |
| 2-15 | 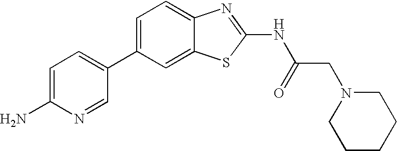 | ++++ | N/D | N/D | N-[6-(6-Amino- pyridin-3-yl)- benzothiazol-2- yl]-2-piperidin-1- yl-acetamide | 368.1, 1.61 |
| 2-16 | 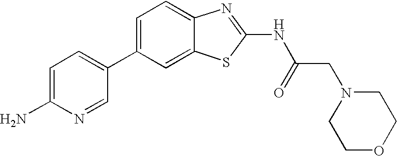 | ++++ | ++++ | ++ | N-[6-(6-Amino- pyridin-3-yl)- benzothiazol-2- yl]-2-morpholin-4- yl-acetamide | 370.1, 1.47 |
| 2-17 | 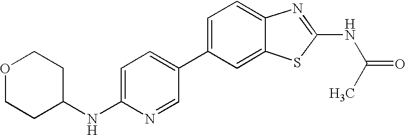 | ++++ | N/D | N/D | N-{6-[6- (Tetrahydro- pyran-4-ylamino)- pyridin-3-yl]- benzothiazol-2- yl}-acetamide | 369.1, 1.82 |
| 2-18 | 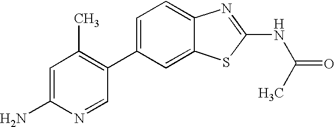 | ++++ | +++ | ++ | N-[6-(6-Amino-4- methyl-pyridin-3- yl)-benzothiazol- 2-yl]-acetamide | 299.1, 1.78 |
| 2-19 | 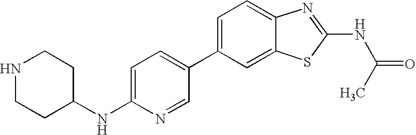 | ++++ | N/D | N/D | N-{6-[6-(Piperidin- 4-ylamino)- pyridin-3-yl]- benzothiazol-2- yl}-acetamide | 368.1, 1.66 |
| 2-20 | 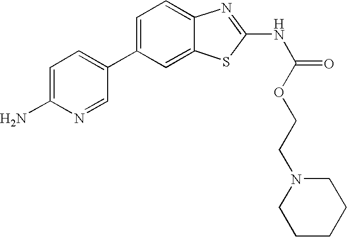 | ++++ | N/D | N/D | [6-(6-Amino- pyridin-3-yl)- benzothiazol-2- yl]-carbamic acid 2-piperidin-1-yl- ethyl ester | 398.1, 1.64 |
| 2-21 | 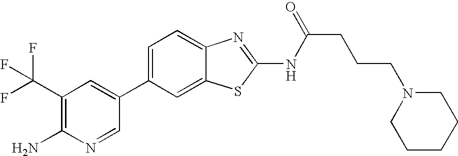 | ++++ | ++++ | +++ | N-[6-(6-Amino-5- trifluoromethyl- pyridin-3-yl)- benzothiazol-2- yl]-4-piperidin-1- yl-butyramide | 464.0, 1.98 |
| 2-22 | 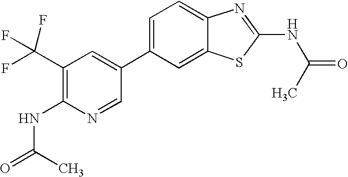 | ++++ | N/D | N/D | N-[6-(6- Acetylamino-5- trifluoromethyl- pyridin-3-yl)- bezothiazol-2- yl]-acetamide | 395.0, 2.16 |
| 2-23 | 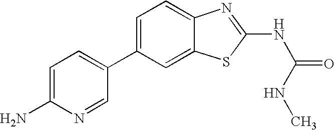 | ++++ | ++++ | +++ | 1-[6-(6-Amino- pyridin-3-yl)- benzothiazol-2- yl]-3-methyl-urea | 300.0, 1.69 |
| 2-24 | 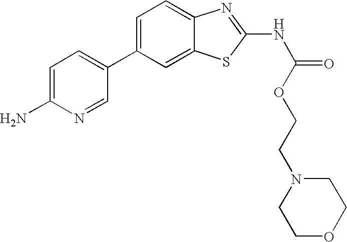 | ++++ | +++ | ++ | [6-(6-Amino- pyridin-3-yl)- benzothiazol-2- yl]-carbamic acid 2-morpholin-4-yl- ethyl ester | 400.0, 1.56 |
| 2-25 | 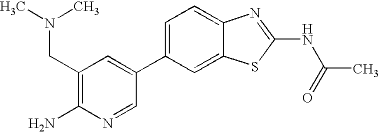 | ++++ | ++++ | +++ | N-[6-(6-Amino-5- dimethylamino- methyl-pyridin-3-yl)- benzothiazol-2- yl]-acetamide | 342.1, 1.52 |
| 2-26 | 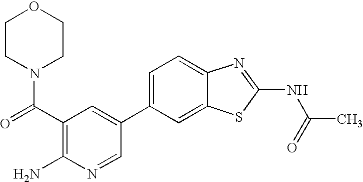 | ++++ | ++++ | +++ | N-{6-[6-Amino-5- (morpholine-4- carbonyl)-pyridin- 3-yl]- benzothiazol-2- yl}-acetamide | 398.1, 1.72 |
| 2-27 | 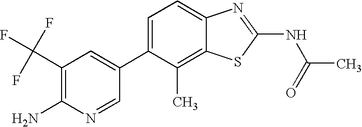 | ++++ | ++++ | +++ | N-[6-(6-Amino-5- trifluoromethyl- pyridin-3-yl)-7- methyl- benzothiazol-2- yl]-acetamide | 367.0, 2.17 |
| 2-28 | 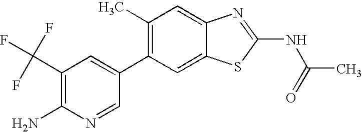 | ++++ | N/D | ++ | N-[6-(6-Amino-5- trifluoromethyl- pyridin-3-yl)-5- methyl- benzothiazol-2- yl]-acetamide | 366.9, 21.4 |
| 2-29 | 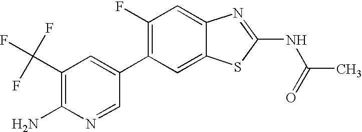 | ++++ | N/D | ++ | N-[6-(6-Amino-5- trifluoromethyl- pyridin-3-yl)-5- fluoro- benzothiazol-2- yl]-acetamide | 371.0, 2.25 |
| 2-30 | 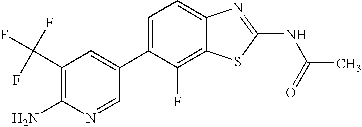 | ++++ | ++++ | +++ | N-[6-(6-Amino-5- trifluoromethyl- pyridin-3-yl)-7- fluoro- benzothiazol-2- yl]-acetamide | 371.0, 2.30 |
| 2-31 | 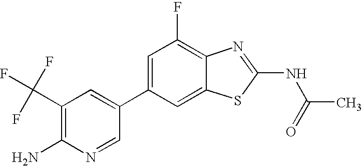 | ++++ | ++++ | ++ | N-[6-(6-Amino-5- trifluoromethyl- pyridin-3-yl)-4- fluoro- benzothiazol-2- yl]-acetamide | 371.0, 2.25 |
| 2-32 | 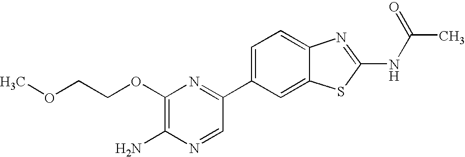 | ++++ | ++++ | +++ | N-(6-(5-amino-6- (2- methoxyethoxy) pyrazin-2- yl)benzo[d]thiazol- 2-yl)acetamide | 359.8, 1.67 |
| 2-33 | 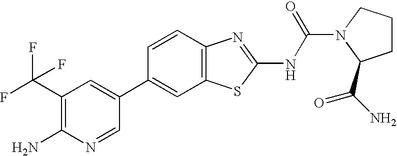 | ++++ | ++++ | +++ | (S)-N1-(6-(6- amino-5- (trifluoromethyl) pyridin-3- yl)benzo[d]thiazol- 2-yl)pyrrolidine- 1,2- dicarboxamide | 451.0, 1.96 |
| 2-34 | 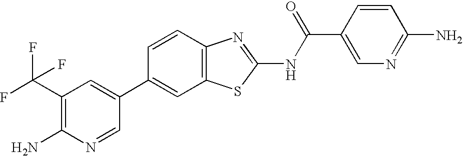 | ++++ | N/D | ++++ | 6-amino-N-(6-(6- amino-5- (trifluoromethyl) pyridin-3- yl)benzo[d]thiazol- 2-yl)nicotinamide | 431.0, 1.94 |
| 2-35 | 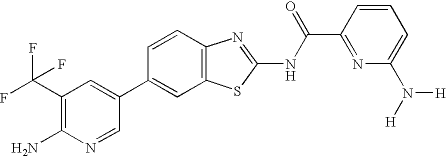 | +++ | N/D | N/D | 6-amino-N-(6-(6- amino-5- (trifluoromethyl) pyridin-3- yl)benzo[d]thiazol- 2-yl)picolinamide | 431.0, 2.14 |
| 2-36 | 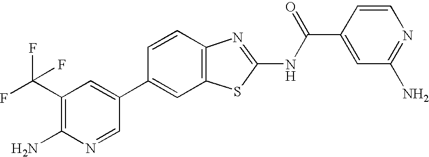 | ++++ | N/D | N/D | 2-amino-N-(6-(6- amino-5- (trifluoromethyl) pyridin-3- yl)benzo[d]thiazol- 2-yl)isonicotinamide | 431.0, 1.98 |
| 2-37 | 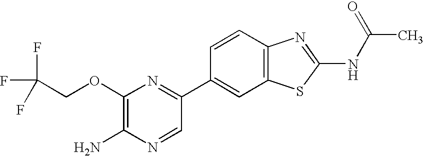 | ++++ | ++++ | ++++ | N-(6-(5-amino-6- (2,2,2- trifluoroethoxy) pyrazin-2- yl)benzo[d]thiazol- 2-yl)acetamide | 384.0, 2.07 |

Each of the compounds screened in Table 2 exhibited an IC₅₀ value of less than about 25 μM with respect to inhibition of PI3K. Many of the Examples of Table 2 exhibited IC₅₀ values of less than about 10 μM, and less than about 1 μM, and even less than about 0.1 μM with respect to inhibition of PI3K. For this reason, each of the compounds is individually preferred and preferred as a member of a group.

### Compounds of Formula IV and V

### Example 52

### Preparation of 1-[6-(6-Amino-5-trifluoromethyl-pyridin-3-yl)-imidazo[1,2-a]pyridin-2-yl]-3-[2-(5-ethyl-oxazol-2-yl)-ethyl]-urea)

A microwave vial was charged with 5-(4,4,5,5-tetramethyl-[1,3,2]dioxaborolan-2-yl)-3-trifluoromethyl-pyridin-2-ylamine (0.046 g, 0.16 mmol), aqueous sodium carbonate (2M, 0.5 mL) and DME (2 mL). Argon was bubbled through the stirred mixture for 30 minutes at room temperature. 1-(6-Bromo-imidazo[1,2-a]pyridin-2-yl)-3-[2-(5-ethyl-oxazol-2-yl)-ethyl]-urea (Intermediate E4)(0.05 g, 0.13 mmol) and Pd(dppf)Cl₂.DCM (0.016 g, 0.02 mmol) were added and the reaction mixture was heated in a microwave oven at 100° C. for 15 minutes. The reaction mixture was diluted with EtOAc (150 ml), washed with saturated aqueous sodium bicarbonate (30 ml) followed by brine (30 ml) and dried (MgSO₄). The crude product was absorbed on silica and purified by chromatography on silica, eluting with methanol in DCM (2.5% increasing to 10%) to afford the title compound.

The compounds of Table 3 are prepared analogously to Example 50 from the appropriate imidazole-urea bromo intermediate and boronic acids/boronate esters.

TABLE 3

| | | | LC/MS |
|---|---|---|---|
| Compound | Structure | Name | (m/z) |
| 3-1 | 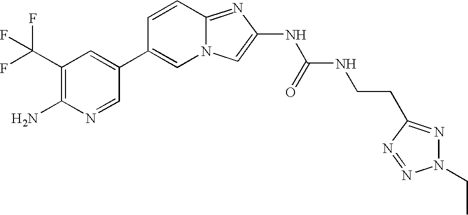 | 1-[6-(6-Amino-5- trifluoromethyl-pyridin- 3-yl)-imidazo[1,2- a]pyridin-2-yl]-3-[2-(2- ethyl-2H-tetrazol-5- yl)-ethyl]-urea | 460.69 |
| 3-2 | 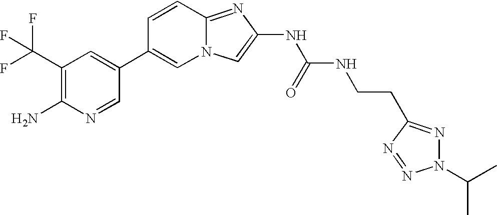 | 1-[6-(6-Amino-5- trifluoromethyl-py- ridin-3-yl)-imidazo[1,2- a]pyridin-2-yl]-3-[2-(2- isopropyl-2H-tetrazol- 5-yl)-ethyl]-urea | 475.17 |
| 3-3 | 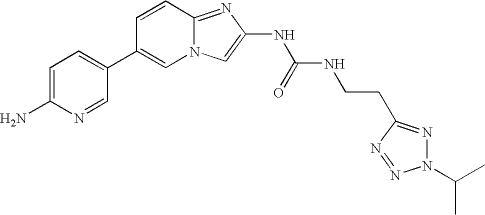 | 1-[6-(6-Amino- pyridin-3-yl)- imidazo[1,2-a]pyridin- 2-yl]-3-[2-(2- isopropyl-2H-tetrazol- 5-yl)-ethyl]-urea | 407.22 |
| 3-4 | 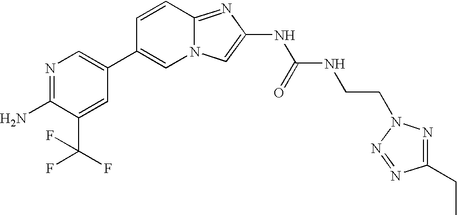 | 1-[6-(6-Amino-5- trifluoromethyl-py- ridin-3-yl)-imidazo[1,2- a]pyridin-2-yl]-3-[2-(5- ethyl-tetrazol-2-yl)- ethyl]-urea | 460.97 |
| 3-5 | 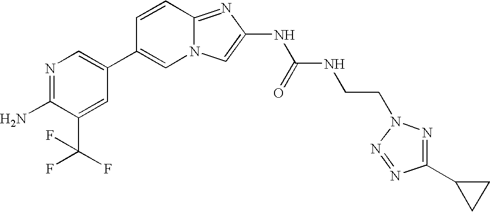 | 1-[6-(6-Amino-5- trifluoromethyl-py- ridin-3-yl)-imidazo[1,2- a]pyrid in-2-yl]-3-[2-(5- cyclopropyl-tetrazol- 2-yl)-ethyl]-urea | 473.12 |
| 3-6 | 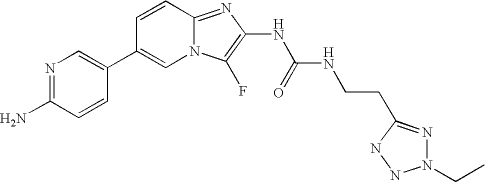 | 1-[6-(6-Amino- pyridin-3-yl)-3-fluoro- imidazo[1,2- a]pyridin-2-yl]-3-[2- (2-ethyl-2H-tetrazol- 5-yl)-ethyl]-urea | |
| 3-7 | 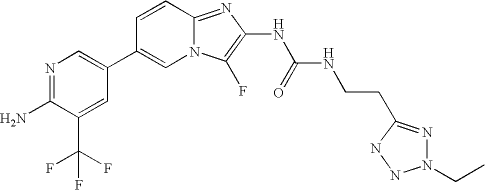 | 1-[6-(6-Amino-5- trifluoromethyl-py- ridin-3-yl)-3-fluoro- imidazo[1,2-a] pyridin-2-yl]-3-[2-(2- ethyl-2H-tetra zol-5-yl)-ethyl]-urea | |
| 3-8 | 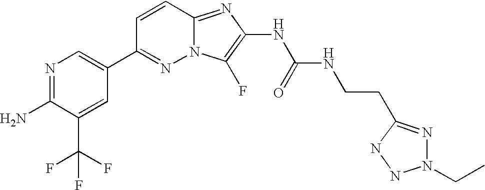 | 1-[6-(6-Amino-5- trifluoromethyl-py- ridin-3-yl)-imidazo[1,2- b]pyridazin- 2-yl]-3-[2-(2-ethyl-2H- tetrazol-5-yl)- ethyl]-urea | |
| 3-9 | 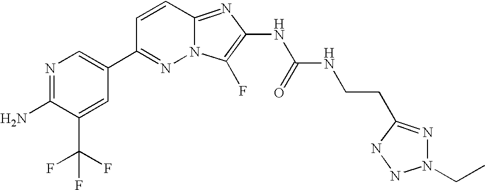 | 1-[6-(6-Amino-5- trifluoromethyl-py- ridin-3-yl)-imidazo[1,2- b]pyridazin- 2-yl]-3-[2-(5-ethyl- tetrazol-2-yl)- ethyl]-urea | |
| 3-10 | 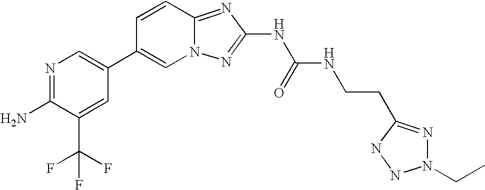 | 1-[6-(6-Amino-5- trifluoromethyl-py- ridin-3-yl)- 1,2,4]triazolo[1,5-a] pyridin-2-yl]-3-[2-(2- ethyl-2H-tetrazol-5- yl)-ethyl]-urea | |
| 3-11 | 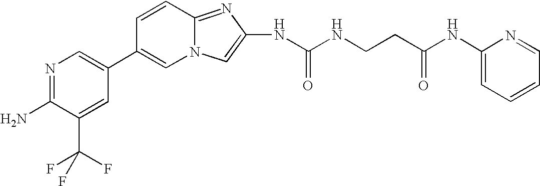 | 3-{3-[6-(6-Amino-5- trifluoromethyl- pyridin-3-yl)- imidazo[1,2-a]pyridin- 2-yl]-ureido}-N- pyridin-2-yl- propionamide | |
| 3-12 | 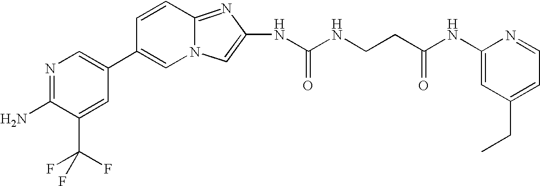 | 3-{3-[6-(6-Amino-5- trifluoromethyl- pyridin-3-yl)- imidazo[1,2-a]pyridin- 2-yl]-ureido}-N-(4- ethyl-pyridin-2-yl)- propionamide | |

Ki values of the compounds in Table 3 were determined according to Biological Method 4 and shown in Table 4 when assayed for inhibition with respect to PI3 kinase isoforms alpha, beta, gamma and delta where * * * * denotes Ki of less than 1 μM and * * * denotes Ki of less than 10 μM.

TABLE 4

| Compound | Gamma | Alpha | Delta | Beta |
|---|---|---|---|---|
| Example 52 | **** | **** | **** | **** |
| 3-1 | **** | **** | **** | **** |
| 3-2 | **** | **** | **** | **** |
| 3-3 | **** | *** | **** | **** |
| 3-4 | **** | **** | **** | **** |
| 3-5 | **** | **** | **** | **** |

Each of the compounds listed in Table 4 exhibited an IC₅₀ value of less than 10 μM with respect to inhibition of PI3K. Most of the Examples of Table 1 exhibited IC₅₀ values of less than about 1 μM, and some and even less than about 0.1 μM with respect to inhibition of PI3K. For this reason, each of the compounds is individually preferred and preferred as a member of a group. In particular, the compounds of Table 4 were all found to be selective for the gamma isoform by approximately approximately 19 to 91 times relative to the alpha isoform, approximately 5 to 54 times relative to the delta isoform, and approximately 1.5 to 5 times relative to the beta isoform.

### BIOLOGICAL EXAMPLES

### Biological Method 1

### Phosphorylation Assays

### Assay 1: Homogenous Solution Phase Assay

Compounds to be tested are dissolved in DMSO and directly distributed into 384-well flashplates at 1.25 μL per well. To start the reaction, 20 μL of 6 nM PI3 kinase are added into each well followed by 20 μL of 400 nM ATP containing a trace of radiolabeled ATP and 900 nM 1-alpha-phosphatidylinositol (PI). The plates are briefly centrifuged to remove any air gap. The reaction is performed for 15 minutes and then stopped by the addition of 20 μL of 100 mM EDTA. The stopped reaction is incubated overnight at RT to allow the lipid substrate to bind by hydrophobic interaction to the surface of the flashplate. The liquid in the wells is then washed away, and the labeled substrate is detected with scintillation counting.

### Assay 2: One Step Solid Phase Assay

This method is similar to Assay 1 except that the lipid substrate (1-alpha-phosphatidylinositol (PI)) is first dissolved in a coating buffer and incubated on flashplate at room temperature overnight to allow the lipid substrate to bind by hydrophobic interaction to the surface of the flashplate. Unbound substrate is then washed away. On the day of assay, 20 μL of 6 nM PI3 kinase are added into each well followed by 20 μL of 400 nM ATP containing trace of radiolabeled ATP. Compounds are added together with enzyme and ATP to the lipid-coated plates. The plates are briefly centrifuged to remove any air gap. The reaction is performed for two to three hours. The reaction is stopped by addition of 20 μL of 100 mM EDTA or by immediate plate washing. Phosphorylated lipid substrate is detected by scintillation counting.

### Assay 3: ATP Depletion Assay

Compounds to be tested are dissolved in DMSO and directly distributed into a black 384-well plate at 1.25 μL per well. To start the reaction, 25 μL of 10 nM PI3 kinase and 5 μg/mL 1-alpha-phosphatidylinositol (PI) are added into each well followed by 25 μL of 2 μM ATP. The reaction is performed until approx 50% of the ATP is depleted, and then stopped by the addition of 25 μL of KinaseGlo solution. The stopped reaction is incubated for 5 minutes and the remaining ATP is then detected via luminescence. IC50 values were then determined and are shown in Tables 1 and 2 in the column labeled “PI3 K Alpha IC50”.

### Biological Method 2

pSer⁴⁷³ Akt Assays to Monitor PI3K Pathway

In this method, an assay for measuring the PI3K-mediated pSer⁴⁷³-Akt status after treatment with representative inhibitor compounds of the preferred embodiments is described.

A2780 cells were cultured in DMEM supplemented with 10% FBS, L-glutamine, sodium pyruvate, and antibiotics. Cells were plated in the same medium at a density of 15,000 cells per well into 96 well tissue culture plates, with outside wells vacant, and allowed to adhere overnight.

Test compounds supplied in DMSO were diluted further into DMSO at 500 times the desired final concentrations before dilution into culture media to 2 times the final concentrations. Equal volumes of 2× compounds in media were added to the cells in 96 well plates and incubated at 37° C. for one hour. The media and compounds were then removed, the plates chilled and cells lysed in a lysis buffer (150 mM NaCl, 20 mM Tris pH 7.5, 1 mM EDTA, 1 mM EGTA, 1% Triton X-100) supplemented with phosphatase and protease inhibitors. After thorough mixing, lysates were transferred to both pSer473Akt and total Akt assay plates from Meso Scale Discovery (MSD), and incubated overnight with shaking at 4° C. The plates were washed with 1×MSD wash buffer and the captured analytes detected with secondary antibodies. After incubation with the secondary antibody at room temperature for 1-2 hours, the plates were washed again and 1.5× concentration of Read Buffer T (MSD) was added to the wells.

The assays were read on a SECTOR Imager 6000 instrument (Meso Scale Discovery). Ratios of the signal from pSer473Akt and total Akt assays were used to correct for any variability and the percent inhibition of pSer473Akt from the total signal seen in cells treated with compound versus DMSO alone was calculated and used to determine EC₅₀ values for each compound as shown in Tables 1 and 2 in the column labeled “A2780 pAKT473 EC50”.

### Biological Method 3

### Viability Assay in A2780

Cell viability was assessed with Cell Titer Glo assay, Promega. Cells were seeded in TC treated 96-well plates at a density of 1,000 (A2780 cells) per well in DMEM with 10% FBS, 1% Sodium Pyruvate, and 1% Penicillin Streptomycin for a minimum of 2 hrs prior to addition of compound. Test compounds were serially diluted (3 fold) in DMSO to 500× the final concentration. For each concentration of test compound, 2 μL (500×) aliquots of compound or 100% DMSO (control) were diluted in 500 μL of culture medium to 2× final concentration then diluted 1× on the cells. Cells were incubated for 72 hrs at 37° C., 5% CO₂. After which Cell Titer Glo is added to determine viable cells, the assay was performed according to the manufacturer's instruction (Promega Corporation, Madison, Wis. USA). Each experimental condition was performed in duplicate. Raw data was imported in Abase and EC50s calculated with XL-fit data analysis software and are shown in Tables 1 and 2 in the column labeled “A2780 Cell prolif. EC50”.

### Biological Method 4

Activity of compounds in Table 4, expressed as Ki the dissociation constant for inhibition binding, were determined using the following test procedures.

Baculovirus expressing different fragments of PI3Kγ fused to GST have been previously described by Stoyanova, S., Bulgarelli-Leva, G., Kirsch, C., Hanck, T., Klinger, R., Wetzker, R., Wymann, M. P. (1997) Lipid- and protein kinase activities of G protein-coupled PI 3-kinase gamma: structure-activity analysis and interactions with wortmannin. Biochem. J., 324:489. Residues 38-1102 of human PI3Kγ are subcloned into the BamH1 and EcoR1 sites of the transfer vector pAcG2T (Pharmingen) to create a GST-P13Kγ lacking the first 37 residues of PI3Kγ. To express the recombinant protein, Sf9 (Spodoptera frugiperda 9) insect cells are routinely maintained at densities between 3×10⁵ and 3×10⁶ cells/ml in serum containing TNMFH medium (Sigma). Sf9 cells, at a density of 2×10⁶ are infected with human GST-PI3γΔ34 baculovirus at a multiplicity of infection (m.o.i.) of 1 for 72 hours. The infected cells are harvested by centrifugation at 1400 g for 4 minutes at 4° C. and the cell pellets are frozen at −80° C. Both Sf9 and Sf21 cells work equally well. Sf9 cells (1×10⁹) are resuspended in 100 ml cold (4° C.) lysis buffer (50 mM Tris-HCl pH 7.5, 1% Triton X-100, 150 mM NaCl, 1 mM NaF, 2 mM DTT and protease inhibitors. Cells are incubated on ice for 30 minutes then centrifuged at 15000 g for 20 minutes at 4° C. Purification of the supernatant sample is carried out at 4° C. by affinity chromatography using SEPHAROSE™ agarose gel beads coupled to glutathione (from Amersham Pharmacia Biotech). A cell lysate/GST resin ratio of 50:1 is used. The GST resin is firstly pre-rinsed to remove ethanol preservative and then equilibrated with lysis buffer. Cell lysate (supernatant) is added (usually as 50 ml lysate to 1 ml GST resin in 50 ml tubes) and gently rotated on a mixer at 4° C. for 2-3 hours. The unbound flow through sample is collected by centrifugation at 1000 g for 5 minutes at 4° C. using a DENLEY™ centrifuge. The 1 ml GST resin containing bound material is transferred to a 15 ml FALCON™ centrifuge tube for subsequent washing and elution steps. Firstly a series of 3 cycles of washings (mixing by gentle inversion) is performed with 15 ml ice cold wash Buffer A (50 mM Tris-HCl pH 7.5, 1% Triton X-100, 2 mM DTT) interspersed with centrifugation at 1000 g for 5 minutes at 4° C. A final single wash step is performed with 15 ml ice cold wash Buffer B (50 mM Tris-HCl pH 7.5, 2 mM DTT) and then centrifuged at 1000 g for 5 minutes at 4° C. The washed GST resin is finally eluted with 4 cycles of 1 ml ice cold elution buffer (50 mM Tris-HCl pH 7.5, 10 mM reduced glutathione, 2 mM DTT, 150 mM NaCl, 1 mM NaF, 50% ethylene glycol and protease inhibitors) interspersed with centrifugation at 1000 g for 5 minutes at 4° C. Samples are aliquoted and stored at −20° C. The isoforms in Table 4 were similarly purified.

An in vitro kinase assay was established that measures the transfer of the terminal phosphate of adenosine triphosphate to phosphatidylinositol. The kinase reaction is performed in a white 96 well microtitre plate as a Scintillation Proximity Assay. Each well contains 10 μl test compound in 5% dimethylsulphoxide and 20 μl assay mix (40 mM Tris, 200 mM NaCl, 2 mM ethyleneglycol-aminoethyl-tetraacetic acid (EGTA), 15 μg/ml phosphatidylinositol, 12.5 μM adenosine triphosphate (ATP), 25 mM MgCl₂, 0.1 μCi [³³P]ATP). The reaction is started by the addition of 20 μl of enzyme mix (40 mM Tris, 200 mM NaCl, 2 mM EGTA containing recombinant GST-p110γ). The plate is incubated at room temperature for 60 minutes and the reaction terminated by the adding 150 μl of WGA-bead stop solution (40 mM Tris, 200 mM NaCl, 2 mM EGTA, 1.3 mM ethylene diamine tetraacetic acid (EDTA), 2.6 μM ATP and 0.5 mg of Wheat Germ Agglutinin-SPA beads (Amersham Biosciences) to each well. The plate is sealed, incubated at room temperature for 60 minutes, centrifuged at 1200 rpm and then counted for 1 minute using a scintillation counter. Total activity is determined by adding 10 μl of 5% dimethylsulphoxide (DMSO) and non-specific activity is determined by adding 10 μl 50 mM EDTA in place of the test compound.

### Biological Method 5

### In Vivo Assay

The pharmacology of Compound 57 was profiled in the A2780 (PTEN mutated) human ovarian xenograft model in nude mice.

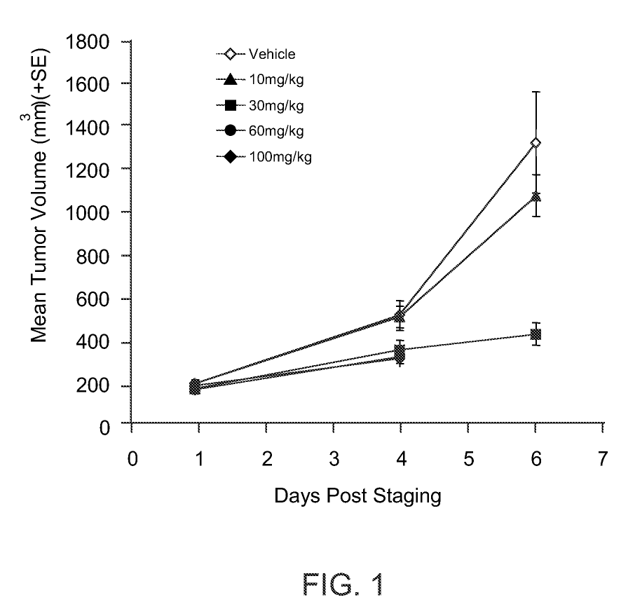

Compound 57 (3, 10, 30, or 60 mg/kg) was dosed orally to A2780 tumor-bearing mice and tumors were harvested at select times post-dose. Tumors from mice treated with vehicle were also harvested as controls. FIG. 1 shows the efficacy of Compound 57 against the A2780 xenograft tumor model. Compound 57 30 mg/kg significantly inhibited tumor growth (day 6: 79%, p<0.001 vs vehicle, ANOVA).

All of the references, patents, and patent applications cited herein are hereby incorporated by reference in their entirety.

While a number of preferred embodiments of the invention and variations thereof have been described in detail, other modifications and methods of use will be readily apparent to those of skill in the art. Accordingly, it should be understood that various applications, modifications and substitutions may be made of equivalents without departing from the spirit of the invention or the scope of the claims.

1. A compound of Formula I or a stereoisomer, tautomer, or solvate or pharmaceutically acceptable salt thereof 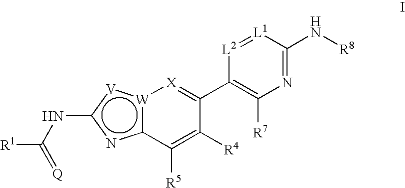
wherein:
Q is O or S;
X is CR³ or N;
W is C or N;
V is CR², O or S;
L¹ is CR⁹ or N;
L² is CR⁶ or N;
R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino;
R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;
R⁴, R⁵, and R⁶ are independently selected from the group consisting of hydrogen, halogen, cyano, nitro, amino, substituted amino, alkoxy, substituted alkoxy, alkyl, and substituted alkyl;
R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and
R^{8a} is selected from the group consisting of alkyl, substituted alkyl, alkoxy, substituted alkoxy, amino, substituted amino, and alkylamino.

2. A compound of claim 1 of Formula Ia or a stereoisomer, tautomer, or pharmaceutically acceptable salt thereof 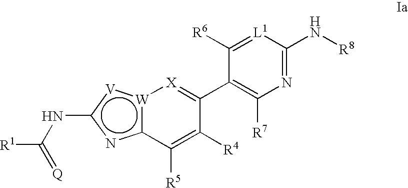
wherein R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio.

3. A compound of claim 1 of Formula II or a stereoisomer, tautomer, or solvate or pharmaceutically acceptable salt thereof

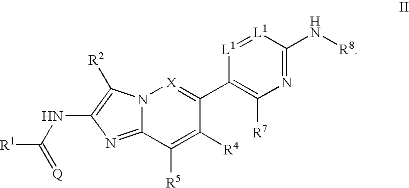

4. A compound of claim 3 of Formula IIa or a stereoisomer, tautomer, or pharmaceutically acceptable salt thereof 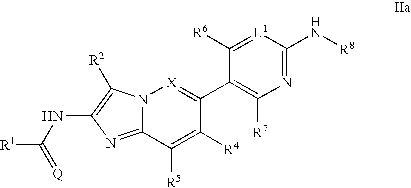
wherein R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio.

5. A compound of claim 4 having one or more of (a)-(g):
(a) R⁸ is hydrogen;
(b) L² is N or CR⁶ where R⁶ is H;
(c) R⁷ is hydrogen, alkyl, or amino;
(d) X is N or CR³ where R³ is hydrogen, alkyl, hydroxy, or alkoxy;
(e) R⁴ is hydrogen, halo, or alkyl;
(f) R⁵ is hydrogen, halo, or alkyl; and
(g) Q is O.

6. A compound of claim 4, wherein R¹ is methyl or trifluoromethyl.

7-18. (canceled)

19. A compound of claim 1 that is a compound selected from Table 1 or 3 or a stereoisomer, tautomer, or pharmaceutically acceptable salt thereof.

20. A compound of Formula III, or a stereoisomer, tautomer, or solvate or pharmaceutically acceptable salt thereof; 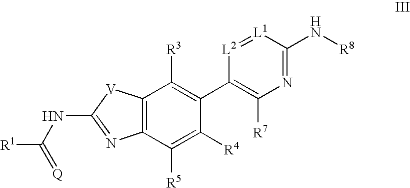
wherein:
Q is O or S;
V is O or S;
L¹ is CR⁹ or N;
L² is CR⁶ or N;
R¹ is selected from the group consisting of hydrogen, alkyl, substituted alkyl, alkenyl, substituted alkenyl, alkynyl, substituted alkynyl, alkoxy, substituted alkoxy, amino, substituted amino, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, and alkylamino; R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, imino, nitro, SO₃H, substituted sulfonyl, sulfonyloxy, thioacyl, thiol, alkylthio, and substituted alkylthio;
R⁴, R⁵, and R⁶ are independently selected from the group consisting of hydrogen, halogen, cyano, nitro, amino, substituted amino, alkoxy, substituted alkoxy, alkyl, and substituted alkyl;
R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and
R^{8a} is selected from the group consisting of alkyl, substituted alkyl, alkoxy, substituted alkoxy, amino, substituted amino, and alkylamino.

21. A compound of claim 20 of Formula IIIa, or a stereoisomer, tautomer, or pharmaceutically acceptable salt thereof 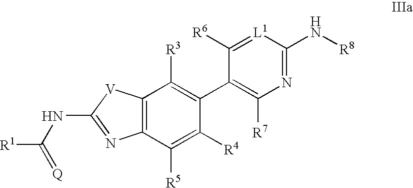
wherein R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio.

22. A compound of claim 21, wherein R¹ is selected from the group consisting of methyl, methoxy, morpholinyl-N-propyl, piperidyl-N-methyl, morpholinyl-N-methyl, piperidyl-N-ethoxy, piperidyl-N-propyl, methylamino, and morpholinyl-N-ethoxy.

23-32. (canceled)

33. A compound of claim 21 that is a compound selected from Table 2 or a stereoisomer, tautomer, or pharmaceutically acceptable salt thereof.

34. A compound of Formula IV, or a stereoisomer, tautomer, or solvate or pharmaceutically acceptable salt thereof, 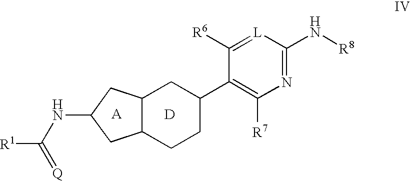
wherein,
ring AD is selected from 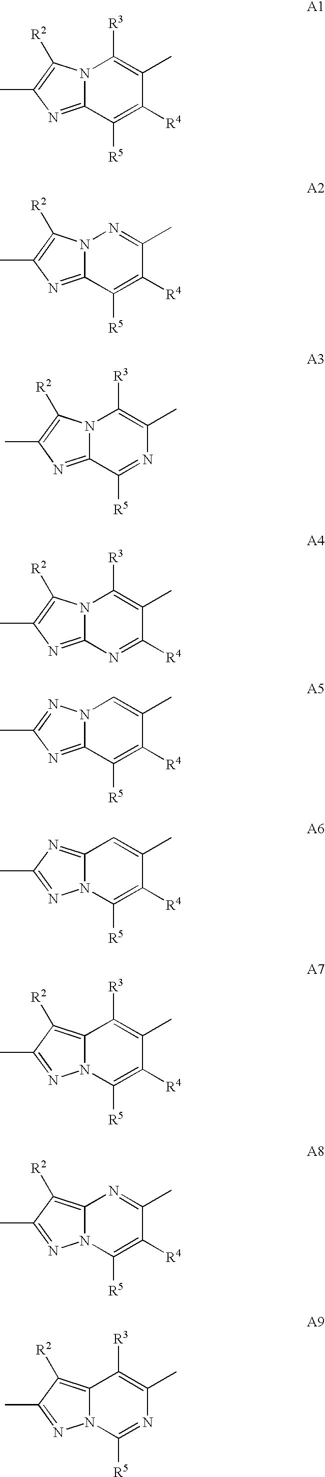 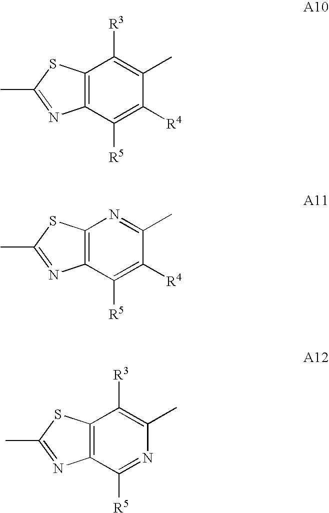
Q is O or S;
L is CR⁹ or N;
R¹ represents —Z—Y—R¹⁰;
Z is —NHCH₂C(R¹¹)R¹²—;
Y is a bond or —CON(R¹³)—;
R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;
R⁴, R⁵, and R⁶ are independently selected from the group consisting of hydrogen, halogen, cyano, nitro, amino, substituted amino, alkoxy, substituted alkoxy, alkyl, and substituted alkyl;
R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and
R^{8a} is selected from the group consisting of alkyl, substituted alkyl, alkoxy, substituted alkoxy, amino, substituted amino, and alkylamino.
R¹⁰ is C₁-C₆-alkylaminocarbonyl, C₁-C₆-alkoxycarbonyl, where each alkyl is independently optionally substituted by one or more halo, hydroxyl or C₁-C₆-alkoxy groups groups, or R¹⁰ is a mono-cyclic heteroaromatic ring having one or more ring heteroatoms selected from the group consisting of oxygen, nitrogen and sulphur, said ring being optionally substituted by one or more halo, hydroxyl, C₁-C₆-alkyl or C₁-C₆-alkoxy groups, where said alkyl and alkoxy are optionally further substituted by one or more halo, hydroxyl or C₁-C₆-alkoxy groups;
R¹¹ and R¹² are independently selected from hydrogen, halo, hydroxy and C₁-C₆-alkyl where said alkyl group is optionally substituted by one or more halo, hydroxyl or C₁-C₆-alkoxy groups; and
R¹³ is hydrogen or C₁-C₆-alkyl.

35. A compound of claim 34 of Formula V, or a stereoisomer, tautomer, or solvate or pharmaceutically acceptable salt thereof, 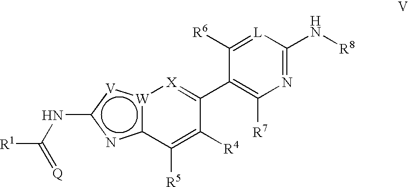
wherein:
Q is O or S;
X is CR³ or N;
W is C or N;
V is CR², O, N, or S;
L is CR⁹ or N;
R¹ represents —Z—Y—R¹⁰;
Z is —NHCH₂C(R¹¹)R¹²—;
Y is a bond or —CON(R¹³)—;
R², R³, R⁷, and R⁹ are independently selected from the group consisting of hydrogen, alkyl, substituted alkyl, alkenyl, substituted alkenyl, alkynyl, substituted alkynyl, alkoxy, substituted alkoxy, aryl, substituted aryl, heteroaryl, substituted heteroaryl, heterocyclyl, cycloalkyl, substituted cycloalkyl, substituted heterocyclyl, aryloxy, substituted aryloxy, heteroaryloxy, substituted heteroaryloxy, heterocyclyloxy, substituted heterocyclyloxy, cycloalkyloxy, substituted cycloalkyloxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, carboxyl, carboxyl ester, (carboxyl ester)amino, (carboxyl ester)oxy, cyano, halo, hydroxy, nitro, SO₃H, substituted sulfonyl, sulfonyloxy, thioacyl, thiol, alkylthio, and substituted alkylthio;
R⁴, R⁵, and R⁶ are independently selected from the group consisting of hydrogen, halogen, cyano, nitro, amino, substituted amino, alkoxy, substituted alkoxy, alkyl, and substituted alkyl;
R⁸ is selected from the group consisting of hydrogen, alkyl, —CO—R^{8a}, substituted alkyl, and a three- to seven-membered ring selected from the group consisting of cycloalkyl, substituted cycloalkyl, heterocyclyl, and substituted heterocyclyl; and
R^{8a} is selected from the group consisting of alkyl, substituted alkyl, alkoxy, substituted alkoxy, amino, substituted amino, and alkylamino.
R¹⁰ is C₁-C₆-alkylaminocarbonyl, C₁-C₆-alkoxycarbonyl, where each alkyl is independently optionally substituted by one or more halo, hydroxyl or C₁-C₆-alkoxy groups groups, or R¹⁰ is a mono-cyclic heteroaromatic ring having one or more ring heteroatoms selected from the group consisting of oxygen, nitrogen and sulphur, said ring being optionally substituted by one or more halo, hydroxyl, C₁-C₆-alkyl or C₁-C₆-alkoxy groups, where said alkyl and alkoxy are optionally further substituted by one or more halo, hydroxyl or C₁-C₆-alkoxy groups;
R¹¹ and R¹² are independently selected from hydrogen, halo, hydroxy and C₁-C₆-alkyl where said alkyl group is optionally substituted by one or more halo, hydroxyl or C₁-C₆-alkoxy groups; and
R¹³ is hydrogen or C₁-C₆-alkyl.

36. A compound according to claim 35 where Q is O.

37. A compound according to claim 35 where X is CH or N.

38. A compound according to claim 35 where W is N.

39. A compound according to claim 35 where V is CH.

40-46. (canceled)

47. A compound according to claim 35 which is selected from the group consisting of Formula Va where R¹ is NHR^{1a}:

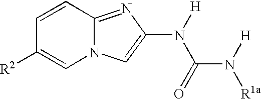

| R² | R^{1a} |
|---|---|
| 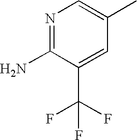 | 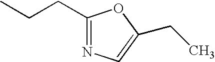 |
| 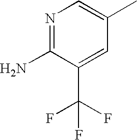 | 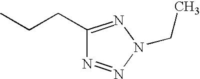 |
| 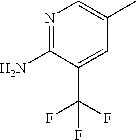 | 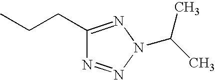 |
| 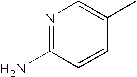 | 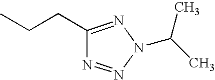 |
| 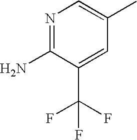 | 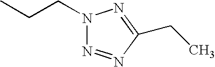 |
| 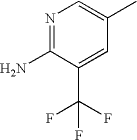 | 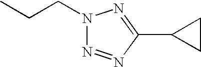 |
| 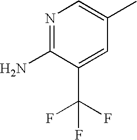 | 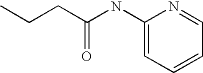 |
| 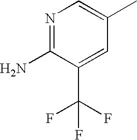 | 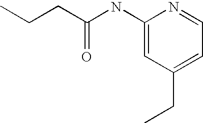 |

48. A pharmaceutical composition comprising a pharmaceutically acceptable carrier and a therapeutically effective amount of a compound of claim 1.

49-56. (canceled)

57. A method for treating a cancer disorder in a patient, comprising administering to the patient a composition comprising an amount of a compound of claim 1 effective to inhibit PI3-K activity.

58-61. (canceled)
